SEQUENCE LISTING (3 sequences)

SEQ ID NO: 1 - RNA, 54 nt, synthetic construct
  source 1..54: mol_type = other RNA
gagaataaactagtattcttctggtccccacagactcagagagaacccgccacc

SEQ ID NO: 2 - RNA, 295 nt, synthetic construct
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 3 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0216313 A1
(43) Pub. Date: Jul. 30, 2026

(54) LIPID PARTICLE COMPOSITIONS AND METHODS OF USE THEREOF

(71) Applicant: PFIZER INC., NEW YORK, NY (US)

(72) Inventors: Bakul Subodh BHATNAGAR, North Reading, MA (US); Ramin DARVARI, Lexington, MA (US); Miguel Angel GARCIA, Midland Park, NJ (US); Pengbo GUO, Tenafly, NJ (US); Shuai SHI, Whippany, NJ (US); Serguei TCHESSALOV, Billerica, MA (US)

(73) Assignee: PFIZER INC., NEW YORK, NY (US)

(21) Appl. No.: 19/141,635

(22) PCT Filed: Dec. 20, 2023
(86) PCT No.: PCT/IB2023/063041

§ 371 Date: Jun. 20, 2025

Related U.S. Application Data
(60) Provisional application No. 63/477,170, filed on Dec. 23, 2022.
(60) Provisional application No. 63/606,205, filed on Dec. 5, 2023.

(51) Int. Cl. A61K 39/145; A61K 9/1272; A61K 9/1277; A61K 9/19; A61K 9/51; A61K 31/7105; A61K 39/00; A61K 47/10; A61K 47/22; A61K 47/26; A61P 31/16; B82Y 5/00; C12N 7/00

(52) U.S. Cl. CPC A61K 39/145; A61K 9/1272; A61K 9/1277; A61K 9/19; A61K 9/5123; A61K 9/5192; A61K 31/7105; A61K 47/10; A61K 47/22; A61K 47/26; A61P 31/16; C12N 7/00; A61K 2039/53; A61K 2039/55555; B82Y 5/00; C12N 2760/16134; C12N 2760/16234

(57) ABSTRACT

The invention relates to compositions and methods for the preparation, manufacture and therapeutic use of ribonucleic acid immunogenic compositions and/or vaccines comprising polynucleotide molecules preferably encoding one or more influenza antigens, such as hemagglutinin antigens, wherein the composition is frozen or lyophilized.

### RELATED APPLICATIONS

This application claims priority to each of the following applications, the disclosure of each of which is hereby incorporated by reference in its entirety: U.S. Provisional Patent Application No. 63/477,170 filed on Dec. 23, 2022; and U.S. Provisional Patent Application No. 63/606,205 filed on Dec. 5, 2023.

### REFERENCE TO SEQUENCE LISTING

The instant application contains a sequence listing which has been submitted electronically in .xml format and is hereby incorporated by reference in its entirety. The .xml file, named “PC072933A_SEQListing_ST26.xml”, was created on Dec. 19, 2023, and is 4 KB in size.

### TECHNICAL FIELD

The disclosure relates to compositions and methods for the preparation, manufacture and therapeutic use of lipid nanoparticle compositions and/or vaccines comprising polynucleotide molecules encoding an antigen, such as hemagglutinin antigens, wherein the compositions are liquid, preferably frozen, or lyophilized.

### BACKGROUND

RNA-based vaccines show great promise to address existing and emerging infectious diseases. However, vaccine RNA molecules may be prone to cleavage by ubiquitous ribonucleases. In addition, RNA alone does not readily cross a cell membrane to enter target cells upon injection. RNA delivery formulations (such as lipid nanoparticles (LNPs)) have been used to help stabilize and protect RNA molecules from degradation by ribonucleases, and enhance efficient cellular uptake and intracellular delivery of the RNA payload. However, maintaining the long-term stability of RNA in the LNP formulations requires storage at subzero temperatures, which may potentially result in detrimental consequences on colloidal stability of the LNPs and the extent of RNA exposure after freeze/thaw.

Accordingly, there remains a need for an effective and thermostable RNA composition. To meet these needs and others, disclosed herein are compositions and methods thereof that may be lyophilizable and thermostable to deliver both mRNA- and replicating RNA-based vaccines effectively, preferably by intramuscular injection.

Vaccines against influenza also have a need for an effective and thermostable RNA composition. Influenza viruses are members of the orthomyxoviridae family, and are classified into three types (A, B, and C), based on antigenic differences between their nucleoprotein (NP) and matrix (M) protein. The genome of influenza A virus includes eight molecules (seven for influenza C virus) of linear, negative polarity, single-stranded RNAs, which encode several polypeptides including: the RNA-directed RNA polymerase proteins (PB2, PB1 and PA) and nucleoprotein (NP), which form the nucleocapsid; the matrix proteins (M1, M2, which is also a surface-exposed protein embedded in the virus membrane); two surface glycoproteins, which project from the lipoprotein envelope: hemagglutinin (HA) and neuraminidase (NA); and nonstructural proteins (NS1 and NS2). Hemagglutinin is the major envelope glycoprotein of influenza A and B viruses, and hemagglutinin-esterase (HE) of influenza C viruses is a protein homologous to HA.

A challenge for therapy and prophylaxis against influenza and other infections using traditional vaccines is the limitation of vaccines in breadth, providing protection only against closely related subtypes. In addition, the length of time required to complete current standard influenza virus vaccine production processes inhibits the rapid development and production of an adapted vaccine in a pandemic situation. There is a need for improved immunogenic compositions against influenza.

### SUMMARY

In one aspect, the present disclosure relates to a composition, such as a liquid, a frozen, or lyophilized composition, comprising tert-butanol (“t-butanol”) and a lipid nanoparticle comprising i) a cationic lipid, ii) a neutral lipid and/or a phospholipid, iii) a steroid, iv) a polymer conjugated lipid. In some embodiments, the lipid nanoparticle encapsulates a ribonucleic acid (RNA) polynucleotide having an open reading frame encoding at least one polypeptide of interest. In some embodiments, the at least one polypeptide of interest comprises an antigen. In some embodiments, the cationic lipid comprises ALC-0315, SM-102, DLinDMA, DLin-MC3-DMA, DLin-KC2-DMA, A9, C12-200, L5, or derivatives and/or combinations thereof. In some embodiments, the neutral lipid and/or phospholipid comprises DSPC, DPPC, DMPC, DOPC, POPC, DOPE, SM, or derivatives and/or combinations thereof. In some embodiments, the steroid comprises cholesterol, alpha-tocopherol, or derivatives and/or combinations thereof. In some embodiments, the polymer conjugated lipid comprises PEG-c-DOMG, PEG-DMG, PEG-DLPE, PEG-DMPE, PEG-DPPC, PEG-DSPE, or derivatives and/or combinations thereof. In some embodiments, the composition further comprises a cryoprotectant. In some embodiments, the cryoprotectant comprises a saccharide, preferable sucrose. In some embodiments, the effective amount of the cryoprotectant is at least about 2% w/v to 30% w/v of the composition, e.g., at least, at most, in between any two of, or exactly 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/v of the composition. In preferred embodiments, the composition comprises at least about 2% w/v to 50% w/v sucrose. In some embodiments, the composition further comprises any one buffer selected from histidine, succinate, citrate, acetate, and succinate. In some embodiments, the composition further comprises histidine.

In some embodiments, the composition is liquid. In some embodiments, the composition is frozen. In some embodiments, the composition is lyophilized. In some embodiments, the composition is lyophilized and reconstituted.

In some embodiments, the RNA comprises a translatable region encoding the antigen and comprises a modified nucleoside comprising 1-methyl-pseudouridine. In some embodiments, the RNA is a self-amplifying RNA comprising a translatable region encoding the antigen. In some embodiments, the RNA comprises an open reading frame encoding at least one influenza virus antigenic polypeptide or an immunogenic fragment thereof. In some embodiments, the composition comprises greater than 70% more encapsulated RNA. In some embodiments, the composition is immunogenic. In some embodiments, the RNA has an integrity of at least 80% prior to lyophilization, as measured by a fragment analyzer. In some embodiments, the RNA has an integrity of at least 80% after lyophilization, as measured by a fragment analyzer.

In some embodiments, the LNP has a polydispersity index of 0.05-0.15 after lyophilization. In some embodiments, the t-butanol is at most 5% w/v of the composition. In some embodiments, the composition comprises about 10 μg/ml saRNA in 15-25 mM histidine, about 10% sucrose, and about 1.5% t-butanol. In some embodiments, the composition comprises about 10 μg/ml saRNA in 10-20 mM histidine, about 10% sucrose, and about 0.75% t-butanol. In some embodiments, the composition comprises about 10 ug/ml saRNA in about 10 mM histidine, about 10% sucrose, and about 0.75% t-butanol. In some embodiments, the composition comprises about 20 μg/ml saRNA in about 20 mM histidine, about 10% sucrose, and about 1.5% t-butanol. In some embodiments, the composition comprises about 20 μg/ml saRNA in about 10 mM histidine, about 10% sucrose, and about 1.5% t-butanol. In some embodiments, the composition comprises about 20 μg/ml saRNA in about 10 mM histidine, about 10% sucrose, and about 1.4% t-butanol. In some embodiments, the composition comprises about 20 μg/ml saRNA in about 10 mM histidine, about 20% sucrose, and about 1.4% t-butanol. In some embodiments, the composition comprises about 20 μg/ml saRNA in about 15 mM histidine, about 10% sucrose, and about 1.4% t-butanol. In some embodiments, the composition comprises about 20 μg/ml saRNA in about 15 mM histidine, about 20% sucrose, and about 1.4% t-butanol.

In some embodiments, a lipid nanoparticle (LNP) encapsulates a ribonucleic acid (RNA) polynucleotide having an open reading frame encoding at least one polypeptide of interest to form an RNA-encapsulating LNP. In some embodiments, the RNA-encapsulating LNP is 50-80 nm in size prior to lyophilization. In some embodiments, the RNA-encapsulating LNP is 80-120 nm in size after lyophilization.

In one aspect, the present disclosure relates to a method for producing a composition comprising an RNA-encapsulating lipid nanoparticle. The method comprises the step of mixing a first composition comprising t-butanol, an ionizable lipid, a polymer-conjugated lipid, phospholipid, and a sterol with a second composition having a pH 4-6 comprising an RNA polynucleotide, aqueous solvent, and buffer, thereby producing a composition comprising RNA-encapsulating lipid nanoparticle and t-butanol. In some embodiments, the method does not comprise a step of exchanging an aqueous phase of the RNA-encapsulating LNPs with a buffer by any one of dialysis, ultrafiltration, and dilution. In some embodiments, the second composition comprises 100-250 mM histidine. In some embodiments, the first and second compositions are not mixed using a flowrate ratio of 1:1-1:3. In some embodiments, the first and second compositions are mixed using a flowrate of 1:7. In some embodiments, the method further comprising a step of mixing the RNA-encapsulating LNP with a cryoprotectant. In some embodiments, the cryoprotectant is sucrose. In some embodiments, the method yields a mixture of RNA-encapsulating LNP having an encapsulation efficiency of at least 70%. In some embodiments, the RNA in the second composition has an integrity of at least 80%, as measured by a fragment analyzer. In some embodiments, the RNA in the RNA-encapsulating LNP has an integrity of at least 80%, as measured by a fragment analyzer. In some embodiments, the RNA-encapsulating LNP has a polydispersity index of 0.05-0.15 after lyophilization. In some embodiments, the t-butanol is at most 5% w/v of the composition.

In one aspect, the present disclosure relates to a method of producing a polypeptide of interest in a cell. The method comprises administering a composition comprising t-butanol and an RNA-encapsulating LNP, as described herein. In some embodiments, the composition is administered to a mammal.

In one aspect, the present disclosure relates to (a) a first lipid nanoparticle; (b) a second lipid nanoparticle; and (c) a cryoprotectant; wherein the first lipid nanoparticle comprises i) a cationic lipid, ii) a neutral lipid and/or a phospholipid, iii) a steroid, and iv) a polymer conjugated lipid; wherein the first lipid nanoparticle encapsulates a ribonucleic acid (RNA) polynucleotide having an open reading frame encoding at least one antigen, preferably an influenza antigen; and the second lipid nanoparticle does not encapsulate a nucleic acid, resulting in increasing the effective concentration of the first lipid nanoparticles without modifying their compositions. In some preferred embodiments, the first LNPs, i.e., the RNA-encapsulating LNPs, are produced by a method described herein in the presence of t-butanol (e.g., any process selected described in, for example, Example 13, Example 15, Example 16, Process A or Process B described below, Example 18, and Example 19).

The effective concentration of the first lipid nanoparticles is a function of availability of the water molecules in the vicinity of their microenvironment. Without wishing to be bound by theory, in some aspects, diluting the formulation with additional formulation buffer solution results in decreased effective concentration of the first lipid nanoparticles, resulting in colloidal instability and increased levels of RNA exposure (i.e. decreased % encapsulation); in some aspects, addition of the second lipid nanoparticles enables an increase in the effective concentration of the first lipid nanoparticles, hence, preventing the detrimental effects on the first lipid nanoparticles as stated above.

In one aspect, the present disclosure relates to an immunogenic composition, such as a liquid, a frozen, or lyophilized immunogenic composition, comprising (a) a first lipid nanoparticle; and an effective amount of a cryoprotectant; wherein the first lipid nanoparticle comprises i) a cationic lipid, ii) a neutral lipid and/or a phospholipid, iii) a steroid, and iv) a polymer conjugated lipid; wherein the first lipid nanoparticle encapsulates a ribonucleic acid (RNA) polynucleotide having an open reading frame encoding at least one polypeptide of interest, wherein the at least one polypeptide of interest comprises an antigen, preferably wherein the antigen is an influenza antigen; wherein the cryoprotectant comprises a saccharide; and wherein the effective amount of the cryoprotectant is at least about 2% w/v to 30% w/v of the composition, e.g., at least, at most, in between any two of, or exactly 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/v of the composition. In some preferred embodiments, the first LNPs, i.e., the RNA-encapsulating LNPs, are produced by a method described herein in the presence of t-butanol (e.g., any process selected described in, for example, Example 13, Example 15, Example 16, Process A or Process B described below, Example 18, and Example 19). In some aspects, the composition further comprises a second lipid nanoparticle, wherein the second lipid nanoparticle does not encapsulate an RNA polynucleotide. In some aspects, inclusion of the second lipid nanoparticle further increases the stability of the composition compared to a composition not comprising the second lipid nanoparticle, when measured under identical conditions.

In some aspects, the total ratio of the RNA-encapsulating lipid nanoparticle and the second lipid nanoparticle is in the range of 1:1 to 1:4999. In some aspects, the total ratio of the RNA-encapsulating lipid nanoparticle and the second lipid nanoparticle is in the range of 1:1 to 1:4999, including 1:4 to 1:4999. See, for example, Table 3.

The ratio is defined as the mass fraction of total lipid components associated with the RNA-encapsulated lipid nanoparticles (reported based on equivalent mass of the RNA payload per unit volume) to the mass fraction of total lipids in the second lipid nanoparticles required to increase the effective concentration of the first lipid nanoparticles to preserve the colloidal stability (particle size and size distribution) and % Encapsulation.

In some aspects, at least 60% of the RNA in the formulation is fully encapsulated in or associated with the RNA-encapsulating lipid nanoparticle. In some aspects, at least 80% of the total RNA in the composition is encapsulated within or associated with the RNA-encapsulating lipid nanoparticle. In some aspects, 0% of the RNA in the formulation is encapsulated in the second nanoparticle.

In some aspects, the second lipid nanoparticle comprises i) a cationic lipid, ii) a neutral lipid and/or a phospholipid, iii) a steroid, and iv) a polymer conjugated lipid. In some aspects, the i) cationic lipid, ii) neutral lipid and/or phospholipid, iii) steroid, and iv) polymer conjugated lipid of the second lipid nanoparticle are the same as the i) cationic lipid, ii) neutral lipid and/or phospholipid, iii) steroid, and iv) polymer conjugated lipid of the first lipid nanoparticle. In some aspects, one or more of the i) cationic lipid, ii) neutral lipid and/or phospholipid, iii) steroid, and/or iv) polymer conjugated lipid of the second lipid nanoparticle are different from the i) cationic lipid, ii) neutral lipid and/or phospholipid, iii) steroid, and/or iv) polymer conjugated lipid of the first lipid nanoparticle. In some aspects, the second lipid nanoparticle is a liposome. In some aspects, the liposome comprises i) a neutral lipid and/or a phospholipid, ii) a steroid, and iii) a polymer conjugated lipid. In some aspects, the liposome further comprises a cationic lipid.

In some aspects, the lipid nanoparticle comprises between 40 and 50 mol percent of the cationic lipid. In some aspects, the composition comprises from 41 to 49 mol percent of the cationic lipid. In some aspects, the composition comprises from 41 to 48 mol percent of the cationic lipid. In some aspects, the composition comprises from 42 to 48 mol percent of the cationic lipid. In some aspects, the composition comprises from 43 to 48 mol percent of the cationic lipid. In some aspects, the composition comprises from 44 to 48 mol percent of the cationic lipid. In some aspects, the composition comprises from 45 to 48 mol percent of the cationic lipid. In some aspects, the composition comprises from 46 to 48 mol percent of the cationic lipid. In some aspects, the composition comprises from 47 to 48 mol percent of the cationic lipid. In some aspects, the composition comprises from 47.2 to 47.8 mol percent of the cationic lipid. In some aspects, the composition comprises about 47.0, 47.1, 47.2, 47.3, 47.4, 47.5, 47.6, 47.7, 47.8, 47.9 or 48.0 mol percent of the cationic lipid. In other aspects, the lipid nanoparticle comprises from 0 to 10 mol percent of the cationic lipid. In certain specific aspects, the lipid nanoparticle comprises at least about, at most about, between any two of, or exactly 0, 1, 2, 3, 4, 5, 6, 7, 8, 9, or 10 mol percent of the cationic lipid.

In some aspects, the phospholipid and/or neutral lipid is present in a concentration ranging from 5 to 15 mol percent. In some aspects, the phospholipid and/or neutral lipid is present in a concentration ranging from 7 to 13 mol percent. In some aspects, the phospholipid and/or neutral lipid is present in a concentration ranging from 9 to 11 mol percent. In some aspects, the phospholipid and/or neutral lipid is present in a concentration of about 9.5, 10 or 10.5 mol percent. In other aspects, the phospholipid and/or neutral lipid is present in a concentration ranging from 40 to 60 mol %. In certain aspects, the phospholipid and/or neutral lipid is present in a concentration of 40 to 60 mol percent. In certain specific aspects, the phospholipid and/or neutral lipid is present in a concentration of about 48, 49, or 50 mol percent. In some aspects, the molar ratio of the cationic lipid to the phospholipid and/or neutral lipid ranges from about 4.1:1.0 to about 4.9:1.0, from about 4.5:1.0 to about 4.8:1.0, or from about 4.7:1.0 to 4.8:1.0.

In some aspects, the steroid is present in a concentration ranging from 32 to 40 mol percent. In some aspects, the steroid is present in a concentration ranging from 39 to 49 mol percent. In some aspects, the steroid is present in a concentration of about 40, 41, 42, 43, 44, 45 or 46 mol percent. In certain aspects, the steroid is present in a concentration of 40 to 60 mol percent. In certain specific aspects, the steroid is present in a concentration of about 48, 49, or 50 mol percent. In some aspects, the molar ratio of cationic lipid to the steroid ranges from 1.0:0.9 to 1.0:1.2. In some aspects, the molar ratio of the total cationic lipid to steroid ranges from 1.0:1.0 to 1.0:1.2.

In some aspects, the lipid nanoparticle comprises i) a cationic lipid having an effective pKa greater than 6.0; ii) from 5 to 15 mol percent of a neutral lipid; iii) from 1 to 15 mol percent of an anionic lipid; iv) from 30 to 45 mol percent of a steroid; v) a polymer conjugated lipid; and vi) a therapeutic agent, or a pharmaceutically acceptable salt or prodrug thereof, encapsulated within or associated with the lipid nanoparticle, wherein the mol percent is determined based on total moles of lipid present in the lipid nanoparticle. In some aspects, the cationic lipid has an effective pKa greater than 6.25. In some aspects, the lipid nanoparticle comprises from 40 to 55 mol percent of the cationic lipid. In some aspects, the lipid nanoparticle comprises: i) from 45 to 55 mol percent of the cationic lipid; ii) from 5 to 10 mol percent of the neutral lipid; iii) from 1 to 5 mol percent of the anionic lipid; and iv) from 32 to 40 mol percent of the steroid.

In some aspects, the composition comprises ALC-0315 (4-hydroxybutyl) azanediyl)bis(hexane-6,1-diyl)bis(2-hexyldecanoate). In some aspects, the composition comprises ALC-0159 (2-[(polyethylene glycol)-2000]-N,N-ditetradecylacetamide). In some aspects, the composition comprises 1,2-Distearoyl-sn-glycero-3-phosphocholine (DSPC). In some aspects, the composition comprises cholesterol.

In some aspects, the composition comprises 0.9-1.85 mg/mL ALC-0315; 0.11-0.24 mg/mL ALC-0159; 0.18-0.41 mg/mL DSPC; and 0.36-0.78 mg/mL cholesterol.

In some aspects, the lipid nanoparticle size is at least 40 nm. In some aspects, the lipid nanoparticle size is at most 180 nm. In some aspects, the second lipid nanoparticle has a size that is 50% less than the first lipid nanoparticle. In some aspects, the second lipid nanoparticle has a size that is 50% greater than the first lipid nanoparticle. In some aspects, the composition has been freeze-thawed at least 2 times. In some aspects, the composition has been freeze-thawed at least 5 times. In some aspects, the mixture of the first lipid and second nanoparticles after freeze-thaw cycling and/or freeze-drying has a size preferable in the range of 20 to 180 nm, more preferably in the range of 30 to 150 nm, and most preferably in the range of 40 to 120 nm.

In some aspects, the cryoprotectant is a saccharide. In some aspects, the cryoprotectant is a disaccharide. In some aspects, the cryoprotectant is sucrose. In some aspects, the cryoprotectant comprises sucrose, and the composition comprises at least about 2% w/v to 30% w/v sucrose, e.g., at least, at most, in between any two of, or exactly 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/v sucrose. In some aspects, the cryoprotectant comprises sucrose, and the composition comprises at least about 10% w/v to 25% w/v sucrose. In some aspects, the cryoprotectant comprises sucrose, and the composition comprises at least about 10.3% w/v to 20.5% w/v sucrose. In some aspects, the concentration of the cryoprotectant is 10 to 600 mg/mL in the composition before freezing and/or freeze-drying.

In some aspects, the composition is frozen. In some aspects, the composition is lyophilized. In some aspects, the composition is lyophilized by spray freeze drying. In some aspects, the composition is lyophilized and reconstituted. In some aspects, the composition is liquid. In some aspects, the composition has a water content of less than about 10% of the total composition. In some aspects, the composition has a water content between about 0.1% and 5% of the total composition.

In some aspects, the composition further comprises a pharmaceutically acceptable buffer. In some aspects, the composition comprises Tris. In some aspects, the composition comprises sucrose. In some aspects, the composition does not further comprise sodium chloride. In some aspects, the composition comprises 10 mM Tris. In some aspects, the composition comprises 300 mM sucrose. In some aspects, the composition has a pH 7.4. In some aspects, the composition has less than or equal to 12.5 EU/mL of bacterial endotoxins.

In some aspects, the composition is configured to be stable for at least about two weeks after storage as a frozen liquid composition, or a lyophilized composition at temperatures less than or equal to refrigerated storage. In some aspects, the composition is configured to be stable for at least 1 month after storage as a frozen liquid composition, or a lyophilized composition at temperatures less than or equal to refrigerated storage. In some aspects, the composition is configured to be stable for at least about two weeks after storage as a frozen liquid composition, or a lyophilized composition at temperatures about 2° C. to 30° C. In some aspects, the composition is configured to be stable for at least about four weeks after storage as a frozen liquid composition, or a lyophilized composition at temperatures about 2° C. to 30° C. In some aspects, the composition is configured to be stable for about 2 weeks to about 1 month, 2 months, 3 months, 4 months, 5 months, 6 months, 1 year, or 2 years after storage as a frozen liquid composition, or a lyophilized composition at temperatures less than or equal to refrigerated storage. In some aspects, the composition is configured to be stable for at least 1 week, preferably for at least 2 weeks, more preferably for at least 3 weeks, most preferably for at least 4 weeks after storage as a liquid at about 25° C. In some aspects, the composition is configured to be stable for at least 1 day, preferably for at least 2 days, more preferably for at least 3 days, most preferably for at least 4 days after storage as a liquid at about 40° C.

In some aspects, the composition has been freeze-thawed at least 2 times. In some aspects, the composition has been freeze-thawed at least 3 times. In some aspects, the composition has been freeze-thawed at least 4 times. In some aspects, the composition has been freeze-thawed at least 5 times. In some aspects, the stability of the composition after being frozen and/or when reconstituted after being freeze-dried is greater as compared to a composition comprising the RNA-encapsulating lipid nanoparticle and not comprising the second lipid nanoparticle, when measured under identical conditions. In some aspects, the stability of the composition after being freeze-thawed at least one time is greater as compared to a composition comprising the RNA-encapsulating lipid nanoparticle and not comprising the second lipid nanoparticle, when measured under identical conditions. In some aspects, the stability of the composition after being freeze-thawed at least two times is greater as compared to a composition comprising the RNA-encapsulating lipid nanoparticle and not comprising the second lipid nanoparticle, when measured under identical conditions. In some aspects, the stability of the composition after being freeze-thawed at least three times is greater as compared to a composition comprising the RNA-encapsulating lipid nanoparticle and not comprising the second lipid nanoparticle, when measured under identical conditions. In some aspects, the stability of the composition after being freeze-thawed at least four times is greater as compared to a composition comprising the RNA-encapsulating lipid nanoparticle and not comprising the second lipid nanoparticle, when measured under identical conditions. In some aspects, the stability of the composition after being freeze-thawed at least five times is greater as compared to a composition comprising the RNA-encapsulating lipid nanoparticle and not comprising the second lipid nanoparticle, when measured under identical conditions.

In some aspects, the concentration of the RNA is in a range from about 10 μg/ml to about 10 mg/ml, preferably in a range from about 0.1 μg/mL to 0.5 mg/mL.

In some aspects, the nucleic acid is RNA and the composition is configured to have at least 60%, 65%, 70%, 75%, 80%, 85%, 90% or 95% of the RNA intact at least about two weeks after storage as a frozen liquid composition, or a lyophilized composition at temperatures less than or equal to refrigerated storage. In some aspects, the composition is configured to have at least 60%, 65%, 70%, 75%, 80%, 85%, 90% or 95% of the RNA intact at least 1 month after storage as a frozen liquid composition, or a lyophilized composition at temperatures less than or equal to refrigerated storage. In some aspects, the composition is configured to have at least 60%, 65%, 70%, 75%, 80%, 85%, 90% or 95% of the RNA intact about 2 weeks to about 1 month, 2 months, 3 months, 4 months, 5 months, 6 months, 1 year, or 2 years after storage as a frozen liquid composition, or a lyophilized composition at temperatures less than or equal to refrigerated storage. In some aspects, the composition is configured to have at least 80% of the RNA intact after about two weeks of storage as a frozen liquid composition, or a lyophilized composition at temperatures less than or equal to refrigerated storage.

In some aspects, the RNA in an RNA-encapsulating LNP prepared in the presence of t-butanol as described herein has an RNA integrity of at least about 50% or greater, preferably of at least about 60% or greater, more preferably of at least about 70% or greater, most preferably of at least about 80% or greater, as compared to a composition comprising the RNA-encapsulating lipid nanoparticle that was not prepared in the presence of t-butanol, when measured under identical conditions. In some aspects, the RNA in an RNA-encapsulating LNP prepared in the presence of t-butanol as described herein has an RNA integrity of at least about 50% greater, preferably of at least about 60% greater, more preferably of at least about 70% greater, most preferably of at least about 80% greater, as compared to a composition comprising an RNA-encapsulating LNP that was not prepared in the presence of t-butanol, when measured under identical conditions. In some aspects, the identical conditions are after storage as a frozen liquid composition, or a lyophilized composition at temperatures less than or equal to refrigerated storage.

In some aspects, the RNA has an RNA integrity of at least about 50% or greater, preferably of at least about 60% or greater, more preferably of at least about 70% or greater, most preferably of at least about 80% or greater, as compared to a composition comprising the RNA-encapsulating lipid nanoparticle and not comprising the second lipid nanoparticle, when measured under identical conditions.

In some aspects, the RNA in an RNA-encapsulating LNP prepared in the presence of t-butanol as described herein has an RNA integrity of at least about 10% greater, 20% greater, 30% greater, 40% greater, 50% greater, preferably of at least about 60% greater, more preferably of at least about 70% greater, most preferably of at least about 80% greater, as compared to a composition comprising RNA-encapsulating LNPs that were not prepared in the presence of t-butanol, when measured under identical conditions. In some aspects, the RNA has an RNA integrity of at least about 50% greater, preferably of at least about 60% greater, more preferably of at least about 70% greater, most preferably of at least about 80% greater, as compared to a composition comprising the first lipid nanoparticle and not comprising the second lipid nanoparticle, when measured under identical conditions. In some aspects, the identical conditions are after storage as a frozen liquid composition, or a lyophilized composition at temperatures less than or equal to refrigerated storage.

In some aspects, the composition comprises less than about 20% less free RNA, preferably less than about 15% less free RNA, more preferably less than about 10% less free RNA %, as compared to a composition comprising the first lipid nanoparticle and not comprising the second lipid nanoparticle, when measured under identical conditions.

In some aspects, the composition comprising RNA-encapsulating LNPs prepared in the presence of t-butanol comprises greater than 60% more encapsulated RNA, preferably greater than 70% more encapsulated RNA, more preferably greater than 80% more encapsulated RNA, and most preferably greater than 90% more encapsulated RNA, as compared to a composition comprising the first lipid nanoparticle and not comprising the second lipid nanoparticle, when measured under identical conditions. In some aspects, the composition comprises greater than 60% encapsulated RNA, preferably greater than 70% encapsulated RNA, more preferably greater than 80% encapsulated RNA, and most preferably greater than 90% encapsulated RNA, as compared to a composition comprising the first lipid nanoparticle and not comprising the second lipid nanoparticle, when measured under identical conditions.

In some aspects, the composition comprises greater than 60% more encapsulated RNA, preferably greater than 70% more encapsulated RNA, more preferably greater than 80% more encapsulated RNA, and most preferably greater than 90% more encapsulated RNA, as compared to a composition comprising the first lipid nanoparticle and not comprising the second lipid nanoparticle, when measured under identical conditions. In some aspects, the composition comprises greater than 60% encapsulated RNA, preferably greater than 70% encapsulated RNA, more preferably greater than 80% encapsulated RNA, and most preferably greater than 90% encapsulated RNA, as compared to a composition comprising the first lipid nanoparticle and not comprising the second lipid nanoparticle, when measured under identical conditions.

In some aspects, the integrity of the RNA decreases less than about 30%, preferably less than about 20%, more preferably less than about 10%, as compared to a composition comprising lipid nanoparticles that were not prepared in the presence of t-butanol, when measured under identical conditions. In some aspects, the amount of free RNA does not increase by more than 10%, preferably by not more than 5%, as compared to a composition comprising lipid nanoparticles that were not prepared in the presence of t-butanol, when measured under identical conditions. In some aspects, the integrity of the RNA decreases less than about 30%, preferably less than about 20%, more preferably less than about 10%, as compared to a composition comprising the first lipid nanoparticle and not comprising the second lipid nanoparticle, when measured under identical conditions. In some aspects, the amount of free RNA does not increase by more than 10%, preferably by not more than 5%, as compared to a composition comprising the first lipid nanoparticle and not comprising the second lipid nanoparticle, when measured under identical conditions.

In some aspects, the Z-average size of the lipid-based carriers encapsulating the RNA does not increase by more than 20%, preferably by not more than 10%, as compared to a composition comprising lipid nanoparticles that were not prepared in the presence of t-butanol, when measured under identical conditions. In some aspects, the turbidity of the composition does not increase by more than 20%, preferably by not more than 10%, as compared to a composition comprising lipid nanoparticles that were not prepared in the presence of t-butanol. In some aspects, the Z-average size of the lipid-based carriers encapsulating the RNA does not increase by more than 20%, preferably by not more than 10%, as compared to a composition comprising the first lipid nanoparticle and not comprising the second lipid nanoparticle, when measured under identical conditions. In some aspects, the turbidity of the composition does not increase by more than 20%, preferably by not more than 10%, as compared to a composition comprising the first lipid nanoparticle and not comprising the second lipid nanoparticle, when measured under identical conditions.

In some aspects, the pH and/or the osmolality does not increase or decrease by more than 20%, preferably by not more than 10%, as compared to a composition comprising lipid nanoparticles that were not prepared in the presence of t-butanol. In some aspects, the pH and/or the osmolality does not increase or decrease by more than 20%, preferably by not more than 10%, as compared to a composition comprising the first lipid nanoparticle and not comprising the second lipid nanoparticle, when measured under identical conditions.

In some aspects, the potency of the composition decreases less than about 30%, preferably less than about 20%, more preferably less than about 10%, as compared to a composition comprising lipid nanoparticles that were not prepared in the presence of t-butanol. In some aspects, the potency of the composition decreases less than about 30%, preferably less than about 20%, more preferably less than about 10%, as compared to a composition comprising the first lipid nanoparticle and not comprising the second lipid nanoparticle, when measured under identical conditions.

In some aspects, the RNA has been purified by at least one purification step, and wherein the first lipid nanoparticle has been purified by at least one purification step, preferably by at least one step of tangential flow filtration (TFF) and/or at least one step of clarification and/or at least one step of filtration. In some aspects, the RNA is a purified RNA, preferably an RP-HPLC purified RNA and/or a tangential flow filtration (TFF) purified RNA.

In some aspects, the composition comprises an effective amount of RNA to produce a polypeptide of interest in a cell. In some aspects, the RNA comprises an open reading frame, and the open reading frame is codon-optimized. In some aspects, the RNA comprises an open reading frame encoding at least one influenza virus antigenic polypeptide or an immunogenic fragment thereof.

In some aspects, the antigen is influenza hemagglutinin 1 (HA1), hemagglutinin 2 (HA2), an immunogenic fragment of HA1 or HA2, or a combination of any two or more of the foregoing. In some aspects, the RNA encodes at least two antigenic polypeptides or immunogenic fragments thereof, wherein a first antigen is HA1, HA2, or a combination of HA1 and HA2, and wherein a second antigen is neuraminidase (NA), nucleoprotein (NP), matrix protein 1 (M1), matrix protein 2 (M2), non-structural protein 1 (NS1) and non-structural protein 2 (NS2). In some aspects, the RNA encodes at least two antigenic polypeptides or immunogenic fragments thereof, wherein a first antigen is HA1, HA2, or a combination of HA1 and HA2, and wherein a second antigen is neuraminidase (NA).

In some aspects, the antigen is a polypeptide or an immunogenic fragment thereof from an arenavirus; an astrovirus; a bunyavirus; a calicivirus; a coronavirus; a filovirus; a flavivirus; a hepadnavirus; a hepevirus; an orthomyxovirus; a paramyxovirus; a picornavirus; a reovirus; a retrovirus; a rhabdovirus; a togavirus; or a combination of any two or more of the foregoing.

In some aspects, the antigen is a polypeptide or an immunogenic fragment thereof from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 01 57:H7, 01 1 1 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowled, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B1 9, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, Yersinia pseudotuberculosis, or a combination of any two or more of the foregoing.

In some aspects, the composition comprises a) at least one ribonucleic acid (RNA) polynucleotide having an open reading frame encoding influenza hemagglutinin 1 (HA1) or an immunogenic fragment thereof; b) at least one ribonucleic acid (RNA) polynucleotide having an open reading frame encoding hemagglutinin 2 (HA2) or an immunogenic fragment thereof; c) at least one ribonucleic acid (RNA) polynucleotide having an open reading frame encoding at least one antigenic polypeptide, wherein an antigen is neuraminidase (NA), nucleoprotein (NP), matrix protein 1 (M1), matrix protein 2 (M2), non-structural protein 1 (NS1) and non-structural protein 2 (NS2), or an immunogenic fragment thereof; and d) at least one ribonucleic acid (RNA) polynucleotide having an open reading frame encoding at least one antigenic polypeptide, wherein an antigen is neuraminidase (NA), nucleoprotein (NP), matrix protein 1 (M1), matrix protein 2 (M2), non-structural protein 1 (NS1) and non-structural protein 2 (NS2), or an immunogenic fragment thereof.

In some aspects, the composition further comprises a cationic lipid. In some aspects, the composition comprises a) a lipid nanoparticle encompassing at least one ribonucleic acid (RNA) polynucleotide having an open reading frame encoding influenza hemagglutinin 1 (HA1), or an immunogenic fragment thereof; b) a lipid nanoparticle encompassing at least one ribonucleic acid (RNA) polynucleotide having an open reading frame encoding hemagglutinin 2 (HA2), or an immunogenic fragment thereof; c) a lipid nanoparticle encompassing at least one ribonucleic acid (RNA) polynucleotide having an open reading frame encoding at least one antigenic polypeptide, wherein an antigen is neuraminidase (NA), nucleoprotein (NP), matrix protein 1 (M1), matrix protein 2 (M2), non-structural protein 1 (NS1) and non-structural protein 2 (NS2), or an immunogenic fragment thereof; and d) a lipid nanoparticle encompassing at least one ribonucleic acid (RNA) polynucleotide having an open reading frame encoding at least one antigenic polypeptide, wherein an antigen is neuraminidase (NA), nucleoprotein (NP), matrix protein 1 (M1), matrix protein 2 (M2), non-structural protein 1 (NS1) and non-structural protein 2 (NS2), or an immunogenic fragment thereof, wherein the composition further comprises t-butanol, preferably in residual amounts, e.g., less than 0.5% w/v.

In some aspects, the RNA further comprises a modified nucleotide. In some aspects, the RNA comprises a modified nucleotide comprising N1-Methylpseudourodine-5′-triphosphate (m1ψTP). In some aspects, the RNA comprises a translatable region encoding the antigen and comprises a modified nucleoside comprising 1-methyl-pseudouridine.

In some aspects, the RNA further comprises a 5′ cap analog. In some aspects, the RNA further comprises a 5′ cap analog, and the 5′ cap analog comprises m₂⁷·3′^{O}Gppp (m₁²′^{O}) ApG. In some aspects, the RNA polynucleotide comprises a 5′ cap, 5′ UTR, 3′ UTR, histone stem-loop, and poly-A tail.

Disclosed herein, in some aspects, is a method of producing a polypeptide of interest in a cell, the method comprising administering a composition described herein, wherein the composition produces an increased amount of the polypeptide, as compared to a composition comprising lipid nanoparticles that were not prepared in the presence of t-butanol. Also disclosed herein, in some aspects, is a method of producing a polypeptide of interest in a cell, the method comprising administering a composition described herein, wherein the composition produces an increased amount of the polypeptide, as compared to a composition comprising the first lipid nanoparticle and not comprising the second lipid nanoparticle, when measured under identical conditions.

Disclosed herein, in some aspects, is a method of producing a polypeptide of interest in a cell, comprising administering a composition described herein, wherein the composition produces an increased amount of the polypeptide, as compared to a composition comprising lipid nanoparticles that were not prepared in the presence of t-butanol. Also disclosed herein, in some aspects, is a method of producing a polypeptide of interest in a cell, comprising administering a composition according to any one of claims 91-151, wherein the composition produces an increased amount of the polypeptide, as compared to a composition comprising the first lipid nanoparticle and not comprising the effective amount of the cryoprotectant, when measured under identical conditions.

In some aspects of the methods, the composition is administered to a mammal. In some aspects of the method, the composition is administered to a human. In some aspects of the method, the composition is administered to a mammal at risk of having influenza.

Also disclosed herein, in some aspects, is a method of increasing the stability of a composition comprising a first lipid nanoparticle and a second lipid nanoparticle, the first lipid nanoparticle comprising i) a cationic lipid, ii) a neutral lipid and/or phospholipid, iii) a steroid, iv) a polymer conjugated lipid, and v) a ribonucleic acid (RNA) polynucleotide encapsulated in the first lipid nanoparticle, the second lipid nanoparticle lacking a ribonucleic acid (RNA) polynucleotide encapsulated in the second lipid nanoparticle, and the method comprising purifying the composition to remove a first portion of a plurality of the second lipid nanoparticle from the composition before freezing and/or freeze-drying, wherein a second portion of the plurality of the second lipid nanoparticle remains in the composition, wherein the composition further comprises t-butanol, preferably a residual amount, e.g., less than 5% w/v.

Also disclosed herein, in some aspects, is a method of increasing the stability of a composition comprising a first lipid nanoparticle, the first lipid nanoparticle comprising i) a cationic lipid, ii) a neutral lipid and/or phospholipid, iii) a steroid, iv) a polymer conjugated lipid, and v) a ribonucleic acid (RNA) polynucleotide encapsulated in the first lipid nanoparticle, the method comprising contacting the composition with a second lipid nanoparticle, wherein the second lipid nanoparticle does not encapsulate a ribonucleic acid (RNA) polynucleotide, wherein the composition further comprises t-butanol, preferably in a residual amount, e.g., less than 5% w/v.

Also disclosed herein, in some aspects, is a method of increasing the stability of a composition comprising a first lipid nanoparticle, the first lipid nanoparticle comprising i) a cationic lipid, ii) a neutral lipid and/or phospholipid, iii) a steroid, iv) a polymer conjugated lipid, and v) a ribonucleic acid (RNA) polynucleotide encapsulated in the first lipid nanoparticle, the method comprising contacting the composition with an effective amount of a cryoprotectant, wherein the cryoprotectant comprises a saccharide, and wherein the effective amount of the cryoprotectant is at least about 2% w/v to 30% w/v of the composition, wherein the composition further comprises t-butanol, preferably in a residual amount, e.g., less than 5% w/v. In some aspects of the method, the composition further comprises a second lipid nanoparticle, and wherein the second lipid nanoparticle does not encapsulate an RNA polynucleotide.

In some aspects of the methods, the second lipid nanoparticle comprises i) a cationic lipid, ii) a neutral lipid and/or phospholipid, iii) a steroid, and iv) a polymer conjugated lipid. In some aspects of the methods, the i) cationic lipid, ii) neutral lipid and/or phospholipid, iii) steroid, and iv) polymer conjugated lipid of the second lipid nanoparticle are the same as the i) cationic lipid, ii) neutral lipid and/or phospholipid, iii) steroid, and iv) polymer conjugated lipid of the first lipid nanoparticle. In some aspects of the methods, one or more of the i) cationic lipid, ii) neutral lipid and/or phospholipid, iii) steroid, and/or iv) polymer conjugated lipid of the second lipid nanoparticle are different from the i) cationic lipid, ii) neutral lipid and/or phospholipid, iii) steroid, and/or iv) polymer conjugated lipid of the first lipid nanoparticle. In some aspects of the methods, the second lipid nanoparticle comprises a liposome. In some aspects of the method, the liposome comprises i) a phospholipid and/or a neutral lipid, ii) a steroid, and/or iii) a polymer conjugated lipid. In some aspects of the method, the liposome further comprises a cationic lipid.

In some aspects of the methods, the cryoprotectant comprises a disaccharide. In some aspects of the methods, the cryoprotectant comprises sucrose. In some aspects of the methods, the effective amount of the cryoprotectant is at least about 2% w/v to 30% w/v sucrose, e.g., at least, at most, in between any two of, or exactly 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/v of the composition. In some aspects, of the methods, the effective amount of the cryoprotectant is at least about 15% w/v to 25% w/v of the composition. In some aspects of the methods, the effective amount of the cryoprotectant is at least about 10% w/v to 25% w/v of the composition. In some aspects of the methods, the effective amount of the cryoprotectant is at least about 10.3% w/v to 20.5% w/v of the composition. In some aspects of the methods, the concentration of the cryoprotectant is 5 to 600 mg/mL in the composition before freezing and/or freeze-drying.

In some aspects of the methods, the stability increase comprises the storage stability of the composition when frozen and/or when freeze-dried. In some aspects of the methods, the stability increase comprises the stability of the composition when thawed after being frozen and/or when reconstituted after being freeze-dried. In some aspects of the methods, the stability increase comprises the stability of the composition when the composition has been freeze-thawed at least 1 time. In some aspects of the methods, the stability increase comprises the stability of the composition when the composition has been freeze-thawed at least 2 times. In some aspects of the methods, the stability increase comprises the stability of the composition when the composition has been freeze-thawed at least 3 times. In some aspects of the methods, the stability increase comprises the stability of the composition when the composition has been freeze-thawed at least 4 times. In some aspects of the methods, the stability increase comprises the stability of the composition when the composition has been freeze-thawed at least 5 times.

In some aspects of the methods, contacting the composition with a second lipid nanoparticle forms an immunogenic composition disclosed herein. In some aspects of the methods, purifying the composition to remove a first portion of a plurality of the second lipid nanoparticle from the composition before freezing and/or freeze-drying forms an immunogenic composition disclosed herein. In some aspects of the methods, contacting the composition with an effective amount of the cryoprotectant forms an immunogenic composition disclosed herein. In some aspects of the methods, contacting the composition with an effective amount of t-butanol forms an immunogenic composition disclosed herein.

Other objects, features and advantages of the present disclosure will become apparent from the following figures, detailed description, and examples. It should be understood, however, that the figures, detailed description, and examples, while indicating specific aspects of the invention, are given by way of illustration only and are not meant to be limiting. Aspects described herein are understood to be aspects of the disclosure that are applicable to other aspects of the disclosure. Any aspect discussed with respect to one aspect of the disclosure applies to other aspects of the invention as well and vice versa. Additionally, it is contemplated that changes and modifications within the spirit and scope of the disclosure will become apparent to those skilled in the art from this detailed description. In further aspects, features from specific aspects may be combined with features from other aspects and/or implemented with respect to any method or composition of the invention, and vice versa. For example, features from one aspect may be combined with features from any of the other aspects. In further aspects, additional features may be added to the specific aspects described herein. Furthermore, compositions and systems of the disclosure can be used to achieve methods of the disclosure.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIG. 1 illustrates the effect of up to five freeze-thaw cycles (1 FT, 2 FT, 3 FT, 4 FT, 5 FT) on LNP particle size (Z-average) of flu mRNA formulations (0.001 to 0.5 mg/ml mRNA) containing 10.3% w/v Sucrose (F1-F4) in the absence of blank lipid nanoparticles.

FIG. 2 illustrates the effect of up to five freeze-thaw cycles (1 FT, 2 FT, 3 FT, 4 FT, 5 FT) on LNP polydispersity index (PDI) of flu mRNA formulations (0.001 to 0.5 mg/ml mRNA) containing 10.3% w/v Sucrose (F1-F4) in the absence of blank lipid nanoparticles.

FIG. 3 illustrates the effect of up to five freeze-thaw cycles (1 FT, 2 FT, 3 FT, 4 FT, 5 FT) on mRNA encapsulation efficiency (Encapsulation %) of flu mRNA LNP formulations (0.001 to 0.5 mg/ml mRNA) containing 10.3% w/v Sucrose (F1-F4) in the absence of blank lipid nanoparticles.

FIG. 4 illustrates the effect of up to five freeze-thaw cycles (1 FT, 2 FT, 3 FT, 4 FT, 5 FT) on LNP particle size (Z-average) of flu mRNA formulations (0.001 to 0.5 mg/ml mRNA) containing 20.5% w/v Sucrose (F10-F13) in the absence of blank lipid nanoparticles.

FIG. 5 illustrates the effect of up to five freeze-thaw cycles (1 FT, 2 FT, 3 FT, 4 FT, 5 FT) on LNP PDI of flu mRNA formulations (0.001 to 0.5 mg/ml mRNA) containing 20.5% w/v Sucrose (F10-F13) in the absence of blank lipid nanoparticles.

FIG. 6 illustrates the effect of up to five freeze-thaw cycles (1 FT, 2 FT, 3 FT, 4 FT, 5 FT) on mRNA encapsulation efficiency (Encapsulation %) of flu mRNA LNP formulations (0.001 to 0.5 mg/ml mRNA) containing 20.5% w/v Sucrose (F10-F13) in the absence of blank lipid nanoparticles.

FIG. 7 illustrates the effect of up to five freeze-thaw cycles (1 FT, 2 FT, 3 FT, 4 FT, 5 FT) on LNP particle size (Z-average) of flu mRNA formulations (0.001 to 0.1 mg/mL mRNA) containing 10.3% w/v Sucrose (F5-F9) in the presence of blank lipid nanoparticles.

FIG. 8 illustrates the effect of up to five freeze-thaw cycles (1 FT, 2 FT, 3 FT, 4 FT, 5 FT) on LNP PDI of flu mRNA formulations (0.001 to 0.1 mg/ml mRNA) containing 10.3% w/v Sucrose (F5-F9) in the presence of blank lipid nanoparticles.

FIG. 9 illustrates the effect of up to five freeze-thaw cycles (1 FT, 2 FT, 3 FT, 4 FT, 5 FT) on mRNA encapsulation efficiency (Encapsulation %) of flu mRNA LNP formulations (0.001 to 0.1 mg/ml mRNA) containing 10.3% w/v Sucrose (F5-F9) in the presence of blank lipid nanoparticles.

FIG. 10 illustrates the effect of up to five freeze-thaw cycles (1 FT, 2 FT, 3 FT, 4 FT, 5 FT) on LNP particle size (Z-average) of flu mRNA formulations (0.001 to 0.01 mg/mL mRNA) containing 20.5% w/v Sucrose (F14-F15) in the presence of blank lipid nanoparticles.

FIG. 11 illustrates the effect of up to five freeze-thaw cycles (1 FT, 2 FT, 3 FT, 4 FT, 5 FT) on LNP PDI of flu mRNA formulations (0.001 to 0.01 mg/mL mRNA) containing 20.5% w/v Sucrose (F14-F15) in the presence of blank lipid nanoparticles.

FIG. 12 illustrates the effect of up to five freeze-thaw cycles (1 FT, 2 FT, 3 FT, 4 FT, 5 FT) on mRNA encapsulation efficiency (Encapsulation %) of flu mRNA LNP formulations (0.001 to 0.01 mg/ml mRNA) containing 20.5% w/v Sucrose (F14-F15) in the presence of blank lipid nanoparticles.

FIG. 13A-E illustrates VSVg saRNA Lyo Drug Product Stability Data: (A) Fragment analyzer data, % Integrity by FA over Time (months); (B) % Expression over Time (months); (C) Encapsulation Efficiency (EE) in % Encapsulation over Time (months); (D) Size over Time (months); and (E) polydispersity index (PDI) over Time (months).

FIG. 14 illustrates the aspect of increasing the effective concentration of the first lipid nanoparticle, which encapsulates RNA, in the presence of the second lipid nanoparticle, i.e., blank LNP, to preserve the colloidal stability and percent encapsulation of the first lipid nanoparticle.

FIG. 15A-E illustrate results from HA saRNA LNP lyophilization. HA saRNA LNP at 10 ug/ml in the respective matrices were lyophilized. The 2 mL vials were filled at 0.35 mL and reconstituted with water and saline at 0.33 mL. Matrix “10T10S” refers to 10 mM Tris in 10% sucrose. Matrix “10T20S” refers to 10 mM Tris in 20% sucrose. Both the lyophilized material and pre-lyophilized material have an additional freeze-thaw. FIG. 15(A) Encapsulation Efficiency (EE) in % Encapsulation over Time (months); (B) Legend for pre-Lyo and Post-Lyo data; (C) Fragment analyzer data, % Integrity by FA over Time (months); (D) Size over Time (months); and (E) polydispersity index (PDI) over Time (months).

FIG. 16 illustrates the effect of lyophilization (with and without annealing during freezing) and reconstitution (in water or saline) on LNP particle size (Z-average) of flu mRNA formulations (0.001 to 0.5 mg/mL mRNA) containing 10.3% w/v Sucrose (F1-F4) in the absence of blank lipid nanoparticles.

FIG. 17 illustrates the effect of lyophilization (with and without annealing during freezing) and reconstitution (in water or saline) on LNP polydispersity index (PDI) of flu mRNA formulations (0.001 to 0.5 mg/ml mRNA) containing 10.3% w/v Sucrose (F1-F4) in the absence of blank lipid nanoparticles.

FIG. 18 illustrates the effect of lyophilization (with and without annealing during freezing) and reconstitution (in water or saline) on mRNA encapsulation efficiency (Encapsulation %) of flu mRNA formulations (0.001 to 0.5 mg/mL mRNA) containing 10.3% w/v Sucrose (F1-F4) in the absence of blank lipid nanoparticles.

FIG. 19 illustrates the effect of lyophilization (with and without annealing during freezing) and reconstitution (in water or saline) on LNP particle size (Z-average) of flu mRNA formulations (0.001 to 0.5 mg/ml mRNA) containing 20.5% w/v Sucrose (F10-F13) in the absence of blank lipid nanoparticles.

FIG. 20 illustrates the effect of lyophilization (with and without annealing during freezing) and reconstitution (in water or saline) on LNP polydispersity index (PDI) of flu mRNA formulations (0.001 to 0.5 mg/ml mRNA) containing 20.5% w/v Sucrose (F10-F13) in the absence of blank lipid nanoparticles.

FIG. 21 illustrates the effect of lyophilization (with and without annealing during freezing) and reconstitution (in water or saline) on mRNA encapsulation efficiency (Encapsulation %) of flu mRNA formulations (0.001 to 0.5 mg/ml mRNA) containing 20.5% w/v Sucrose (F10-F13) in the absence of blank lipid nanoparticles.

FIG. 22 illustrates the effect of lyophilization (with and without annealing during freezing) and reconstitution (in water or saline) on LNP particle size (Z-average) of flu mRNA formulations (0.001 to 0.1 mg/mL mRNA) containing 10.3% w/v Sucrose (F5-F9) in the presence of blank lipid nanoparticles.

FIG. 23 illustrates the effect of lyophilization (with and without annealing during freezing) and reconstitution (in water or saline) on LNP polydispersity index (PDI) of flu mRNA formulations (0.001 to 0.1 mg/ml mRNA) containing 10.3% w/v Sucrose (F5-F9) in the presence of blank lipid nanoparticles.

FIG. 24 illustrates the effect of lyophilization (with and without annealing during freezing) and reconstitution (in water or saline) on mRNA encapsulation efficiency (Encapsulation %) of flu mRNA formulations (0.001 to 0.1 mg/ml mRNA) containing 10.3% w/v Sucrose (F5-F9) in the presence of blank lipid nanoparticles.

FIG. 25 illustrates the effect of lyophilization (with and without annealing during freezing) and reconstitution (in water or saline) on LNP particle size (Z-average) of flu mRNA formulations (0.001 to 0.01 mg/ml mRNA) containing 20.5% w/v Sucrose (F14-F15) in the presence of blank lipid nanoparticles.

FIG. 26 illustrates the effect of lyophilization (with and without annealing during freezing) and reconstitution (in water or saline) on LNP polydispersity index (PDI) of flu mRNA formulations (0.001 to 0.01 mg/mL mRNA) containing 20.5% w/v Sucrose (F14-F15) in the presence of blank lipid nanoparticles.

FIG. 27 illustrates the effect of lyophilization (with and without annealing during freezing) and reconstitution (in water or saline) on mRNA encapsulation efficiency (Encapsulation %) of flu mRNA formulations (0.001 to 0.01 mg/ml mRNA) containing 20.5% w/v Sucrose (F14-F15) in the absence of blank lipid nanoparticles.

FIG. 28 illustrates the effect of up to four freeze-thaw cycles (1 FT, 2 FT, 3 FT, 4 FT) on LNP particle size (Z-average) of flu mRNA LNP formulations (0.01 mg/ml mRNA) in the presence of blank lipid nanoparticles or increasing sucrose concentration (15.4% or 20.5% w/v).

FIG. 29 illustrates the effect of up to four freeze-thaw cycles (1 FT, 2 FT, 3 FT, 4 FT) on LNP particle size (Z-average) of flu mRNA LNP formulations (0.005 mg/ml mRNA) in the presence of blank lipid nanoparticles or increasing sucrose concentration (15.4% or 20.5% w/v).

FIG. 30 illustrates the effect of up to four freeze-thaw cycles (1 FT, 2 FT, 3 FT, 4 FT) on LNP polydispersity index (PDI) of flu mRNA LNP formulations (0.01 mg/ml mRNA) in the presence of blank lipid nanoparticles or increasing sucrose concentration (15.4% or 20.5% w/v).

FIG. 31 illustrates the effect of up to four freeze-thaw cycles (1 FT, 2 FT, 3 FT, 4 FT) on LNP polydispersity index (PDI) of flu mRNA LNP formulations (0.005 mg/ml mRNA) in the presence of blank lipid nanoparticles or increasing sucrose concentration (15.4% or 20.5% w/v).

FIG. 32 illustrates the effect of up to four freeze-thaw cycles (1 FT, 2 FT, 3 FT, 4 FT) on mRNA encapsulation efficiency (Encapsulation %) of flu mRNA LNP formulations (0.01 mg/mL mRNA) in the presence of blank lipid nanoparticles or increasing sucrose concentration (15.4% or 20.5% w/v).

FIG. 33 illustrates the effect of up to four freeze-thaw cycles (1 FT, 2 FT, 3 FT, 4 FT) on mRNA encapsulation efficiency (Encapsulation %) of flu mRNA LNP formulations (0.005 mg/mL mRNA) in the presence of blank lipid nanoparticles or increasing sucrose concentration (15.4% or 20.5% w/V).

FIG. 34 illustrates the effect of up to five freeze-thaw cycles (1 FT, 3 FT, 5 FT) on LNP particle size and LNP polydispersity index (PDI) of flu mRNA LNP formulations (3, 0.1, 0.01, and 0.001 mg/ml mRNA encapsulated by ALC-0159, DSPC, cholesterol, and MC3) in the presence of blank lipid nanoparticles comprised of ALC-0159, DSPC, cholesterol, and MC3.

FIG. 35 illustrates the effect of up to five freeze-thaw cycles (1 FT, 3 FT, 5 FT) mRNA encapsulation efficiency (Encapsulation %) of flu mRNA LNP formulations (3, 0.1, 0.01, and 0.001 mg/ml mRNA encapsulated by ALC-0159, DSPC, cholesterol, and MC3) in the presence of blank lipid nanoparticles comprised of ALC-0159, DSPC, cholesterol, and MC3.

FIG. 36 illustrates the effect of up to five freeze-thaw cycles (1 FT, 3 FT, 5 FT) on LNP particle size and LNP polydispersity index (PDI) of flu mRNA LNP formulations (3, 0.1, 0.01, and 0.001 mg/mL mRNA encapsulated by ALC-0159, DSPC, cholesterol, and A9) in the presence of blank lipid nanoparticles comprised of ALC-0159, DSPC, cholesterol, and A9.

FIG. 37 illustrates the effect of up to five freeze-thaw cycles (1 FT, 3 FT, 5 FT) mRNA encapsulation efficiency (Encapsulation %) of flu mRNA LNP formulations (3, 0.1, 0.01, and 0.001 mg/ml mRNA encapsulated by ALC-0159, DSPC, cholesterol, and A9) in the presence of blank lipid nanoparticles comprised of ALC-0159, DSPC, cholesterol, and A9.

FIG. 38 illustrates the effect of up to five freeze-thaw cycles (1 FT, 3 FT, 5 FT) on LNP particle size and LNP polydispersity index (PDI) of flu mRNA LNP formulations (3, 0.1, 0.01, and 0.001 mg/mL mRNA encapsulated by ALC-0159, DSPC, cholesterol, and ALC-0315) in the presence of liposomes comprised of ALC-0159, DSPC, and cholesterol.

FIG. 39 illustrates the effect of up to five freeze-thaw cycles (1 FT, 3 FT, 5 FT) mRNA encapsulation efficiency (Encapsulation %) of flu mRNA LNP formulations (3, 0.1, 0.01, and 0.001 mg/ml mRNA encapsulated by ALC-0159, DSPC, cholesterol, and ALC-0315) in the presence of liposomes comprised of ALC-0159, DSPC, and cholesterol.

FIG. 40 illustrates the effect of up to five freeze-thaw cycles (1 FT, 3 FT, 5 FT) on LNP particle size and LNP polydispersity index (PDI) of flu mRNA LNP formulations (3, 0.1, 0.01, and 0.001 mg/ml mRNA encapsulated by ALC-0159, DSPC, cholesterol, and MC3) in the presence of liposomes comprised of ALC-0159, DSPC, and cholesterol.

FIG. 41 illustrates the effect of up to five freeze-thaw cycles (1 FT, 3 FT, 5 FT) mRNA encapsulation efficiency (Encapsulation %) of flu mRNA LNP formulations (3, 0.1, 0.01, and 0.001 mg/ml mRNA encapsulated by ALC-0159, DSPC, cholesterol, and MC3) in the presence of liposomes comprised of ALC-0159, DSPC, and cholesterol.

FIGS. 42A-42B illustrate the effect of lyophilization (with and without annealing during freezing) and reconstitution (in water or saline) on LNP particle size (FIG. 42A) and LNP polydispersity index (PDI) (FIG. 42B) of flu mRNA LNP formulations (3, 0.1, 0.01, and 0.001 mg/ml mRNA encapsulated by ALC-0159, DSPC, cholesterol, and MC3) in the presence of blank lipid nanoparticles comprised of ALC-0159, DSPC, cholesterol, and MC3.

FIG. 43 illustrates the effect of lyophilization (with and without annealing during freezing) and reconstitution (in water or saline) encapsulation efficiency (Encapsulation %) of flu mRNA LNP formulations (3, 0.1, 0.01, and 0.001 mg/ml mRNA encapsulated by ALC-0159, DSPC, cholesterol, and MC3) in the presence of blank lipid nanoparticles comprised of ALC-0159, DSPC, cholesterol, and MC3.

FIGS. 44A-44B illustrate the effect of lyophilization (with and without annealing during freezing) and reconstitution (in water or saline) on LNP particle size (FIG. 44A) and LNP polydispersity index (PDI) (FIG. 44B) of flu mRNA LNP formulations (3, 0.1, 0.01, and 0.001 mg/ml mRNA encapsulated by ALC-0159, DSPC, cholesterol, and A9) in the presence of blank lipid nanoparticles comprised of ALC-0159, DSPC, cholesterol, and A9.

FIG. 45 illustrates the effect of lyophilization (with and without annealing during freezing) and reconstitution (in water or saline) encapsulation efficiency (Encapsulation %) of flu mRNA LNP formulations (3, 0.1, 0.01, and 0.001 mg/ml mRNA encapsulated by ALC-0159, DSPC, cholesterol, and A9) in the presence of blank lipid nanoparticles comprised of ALC-0159, DSPC, cholesterol, and A9.

FIGS. 46A-46B illustrate the effect of lyophilization (with and without annealing during freezing) and reconstitution (in water or saline) on LNP particle size (FIG. 46A) and LNP polydispersity index (PDI) (FIG. 46B) of flu mRNA LNP formulations (3, 0.1, 0.01, and 0.001 mg/mL mRNA encapsulated by ALC-0159, DSPC, cholesterol, and ALC-0315) in the presence of liposomes comprised of ALC-0159, DSPC, and cholesterol.

FIG. 47 illustrates the effect of lyophilization (with and without annealing during freezing) and reconstitution (in water or saline) encapsulation efficiency (Encapsulation %) of flu mRNA LNP formulations (3, 0.1, 0.01, and 0.001 mg/mL mRNA encapsulated by ALC-0159, DSPC, cholesterol, and ALC-0315) in the presence of liposomes comprised of ALC-0159, DSPC, and cholesterol.

FIGS. 48A-48B illustrate the effect of lyophilization (with and without annealing during freezing) and reconstitution (in water or saline) on LNP particle size (FIG. 48A) and LNP polydispersity index (PDI) (FIG. 48B) of flu mRNA LNP formulations (3, 0.1, 0.01, and 0.001 mg/ml mRNA encapsulated by ALC-0159, DSPC, cholesterol, and MC3) in the presence of liposomes comprised of ALC-0159, DSPC, and cholesterol.

FIG. 49 illustrates the effect of lyophilization (with and without annealing during freezing) and reconstitution (in water or saline) encapsulation efficiency (Encapsulation %) of flu mRNA LNP formulations (3, 0.1, 0.01, and 0.001 mg/ml mRNA encapsulated by ALC-0159, DSPC, cholesterol, and MC3) in the presence of liposomes comprised of ALC-0159, DSPC, and cholesterol.

### DETAILED DESCRIPTION

The inventors surprisingly conceived compositions and methods thereof that relate to liquid, frozen, or lyophilized lipid nanoparticles encapsulating or associated with RNA in the presence of a cryoprotectant, preferably a carbohydrate cryoprotectant, and t-butanol. The RNA-encapsulating lipid nanoparticles that were produced in the presence of t-butanol surprisingly resulted in post-lyophilization LNP size changes being limited to about 20 nm when compared to the LNP size pre-lyophilization. The processes involving t-butanol used to manufacture RNA-encapsulating LNP also resulted in low polydispersity indexes (PDIs) less than 0.15. Encapsulation of the RNA payload, including a self-amplifying RNA payload, surprisingly remained at or greater than 95%, when compared to the encapsulation of the RNA payload pre-lyophilization. Moreover, RNA integrity surprisingly remained high as the RNA-encapsulating LNPs underwent limited processing and hold times as a result of the LNP production process involving t-butanol. In some embodiments, the compositions comprising RNA-encapsulating lipid nanoparticles prepared in the presence of t-butanol described herein may further be in the presence of lipid nanoparticles that are devoid of nucleic acid, e.g., not encapsulating and not associated with RNA (also referred herein as “blank” LNPs). In some embodiments, the compositions comprising RNA-encapsulating lipid nanoparticles prepared in the presence of t-butanol described herein may further be in the presence of liposomes. In some embodiments, the compositions comprising RNA-encapsulating lipid nanoparticles prepared in the presence of t-butanol described herein may further be in the presence of a higher cryoprotectant concentration. Such compositions may result in a composition comprising LNPs encapsulating RNA or associated with RNA that is characterized by, among other things, an improved integrity of the RNA after completion of the respective freezing or lyophilization process and which is further characterized by increased storage stability, such as, for example, with respect to storage for extended periods and/or under non-cooling conditions, as compared to a composition comprising lipid nanoparticles encapsulating or associated with RNA that were not prepared in the presence of t-butanol and/or that are in the absence of the blank LNPs, or liposomes, or a higher cryoprotectant concentration when assessed under identical conditions. In other words, in some aspects, the compositions include a mixture of a lipid nanoparticle encapsulating or associated with RNA, preferably produced by a process comprising t-butanol as described herein, and a cryoprotectant, wherein the composition may further comprise t-butanol, preferably in an amount less than 5% w/v, that results in improved characteristics of the encapsulated RNA after freezing or lyophilization processes, preferably for use as a pharmaceutical composition, such as, for example, an immunogenic composition or vaccine. In some aspects, the compositions include a mixture of a first lipid nanoparticle encapsulating or associated with RNA, preferably produced by a process comprising t-butanol as described herein, a second lipid nanoparticle that is devoid of nucleic acid, and a cryoprotectant that results in improved characteristics of the encapsulated RNA after freezing or lyophilization processes, preferably for use as a pharmaceutical composition, such as, for example, an immunogenic composition or vaccine.

In some aspects, the compositions include a mixture of a first lipid nanoparticle encapsulating or associated with RNA and an increased cryoprotectant concentration and t-butanol, preferably in residual amount, e.g., less than 5% w/v, that results in improved characteristics of the encapsulated RNA after freezing or lyophilization processes, preferably for use as a pharmaceutical composition, such as, for example, an immunogenic composition or vaccine. In some aspects, the compositions include a mixture of a first lipid nanoparticle encapsulating or associated with RNA, wherein the RNA-encapsulating LNP was prepared in the presence of t-butanol, and a second lipid nanoparticle that is devoid of nucleic acid that results in improved characteristics of the encapsulated RNA after freezing or lyophilization processes, preferably for use as a pharmaceutical composition, such as, for example, an immunogenic composition or vaccine. In some aspects, the compositions include a mixture of a first lipid nanoparticle encapsulating or associated with RNA, wherein the RNA-encapsulating LNP was prepared in the presence of t-butanol, and a liposome that results in improved characteristics of the encapsulated RNA after freezing or lyophilization processes, preferably for use as a pharmaceutical composition, such as, for example, an immunogenic composition or vaccine. In some aspects, the compositions include a mixture of a lipid nanoparticle encapsulating or associated with RNA, wherein the LNP was prepared in the presence of t-butanol, and an increased cryoprotectant concentration, wherein the composition further comprises t-butanol, which results in improved characteristics of the encapsulated RNA after freezing or lyophilization processes, preferably for use as a pharmaceutical composition, such as, for example, an immunogenic composition or vaccine. In some aspects, the compositions include a mixture of a first lipid nanoparticle encapsulating or associated with RNA, wherein the first LNP was prepared in the presence of t-butanol, a liposome, and an increased cryoprotectant concentration, wherein the composition further comprises t-butanol, which results in improved characteristics of the encapsulated RNA after freezing or lyophilization processes, preferably for use as a pharmaceutical composition, such as, for example, an immunogenic composition or vaccine.

Advantageously, the compositions and methods thereof described herein are suitable for use at an industrial scale. The methods described herein may be used to produce, for example, a frozen or lyophilized composition comprising LNPs encapsulating or associated with RNA having the above-mentioned properties in a reproducible and cost-effective manner. The composition comprising LNPs encapsulating or associated with RNA may advantageously be stored, shipped and applied, e.g., for example as a vaccine, without a cold chain, while the integrity and the biological activity of the RNA in the composition remain unexpectedly high.

A frozen composition refers to a composition that has undergone a freezing process, such that the composition has a temperature, for example, at least below 0° C. and greater than about −80° C., at least below 0° C. and greater than about −90° C. or at least below 0° C. and greater than about −40° C., or a temperature of less than −30° C., e.g., about −40° C. to about −30° C., or about −40° C. to about −15° C., or about −40° C. to about −20° C., thereby forming a frozen composition. Lyophilization, or freeze-drying, is a process widely used in the pharmaceutical industry for the preservation of biological and pharmaceutical materials. In lyophilization, water present in a material is converted to ice during a freezing step and then removed from the material by direct sublimation under low-pressure conditions during a primary drying step. During freezing, however, not all of the water is transformed to ice. Some portion of the water is trapped in a matrix of solids containing, for example, formulation components and/or the active ingredient. The excess bound water within the matrix can be reduced to a desired level of residual moisture during a secondary drying step. All lyophilization steps, freezing, primary drying and secondary drying, are determinative of the final product properties.

Aspects of the present disclosure provide RNA (e.g., mRNA) vaccines that include polynucleotide encoding an influenza virus antigen. Influenza virus RNA vaccines, as provided herein may be used to induce a balanced immune response, comprising both cellular and humoral immunity, without many of the risks associated with DNA vaccination.

In some aspects, the virus is a strain of Influenza A or Influenza B or combinations thereof.

In some aspects, the antigenic polypeptide encodes a hemagglutinin protein or immunogenic fragment thereof. In some aspects, the hemagglutinin protein is H1, H2, H3, H4, H5, H6, H7, H8, H9, H10, H11, H12, H13, H14, H15, H16, H17, H18, or an immunogenic fragment thereof. In some aspects, the hemagglutinin protein does not comprise a head domain. In some aspects, the hemagglutinin protein comprises a portion of the head domain. In some aspects, the hemagglutinin protein does not comprise a cytoplasmic domain. In some aspects, the hemagglutinin protein comprises a portion of the cytoplasmic domain. In some aspects, the truncated hemagglutinin protein comprises a portion of the transmembrane domain.

Some aspects provide influenza vaccines comprising one or more RNA polynucleotides having an open reading frame encoding a hemagglutinin protein and a pharmaceutically acceptable carrier or excipient, formulated within a cationic lipid nanoparticle. In some aspects, the hemagglutinin protein is selected from H1, H7 and H10. In some aspects, the RNA polynucleotide further encodes neuraminidase protein. In some aspects, the hemagglutinin protein is derived from a strain of Influenza A virus or Influenza B virus or combinations thereof. In some aspects, the Influenza virus is selected from H1N1, H3N2, H7N9, and H10N8.

Some aspects provide methods of preventing or treating influenza viral infection comprising administering to a subject any of the immunogenic compositions and/or vaccines described herein. In some aspects, the antigen specific immune response comprises a T cell response. In some aspects, the antigen specific immune response comprises a B cell response. In some aspects, the antigen specific immune response comprises both a T cell response and a B cell response. In some aspects, the method of producing an antigen specific immune response involves a single administration of the immunogenic compositions and/or vaccine. In some aspects, the immunogenic compositions and/or vaccine is administered to the subject by intradermal, intramuscular injection, subcutaneous injection, intranasal inoculation, or oral administration.

In some aspects, the RNA (e.g., mRNA) polynucleotides or portions thereof may encode one or more polypeptides or fragments thereof of an influenza strain as an antigen.

### 1. Examples of Definitions

Throughout this application, the term “about” is used according to its plain and ordinary meaning in the area of cell and molecular biology to indicate that a value includes the inherent variation or standard deviation of error for the measurement or quantitation method being employed to determine the value. For example, in some aspects, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the measurement or quantitation.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The phrase “essentially all” is defined as “at least 95%”; if essentially all members of a group have a certain property, then at least 95% of members of the group have that property. In some instances, essentially all means equal to any one of, at least any one of, or between any two of 95, 96, 97, 98, 99, or 100% of members of the group have that property.

The compositions and methods for their use can “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting” or “reduce” or “decrease” or any variation of these terms includes any measurable decrease or complete inhibition to achieve a desired result. The terms “promote” or “increase” or any variation of these terms includes any measurable increase to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “RNA,” as used herein, means a nucleic acid molecule that includes ribonucleotide residues (such as containing the nucleotide base(s) adenine (A), cytosine (C), guanine (G) and/or uracil (U)). For example, RNA can contain all, or a majority of, ribonucleotide residues. As used herein, the term “ribonucleotide” means a nucleotide with a hydroxyl group at the 2′ position of a β-D-ribofuranosyl group. In one aspect, RNA can be messenger RNA (mRNA) that relates to a RNA transcript which encodes a peptide or protein. As known to those of skill in the art, mRNA generally contains a 5′ untranslated region (5′-UTR), a polypeptide coding region, and a 3′ untranslated region (3′-UTR). Without any limitation, RNA can encompass double stranded RNA, antisense RNA, single stranded RNA, isolated RNA, synthetic RNA, RNA that is recombinantly produced, and modified RNA (modRNA).

As contemplated herein, without any limitations, RNA can be used as a therapeutic modality to treat and/or prevent a number of conditions in mammals, including humans. Methods described herein comprise administration of the RNA described herein to a mammal, such as a human. For example, in one aspect such methods of use for RNA include an antigen-coding RNA vaccine to induce robust neutralizing antibodies and accompanying/concomitant T-cell response to achieve protective immunization with preferably minimal vaccine doses. The RNA administered is preferably in vitro transcribed RNA.

For example, such RNA can be used to encode at least one antigen intended to generate an immune response in said mammal. Antigens can be a peptide or protein from a cancer, a pathogen, a mutant protein, a misfolded protein, a prion, etc. Pathogenic antigens can be a peptide or protein antigens derived from a pathogen associated with infectious disease which are preferably selected from antigens derived from the pathogens Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 01 57:H7, 01 1 1 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBOV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowled, Necator americanus, Neisseria gonorrhoeae, Neisseria meningitidis, Nocardia asteroides, Nocardia spp, Onchocerca volvulus, Orientia tsutsugamushi, Orthomyxoviridae family ((Influenza), Paracoccidioides brasiliensis, Paragonimus spp, Paragonimus westermani, Parvovirus B1 9, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

Conditions and/or diseases that can be treated with such RNA therapeutics include, but are not limited to, cancer, overproduction of a protein, production of a mutant protein, misfolding of a protein, and/or those caused and/or impacted by a pathogen, such as a viral infection. A representative but non-limiting list of cancers that the disclosed RNA therapeutics can be used to treat include, but are not limited to, lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer. A representative but non-limiting list of viruses that the disclosed RNA therapeutics can be used to treat include, but are not limited to, an arenavirus (such as Lassa virus, or lymphocytic choriomeningitis virus (LCMV)); an astrovirus; a bunyavirus (such as a Hantavirus); a calicivirus; a coronavirus (such as a severe acute respiratory syndrome virus (SARS)—e.g., SARS-CoV-1, or a middle east respiratory syndrome (MERS) virus); a filovirus (such as Ebola virus or Marburg virus); a flavivirus (such as Yellow Fever virus, West Nile virus, or Hepatitis C virus (HCV)); a hepadnavirus; a hepevirus; an orthomyxovirus (such as Influenza A virus, Influenza B virus, or Influenza C virus); a paramyxovirus (such as Rubeola virus, or Rubulavirus); a picornavirus (such as Poliovirus, Hepatitis A virus, or Rhinovirus); a reovirus (such as Rotavirus); a retrovirus (such as Human Immunodeficiency Virus (HIV), or Human T-lymphotropic virus (HTLV)); a rhabdovirus (such as Rabies virus or Rabies lyssavirus); a togavirus (such as Sindbis virus (SINV), Eastern Equine Encephalitis virus (EEEV), Western Equine Encephalitis virus (WEEV), or Rubella virus).

An “isolated RNA” is defined as an RNA molecule that can be recombinant or has been isolated from total genomic nucleic acid.

A “modified RNA” or “modRNA” refers to an RNA molecule, e.g., an mRNA molecule, having at least one addition, deletion, substitution, and/or alteration of one or more nucleotides as compared to naturally occurring RNA. Such alterations can refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide can replace one or more uridine and/or cytidine nucleotides. For example, these replacements can occur for every instance of uridine and/or cytidine in the RNA sequence, or can occur for only select uridine and/or cytidine nucleotides. Such alterations to the standard nucleotides in RNA can include non-standard nucleotides, such as chemically synthesized nucleotides or deoxynucleotides. For example, at least one uridine nucleotide can be replaced with 1-methylpseudouridine in an RNA sequence. Other such altered nucleotides are known to those of skill in the art. Such altered RNAs are considered analogs of naturally-occurring RNA. In some aspects, the RNA is produced by in vitro transcription using a DNA template, where DNA refers to a nucleic acid that contains deoxyribonucleotides. In some aspects, the RNA can be replicon RNA (replicon), in particular self-replicating RNA, or self-amplifying RNA (saRNA).

The term “DNA,” as used herein, means a nucleic acid molecule that includes deoxyribonucleotide residues (such as containing the nucleotide base(s) adenine (A), cytosine (C), guanine (G) and/or thymine (T)). For example, DNA can contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA can encompass double stranded DNA, antisense DNA, single stranded DNA, isolated DNA, synthetic DNA, DNA that is recombinantly produced, and modified DNA.

As used herein, a “protein,” “polypeptide,” or “peptide” refers to a molecule comprising at least two amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. Where a protein is specifically mentioned herein, it is in general a reference to a native (wild-type) or recombinant (modified) protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

### II. mRNA Molecules

The present invention relates to mRNA immunogenic compositions and/or vaccines in general. A number of mRNA vaccine platforms are available in the prior art. The basic structure of in vitro transcribed (IVT) mRNA generally closely resembles “mature” eukaryotic mRNA, and consists of (i) a protein-encoding open reading frame (ORF), flanked by (ii) 5′ and 3′ untranslated regions (UTRs), and at the end sides (iii) a 7-methyl guanosine 5′ cap structure and (iv) a 3′ poly(A) tail. The mRNA molecule may include one (monocistronic), two (bicistronic), or more (multicistronic) open reading frames (ORFs), which may be a sequence of codons that is translatable into a polypeptide or protein of interest. The mRNA molecule may encode one polypeptide of interest or more, such as an antigen or more than one antigen, e.g., two, three, four, five, six, seven, eight, nine, ten, or more polypeptides. Alternatively, or in addition, one mRNA molecule may also encode more than one polypeptide of interest, such as an antigen, e.g., a bicistronic, or tricistronic mRNA molecule that encodes different or identical antigens. The non-coding structural features play important roles in the pharmacology of mRNA and can be individually optimized to modulate the mRNA stability, translation efficiency, and immunogenicity.

By incorporating modified nucleosides, mRNA transcripts referred to as “nucleoside-modified mRNA” or “modRNA” can be produced with reduced immunostimulatory activity, and therefore an improved safety profile can be obtained. In addition, modified nucleosides allow the design of mRNA immunogenic compositions and/or vaccines with strongly enhanced stability and translation capacity, as they can avoid the direct antiviral pathways that are induced by type IFNs and are programmed to degrade and inhibit invading mRNA. For instance, the replacement of uridine with pseudouridine in IVT mRNA reduces the activity of 2′-5′-oligoadenylate synthetase, which regulates the mRNA cleavage by RNase L. In addition, lower activities are measured for protein kinase R, an enzyme that is associated with the inhibition of the mRNA translation process.

Besides the incorporation of modified nucleotides, other approaches have been validated to increase the translation capacity and stability of mRNA. One example is the development of “sequence-engineered mRNA”. Here, mRNA expression can be strongly increased by sequence optimizations in the ORF and UTRs of mRNA, for instance by enriching the GC content, or by selecting the UTRs of natural long-lived mRNA molecules. Another approach is the design of “self-amplifying mRNA” constructs. These are mostly derived from alphaviruses, and contain an ORF that is replaced by the antigen of interest together with an additional ORF encoding viral replicase. The latter drives the intracellular amplification of mRNA, and can therefore significantly increase the antigen expression capacity. Also, several modifications have been implemented at the end structures of mRNA. Anti-reverse cap (ARCA) modifications can ensure the correct cap orientation at the 5′ end, which yields almost complete fractions of mRNA that can efficiently bind the ribosomes. Other cap modifications, such as phosphorothioate cap analogs, may further improve the affinity towards the eukaryotic translation initiation factor 4E, and increase the resistance against the RNA decapping complex.

Conversely, by modifying its structure, the potency of mRNA to trigger innate immune responses may be further improved, but to the detriment of translation capacity. By stabilizing the mRNA with either a phosphorothioate backbone, or by its precipitation with the cationic protein protamine, antigen expression can be diminished, but stronger immune-stimulating capacities can be obtained.

In one aspect the invention relates to an immunogenic composition comprising an mRNA molecule that encodes one or more polypeptides or fragments thereof of an influenza strain as an antigen. In some aspects, the mRNA molecule comprises a nucleoside-modified mRNA. In some aspects, the mRNA molecule does not comprise a nucleoside-modified mRNA, for example, a self-amplifying RNA that does not incorporate modified nucleosides. In some aspects, the mRNA molecule is a self-amplifying RNA molecule.

mRNA useful in the disclosure typically include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some aspects, the mRNA of the invention further includes a poly-A region or a Kozak sequence (e.g., in the 5′-UTR). In some cases, mRNA of the invention may contain one or more intronic nucleotide sequences capable of being excised from the polynucleotide. In some aspects, mRNA of the invention may include a 5′ cap structure, a chain terminating nucleotide, a stem loop, a poly A sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine), and/or a 5-substituted cytidine (e.g., 5-methyl-cytidine).

In some aspects, the mRNA molecule includes equal to any one of, at least any one of, at most any one of, or between any two of from about 20 to about 100,000 nucleotides (e.g., equal to any one of, at least any one of, at most any one of, or between any two of from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000). In some aspects, the mRNA molecule includes at least 100 nucleotides. For example, in some aspects, the mRNA has a length between 100 and 15,000 nucleotides; between 7,000 and 16,000 nucleotides; between 8,000 and 15,000 nucleotides; between 9,000 and 12,500 nucleotides; between 11,000 and 15,000 nucleotides; between 13,000 and 16,000 nucleotides; between 7,000 and 25,000 nucleotides. In some aspects, the mRNA length is at least, at most, between any two of, or exactly 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, or 100000 nucleotides. In some aspects, one or more of the nucleotide size ranges in the list may be excluded.

In some aspects, a LNP includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain aspects, the N:P ratio may be from about 2:1 to about 8:1. In other aspects, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

mRNA of the disclosure may include one or more naturally occurring components, including any of the canonical nucleotides A (adenosine), G (guanosine), C (cytosine), U (uridine), or T (thymidine). In one aspect, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

In some aspects, the RNA molecule is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

Modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages, in some instances, are intended to replace naturally-occurring phosphodiester-5′-methylene linkages with four atom linking groups to confer nuclease resistance and enhanced cellular uptake to the resulting compound.

mRNA of the invention may include one or more alternative components, as described herein, which impart useful properties including increased stability and/or the lack of a substantial induction of the innate immune response of a cell into which the polynucleotide is introduced. For example, a mRNA or a modRNA may exhibit reduced degradation in a cell into which the respective mRNA or modRNA is introduced, relative to a corresponding unaltered mRNA or an mRNA or modRNA that does not contain or is not introduced with the alternative components. These alternative species may enhance the efficiency of protein production, intracellular retention of the polynucleotides, and/or viability of contacted cells, as well as possess reduced immunogenicity. In some instances, nucleic acids do not substantially induce an innate immune response of a cell into which the polynucleotide (e.g., mRNA) is introduced. Features of an induced innate immune response can include 1) increased expression of pro-inflammatory cytokines, 2) activation of intracellular PRRs (RIG-I, MDA5, etc.), and/or 3) termination or reduction in protein translation.

mRNA of the invention may include one or more modified (e.g., altered or alternative) nucleobases, nucleosides, nucleotides, or combinations thereof. The mRNA useful in a LNP can include any useful modification or alteration, such as to the nucleobase, the sugar, or the internucleoside linkage (e.g., to a linking phosphate/to a phosphodiester linkage/to the phosphodiester backbone). In certain aspects, alterations (e.g., one or more alterations) are present in each of the nucleobase, the sugar, and the internucleoside linkage. Alterations according to the present disclosure may be alterations of ribonucleic acids (RNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

mRNA of the invention may or may not be uniformly altered along the entire length of the molecule. For example, one or more or all types of nucleotide (e.g., purine or pyrimidine, or any one or more or all of A, G, U, C) may or may not be uniformly altered in a mRNA, or in a given predetermined sequence region thereof. In some instances, all nucleotides X in a mRNA (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some aspects, the polynucleotide includes an alteration at the 3′-terminus. The polynucleotide may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%) (e.g., at least, at most, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% alternative nucleotides). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some aspects, at least 5%, at least 10%, at least 25%, at least 50%, at least 80%, at least 90%, or 100% (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%) of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%) of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some aspects, the mRNA comprises one or more alternative nucleoside or nucleotides. The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

In some aspects, the nucleobase is an alternative uracil. Exemplary nucleobases and nucleosides having an alternative uracil include pseudouridine (w), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (m U), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5s2U), 5-aminomethyl-2-thio-uracil (nmVu), 5-methylaminomethyl-uracil (mnm5U), 5-methylaminomethyl-2-thio-uracil (mnmVu), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnmVu), 5-propynyl-uracil, 1-propynyl-pseudouracil, 5-taurinomethyl-uracil (xm5U), 1-taurinomethyl-pseudouridine, 5-taurinomethyl-2-thio-uracil (xm5s2U), 1-taurinomethyl-4-thio-pseudouridine, 5-methyl-uracil (m5U, i.e., having the nucleobase deoxythymine), 1-methyl-pseudouridine (mV), 5-methyl-2-thio-uracil (m5s2U), I-methyl-4-thio-pseudouridine (m xj/), 4-thio-1-methyl-pseudouridine, 3-methyl-pseudouridine (m \/), 2-thio-1-methyl-pseudouridine, 1-methyl-1-deaza-pseudouri dine, 2-thio-1-methyl-1-deaza-pseudouri dine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, NI-methyl-pseudouridine, 3-(3-amino-3-carboxypropyl) uracil (acp U), I-methyl-3-(3-amino-3-carboxypropyl) pseudouridine (acp w), 5-(isopentenylaminomethyl) uracil (inm5U), 5-(isopentenylaminomethyl)-2-thio-uracil (inm5s2U), 5,2′-O-dimethyl-uridine (m5Um), 2-thio-2′-O-methyl-uridine (s2Um), 5-methoxycarbonylmethyl-2′-O-methyl-uridine (mem Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m Um), and 5-(isopentenylaminomethyl)-2′-O-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(I-E-propenylamino)]uracil.

In some aspects, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocy tidine, 4-thio-1-methy 1-pseudoisocytidine, 4-thio-1-methyl-1-deaza-pseudoisocytidine, 1-methyl-1-deaza-pseudoisocyti dine, zebularine, 5-aza-zebularine, 5-methy 1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some aspects, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methy 1-adenine (mI A), 2-methyl-adenine (m2A), N6-methyl-adenine (m6A), 2-methylthio-N6-methyl-adenine (ms2m6A), N6-isopentenyl-adenine (16A), 2-methylthio-N6-isopentenyl-adenine (ms2i6A), N6-(cis-hydroxyisopentenyl) adenine (io6A), 2-methylthio-N6-(cis-hydroxyisopentenyl) adenine (ms2io6A), N6-glycinylcarbamoyl-adenine (g6A), N6-threonylcarbamoyl-adenine (16A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (mlAm), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some aspects, the nucleobase is an alternative guanine. Exemplary nucleobases and nucleosides having an alternative guanine include inosine (I), 1-methyl-inosine (mil), wyosine (imG), methylwyosine (mimG), 4-demethyl-wyosine (imG-14), isowyosine (imG2), wybutosine (yW), peroxywybutosine (02yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQI), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2,N2-dimethyl-guanine (m22G), N2,7-dimethyl-guanine (m2,7G), N2,N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m2,2Gm), 1-methyl-2′-O-methyl-guanosine (mlGm), N2,7-dimethyl-2′-O-methyl-guanosine (m2,7Gm), 2′-O-methyl-inosine (Im), 1,2′-O-dimethyl-inosine (mllm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another aspect, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a] 1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

The 5′ untranslated regions (UTR) is a regulatory region of DNA situated at the 5′ end of a protein coding sequence that is transcribed into mRNA but not translated into protein. 5′ UTRs may contain various regulatory elements, e.g., 5′ cap structure, stem-loop structure, and an internal ribosome entry site (IRES), which may play a role in the control of translation initiation. The 3′ UTR, situated downstream of a protein coding sequence, may be involved in regulatory processes including transcript cleavage, stability and polyadenylation, translation, and mRNA localization. In some aspects, the UTR is derived from an mRNA that is naturally abundant in a specific tissue (e.g., lymphoid tissue), to which the mRNA expression is targeted. In some aspects, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). According, the UTR sequence may prolong protein synthesis in a tissue-specific manner. In some aspects, the 5′ UTR and the 3′ UTR sequences are computationally derived. In some aspects, the 5′ UTR and the 3′ UTRs are derived from a naturally abundant mRNA in a tissue. The tissue may be, for example, liver, a stem cell, or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte.

The mRNA may include a 5′-cap structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing. Endogenous polynucleotide molecules may be 5′-end capped generating a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-O-methylated. 5′-decapping through hydrolysis and cleavage of the guanylate cap structure may target a polynucleotide molecule, such as an mRNA molecule, for degradation. Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional alternative guanosine nucleotides may be used such as α-methyl-phosphonate and seleno-phosphate nucleotides. Additional alterations include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of an mRNA molecule.

Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide. For example, the Anti-Reverse Cap Analog (ARCA) cap contains two guanosines linked by a 5′-5′-triphosphate group, wherein one guanosine contains an N7-methyl group as well as a 3′-O-methyl group (i.e., N7, ‘—O-dimethyl-guanosine-5’-triphosphate-5′-guanosine, m7G-3′mppp-G, which may equivalently be designated 3′ O-Me-m7G (5′)ppp(5′) G). The 3′-O atom of the other, unaltered, guanosine becomes linked to the 5′-terminal nucleotide of the capped polynucleotide (e.g., an mRNA). The N7- and 3′-O-methylated guanosine provides the terminal moiety of the capped polynucleotide (e.g., mRNA). Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference. Alternatively, a cap analog may be a N7-(4-chlorophenoxy ethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G (5) ppp(5′) G and a N7-(4-chlorophenoxyethyl)-m3′-OG (5)ppp(5′) G cap analog (see, e.g., the various cap analogs and the methods of synthesizing cap analogs described in Kore et al. Bioorganic & Medicinal Chemistry 2013 21:4570-4574; the cap structures of which are herein incorporated by reference). In other instances, a cap analog useful in the polynucleotides of the present disclosure is a 4-chloro/bromophenoxy ethyl analog. While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in the polynucleotides of the present disclosure are those which, among other things, have enhanced binding of cap binding proteins, increased half-life, reduced susceptibility to 5′ endonucleases, and/or reduced 5′ decapping, as compared to synthetic 5′-cap structures known in the art (or to a wild-type, natural or physiological 5′-cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the Cap 1 structure. This cap results in a higher translational-competency, cellular stability, and a reduced activation of cellular pro-inflammatory cytokines, as compared, e.g., to other 5′ cap analog structures known in the art. Other exemplary cap structures include 7 mG(5′)ppp(5′)N,pN2p(Cap 0), 7mG(5′)ppp(5′) NImpNp (Cap 1), 7mG(5′)-ppp(5′)NImpN2mp(Cap 2), and m(7)Gpppm(3)(6,6,2′)Apm(2′)Apm(2′)Cpm(2) (3,2′)Up (Cap 4). Because the alternative polynucleotides may be capped post-transcriptionally, and because this process is more efficient, nearly 100% of the mRNA may be capped. This is in contrast to −80% when a cap analog is linked to a polynucleotide in the course of an in vitro transcription reaction.

5′-terminal caps may include endogenous caps or cap analogs. A 5′-terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5′-cap. A modification on the 5′-cap may increase the stability of polynucleotide, increase the half-life of the polynucleotide, and could increase the polynucleotide translational efficiency. The modified 5′-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

A 5′-UTR may be provided as a flanking region to the mRNA. A 5′-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization. To alter one or more properties of an mRNA, 5′-UTRs which are heterologous to the coding region of an mRNA may be engineered. The mRNA may then be administered to cells, tissue or organisms and outcomes such as protein level, localization, and/or half-life may be measured to evaluate the beneficial effects the heterologous 5′-UTR may have on the mRNA. Variants of the 5′-UTRs may be utilized wherein one or more nucleotides are added or removed to the termini, including A, T, C or G. 5′-UTRs may also be codon-optimized, or altered in any manner described herein.

In some aspects, the capping region may include a single cap or a series of nucleotides forming the cap. In this aspect the capping region may be equal to any one of, at least any one of, at most any one of, or between any two of 1, 2, 3, 4, 5, 6, 7, 8, 9, 10, or at least 2, or 10 or fewer nucleotides in length. In some aspects, the cap is absent. In some aspects, the first and second operational regions may be equal to any one of, at least any one of, at most any one of, or between any two of 3 to 40, e.g., 5-30, 10-20, 15, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or at least 4, or 30 or fewer nucleotides in length and may comprise, in addition to a Start and/or Stop codon, one or more signal and/or restriction sequences.

mRNAs may include a stem loop such as, but not limited to, a histone stem loop. The stem loop may be a nucleotide sequence that is about 25 or about 26 nucleotides in length. The histone stem loop may be located 3′-relative to the coding region (e.g., at the 3′-terminus of the coding region). As a non-limiting example, the stem loop may be located at the 3′-end of a polynucleotide described herein. In some cases, an mRNA includes more than one stem loop (e.g., two stem loops). A stem loop may be located in a second terminal region of a polynucleotide. As a non-limiting example, the stem loop may be located within an untranslated region (e.g., 3′-UTR) in a second terminal region.

In some cases, a mRNA which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide. In other cases, a mRNA, which includes the histone stem loop may be stabilized by an alteration to the 3′-region of the polynucleotide that can prevent and/or inhibit the addition of oligio (U). In yet other cases, a mRNA, which includes the histone stem loop may be stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

In some instances, the mRNA of the present disclosure may include a histone stem loop, a poly-A region, and/or a 5′-cap structure. The histone stem loop may be before and/or after the poly-A region. The polynucleotides including the histone stem loop and a poly-A region sequence may include a chain terminating nucleoside described herein. In other instances, the polynucleotides of the present disclosure may include a histone stem loop and a 5′-cap structure. The 5′-cap structure may include, but is not limited to, those described herein and/or known in the art. In some cases, the conserved stem loop region may include a miR sequence described herein. As a non-limiting example, the stem loop region may include the seed sequence of a miR sequence described herein. In another non-limiting example, the stem loop region may include a miR-122 seed sequence. mRNA may include at least one histone stem-loop and a poly-A region or polyadenylation signal. In certain cases, the polynucleotide encoding for a histone stem loop and a poly-A region or a polyadenylation signal may code for a pathogen antigen or fragment thereof. In other cases, the polynucleotide encoding for a histone stem loop and a poly-A region or a polyadenylation signal may code for a therapeutic protein. In some cases, the polynucleotide encoding for a histone stem loop and a poly-A region or a polyadenylation signal may code for a tumor antigen or fragment thereof. In other cases, the polynucleotide encoding for a histone stem loop and a poly-A region or a polyadenylation signal may code for an allergenic antigen or an autoimmune self-antigen.

An mRNA may include a poly-A sequence and/or polyadenylation signal. A poly-A sequence may be comprised entirely or mostly of adenine nucleotides or analogs or derivatives thereof. A poly-A sequence may be a tail located adjacent to a 3′ untranslated region of a nucleic acid. During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the alternative polynucleotides of the present disclosure.

Generally, the length of a poly-A region of the present disclosure is at least 30 nucleotides in length. In another aspect, the poly-A region is at least 35 nucleotides in length. In another aspect, the length is at least 40 nucleotides. In another aspect, the length is at least 45 nucleotides. In another aspect, the length is at least 55 nucleotides. In another aspect, the length is at least 60 nucleotides. In another aspect, the length is at least 70 nucleotides. In another aspect, the length is at least 80 nucleotides. In another aspect, the length is at least 90 nucleotides. In another aspect, the length is at least 100 nucleotides. In another aspect, the length is at least 120 nucleotides. In another aspect, the length is at least 140 nucleotides. In another aspect, the length is at least 160 nucleotides. In another aspect, the length is at least 180 nucleotides. In another aspect, the length is at least 200 nucleotides. In another aspect, the length is at least 250 nucleotides. In another aspect, the length is at least 300 nucleotides. In another aspect, the length is at least 350 nucleotides. In another aspect, the length is at least 400 nucleotides. In another aspect, the length is at least 450 nucleotides. In another aspect, the length is at least 500 nucleotides. In another aspect, the length is at least 600 nucleotides. In another aspect, the length is at least 700 nucleotides. In another aspect, the length is at least 800 nucleotides. In another aspect, the length is at least 900 nucleotides. In another aspect, the length is at least 1000 nucleotides. In another aspect, the length is at least 1100 nucleotides. In another aspect, the length is at least 1200 nucleotides. In another aspect, the length is at least 1300 nucleotides. In another aspect, the length is at least 1400 nucleotides. In another aspect, the length is at least 1500 nucleotides. In another aspect, the length is at least 1600 nucleotides. In another aspect, the length is at least 1700 nucleotides. In another aspect, the length is at least 1800 nucleotides. In another aspect, the length is at least 1900 nucleotides. In another aspect, the length is at least 2000 nucleotides. In another aspect, the length is at least 2500 nucleotides. In another aspect, the length is at least 3000 nucleotides.

In some instances, the poly-A region may be 80 nucleotides, 120 nucleotides, 160 nucleotides in length on an alternative polynucleotide molecule described herein. In other instances, the poly-A region may be 20, 40, 80, 100, 120, 140 or 160 nucleotides in length on an alternative polynucleotide molecule described herein.

In some aspects, the length of a poly-A region of the present disclosure is at least, at most, in between any two of, or exactly 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, or 3000 nucleotides.

In some cases, the poly-A region is designed relative to the length of the overall alternative polynucleotide. This design may be based on the length of the coding region of the alternative polynucleotide, the length of a particular feature or region of the alternative polynucleotide (such as mRNA), or based on the length of the ultimate product expressed from the alternative polynucleotide. When relative to any feature of the alternative polynucleotide (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the alternative polynucleotide to which it belongs. In this context, the poly-A region may be 10, 20, 30, 40, 50, 60, 70, 80, or 90% or more of the total length of the construct or the total length of the construct minus the poly-A region.

In certain cases, engineered binding sites and/or the conjugation of mRNA for poly-A binding protein may be used to enhance expression. The engineered binding sites may be sensor sequences which can operate as binding sites for ligands of the local microenvironment of the mRNA. As a non-limiting example, the mRNA may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct mRNA may be linked together to the PABP (poly-A binding protein) through the 3′-end using alternative nucleotides at the 3′-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site. In certain cases, a poly-A region may be used to modulate translation initiation. While not wishing to be bound by theory, the poly-A region recruits PABP which in turn can interact with translation initiation complex and thus may be essential for protein synthesis. In some cases, a poly-A region may also be used in the present disclosure to protect against 3′-5′-exonuclease digestion.

In some instances, an mRNA may include a poly-A-G Quartet. The G-quartet is a cyclic hydrogen bonded array of four guanosine nucleotides that can be formed by G-rich sequences in both DNA and RNA. In this aspect, the G-quartet is incorporated at the end of the poly-A region. The resultant mRNA may be assayed for stability, protein production and other parameters including half-life at various time points. It has been discovered that the poly-A-G quartet results in protein production equivalent to at least 75% of that seen using a poly-A region of 120 nucleotides alone.

In some cases, mRNA may include a poly-A region and may be stabilized by the addition of a 3′-stabilizing region. The mRNA with a poly-A region may further include a 5′-cap structure. In other cases, mRNA may include a poly-A-G Quartet. The mRNA with a poly-A-G Quartet may further include a 5′-cap structure. In some cases, the 3′-stabilizing region which may be used to stabilize mRNA includes a poly-A region or poly-A-G Quartet. In other cases, the 3′-stabilizing region which may be used with the present disclosure include a chain termination nucleoside such as 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside. In other cases, mRNA which includes a poly-A region or a poly-A-G Quartet may be stabilized by an alteration to the 3′-region of the polynucleotide that can prevent and/or inhibit the addition of oligo (U). In yet other instances, mRNA which includes a poly-A region or a poly-A-G Quartet may be stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

Modifications to the mRNA molecules described herein may be achieved using solid supports which may be manually manipulated or used in conjunction with a DNA or RNA synthesizer using methodology commonly known to those skilled in DNA or RNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA or RNA synthesis techniques. One of the most popular processes is the phosphoramidite technique.

Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TENTAGEL® Support—an aminopolyethyleneglycol derivatized support or Poros -a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some aspects, the oligonucleotide may be further defined as having one or more locked nucleotides, ethylene bridged nucleotides, peptide nucleic acids, or a 5′ (E)-vinyl-phosphonate (VP) modification. In some aspects, the oligonucleotides has one or more phosphorothioated DNA or RNA bases.

The mRNA molecules described herein may be analyzed and characterized using various methods. Analysis may be performed before or after capping. Alternatively, analysis may be performed before or after poly-A capture-based affinity purification. In another aspect, analysis may be performed before or after additional purification steps, e.g., anion exchange chromatography and the like. For example, mRNA quality may be determined using Bioanalyzer chip based electrophoresis system. In other aspects, mRNA purity is analyzed using analytical reverse phase HPLC. Capping efficiency may be analyzed using, e.g., total nuclease digestion followed by MS/MS quantitation of the dinucleotide cap species vs. uncapped GTP species. In vitro efficacy may be analyzed by, e.g., transfecting mRNA molecule into a human cell line. Protein expression of the polypeptide of interest may be quantified using methods such as ELISA or flow cytometry. Immunogenicity may be analyzed by, e.g., transfecting mRNA molecules into cell lines that indicate innate immune stimulation, e.g., PBMCs. Cytokine induction may be analyzed using, e.g., methods such as ELISA to quantify a cytokine, e.g., Interferon-α.

In some aspects, the RNA molecule is an saRNA. “saRNA,” “self-amplifying RNA,” and “replicon” refer to RNA with the ability to replicate itself. Self-amplifying RNA molecules may be produced by using replication elements derived from a virus or viruses, e.g., alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNAs. These daughter RNAs, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the protein of interest, e.g., an antigen. The overall result of this sequence of transcriptions is an amplification in the number of the introduced saRNAs and so the encoded gene of interest, e.g., a viral antigen, can become a major polypeptide product of the cells.

In some aspects, the self-amplifying RNA includes at least one or more genes selected from any one of viral replicases, viral proteases, viral helicases and other nonstructural viral proteins. In some aspects, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In some aspects, the self-amplifying RNA molecule is not encapsulated in a virus-like particle. Self-amplifying RNA molecules described herein may be designed so that the self-amplifying RNA molecule cannot induce production of infectious viral particles. This may be achieved, for example, by omitting one or more viral genes encoding structural proteins that are necessary to produce viral particles in the self-amplifying RNA. For example, when the self-amplifying RNA molecule is based on an alphavirus, such as Sinbis virus (SIN), Semliki forest virus and Venezuelan equine encephalitis virus (VEE), one or more genes encoding viral structural proteins, such as capsid and/or envelope glycoproteins, may be omitted.

In some aspects, a self-amplifying RNA molecule described herein encodes (i) an RNA-dependent RNA polymerase that may transcribe RNA from the self-amplifying RNA molecule and (ii) a polypeptide of interest, e.g., a viral antigen. In some aspects, the polymerase may be an alphavirus replicase, e.g., including any one of alphavirus protein nsP1, nsP2, nsP3, nsP4, and any combination thereof. In some aspects, the self-amplifying RNA molecules described herein may include one or more modified nucleotides (e.g., pseudouridine, N6-methyladenosine, 5-methylcytidine, 5-methyluridine). In some aspects, the self-amplifying RNA molecules does not include a modified nucleotide (e.g., pseudouridine, N6-methyladenosine, 5-methylcytidine, 5-methyluridine).

The saRNA construct may encode at least one non-structural protein (NSP), disposed 5′ or 3′ of the sequence encoding at least one peptide or polypeptide of interest. In some aspects, the sequence encoding at least one NSP is disposed 5′ of the sequences encoding the peptide or polypeptide of interest. Thus, the sequence encoding at least one NSP may be disposed at the 5′ end of the RNA construct. In some aspects, at least one non-structural protein encoded by the RNA construct may be the RNA polymerase nsP4. In some aspects, the saRNA construct encodes nsP1, nsP2, nsP3 and, nsP4. As is known in the art, nsP1 is the viral capping enzyme and membrane anchor of the replication complex (RC). nsP2 is an RNA helicase and the protease responsible for the ns polyprotein processing. nsP3 interacts with several host proteins and may modulate protein poly- and mono-ADP-ribosylation. nsP4 is the core viral RNA-dependent RNA polymerase. In some aspects, the polymerase may be an alphavirus replicase, e.g., comprising one or more of alphavirus proteins nsP1, nsP2, nsP3, and nsP4.

Whereas natural alphavirus genomes encode structural virion proteins in addition to the non-structural replicase polypeptide, in some aspects, the self-amplifying RNA molecules do not encode alphavirus structural proteins. In some aspects, the self-amplifying RNA may lead to the production of genomic RNA copies of itself in a cell, but not to the production of RNA that includes virions. Without being bound by theory or mechanism, the inability to produce these virions means that, unlike a wild-type alphavirus, the self-amplifying RNA molecule cannot perpetuate itself in infectious form. The alphavirus structural proteins which are necessary for perpetuation in wild-type viruses can be absent from self-amplifying RNAs of the present disclosure and their place can be taken by gene(s) encoding the immunogen of interest, such that the subgenomic transcript encodes the immunogen rather than the structural alphavirus virion proteins.

In some aspects, the self-amplifying RNA molecule may have two open reading frames. The first (5′) open reading frame can encode a replicase; the second (3′) open reading frame can encode a polypeptide comprising an antigen of interest. In some aspects the RNA may have additional (e.g., downstream) open reading frames, e.g., to encode further antigens or to encode accessory polypeptides.

In some aspects, the saRNA molecule further includes (1) an alphavirus 5′ replication recognition sequence, and (2) an alphavirus 3′ replication recognition sequence. In some aspects, the 5′ sequence of the self-amplifying RNA molecule is selected to ensure compatibility with the encoded replicase.

Optionally, self-amplifying RNA molecules described herein may also be designed to induce production of infectious viral particles that are attenuated or virulent, or to produce viral particles that are capable of a single round of subsequent infection.

In some aspects, the saRNA molecule is alphavirus-based. Alphaviruses include a set of genetically, structurally, and serologically related arthropod-borne viruses of the Togaviridae family. Exemplary viruses and virus subtypes within the alphavirus genus include Sindbis virus, Semliki Forest virus, Ross River virus, and Venezuelan equine encephalitis virus. As such, the self-amplifying RNA described herein may incorporate an RNA replicase derived from any one of semliki forest virus (SFV), sindbis virus (SIN), Venezuelan equine encephalitis virus (VEE), Ross-River virus (RRV), or other viruses belonging to the alphavirus family. In some aspects, the self-amplifying RNA described herein may incorporate sequences derived from a mutant or wild-type virus sequence, e.g., the attenuated TC83 mutant of VEEV has been used in saRNAs.

Alphavirus-based saRNAs are (+)-stranded saRNAs that may be translated after delivery to a cell, which leads to translation of a replicase (or replicase-transcriptase). The replicase is translated as a polyprotein which auto-cleaves to provide a replication complex which creates genomic (−)-strand copies of the (+)-strand delivered RNA. These (−)-strand transcripts may themselves be transcribed to give further copies of the (+)-stranded parent RNA and also to give a subgenomic transcript which encodes the desired gene product. Translation of the subgenomic transcript thus leads to in situ expression of the desired gene product by the infected cell. Suitable alphavirus saRNAs may use a replicase from a sindbis virus, a semliki forest virus, an eastern equine encephalitis virus, a Venezuelan equine encephalitis virus, or mutant variants thereof.

In some aspects, the self-amplifying RNA molecule is derived from or based on a virus other than an alphavirus, such as a positive-stranded RNA virus, and in particular a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375). In some aspects, one or more of the alphaviruses in the list may be excluded.

In some aspects, the self-amplifying RNA molecules described herein are larger than other types of RNA (e.g., mRNA). Typically, the self-amplifying RNA molecules described herein include at least about 4 kb. For example, the self-amplifying RNA may be equal to any one of, at least any one of, at most any one of, or between any two of 3 kb, 4 kb, 5 kb, 6 kb, 7 kb, 8 kb, 9 kb, 10 kb, 11 kb, 12 kb, 13 kb, 14 kb, 15 kb, 16 kb. In some instances the self-amplifying RNA may include at least about 5 kb, at least about 6 kb, at least about 7 kb, at least about 8 kb, at least about 9 kb, at least about 10 kb, at least about 11 kb, at least about 12 kb, or more than 12 kb. In certain examples, the self-amplifying RNA is about 4 kb to about 12 kb, about 5 kb to about 12 kb, about 6 kb to about 12 kb, about 7 kb to about 12 kb, about 8 kb to about 12 kb, about 9 kb to about 12 kb, about 10 kb to about 12 kb, about 11 kb to about 12 kb, about 5 kb to about 11 kb, about 5 kb to about 10 kb, about 5 kb to about 9 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb.

In some aspects, the self-amplifying RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-amplifying RNA may then be produced as a fusion polypeptide or engineered in such a manner to result in separate polypeptide or peptide sequences.

In some aspects, the self-amplifying RNA described herein may encode one or more polypeptide antigens that include a range of epitopes. In some aspects, the self-amplifying RNA described herein may encode epitopes capable of eliciting either a helper T-cell response or a cytotoxic T-cell response or both.

### III. Lipids and Lipid Nanoparticles

In an aspect, the compositions disclosed herein comprise lipids. For example, compositions can include lipids and mRNA (e.g., modRNA), and the lipids and mRNA (e.g., modRNA) can together form nanoparticles, thereby producing mRNA-containing nanoparticles comprising lipids. The lipids can encapsulate or associate with the mRNA in the form of a lipid nanoparticle (LNP) to aid stability, cell entry, and intracellular release of the RNA/lipid nanoparticles.

In some instances, a LNP comprises a micelle, a solid lipid nanoparticle, a nanoemulsion, a liposome, etc., or a combination thereof.

The lipid component of a LNP may include, for example, a cationic lipid, a phospholipid (such as an unsaturated lipid, e.g., DOPE or DSPC), a polymer-lipid conjugate (e.g., a PEGylated lipid), a structural lipid, an ionizable lipid, a neutral lipid, or any combination thereof. The elements of the lipid component may be provided in specific fractions. Suitable cationic lipids, phospholipids, polymer-lipid conjugates, structural lipids, ionizable lipids, and neutral lipids for the methods of the present disclosure are further disclosed herein.

In some aspects, the lipid component of a LNP includes any one or more of a cationic lipid, a phospholipid, a polymer-lipid conjugate, a structural lipid, an ionizable lipid, and/or a neutral lipid. In certain aspects, the lipid component of the lipid nanoparticle includes about 0 mol % to about 60 mol % cationic lipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % cationic lipid); about 0 mol % to about 60 mol % phospholipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % phospholipid); about 0 mol % to about 60 mol % structural lipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 mol % structural lipid); about 0 mol % to about 60 mol % of polymer-lipid conjugate (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % polymer-lipid conjugate); about 0 mol % to about 60 mol % ionizable lipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % ionizable lipid); and/or about 0 mol % to about 60 mol % neutral lipid (e.g., at least about, at most about, between any two of, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % neutral lipid). The LNP can have any amount of the foregoing lipid components, provided that the total mol % does not exceed 100%. As used herein, “mol percent” refers to a component's molar percentage relative to total mols of all lipid components in the LNP (i.e., total mols of cationic lipid(s), the neutral lipid, the steroid and the polymer conjugated lipid).

In some aspects, the lipid component of the lipid nanoparticle includes about 35 mol % to about 55 mol % compound of cationic lipid, about 5 mol % to about 25 mol % phospholipid, about 30 mol % to about 50 mol % structural lipid, and about 0 mol % to about 10 mol % of polymer-lipid conjugate. In a particular aspect, the lipid component includes about 50 mol % said cationic lipid, about 10 mol % phospholipid, about 40 mol % structural lipid, and about 1.5 mol % of polymer-lipid conjugate. In another particular aspect, the lipid component includes about 40 mol % said cationic lipid, about 20 mol % phospholipid, about 40 mol % structural lipid, and about 1.5 mol % of polymer-lipid conjugate. In a particular aspect, the lipid component of the lipid nanoparticle includes cationic lipid, phospholipid, structural lipid, and polymer-lipid conjugate at a molar ratio of about 47.5:10:40.7:1.8.

In some aspects, the lipid component of the lipid nanoparticle includes about 0 mol % to about 10 mol % compound of cationic lipid, about 40 mol % to about 60 mol % phospholipid, and about 40 mol % to about 60 mol % structural lipid. In a particular aspect, the lipid component includes about 2 mol % said cationic lipid, about 49 mol % phospholipid, and about 49 mol % structural lipid. In a particular aspect, the lipid component of the lipid nanoparticle includes cationic lipid, phospholipid, and structural lipid at a molar ratio of about 1.8:49.1:49.1.

In some aspects, the phospholipid may be DOPE or DSPC. In other aspects, the polymer-lipid conjugate may be PEG-DMG and/or the structural lipid may be cholesterol. In other aspects, the polymer-lipid conjugate may be PEG-2000 DMG and/or the structural lipid may be cholesterol.

In some aspects, the lipid nanoparticle includes:
i) between 40 and 50 mol percent of a cationic lipid;
ii) a phospholipid and/or a neutral lipid;
iii) a structural lipid;
iv) a polymer conjugated lipid; and
v) a therapeutic agent (namely, RNA) encapsulated within or associated with the lipid nanoparticle.

In some aspects, the lipid nanoparticle includes:
i) between 0 and 10 mol % of a cationic lipid;
ii) a phospholipid and/or a neutral lipid; and
iii) a steroid.

In some aspects, the lipid nanoparticle comprises from 41 to 50 mol percent, from 42 to 50 mol percent, from 43 to 50 mol percent, from 44 to 50 mol percent, from 45 to 50 mol percent, from 46 to 50 mol percent, or from 47 to 50 mol percent of the cationic lipid. In certain specific aspects, the lipid nanoparticle comprises at least about, at most about, between any two of, or exactly 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50 mol percent of the cationic lipid.

In other aspects, the lipid nanoparticle comprises from 0 to 10 mol percent of the cationic lipid. In certain specific aspects, the lipid nanoparticle comprises at least about, at most about, between any two of, or exactly 0, 1, 2, 3, 4, 5, 6, 7, 8, 9, or 10 mol percent of the cationic lipid.

In some aspects, the phospholipid and/or neutral lipid is present in a concentration ranging from 5 to 15 mol percent, 7 to 13 mol percent, or 9 to 11 mol percent. In certain aspects, the phospholipid and/or neutral lipid is present in a concentration of at least about, at most about, in between any two of, or exactly 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, or 15 mol percent. In certain specific aspects, the phospholipid and/or neutral lipid is present in a concentration of about 9.5, 10 or 10.5 mol percent.

In other aspects, the phospholipid and/or neutral lipid is present in a concentration ranging from 40 to 60 mol %. In certain aspects, the phospholipid and/or neutral lipid is present in a concentration of at least about, at most about, in between any two of, or exactly 40, 41, 42, 43, 44, 45, 46, 47, 48, 49, 50, 51, 52, 53, 54, 55, 56, 57, 58, 59, or 60 mol percent. In certain specific aspects, the phospholipid and/or neutral lipid is present in a concentration of about 48, 49, or 50 mol percent.

In some aspects, the molar ratio of the cationic lipid to the phospholipid and/or neutral lipid ranges from about 4.1:1.0 to about 4.9:1.0, from about 4.5:1.0 to about 4.8:1.0, or from about 4.7:1.0 to 4.8:1.0. In other aspects, the molar ratio of the phospholipid and/or neutral lipid to the cationic lipid is 1:4.1, 1:4.2, 1:4.3, 1:4.4, 1:4.5, 1:4.6, 1:4.7, 1:4.8, or 1:4.9.

In some aspects, the structural lipid is a steroid. In some aspects, the steroid is cholesterol. In some aspects, the structural lipid is present in a concentration ranging from 39 to 49 molar percent, 40 to 46 molar percent, from 40 to 44 molar percent, from 40 to 42 molar percent, from 42 to 44 molar percent, or from 44 to 46 molar percent. In certain specific aspects, the structural lipid is present in a concentration of at least about, at most about, in between any two of, or exactly 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, or 49 mol percent. In certain specific aspects, the structural lipid is present in a concentration of 40, 41, 42, 43, 44, 45, or 46 molar percent.

In other aspects, the structural lipid is present in a concentration ranging from 40 to 60 mol %. In certain aspects, the structural lipid is present in a concentration of at least about, at most about, in between any two of, or exactly 40, 41, 42, 43, 44, 45, 46, 47, 48, 49, 50, 51, 52, 53, 54, 55, 56, 57, 58, 59, or 60 mol percent. In certain specific aspects, the structural lipid is present in a concentration of about 48, 49, or 50 mol percent.

In certain aspects, the molar ratio of cationic lipid to the structural lipid ranges from 1.0:0.9 to 1.0:1.2, or from 1.0:1.0 to 1.0:1.2, e.g., 1:0.9, 1:1, 1:1.1, or 1:1.2.

In preferred aspects, the cationic lipid is a compound having the following structure (IE):

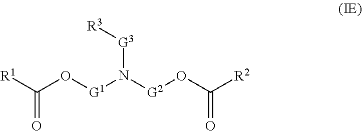

or a pharmaceutically acceptable salt or stereoisomer thereof, wherein:
G¹ and G² are each independently unsubstituted alkylene;
G³ is unsubstituted C1-C12 alkylene;
R¹ and R² are each independently C₆-C₂₄ alkyl;
R³ is OR⁵, CN, —C(═O)OR⁴, —OC(═O)R⁴ or NR⁵C(═O)R⁴;
R⁴ is C₁-C₁₂ alkyl; and
R⁵ is H or C1-C6 alkyl.

In some aspects, the compound includes the following structure:

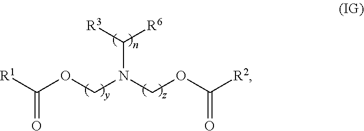

wherein R⁶ is, at each occurrence, H; n is an integer ranging from 2 to 12; and y and z are each independently integers ranging from 6 to 9. In some aspects, n is 3, 4, 5 or 6. 4. In some aspects, y and z are each 6. In some aspects, y and z are each 9. In some aspects, R¹ and R² each, independently has the following structure wherein: R⁷ª and R^{7b} are, at each occurrence, independently H or C1-C12 alkyl; and a is an integer from 2 to 12, wherein R^{7a}, R^{7b} and a are each selected such that R¹ and R² each independently comprise from 6 to 20 carbon atoms. In some aspects, a is an integer from 8 to 12. In some aspects, at least one occurrence of R⁷ª is H. In some aspects, R⁷ª is H at each occurrence. In some aspects, at least one occurrence of R^{7b} is C1-C8 alkyl. In some aspects, C1-C8 alkyl is methyl, ethyl, n-propyl, iso-propyl, n-butyl, iso-butyl, tert-butyl, n-hexyl or n-octyl. In some aspects, R³ is OH. In some aspects, R³ is CN. In some aspects, R³ is —C(═O)OR⁴, —OC(═O) R⁴ or NHC(═O) R⁴. In some aspects, R⁴ is methyl or ethyl. In some aspects, the compound has the following structure:

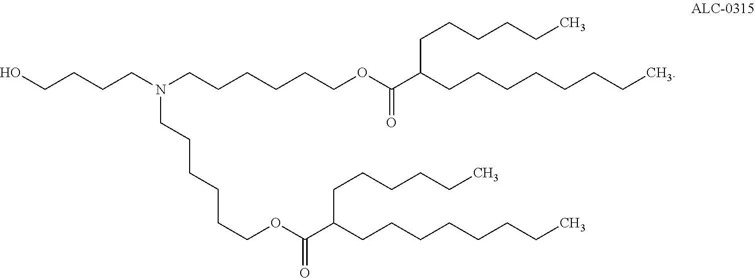 [SMILES: CCCCCCCCC(CCCCCC)C(=O)OCCCCCCN(CCCCO)CCCCCCOC(=O)C(CCCCCC)CCCCCCCC]

Additional exemplary ionizable lipids include:

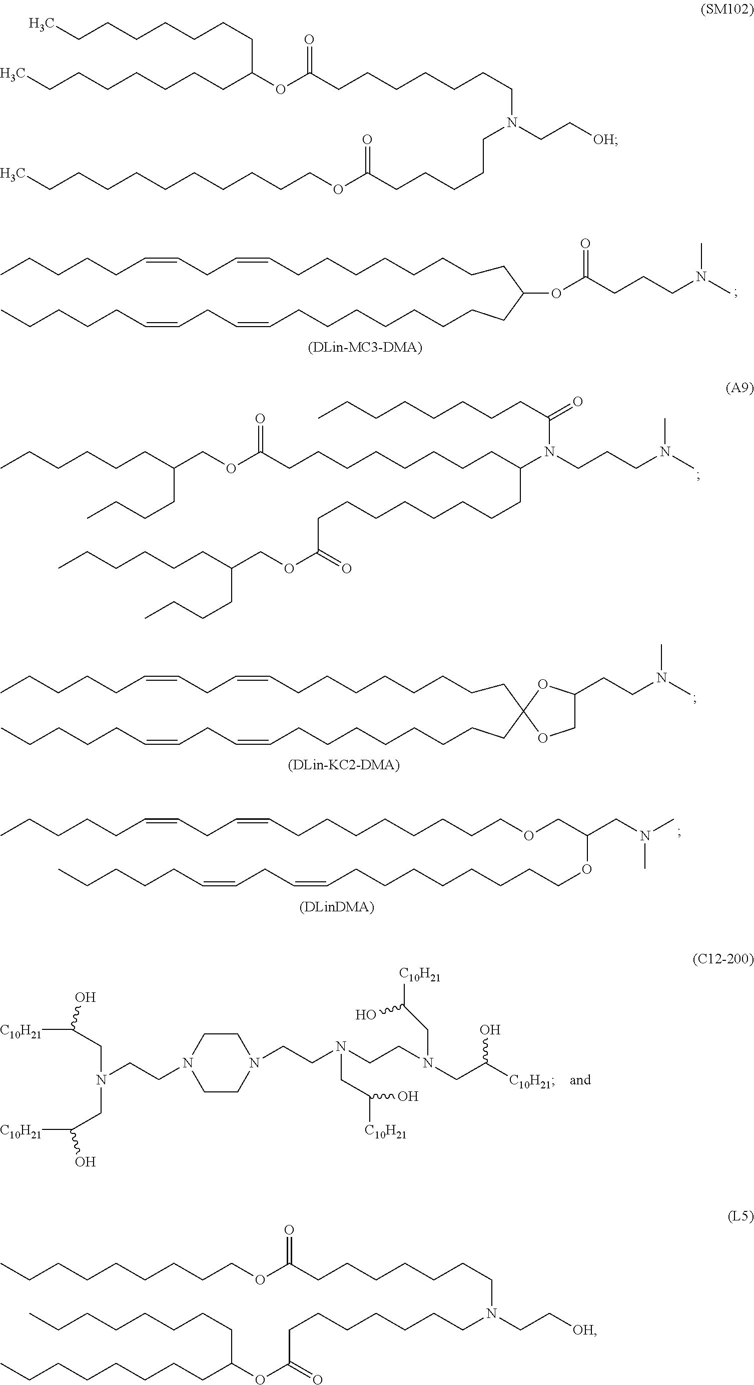

which are known in the art.

The lipid component of a lipid nanoparticle composition may include one or more molecules comprising a polymer such as a polyethylene glycol, e.g., PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some aspects, a PEG lipid may be PEG-c-DOMG, PEG-DMG, PEG-DLPE, PEG-DMPE, PEG-DPPC, or a PEG-DSPE lipid. As used herein, the term “PEG lipid” refers to polyethylene glycol (PEG)-modified lipids. Non-limiting examples of PEG lipids include PEG-modified phosphatidylethanolamine and phosphatidic acid, PEG-ceramide conjugates (e.g., PEG-CerCI4 or PEG-CerC20), PEG-modified dialkylamines and PEG-modified 1,2-diacyloxypropan-3-amines. Such lipids are also referred to as PEGylated lipids. In some aspects, a PEG lipid can be PEG-c-DOMG, PEG-DMG, PEG-DLPE, PEG-DMPE, PEG-DPPC, or a PEG-DSPE lipid. In some aspects, the PEG-modified lipids are a modified form of PEG DMG.

In some aspects, the PEG-modified lipid is PEG lipid with the formula (IV):

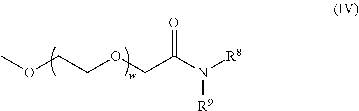

wherein R8 and R9 are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

In some aspects, the polymer-conjugated lipid is a polyoxazoline (POZ) lipid comprising the formula (IV):

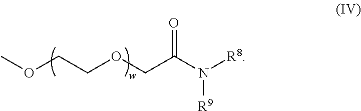

POZ is known in the art and is described in WO/2020/264505, PCT/US2020/040140, filed on Jun. 29, 2020.

In some aspects, the PEGylated lipid has the following structure (II):

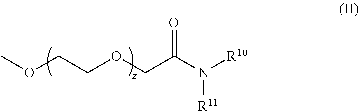

or a pharmaceutically acceptable salt, tautomer or stereoisomer thereof, wherein: R¹⁰ and R¹¹ are each independently a straight or branched, saturated or unsaturated alkyl chain containing from 10 to 30 carbon atoms, wherein the alkyl chain is optionally interrupted by one or more ester bonds; and z has a mean value ranging from 30 to 60; provided that R¹⁰ and R¹¹ are not both n-octadecyl when z is 42. In some aspects of the PEGylated lipid, R¹⁰ and R¹¹ are each independently straight, saturated alkyl chains containing from 12 to 16 carbon atoms. In some aspects, of the pegylated lipid z is about 45.

In some aspects, the PEGylated lipid has one of the following structures:

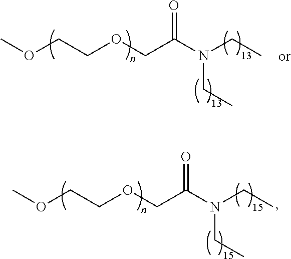

wherein n has a mean value ranging from 40 to 50. In a preferred aspect, the composition comprises the ALC-315 cationic lipid described above and a PEGylated lipid having one of the following structures:

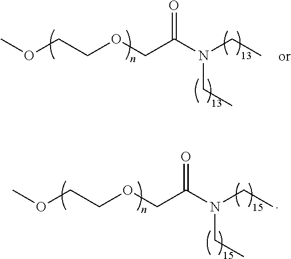

In some aspects of the PEGylated lipid described above, R¹⁰ and R¹¹ are each independently a straight or branched, saturated or unsaturated alkyl chain containing 12 carbon atoms. In some aspects of the PEGylated lipid described above, R¹⁰ and R¹¹ are each independently a straight or branched, saturated or unsaturated alkyl chain containing 14 carbon atoms. In some aspects of the PEGylated lipid described above, R¹⁰ and R¹¹ are each independently a straight or branched, saturated or unsaturated alkyl chain containing 16 carbon atoms. Further exemplary lipids and related formulations thereof are disclosed for example, in U.S. Pat. No. 9,737,619, filed Feb. 14, 2017, U.S. Pat. No. 10,166,298, filed Oct. 28, 2016, and International Patent Application No. PCT/US2017/058619, filed Oct. 26, 2017, the disclosures of which are incorporated herein by reference in their entirety.

In some aspects, the ionizable lipid is a compound of Formula (IL-I):

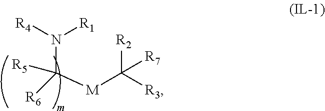

or their N-oxides, or salts or isomers thereof, wherein:
Rⁱ is selected from the group consisting of C₅₋₃₀ alkyl, C₅₋₂₀ alkenyl, —R*YR″, —YR″, and —R″M′R′; R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle; R⁴ is selected from the group consisting of hydrogen, a C₃₋₆ carbocycle, —(CH₂)nQ, —(CH₂)nCHQR, -CHQR, -CQ(R)₂, and unsubstituted C₁₋₆ alkyl, where Q is selected from a carbocycle, heterocycle, —OR, —O(CH₂)nN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)Re, N(R)S(O)₂R⁸, —O(CH₂)nOR, —N(R)C(═NR₉)N(R)₂, —N(R)C(═CHR₉)N(R)₂, —OC(O)N(R)₂J-N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR) C(═CHR₉) N(R)₂, —C(═NR₉)N(R)₂, —C(═NR₉)R, —C(O)N(R)OR, and —C(R)N(R)₂C(O)OR, and each n is independently selected from 1, 2, 3, 4, and 5; each R⁵ is independently selected from the group consisting of C₁₋₃ alkyl, C₂-3 alkenyl, and H; each R^{e} is independently selected from the group consisting of C₁₋₃ alkyl, C₂-3 alkenyl, and H; M and M′ are independently selected from —C(O)O—, —OC(O)—, —OC(O)-M″-C(O)O—, —C(O) N(R′)—, —N(R′) C(O)—, —C(O)—, —C(S)—, —C(S)S—, —SC(S)—, —CH(OH)—, —P(O) (OR′)O—, —S(O)₂—, —S—S—, an aryl group, and a heteroaryl group, in which M″ is a bond, C₁₋₁₃ alkyl or C₂₋₁₃ alkenyl; R⁷ is selected from the group consisting of C₁₋₃ alkyl, C₂-3 alkenyl, and H; R^{e} is selected from the group consisting of C₃₋₆ carbocycle and heterocycle; R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle; each R is independently selected from the group consisting of C₁₋₃ alkyl, C₂₋₃ alkenyl, and H; each R′ is independently selected from the group consisting of C₁₋₁₅ alkyl, C₂₋₁₅ alkenyl, —R*YR″, —YR″, and H; each R″ is independently selected from the group consisting of C₃₋₁₅ alkyl and C₃₋₁₅ alkenyl; each R* is independently selected from the group consisting of C₁₋₁₂ alkyl and C₂₋₁₂ alkenyl; each Y is independently a C₃₋₆ carbocycle; each X is independently selected from the group consisting of F, Cl, Br, and I; and m is selected from 5, 6, 7, 8, 9, 10, 11, 12, and 13; and wherein when R⁴ is —(CH₂)nQ, —(CH₂)nCHQR, -CHQR, or -CQ(R)₂, then (i) Q is not-N(R) 2 when n is 1, 2, 3, 4 or 5, or (ii) Q is not 5, 6, or 7-membered heterocycloalkyl when n is 1 or 2.

In preferred aspects, the composition further includes a nucleic acid. In preferred aspects, the nucleic acid comprises messenger RNA. In some aspects, the composition further includes one or more excipients selected from neutral lipids and steroids. In some aspects, the composition comprises one or more neutral lipids selected from DSPC, DPPC, DMPC, DOPC, POPC, DOPE and SM. Preferably, in some aspects, the neutral lipid is DSPC. Preferably, in some aspects, the steroid is cholesterol.

A LNP may include one or more components described herein. In some aspects, the LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body. Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain aspects, a LNP may include an mRNA encoding a polypeptide of interest capable of being translated within a cell to produce the polypeptide of interest. Such a composition may be designed to be specifically delivered to a particular organ. In some aspects, a composition may be designed to be specifically delivered to a mammalian liver. In some aspects, a composition may be designed to be specifically delivered to a lymph node. In some aspects, a composition may be designed to be specifically delivered to a mammalian spleen.

In some aspects, a polymer may be included in and/or used to encapsulate or partially encapsulate a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl (meth)acrylate) (PMMA), poly(ethyl (meth)acrylate), poly(butyl (meth)acrylate), poly(isobutyl (meth)acrylate), poly(hexyl(meth)acrylate), poly(isodecyl(meth)acrylate), poly(lauryl (meth)acrylate), poly(phenyl (meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho) esters, poly(butyric acid), poly(valeric acid), poly(lactide-co-caprolactone), trimethylene carbonate, poly(N-acryloylmorpholine) (PACM), poly(2-methyl-2-oxazoline) (PMOX), poly(2-ethyl-2-oxazoline) (PEOZ), and polyglycerol.

In some aspects, a surface altering agent may be included in and/or used to encapsulate or partially encapsulate a LNP. Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN®20], polyoxy ethylene sorbitan [TWEEN® 60], polyoxy ethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®. An exemplary free radical scavenger includes butylated hydroxytoluene (BHT or butylhydroxytoluene) or deferoxamine. In some preferred aspects, the composition does not include a preservative.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, the concentration of the buffer in the composition is about 10 mM. For example, the buffer concentration can be equal to any one of, at least any one of, at most any one of, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. In specific aspects, the buffer concentration is 10 mM. The buffer can be at a neutral pH, pH 6.5 to 8.5, pH 7.0 to pH 8.0, or pH 7.2 to pH 7.6. For example, the buffer can be at pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

In some aspects, the formulation including a LNP may further include a salt, such as a chloride salt. In some aspects, the formulation including a LNP may further includes a sugar such as a disaccharide. In some aspects, the formulation further includes a sugar but not a salt, such as a chloride salt. In some aspects, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some aspects, the structural lipid is a steroid. In some aspects, the structural lipid is cholesterol. In some aspects, the structural lipid is an analog of cholesterol. In some aspects, the structural lipid is alpha-tocopherol.

In some aspects, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some aspects, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some aspects, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. Phospholipids also include phosphosphingolipid, such as sphingomyelin. In some aspects, a phospholipid useful or potentially useful in the present invention is an analog or variant of DSPC.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21 st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some aspects, the composition may comprise a pharmaceutically acceptable carrier and/or vehicle. In some aspects, the composition may further include pyrogen-free water; isotonic saline or buffered (aqueous) solutions, e.g., phosphate, citrate etc. buffered solutions. In some aspects, the composition may include water and/or a buffer containing a sodium salt, such as at least 50 mM of a sodium salt, a calcium salt, in some aspects at least 0.01 mM of a calcium salt, and optionally a potassium salt, in some aspects at least 3 mM of a potassium salt. In some aspects the sodium, calcium and, optionally, potassium salts may occur in the form of their halogenides, e.g., chlorides, iodides, or bromides, in the form of their hydroxides, carbonates, hydrogen carbonates, or sulfates, etc. Examples of sodium salts include e.g., NaCl, NaI, NaBr, Na₂CO₃, NaHCO₃, Na₂SO₄, examples of the potassium salts include e.g., KCl, KI, KBr, K₂CO₃, KHCO₃, K₂SO₄, and examples of calcium salts include e.g., CaCl₂), CaI₂, CaBr₂, CaCO₃, CaSO₄, Ca(OH)₂. In some aspects, organic anions of the aforementioned cations may be contained in the buffer. In some aspects, the composition may include salts selected from sodium chloride (NaCl), calcium chloride (CaCl₂)) and potassium chloride (KCl), wherein further anions may be present additional to the chlorides. CaCl₂) can also be replaced by another salt like KCl. In some aspects, the injection buffer may be hypertonic, isotonic or hypotonic with reference to the specific reference medium, i.e. the buffer may have a higher, identical or lower salt content with reference to the specific reference medium, wherein such concentrations of the afore mentioned salts may be used, which minimizes damage of cells due to osmosis or other concentration effects. The concentration of the salts in the composition can be about 70 mM to about 140 mM. For example, the salt concentration can be equal to any one of, at least any one of, at most any one of, or between any two of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM, or any range or value derivable therein. The salt can be at a neutral pH, pH 6.5 to 8.5, pH 7.0 to pH 8.0, or pH 7.2 to pH 7.6. For example, the salt can be at a pH equal to any one of, at least any one of, at most any one of, or between any two of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein.

In some aspects, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some aspects, a pharmaceutically acceptable excipient is at least 95%, at least 96%, at least 97%, at least 98%, at least 99%, or 100% pure. Examples of excipients, which refer to ingredients in the compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, or colorants. Preservatives for use in the compositions disclosed herein include but are not limited to benzalkonium chloride, chlorobutanol, paraben and thimerosal. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato; and celluloses such as microcrystalline cellulose. The amount of diluent in the composition can range from about 10% to about 90% by weight of the total composition, about 25% to about 75%, about 30% to about 60% by weight, or about 12% to about 60%.

In some aspects, an excipient is approved for use in humans and for veterinary use. In some aspects, an excipient is approved by United States Food and Drug Administration. In some aspects, an excipient is pharmaceutical grade. In some aspects, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

The pH and exact concentration of the various components in a pharmaceutical composition are adjusted according to well-known parameters. The use of such media and agents for pharmaceutical active substances is well known in the art. Except insofar as any conventional media or agent is incompatible with the active ingredients, its use in immunogenic and therapeutic compositions is contemplated.

In certain aspects, the lipid nanoparticles and/or pharmaceutical compositions of the disclosure are refrigerated or frozen for storage and/or shipment (e.g., being stored at a temperature of 10° C. or lower, such as a temperature at about 4°, a temperature between about −150° C. and about 10° C. (e.g., about 10° C., 9° C., 8° C., 7° C., 6° C., 5° C., 4° C., 3° C., 2° C., 1° C., 0° C., −1° C., −2° C., −3° C., −4° C., −5° C., −6° C., −7° C., −8° C., −9° C., −10° C., −15° C., −20° C., −25° C., −30° C., −40° C., −50° C., −60° C., −70° C., −80° C., −90° C., −130° C. or −150° C.) or a temperature between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., −80° C. or −90° C.

In certain aspects, the disclosure also relates to a method of increasing stability of the lipid nanoparticles by adding an effective amount of an amphiphilic polymer and by storing the lipid nanoparticles and/or pharmaceutical compositions thereof at a temperature of 10° C. or lower, such as a temperature at about 4° C., a temperature between about −150° C. and about 10° C. (e.g., about 10° C., 9° C., 8° C., 7° C., 6° C., 5° C., 4° C., 3° C., 2° C., 1° C., 0° C., −1° C., −2° C., −3° C., −4° C., −5° C., −6° C., −7° C., −8° C., −9° C., −10° C., −15° C., −20° C., −25° C., −30° C., −40° C., −50° C., −60° C., −70° C., −80° C., −90° C., −130° C. or −150° C.) or a temperature between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.).

The chemical properties of the LNP, LNP suspension, lyophilized LNP composition, or LNP formulation of the present disclosure may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some aspects, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain aspects, the mean size of a LNP may be from about 70 nm to about 100 nm. In a particular aspect, the mean size may be about 80 nm. In other aspects, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some aspects, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some aspects, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some aspects, the encapsulation efficiency may be at least 80%. In certain aspects, the encapsulation efficiency may be at least 90%. In some aspects, the LNP encapsulation efficiency of the LNP, LNP suspension, lyophilized LNP composition, or LNP formulation of the present disclosure produced in the presence of blank LNPs (e.g., lipid nanoparticles comprising the lipids listed in herein but not encapsulating any nucleic acid) is about 50% or higher, about 55% or higher, about 60% or higher, about 65% or higher, about 70% or higher, about 75% or higher, about 80% or higher, about 8% or higher, about 90% or higher, about 91% or higher, about 92% or higher, about 93% or higher, about 94% or higher, about 95% or higher, about 96% or higher, about 97% or higher, about 98% or higher, or about 99% or higher than the LNP encapsulation efficiency of the LNP, LNP suspension, lyophilized LNP composition, or LNP formulation produced by a comparable method in the presence of a lesser concentration of blank LNPs or in the absence of blank LNPs.

In some aspects, electrophoresis (e.g., capillary electrophoresis) or chromatography (e.g., reverse phase liquid chromatography) may be used to examine the mRNA integrity.

In some aspects, the LNP integrity of the LNP, LNP suspension, lyophilized LNP composition, or LNP formulation of the present disclosure produced in the presence of blank LNPs (e.g., lipid nanoparticles comprising the lipids listed in herein but not encapsulating any nucleic acid) is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher than the LNP integrity of the LNP, LNP suspension, lyophilized LNP composition, or LNP formulation produced by a comparable method in the presence of a lesser concentration of blank LNPs or in the absence of blank LNPs.

In some aspects, the LNP integrity of the LNP, LNP suspension, lyophilized LNP composition, or LNP formulation of the present disclosure produced in the presence of blank LNPs (e.g., lipid nanoparticles comprising the lipids listed in herein but not encapsulating any nucleic acid) is higher than the LNP integrity of the LNP, LNP suspension, lyophilized LNP composition, or LNP formulation produced by a comparable method in the presence of a lesser concentration of blank LNPs or in the absence of blank LNPs by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more. In some aspects, the Txo % of the LNP, LNP suspension, lyophilized LNP composition, or LNP formulation of the present disclosure produced in the presence of blank LNPs (e.g., lipid nanoparticles comprising the lipids listed in herein but not encapsulating any nucleic acid) is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer than the Txo % of the LNP, LNP suspension, lyophilized LNP composition, or LNP formulation produced by a comparable method in the presence of a lesser concentration of blank LNPs or in the absence of blank LNPs. In some aspects, the Txo % of the LNP, LNP suspension, lyophilized LNP composition, or LNP formulation of the present disclosure produced in the presence of blank LNPs (e.g., lipid nanoparticles comprising the lipids listed in herein but not encapsulating any nucleic acid) is longer than the Txo % of the LNP, LNP suspension, lyophilized LNP composition, or LNP formulation produced by a comparable method in the presence of a lesser concentration of blank LNPs or in the absence of blank LNPs by about 5% or higher, about 10% or more, about 15% or more, about 20% or more, about 30% or more, about 40% or more, about 50% or more, about 60% or more, about 70% or more, about 80% or more, about 90% or more, about 1 fold or more, about 2 folds or more, about 3 folds or more, about 4 folds or more, about 5 folds or more.

In some aspects, the T1/2 of the LNP, LNP suspension, lyophilized LNP composition, or LNP formulation of the present disclosure produced in the presence of blank LNPs (e.g., lipid nanoparticles comprising the lipids listed in herein but not encapsulating any nucleic acid) is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer than the T1/2 of the LNP, LNP suspension, lyophilized LNP composition, or LNP formulation produced by a comparable method in the presence of a lesser concentration of blank LNPs or in the absence of blank LNPs.

In some aspects, the T1/2 of the LNP, LNP suspension, lyophilized LNP composition, or LNP formulation of the present disclosure produced in the presence of blank LNPs (e.g., lipid nanoparticles comprising the lipids listed in herein but not encapsulating any nucleic acid) is longer than the T1/2 of the LNP, LNP suspension, lyophilized LNP composition, or LNP formulation produced by a comparable method in the presence of a lesser concentration of blank LNPs or in the absence of blank LNPs by about 5% or higher, about 10% or more, about 15% or more, about 20% or more, about 30% or more, about 40% or more, about 50% or more, about 60% or more, about 70% or more, about 80% or more, about 90% or more, about 1 fold or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T1/2” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (i.e. pharmaceutical substance) and other elements (e.g., lipids) in a LNP may also vary. In some aspects, the wt/wt ratio of the lipid component to a therapeutic and/or prophylactic in a LNP may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain aspects, the wt/wt ratio is about 20:1. The amount of a therapeutic and/or prophylactic in a LNP may, for example, be measured using absorption spectroscopy (e.g., ultraviolet-visible spectroscopy).

In some aspects, the mRNA to lipid ratio in the LNP (i.e., N:P, where N represents the moles of cationic lipid and P represents the moles of phosphate present as part of the nucleic acid backbone) range from 2:1 to 30:1, for example 3:1 to 22:1. In other aspects, N:P ranges from 6:1 to 20:1 or 2:1 to 12:1. Exemplary N:P ranges include about 3:1. About 6:1, about 12:1 and about 22:1.

### IV. RNA Transcription and Encapsulation

In some aspects, the RNA disclosed herein is produced by in vitro transcription. “In vitro transcription” or “IVT” refers to the process whereby transcription occurs in vitro in a non-cellular system to produce a synthetic RNA product for use in various applications, including, e.g., production of protein or polypeptides. Such synthetic RNA products can be translated in vitro or introduced directly into cells, where they can be translated. Such synthetic RNA products include, e.g., but are not limited to mRNAs, saRNAs, antisense RNA molecules, shRNA molecules, long non-coding RNA molecules, ribozymes, aptamers, guide RNAs (e.g., for CRISPR), ribosomal RNAs, small nuclear RNAs, small nucleolar RNAs, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In some aspects, starting material for IVT can include linearized DNA template, nucleotides, RNase inhibitor, pyrophosphatase, and/or T7 RNA polymerase. In some aspects, the IVT process is conducted in a bioreactor. The bioreactor can comprise a mixer. In some aspects, nucleotides can be added into the bioreactor throughout the IVT process.

In some aspects, one or more post-IVT agents are added into the IVT mixture comprising RNA in the bioreactor after the IVT process. Exemplary post-IVT agents can include DNAse I configured to digest the linearized DNA template, and proteinase K configured to digest DNAse I and T7 RNA polymerase. In some aspects, the post-IVT agents are incubated with the mixture in the bioreactor after IVT. In some aspects, the bioreactor can contain any one of, at least any one of, at most any one of, or between any two of 60, 70, 80, 90, 100, 110, 120, 130, 140, 150,160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 or more liters IVT mixture. The IVT mixture can have an RNA concentration at any one of, at least any one of, at most any one of, or between any two of 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0, 10, 11, 12, 13, 14, 15, 16, 17, 18, 19, 20, 30, 40, 50, 60, 70, 80, 90, and 100 mg/mL or more RNA.

In some aspects, the IVT mixture can include residual spermidine, residual DNA, residual proteins, peptides, HEPES, EDTA, ammonium sulfate, cations (e.g., Mg²⁺, Na⁺, Ca²⁺), RNA fragments, residual nucleotides, free phosphates, or any combinations thereof.

In some aspects, at least a portion of the IVT mixture is filtered. The IVT mixture can be filtered via ultrafiltration and/or diafiltration to remove at least some impurities from the IVT mixture and/or to change buffer solution for the at least a portion of IVT mixture to produce a concentrated RNA solution as a retentate.

In some aspects, both “ultrafiltration” and “diafiltration” refer to a membrane filtration process. Ultrafiltration typically uses membranes having pore sizes any one of, at least any one of, at most any one of, or between any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 μm. In some aspects, ultrafiltration membranes are typically classified by molecular weight cutoff (MWCO) rather than pore size. For example, the MWCO can be any one of, at least any one of, at most any one of, or between any two of 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, and 10000 kDa. A skilled artisan will understand that filtration membranes can be of different suitable materials, including, e.g., polymeric, cellulose, ceramic, etc., depending upon the application. In some aspects, membrane filtration may be more desirable for large volume purification process.

In some aspects, ultrafiltration and diafiltration of the IVT mixture for purifying RNA can include (1) Direct Flow Filtration (DFF), also known as “dead-end” filtration, that applies a feed stream perpendicular to the membrane face and attempts to pass 100% of the fluid through the membrane, and/or (2) Tangential Flow Filtration (TFF), also known as crossflow filtration, where a feed stream passes parallel to the membrane face as one portion passes through the membrane (permeate) while the remainder (retentate) is retained and/or recirculated back to the feed tank.

In some aspects, the filtering of the IVT mixture is conducted via TFF that comprises an ultrafiltration step, a first diafiltration step, and a second diafiltration step. In some aspects, the first diafiltration step is conducted in the presence of ammonium sulfate. The first diafiltration step can be configured to remove a majority of impurities from the IVT mixture. In some aspects, the second diafiltration step is conducted without ammonium sulfate. The second diafiltration step can be configured to transfer the RNA into a DS buffer formulation.

A filtration membrane with an appropriate MWCO may be selected for the ultrafiltration in the TFF process. The MWCO of a TFF membrane determines which solutes can pass through the membrane into the filtrate and which are retained in the retentate. The MWCO of a TFF membrane may be selected such that substantially all of the solutes of interest (e.g., desired synthesized RNA species) remains in the retentate, whereas undesired components (e.g., excess ribonucleotides, small nucleic acid fragments such as digested or hydrolyzed DNA template, peptide fragments such as digested proteins and/or other impurities) pass into the filtrate. In some aspects, the retentate comprising desired synthesized RNA species may be re-circulated to a feed reservoir to be re-filtered in additional cycles. In some aspects, a TFF membrane may have a MWCO equal to any one of, at least any one of, at most any one of, or between any two of at least 30 kDa, at least 40 kDa, at least 50 kDa, at least 60 kDa, at least 70 kDa, at least 80 kDa, at least 90 kDa, or more. In some aspects, a TFF membrane may have a MWCO equal to any one of, at least any one of, at most any one of, or between any two of at least 100 kDa, at least 150 kDa, at least 200 kDa, at least 250 kDa, at least 300 kDa, at least 350 kDa, at least 400 kDa, or more. In some aspects, a TFF membrane may have a MWCO of about 250-350 kDa. In some aspects, a TFF membrane (e.g., a cellulose-based membrane) may have a MWCO of about 30-300 kDa; in some aspects about 50-300 kDa, about 100-300 kDa, or about 200-300 kDa.

Diafiltration can be performed either discontinuously, or alternatively, continuously. For example, in continuous diafiltration, a diafiltration solution can be added to a sample feed reservoir at the same rate as filtrate is generated. In this way, the volume in the sample reservoir remains constant but small molecules (e.g., salts, solvents, etc.) that can freely permeate through a membrane are removed. Using solvent removal as an example, each additional diafiltration volume (DV) reduces the solvent concentration further. In discontinuous diafiltration, a solution is first diluted and then concentrated back to the starting volume. This process is then repeated until the desired concentration of small molecules (e.g., salts, solvents, etc.) remaining in the reservoir is reached. Each additional diafiltration volume (DV) reduces the small molecule (e.g., solvent) concentration further. Continuous diafiltration typically requires a minimum volume for a given reduction of molecules to be filtered. Discontinuous diafiltration, on the other hand, permits fast changes of the retentate condition, such as pH, salt content, and the like. In some aspects, the first diafiltration step is conducted with diavolumes equal to any one of, at least any one of, at most any one of, or between any two of at least 2, at least 3, at least 4, at least 5, at least 6, at least 7, at least 8, at least 9, at least 10, or more. In some aspects, the second diafiltration step is conducted with diavolumes equal to any one of, at least any one of, at most any one of, or between any two of at least 5, at least 6, at least 7, at least 8, at least 9, at least 10, at least 11, at least 12, at least 13, at least 14, at least 15, at least 16, at least 17, at least 18, at least 19, at least 20, or more. In some aspects, the first diafiltration step is conducted with 5 diavolumes, and second diafiltration step is conducted with 10 diavolumes.

In some aspects, for the ultrafiltration and/or diafiltration, the IVT mixture is filtered at a rate equal to any one of, at least any one of, at most any one of, or between any two of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 500, at least 600, at least 700, at least 800, at least 900, at least 1000 L/m2 of filter area per hour, or more. The concentrated RNA solution can comprise any one of, at least any one of, at most any one of, or between any two of 2.0, 2.1, 2.2, 2.3, 2.4, and 2.5 mg/mL single stranded RNA.

The bioburden of the concentrated RNA solution via filtration to obtain an RNA product solution may also be reduced, in some aspects. The filtration for reducing bioburden can be conducted using one or more filters. The one or more filters can include a filter with a pore size of 0.2 μm, 0.45 μm, 0.65 μm, 0.8 μm, or any other pore size configured to remove bioburdens.

As one example, reducing the bioburden can include draining a retentate tank containing retentate obtained from the ultrafiltration and/or diafiltration to obtain the retentate. Reducing the bioburden can include flushing a filtration system for ultrafiltration and/or diafiltration using a wash buffer solution to obtain a wash pool solution comprising residue RNA remaining in the filtration system. The retentate can be filtered to obtain a filtered retentate. The wash pool solution can be filtered using a first 0.2 μm filter to obtain a filtered wash pool solution. The retentate can be filtered using the first 0.2 μm filter or another 0.2 μm filter.

The filtered wash pool solution and the filtered retentate can be combined to form a combined pool solution. The combined pool solution can be filtered using a second 0.2 μm filter to obtain a filtered combined pool solution, which is further filtered using a third 0.2 μm filter to produce the RNA product solution.

The RNA in the RNA product solution may be encapsulated, and the RNA solution may further comprise at least one encapsulating agent. In one aspect, the encapsulating agent comprises a lipid, a lipid nanoparticle (LNP), lipoplexes, polymeric particles, polyplexes, and monolithic delivery systems, and a combination thereof. In one aspect, the encapsulating agent is a lipid, and produced is lipid nanoparticle (LNP)-encapsulated RNA. In some aspects, LNPs can be designed to protect RNAs (e.g., saRNA, mRNA) from extracellular RNases and/or can be engineered for systemic delivery of the RNA to target cells. In some aspects, such LNPs may be particularly useful to deliver RNAs (e.g., saRNA, mRNA) when RNAs are intravenously administered to a subject in need thereof. In some aspects, such LNPs may be particularly useful to deliver RNAs (e.g., saRNA, mRNA) when RNAs are intramuscularly administered to a subject in need thereof.

In some aspects, provided RNAs (e.g., saRNA, mRNA) may be formulated with LNPs. In various aspects, such LNPs can have an average size (e.g., mean diameter) equal to any one of, at least any one of, at most any one of, or between any two of about 30 nm to about 150 nm, about 40 nm to about 150 nm, about 50 nm to about 150 nm, about 50 nm to about 130 nm, about 50 nm to about 110 nm, about 50 nm to about 100 nm, about 50 to about 90 nm, or about 60 nm to about 80 nm, or about 60 nm to about 70 nm. In some aspects, LNPs that may be useful in accordance with the present disclosure can have an average size (e.g., mean diameter) equal to any one of, at least any one of, at most any one of, or between any two of about 50 nm to about 100 nm. In some aspects, LNPs may have an average size (e.g., mean diameter) of less than 80 nm, less than 75 nm, less than 70 nm, less than 65 nm, less than 60 nm, less than 55 nm, less than 50 nm, or less than 45 nm. In some aspects, LNPs that may be useful in accordance with the present disclosure can have an average size (e.g., mean diameter) of equal to any one of, at least any one of, at most any one of, or between any two of about 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain aspects, nucleic acids (e.g., RNAs), when present in provided LNPs, are resistant in aqueous solution to degradation with a nuclease. In some aspects, LNPs are liver-targeting lipid nanoparticles. In some aspects, LNPs are cationic lipid nanoparticles comprising one or more cationic lipids (e.g., ones described herein). In some aspects, cationic LNPs may comprise at least one cationic lipid, at least one polymer conjugated lipid, and at least one helper lipid (e.g., at least one neutral lipid).

In some aspects, LNP-encapsulated RNA can be produced by rapid mixing of an RNA solution described herein (e.g., the RNA product solution) and a lipid preparation described herein (comprising, e.g., at least one cationic lipid and optionally one or more other lipid components, in an organic solvent, preferably t-butanol) under conditions such that a sudden change in solubility of lipid component(s) is triggered, which drives the lipids towards self-assembly in the form of LNPs. In some aspects, suitable buffering agents comprise tris, histidine, citrate, acetate, phosphate, or succinate. In a preferred embodiment, the buffering agent is histidine. The pH during preparation of a liquid LNP-encapsulated RNA formulation relates to the pKa of the encapsulating agent (e.g., cationic lipid). The pH of the acidifying buffer may be at least half a pH scale less than the pKa of the encapsulating agent (e.g., cationic lipid), and the pH of the final buffer may be at least half a pH scale greater than the pKa of the encapsulating agent (e.g., cationic lipid). In preferred embodiments, the pH during preparation of the liquid LNP-encapsulated RNA formulation is pH 4-6, more preferably pH 5-6, most preferably pH 5.5. In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, can result in much higher encapsulation efficiency than it is achieved in the absence of interactions between nucleic acids and at least one of the lipid components. In some aspects, the pH during preparation of LNP-encapsulated RNA is different from the pH of the LNP-encapsulated RNA post-preparation of the LNP-encapsulated RNA.

In one aspect, the RNA in the RNA solution is at a concentration of <1 mg/ml. In another aspect, the RNA is at a concentration of at least about 0.05 mg/mL. In another aspect, the RNA is at a concentration of at least about 0.5 mg/mL. In another aspect, the RNA is at a concentration of at least about 1 mg/mL. In another aspect, the RNA concentration is from about 0.05 mg/mL to about 0.5 mg/mL. In another aspect, the RNA is at a concentration of at least 10 mg/mL. In another aspect, the RNA is at a concentration of at least 50 mg/mL. In some aspects, the RNA is at a concentration of equal to any one of, at least any one of, at most any one of, or between any two of about 0.05 mg/mL, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

In a further aspect, the RNA solution and the lipid preparation mixture further comprises a stabilizing agent. In some aspects, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In a specific aspect, the stabilizing agent is sucrose. In a specific aspect, the stabilizing agent is trehalose. In a specific aspect, the stabilizing agent is a combination of sucrose and trehalose. In some aspects, the stabilizing agent concentration includes, but is not limited to, a concentration of about 10 mg/mL to about 400 mg/mL, about 100 mg/mL to about 200 mg/mL, or about 103 mg/mL to about 200 mg/mL. In some aspects, the concentration of the stabilizing agent is equal to any one of, at least any one of, at most any one of, or between any two of 10 mg/mL, 20 mg/mL, 50 mg/mL, 103 mg/mL, 150 mg/mL, 200 mg/mL, 300 mg/mL, 400 mg/mL, or more. In some aspects, the concentration of the stabilizing agent(s) in the composition is about 1% to about 30% w/v. For example, the concentration of the stabilizing agent can be equal to any one of, at least any one of, at most any one of, or between any two of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% w/v or any range or value derivable therein. In specific aspects, the concentration of the stabilizing agent (e.g., sucrose) is 10.3%. In specific aspects, the concentration of the stabilizing agent (e.g., sucrose) is 15.4%. In specific aspects, the concentration of the stabilizing agent (e.g., sucrose) is 20.5%. In specific aspects, the concentration of the stabilizing agent (e.g., sucrose) is about 10%. In specific aspects, the concentration of the stabilizing agent (e.g., sucrose) is about 20%. In specific aspects, the concentration of the stabilizing agent (e.g., sucrose) is about 40%.

In a further aspect, the mass amount of the stabilizing agent and the mass amount of the RNA are in a specific ratio. In one aspect, the ratio of the mass amount of the stabilizing agent and the RNA is no greater than 5000. In another aspect, the ratio of the mass amount of the stabilizing agent and the RNA is no greater than 2000. In another aspect, the ratio of the mass amount of the stabilizing agent and the RNA is no greater than 1000. In another aspect, the ratio of the mass amount of the stabilizing agent and the RNA is no greater than 500. In another aspect, the ratio of the mass amount of the stabilizing agent and the RNA is no greater than 100. In another aspect, the ratio of the mass amount of the stabilizing agent and the pharmaceutical substance is no greater than 50. In another aspect, the ratio of the mass amount of the stabilizing agent and the RNA is no greater than 10. In another aspect, the ratio of the mass amount of the stabilizing agent and the RNA is no greater than 1. In another aspect, the ratio of the mass amount of the stabilizing agent and the RNA is no greater than 0.5. In another aspect, the ratio of the mass amount of the stabilizing agent and the RNA is no greater than 0.1. In another aspect, the stabilizing agent and RNA comprise a mass ratio of about 200-2000 of the stabilizing agent: 1 of the RNA. In a further aspect, the RNA is saRNA and the stabilizing agent is sucrose.

In some aspects, the RNA solution and the lipid preparation mixture further comprises a salt. In one aspect, the salt is a sodium salt. In a specific aspect, the salt is NaCl. In some aspects, the RNA solution and the lipid preparation mixture further comprises a surfactant, a preservative, any other excipient, or a combination thereof. As used herein, “any other excipient” includes, but is not limited to, antioxidants, glutathione, EDTA, methionine, desferal, antioxidants, metal scavengers, or free radical scavengers. In one aspect, the surfactant, preservative, excipient or combination thereof is selected from sterile water for injection (sWFI), bacteriostatic water for injection (BWFI), saline, dextrose solution, polysorbates, poloxamers, Triton, divalent cations, Ringer's lactate, amino acids, sugars, polyols, polymers or cyclodextrins.

In some aspects, the RNA solution and/or the lipid preparation mixture further comprises at least one free amino acid. In certain cases, the at least one free amino acid is internally loaded in the LNP-encapsulated RNAs. For example, in some cases, the at least one free amino acid is soluble in water and is combined with an RNA solution described herein (e.g., the RNA product solution). In some cases, the at least one free amino acid is soluble in ethanol and is combined with a lipid preparation described herein (comprising, e.g., at least one cationic lipid and optionally one or more other lipid components, in an organic solvent). In some cases, the at least one free amino acid is soluble in water and/or ethanol and is combined with both an RNA solution described herein (e.g., the RNA product solution) and a lipid preparation described herein (comprising, e.g., at least one cationic lipid and optionally one or more other lipid components, in an organic solvent). In this way, the at least one free amino acid is comprised in the LNP-encapsulated RNAs.

Alternatively, or in addition to, internally loading the LNP-encapsulated RNAs with the at least one free amino acid, pre-formed LNP-encapsulated RNAs may be externally loaded with the at least one free amino acid. For example, in some cases, the LNP-encapsulated RNAs produced according to the methods described herein are combined with the at least one free amino acid.

In some aspects, each of a buffer, stabilizing agent, salt, surfactant, preservative, and excipient are included in the RNA solution and the lipid preparation mixture. In other aspects, any one or more of a buffer, stabilizing agent, salt, surfactant, preservative, and excipient may be excluded from the RNA solution and the lipid preparation mixture.

### V. RNA Immunogenic Compositions and/or Vaccines

The RNA (e.g., mRNA) immunogenic compositions and/or vaccines may be utilized in various settings depending on the prevalence of the infection or the degree or level of unmet medical need. The RNA immunogenic compositions and/or vaccines may be utilized to treat and/or prevent an influenza virus of various genotypes, strains, and isolates. The RNA immunogenic compositions and/or vaccines typically have superior properties in that they produce much larger antibody titers and produce responses earlier than commercially available anti-viral therapeutic treatments. While not wishing to be bound by theory, it is believed that the RNA immunogenic compositions and/or vaccines, as mRNA polynucleotides, are better designed to produce the appropriate protein conformation upon translation as the RNA immunogenic compositions and/or vaccines co-opt natural cellular machinery. Unlike traditional vaccines, which are manufactured ex vivo and may trigger unwanted cellular responses, RNA (e.g., mRNA) immunogenic compositions and/or vaccines are presented to the cellular system in a more native fashion.

There may be situations in which persons are at risk for infection with more than one strain of influenza virus. RNA (e.g., mRNA) immunogenic compositions, such as therapeutic vaccines are particularly amenable to combination vaccination approaches due to a number of factors including, but not limited to, speed of manufacture, ability to rapidly tailor vaccines to accommodate perceived geographical threat, and the like. Moreover, because the immunogenic compositions and/or vaccines utilize the human body to produce the antigenic protein, the immunogenic compositions and/or vaccines are amenable to the production of larger, more complex antigenic proteins, allowing for proper folding, surface expression, antigen presentation, etc. in the human subject. To protect against more than one strain of influenza, a combination vaccine can be administered that includes RNA (e.g., mRNA) encoding at least one antigenic polypeptide protein (or antigenic portion thereof) of a first influenza virus or organism and further includes RNA encoding at least one antigenic polypeptide protein (or antigenic portion thereof) of a second influenza virus or organism. RNA (e.g., mRNA) can be co-formulated, for example, in a single lipid nanoparticle (LNP) or can be formulated in separate LNPs for co-administration.

Some aspects of the present disclosure provide viral vaccines (or compositions or immunogenic compositions) that include at least one RNA polynucleotide having an open reading frame encoding at least one viral antigenic polypeptide or an immunogenic fragment thereof (e.g., an immunogenic fragment capable of inducing an immune response to the virus). A representative but non-limiting list of viruses for which an RNA polynucleotide having an open reading frame encoding at least one viral antigenic polypeptide or an immunogenic fragment thereof can be provided include, but are not limited to, an arenavirus (such as Lassa virus, or lymphocytic choriomeningitis virus (LCMV)); an astrovirus; a bunyavirus (such as a Hantavirus); a calicivirus; a coronavirus (such as a severe acute respiratory syndrome virus (SARS)—e.g., SARS-CoV-1, or a middle east respiratory syndrome (MERS) virus); a filovirus (such as Ebola virus or Marburg virus); a flavivirus (such as Yellow Fever virus, West Nile virus, or Hepatitis C virus (HCV)); a hepadnavirus; a hepevirus; an orthomyxovirus (such as Influenza A virus, Influenza B virus, or Influenza C virus); a paramyxovirus (such as Rubeola virus, or Rubulavirus); a picornavirus (such as Poliovirus, Hepatitis A virus, or Rhinovirus); a reovirus (such as Rotavirus); a retrovirus (such as Human Immunodeficiency Virus (HIV), or Human T-lymphotropic virus (HTLV)); a rhabdovirus (such as Rabies virus or Rabies lyssavirus); a togavirus (such as Sindbis virus (SINV), Eastern Equine Encephalitis virus (EEEV), Western Equine Encephalitis virus (WEEV), or Rubella virus).

Vaccines may include an RNA polynucleotide having an open reading frame encoding at least one antigenic polypeptide or an immunogenic fragment thereof derived from, e.g., Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 01 57:H7, 01 1 1 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowled, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B1 9, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

Some aspects of the present disclosure provide influenza virus (influenza) vaccines (or compositions or immunogenic compositions) that include at least one RNA polynucleotide having an open reading frame encoding at least one influenza antigenic polypeptide or an immunogenic fragment thereof (e.g., an immunogenic fragment capable of inducing an immune response to influenza).

In some aspects, the at least one antigenic polypeptide is one of the defined antigenic subdomains of HA, termed HA1, HA2, or a combination of HA1 and HA2, and at least one antigenic polypeptide selected from neuraminidase (NA), nucleoprotein (NP), matrix protein 1 (M1), matrix protein 2 (M2), non-structural protein 1 (NS1) and non-structural protein 2 (NS2).

In some aspects, the at least one antigenic polypeptide is HA or derivatives thereof comprising antigenic sequences from HA1 and/or HA2, and at least one antigenic polypeptide selected from NA, NP, M1, M2, NS1 and NS2.

In some aspects, the at least one antigenic polypeptide is HA or derivatives thereof comprising antigenic sequences from HA1 and/or HA2 and at least two antigenic polypeptides selected from NA, NP, M1, M2, NS1 and NS2.

In some aspects, an immunogenic composition and/or a vaccine comprises at least one RNA (e.g., mRNA) polynucleotide having an open reading frame encoding an influenza virus protein, or an immunogenic fragment thereof.

In some aspects, an immunogenic composition and/or a vaccine comprises at least one RNA (e.g., mRNA) polynucleotide having an open reading frame encoding multiple influenza virus proteins, or immunogenic fragments thereof. In some aspects, an immunogenic composition and/or a vaccine comprises at least one RNA (e.g., mRNA) polynucleotide having an open reading frame encoding a HA protein, or an immunogenic fragment thereof (e.g., at least one HA1, HA2, or a combination of both).

In some aspects, an immunogenic composition and/or a vaccine comprises at least one RNA (e.g., mRNA) polynucleotide having an open reading frame encoding a HA protein, or an immunogenic fragment thereof (e.g., at least one HA1, HA2, or a combination of both, of any one of or a combination of any or all of H1, H2, H3, H4, H5, H6, H7, H8, H9, H10, H11, H12, H13, H14, H15, H16, H17, and/or H18) and at least one other RNA (e.g., mRNA) polynucleotide having an open reading frame encoding a protein selected from a NP protein, a NA protein, a M1 protein, a M2 protein, a NS1 protein and a NS2 protein obtained from influenza virus.

In some aspects, an immunogenic composition and/or a vaccine comprises at least one RNA (e.g., mRNA) polynucleotide having an open reading frame encoding a HA protein, or an immunogenic fragment thereof (e.g., at least one any one of or a combination of any or all of H1, H2, H3, H4, H5, H6, H7, H8, H9, H10, H11, H12, H13, H14, H15, H16, H17, and/or H18) and at least two other RNAs (e.g., mRNAs) polynucleotides having two open reading frames encoding two proteins selected from a NP protein, a NA protein, a M1 protein, a M2 protein, a NS1 protein and a NS2 protein obtained from influenza virus.

In some aspects, an immunogenic composition and/or a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least three other RNAs (e.g., mRNAs) polynucleotides having three open reading frames encoding three proteins selected from a NP protein, a NA protein, a M protein, a M2 protein, a NS1 protein and a NS2 protein obtained from influenza virus.

In some aspects, an immunogenic composition and/or a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least four other RNAs (e.g., mRNAs) polynucleotides having four open reading frames encoding four proteins selected from a NP protein, a NA protein, a M1 protein, a M2 protein, a NS1 protein and a NS2 protein obtained from influenza virus.

In some aspects, an immunogenic composition and/or a vaccine comprises at least one RNA (e.g., mRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least five other RNAs (e.g., mRNAs) polynucleotides having five open reading frames encoding five proteins selected from a NP protein, a NA protein, a M1 protein, a M2 protein, a NS1 protein and a NS2 protein obtained from influenza virus.

In some aspects, an immunogenic composition and/or a vaccine comprises at least one RNA (e.g., mRNA) polynucleotide having an open reading frame encoding a HA protein or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18), a NP protein or an immunogenic fragment thereof, a NA protein or an immunogenic fragment thereof, a M1 protein or an immunogenic fragment thereof, a M2 protein or an immunogenic fragment thereof, a NS1 protein or an immunogenic fragment thereof and a NS2 protein or an immunogenic fragment thereof obtained from influenza virus.

Some aspects of the present disclosure provide the following novel influenza virus polypeptide sequences: H1HA10-Foldon_ANglyl; H1HA10TM-PR8 (H1 A/Puerto Rico/8/34 HA); H1HA10-PR8-DS (H1 A/Puerto Rico/8/34 HA; pH1HA10-Cal04-DS (H1 A/California/04/2009 HA); Pandemic H1HA10 from California 04; pH1HA10-ferritin; HA10; Pandemic H1HA10 from California 04; Pandemic H1HA10 from California 04 strain/without foldon and with K68C/R76C mutation for trimerization; H1HA10 from A/Puerto Rico/8/34 strain, without foldon and with Y94D/N95L mutation for trimerization; H1HA10 from A/Puerto Rico/8/34 strain, without foldon and with K68C/R76C mutation for trimerization; H1N1 A/Viet Nam/850/2009; H3N2 A/Wisconsin/67/2005; H7N9 (A/Anhui/1/2013); H9N2 A/Hong Kong/1073/99; H10N8 A/JX346/2013.

Some aspects of the present disclosure provide influenza virus (influenza) immunogenic compositions and/or vaccines that include at least one RNA polynucleotide having an open reading frame encoding at least one influenza antigenic polypeptide or an immunogenic fragment of the novel influenza virus polypeptide sequences described above (e.g., an immunogenic fragment capable of inducing an immune response to influenza). In some aspects, an influenza vaccine comprises at least one RNA (e.g., mRNA) polynucleotide having an open reading frame encoding at least one influenza antigenic polypeptide comprising a modified sequence that is at least 75% (e.g., any number between 75% and 100%, inclusive, e.g., 70%, 80%, 85%, 90%, 95%, 99%, and 100%) identity to an amino acid sequence of the novel influenza virus sequences described above. The modified sequence can be at least 75% (e.g., any number between 75% and 100%, inclusive, e.g., 70%, 80%, 85%, 90%, 95%, 99%, and 100%) identical to an amino acid sequence of the novel influenza virus sequences described above. Some aspects of the present disclosure provide an isolated nucleic acid comprising a sequence encoding the novel influenza virus polypeptide sequences described above; an expression vector comprising the nucleic acid; and a host cell comprising the nucleic acid. The present disclosure also provides a method of producing a polypeptide of any of the novel influenza virus sequences described above. A method may include culturing the host cell in a medium under conditions permitting nucleic acid expression of the novel influenza virus sequences described above, and purifying from the cultured cell or the medium of the cell a novel influenza virus polypeptide. The present disclosure also provides antibody molecules, including full length antibodies and antibody derivatives, directed against the novel influenza virus sequences.

In some aspects, an open reading frame of a RNA (e.g., mRNA) immunogenic composition and/or vaccine is codon-optimized. In some aspects, a RNA (e.g., mRNA) immunogenic composition and/or vaccine further comprises an adjuvant. In some aspects, at least one RNA polynucleotide encodes at least one influenza antigenic polypeptide that attaches to cell receptors. In some aspects, at least one RNA polynucleotide encodes at least one influenza antigenic polypeptide that causes fusion of viral and cellular membranes. In some aspects, at least one RNA polynucleotide encodes at least one influenza antigenic polypeptide that is responsible for binding of the virus to a cell being infected. Some aspects of the present disclosure provide an immunogenic composition and/or a vaccine that includes at least one ribonucleic acid (RNA) (e.g., mRNA) polynucleotide having an open reading frame encoding at least one influenza antigenic polypeptide, at least one 5′ terminal cap and at least one chemical modification, formulated within a lipid nanoparticle.

In some aspects, a 5′ terminal cap is 7mG(5′)ppp(5′)NImpNp. In some aspects, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some aspects, the chemical modification is in the 5-position of the uracil. In some aspects, the chemical modification is a N1-methylpseudouridine. In some aspects, the chemical modification is a N1-ethylpseudouridine.

In some aspects, a lipid nanoparticle comprises a cationic lipid, a PEG-modified lipid, a sterol and a non-cationic lipid. In some aspects, a cationic lipid is an ionizable cationic lipid and the non-cationic lipid is a neutral lipid, and the sterol is a cholesterol. In some aspects, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy) heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530).

Some aspects of the present disclosure provide an immunogenic composition and/or a vaccine that includes at least one RNA (e.g., mRNA) polynucleotide having an open reading frame encoding at least one antigen, such as an influenza antigenic polypeptide, wherein at least 80% (e.g., 85%, 90%, 95%, 98%, 99%) of the uracil in the open reading frame have a chemical modification, optionally wherein the immunogenic composition and/or vaccine is formulated in a lipid nanoparticle (e.g., a lipid nanoparticle comprises a cationic lipid, a PEG-modified lipid, a sterol and a non-cationic lipid). In some aspects, 100% of the uracil in the open reading frame have a chemical modification. In some aspects, a chemical modification is in the 5-position of the uracil. In some aspects, a chemical modification is a N1-methyl pseudouridine. In some aspects, 100% of the uracil in the open reading frame have a N1-methyl pseudouridine in the 5-position of the uracil.

In some aspects, an open reading frame of a RNA (e.g., mRNA) polynucleotide encodes at least one antigen, such as an influenza antigenic polypeptide. In some aspects, the open reading frame encodes at least two, at least five, or at least ten antigenic polypeptides. In some aspects, the open reading frame encodes at least 100 antigenic polypeptides. In some aspects, the open reading frame encodes 1-100 antigenic polypeptides. In some aspects, an immunogenic composition and/or a vaccine comprises at least two RNA (e.g., mRNA) polynucleotides, each having an open reading frame encoding at least one influenza antigenic polypeptide. In some aspects, the immunogenic composition and/or vaccine comprises at least five or at least ten RNA (e.g., mRNA) polynucleotides, each having an open reading frame encoding at least one antigenic polypeptide or an immunogenic fragment thereof. In some aspects, the immunogenic composition and/or vaccine comprises at least 100 RNA (e.g., mRNA) polynucleotides, each having an open reading frame encoding at least one antigenic polypeptide. In some aspects, the immunogenic composition and/or vaccine comprises 2-100 RNA (e.g., mRNA) polynucleotides, each having an open reading frame encoding at least one antigenic polypeptide.

Also provided herein is an influenza RNA (e.g., mRNA) immunogenic composition and/or vaccine of any one of the foregoing paragraphs formulated in a nanoparticle (e.g., a lipid nanoparticle). In some aspects, the nanoparticle has a mean diameter of 50-200 nm. In some aspects, the nanoparticle is a lipid nanoparticle. In some aspects, the lipid nanoparticle comprises a cationic lipid, a PEG-modified lipid, a sterol and a non-cationic lipid. In some aspects, the lipid nanoparticle comprises a molar ratio of about 20-60% cationic lipid, 0.5-15% PEG-modified lipid, 25-55% sterol, and 25% non-cationic lipid. In some aspects, the cationic lipid is an ionizable cationic lipid and the non-cationic lipid is a neutral lipid, and the sterol is a cholesterol. In some aspects, the nanoparticle has a polydispersity value of less than 0.4 (e.g., less than 0.3, 0.2 or 0.1). In some aspects, the nanoparticle has a net neutral charge at a neutral pH value.

Some aspects of the present disclosure provide methods of inducing an antigen specific immune response in a subject, comprising administering to the subject any of the RNA (e.g., mRNA) immunogenic composition and/or vaccine as provided herein in an amount effective to produce an antigen-specific immune response. In some aspects, the RNA (e.g., mRNA) immunogenic composition and/or vaccine is an influenza immunogenic composition and/or vaccine. In some aspects, the RNA (e.g., mRNA) vaccine is a combination vaccine comprising a combination of influenza vaccines (a broad spectrum influenza vaccine). In some aspects, an antigen-specific immune response comprises a T cell response or a B cell response. In some aspects, a method of producing an antigen-specific immune response comprises administering to a subject a single dose (no booster dose) of an influenza RNA (e.g., mRNA) immunogenic composition and/or vaccine of the present disclosure. In some aspects, a method further comprises administering to the subject a second (booster) dose of an influenza RNA (e.g., mRNA) vaccine. Additional doses of an influenza RNA (e.g., mRNA) immunogenic composition and/or vaccine may be administered.

In some aspects, the subjects exhibit a seroconversion rate of at least 80% (e.g., at least 85%, at least 90%, or at least 95%) following the first dose or the second (booster) dose of the immunogenic composition and/or vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

Administration of an immunogenic composition, such as a vaccine, such as an influenza RNA (e.g., mRNA) vaccine, described herein can be carried out via any of the accepted modes of administration of agents for serving similar utilities. Immunogenic compositions may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such immunogenic compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection, or infusion techniques. Immunogenic compositions described herein are formulated so as to allow the active ingredients contained therein to be bioavailable upon administration of the composition to a patient. Immunogenic compositions that will be administered to a subject or patient take the form of one or more dosage units, where for example, a tablet may be a single dosage unit, and a container of a compound in aerosol form may hold a plurality of dosage units. The immunogenic compositions to be administered will, in any event, contain a therapeutically effective amount of a compound within the scope of this disclosure, or a pharmaceutically acceptable salt thereof, for treatment or prevention of a disease or condition of interest in accordance with the teachings described herein.

Immunogenic compositions within the scope of this disclosure may be in the form of a solid or liquid. In one aspect, the carrier(s) are particulate, so that the immunogenic compositions are, for example, in tablet or powder form. The carrier(s) may be liquid, with the immunogenic compositions being, for example, an oral syrup, injectable liquid, or an aerosol, which is useful in, for example, inhalator administration. When intended for oral administration, the immunogenic composition is preferably in either solid or liquid form, where semi-solid, semi-liquid, suspension, and gel forms are included within the forms considered herein as either solid or liquid. As a solid composition for oral administration, the immunogenic composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate, or orange flavoring; and a coloring agent. When the immunogenic composition is in the form of a capsule, for example, a gelatin capsule, it may contain, in addition to materials of the above type, a liquid carrier such as polyethylene glycol or oil. The immunogenic composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant, and flavor enhancer. In an immunogenic compositions intended to be administered by injection, one or more of a surfactant, preservative, wetting agent, dispersing agent, suspending agent, buffer, stabilizer, and isotonic agent may be included or excluded.

Liquid immunogenic compositions, whether they be solutions, suspensions, or other like form, may include or exclude one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation can be enclosed in ampoules, disposable syringes, or multiple dose vials made of glass or plastic. Physiological saline is a preferred adjuvant. An injectable pharmaceutical composition is preferably sterile. A liquid immunogenic composition intended for either parenteral or oral administration should contain an amount of a compound such that a suitable dosage will be obtained.

The immunogenic compositions may be prepared by methodology well known in the pharmaceutical art. For example, a pharmaceutical composition intended to be administered by injection can be prepared by combining the mRNA with sterile, distilled water or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

In some aspects, an immunogenic composition, such as an influenza RNA (e.g., mRNA) vaccine, is administered to a subject by intradermal injection, intramuscular injection, or by intranasal administration. In some aspects, an immunogenic composition, such as an influenza RNA (e.g., mRNA) vaccine is administered to a subject by intramuscular injection. Some aspects, of the present disclosure provide methods of inducing an antigen specific immune response in a subject, including administering to a subject an immunogenic composition, such as an influenza RNA (e.g., mRNA) vaccine, in an effective amount to produce an antigen specific immune response in a subject. Antigen-specific immune responses in a subject may be determined, in some aspects, by assaying for antibody titer (for titer of an antibody that binds to an influenza antigenic polypeptide) following administration to the subject of any of the immunogenic compositions and/or vaccines of the present disclosure. In some aspects, the anti-antigenic polypeptide antibody titer produced in the subject is increased by at least 1 log relative to a control. In some aspects, the anti-antigenic polypeptide antibody titer produced in the subject is increased by 1-3 log relative to a control. In some aspects, the anti-antigenic polypeptide antibody titer produced in a subject is increased at least 2 times relative to a control. In some aspects, the anti-antigenic polypeptide antibody titer produced in the subject is increased at least 5 times relative to a control. In some aspects, the anti-antigenic polypeptide antibody titer produced in the subject is increased at least 10 times relative to a control. In some aspects, the anti-antigenic polypeptide antibody titer produced in the subject is increased 2-10 times relative to a control. In some aspects, the control is an anti-antigenic polypeptide antibody titer produced in a subject who has not been administered an immunogenic composition of the present disclosure, such as a RNA (e.g., mRNA) vaccine of the present disclosure. In some aspects, the control is an anti-antigenic polypeptide antibody titer produced in a subject who has been administered a live attenuated or inactivated influenza, or wherein the control is an anti-antigenic polypeptide antibody titer produced in a subject who has been administered a recombinant or purified influenza protein vaccine. In some aspects, the control is an anti-antigenic polypeptide antibody titer produced in a subject who has been administered an influenza virus-like particle (VLP) vaccine.

An immunogenic composition, such as an RNA (e.g., mRNA) vaccine, of the present disclosure is administered to a subject in an effective amount (an amount effective to induce an immune response). In some aspects, the effective amount is a dose equivalent to an at least 2-fold, at least 4-fold, at least 10-fold, at least 100-fold, at least 1000-fold reduction in the standard of care dose of a standard of care immunogenic composition, such as a recombinant influenza protein vaccine, wherein the anti-antigenic polypeptide antibody titer produced in the subject is equivalent to an anti-antigenic polypeptide antibody titer produced in a control subject administered the standard of care dose of a standard of care immunogenic composition, such as a recombinant influenza protein vaccine, a purified influenza protein vaccine, a live attenuated influenza vaccine, an inactivated influenza vaccine, or an influenza VLP vaccine. In some aspects, the effective amount is a dose equivalent to 2-1000-fold reduction in the standard of care dose of a standard of care immunogenic composition, such as a recombinant influenza protein vaccine, wherein the anti-antigenic polypeptide antibody titer produced in the subject is equivalent to an anti-antigenic polypeptide antibody titer produced in a control subject administered the standard of care dose of a standard of care immunogenic composition, such as a recombinant influenza protein vaccine, a purified influenza protein vaccine, a live attenuated influenza vaccine, an inactivated influenza vaccine, or an influenza VLP vaccine. In some aspects, the control is an anti-antigenic polypeptide antibody titer produced in a subject who has been administered a virus-like particle (VLP) vaccine comprising structural proteins of influenza. In some aspects, the immunogenic composition and/or vaccine is formulated in an effective amount to produce an antigen specific immune response in a subject. In some aspects, the effective amount is a total dose of 25 μg to 1000 μg, or 50 μg to 1000 μg. In some aspects, the effective amount is a total dose of 100 μg. In some aspects, the effective amount is a dose of 25 μg administered to the subject a total of two times. In some aspects, the effective amount is a dose of 100 μg administered to the subject a total of two times. In some aspects, the effective amount is a dose of 400 μg administered to the subject a total of two times. In some aspects, the effective amount is a dose of 500 μg administered to the subject a total of two times. In some aspects, the efficacy (or effectiveness) of an immunogenic composition and/or a vaccine of the present disclosure is greater than 60%. In some aspects, the immunogenic composition and/or vaccine contains RNA (e.g., mRNA) that encodes for at least one Influenza antigenic polypeptide.

Immunogenic composition efficacy may be assessed using standard analyses. For example, immunogenic composition efficacy may be measured by double-blind, randomized, clinical controlled trials. Immunogenic composition efficacy may be expressed as a proportionate reduction in disease attack rate (AR) between unvaccinated (ARU) and vaccinated (ARV) study cohorts that are administered an immunogenic composition of the present disclosure and can be calculated from the relative risk (RR) of disease among the vaccinated group with use of the following formulas: Efficacy=(ARU−ARV)/ARU×100; and Efficacy=(1−RR)×100. Likewise, immunogenic composition effectiveness may be assessed using standard analyses. Immunogenic composition effectiveness is an assessment of how an immunogenic compositions (which may have already proven to have high efficacy) reduce disease in a population. This measure can assess the net balance of benefits and adverse effects of an immunogenic composition administration program, not just the immunogenic composition itself, under natural field conditions rather than in a controlled clinical trial. Immunogenic composition effectiveness is proportional to immunogenic composition efficacy (potency) but is also affected by how well target groups in the population are immunized, as well as by other non-immunogenic composition-related factors that influence the ‘real-world’ outcomes of hospitalizations, ambulatory visits, or costs. For example, a retrospective case control analysis may be used, in which the rates of immunogenic composition administration among a set of infected cases and appropriate controls are compared. Immunogenic composition effectiveness may be expressed as a rate difference, with use of the odds ratio (OR) for developing infection despite administration of the immunogenic composition: Effectiveness=(1−OR)×100. In some aspects, the efficacy (or effectiveness) of an immunogenic composition disclosed herein is at least 65%, at least 70%, at least 75%, at least 80%, at least 85%, or at least 90%.

In some aspects, the immunogenic composition immunizes the subject against the antigen-producing organism, virus, or cell, such as Influenza, for up to 2 years. In some aspects, the immunogenic composition immunizes the subject for more than 2 years, more than 3 years, more than 4 years, or for 5-10 years. In some aspects, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some aspects, the subject is about 12 months or younger (e.g., 12, 11, 10, 9, 8, 7, 6, 5, 4, 3, 2 months or 1 month). In some aspects, the subject is about 6 months or younger. In some aspects, the subject was born full term (e.g., about 37-42 weeks). In some aspects, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some aspects, the subject was born prematurely between about 32 weeks and about 36 weeks of gestation. In such subjects, an immunogenic composition may be administered later in life, for example, at the age of about 6 months to about 5 years, or older. In some aspects, the subject is a young adult between the ages of about 20 years and about 50 years (e.g., about 20, 25, 30, 35, 40, 45 or 50 years old). In some aspects, the subject is an elderly subject about 60 years old, about 70 years old, or older (e.g., about 60, 65, 70, 75, 80, 85 or 90 years old).

In some aspects, the vaccine immunizes the subject against Influenza for up to 2 years. In some aspects, the vaccine immunizes the subject against Influenza for more than 2 years, more than 3 years, more than 4 years, or for 5-10 years. In some aspects, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some aspects, the subject is about 12 months or younger (e.g., 12, 11, 10, 9, 8, 7, 6, 5, 4, 3, 2 months or 1 month). In some aspects, the subject is about 6 months or younger. In some aspects, the subject was born full term (e.g., about 37-42 weeks). In some aspects, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some aspects, the subject was born prematurely between about 32 weeks and about 36 weeks of gestation. In such subjects, a RNA (e.g., mRNA) vaccine may be administered later in life, for example, at the age of about 6 months to about 5 years, or older. In some aspects, the subject is a young adult between the ages of about 20 years and about 50 years (e.g., about 20, 25, 30, 35, 40, 45 or 50 years old). In some aspects, the subject is an elderly subject about 60 years old, about 70 years old, or older (e.g., about 60, 65, 70, 75, 80, 85 or 90 years old).

In some aspects, the subject has been exposed to influenza (e.g., C. trachomatis); the subject is infected with influenza (e.g., C. trachomatis); or subject is at risk of infection by influenza (e.g., C. trachomatis). In some aspects, the subject is immunocompromised (has an impaired immune system, e.g., has an immune disorder or autoimmune disorder). In some aspects, the RNA molecules of the immunogenic compositions of the present disclosure encode a viral polypeptide or fragment thereof, including naturally occurring or engineered variants thereof, for prophylaxis against a virus in humans. In some aspects, the viral polypeptide does not comprise a coronavirus polypeptide. In some aspects, the viral polypeptide does not comprise a severe acute respiratory syndrome (SARS) virus polypeptide. In some aspects, the viral polypeptide does not comprise a SARS-CoV-2 polypeptide.

Thus, in some aspects, the RNA molecules of the immunogenic compositions of the present disclosure do not encode a coronavirus polypeptide or fragment thereof, including naturally occurring or engineered variants thereof. In some aspects, the RNA molecules of the immunogenic compositions of the present disclosure do not encode a SARS virus polypeptide or fragment thereof, including naturally occurring or engineered variants thereof. In some aspects, the RNA molecules of the immunogenic compositions of the present disclosure do not encode a SARS-CoV-2 virus polypeptide or fragment thereof, including naturally occurring or engineered variants thereof.

In further aspects, the RNA molecules of the immunogenic compositions of the present disclosure are not used for prophylaxis against a coronavirus in humans. In some aspects, the RNA molecules of the immunogenic compositions of the present disclosure are not used for prophylaxis against a SARS virus in humans. In some aspects, the RNA molecules of the immunogenic compositions of the present disclosure are not used for prophylaxis against SARS-CoV-2 in humans.

In some aspects, the immunogenic compositions of the present disclosure, such as RNA (e.g., mRNA) vaccines, comprise RNA encoding a viral polypeptide or fragment thereof, including naturally occurring or engineered variants thereof, for prophylaxis against a virus in humans. In some aspects, the viral polypeptide does not comprise a coronavirus polypeptide. In some aspects, the viral polypeptide does not comprise a severe acute respiratory syndrome (SARS) virus polypeptide. In some aspects, the viral polypeptide does not comprise a SARS-CoV-2 polypeptide.

Thus, in some aspects, the immunogenic compositions of the present disclosure, such as RNA (e.g., mRNA) vaccines, do not comprise RNA encoding a coronavirus polypeptide or fragment thereof, including naturally occurring or engineered variants thereof. In some aspects, the immunogenic compositions of the present disclosure do not comprise RNA encoding a severe acute respiratory syndrome (SARS) virus polypeptide or fragment thereof, including naturally occurring or engineered variants thereof. In some aspects, the immunogenic compositions of the present disclosure do not comprise RNA encoding a SARS-CoV-2 virus polypeptide or fragment thereof, including naturally occurring or engineered variants thereof.

In further aspects, the immunogenic compositions of the present disclosure are not used for prophylaxis against a coronavirus in humans. In some aspects, the immunogenic compositions of the present disclosure are not used for prophylaxis against a SARS virus in humans. In some aspects, the immunogenic compositions of the present disclosure are not used for prophylaxis against SARS-CoV-2 in humans.

In some aspects the nucleic acid immunogenic compositions and/or vaccines described herein are chemically modified. In other aspects the nucleic acid immunogenic compositions vaccines are unmodified. Yet other aspects provide compositions for and methods of administering an immunogenic composition to a subject comprising administering to the subject a nucleic acid vaccine comprising one or more RNA polynucleotides having an open reading frame encoding a first virus antigenic polypeptide, wherein the RNA polynucleotide does not include a stabilization element, and wherein an adjuvant is not coformulated or co-administered with the vaccine.

In other aspects the invention is an immunogenic composition for or method of vaccinating a subject comprising administering to the subject an immunogenic composition and/or a vaccine comprising one or more RNA polynucleotides having an open reading frame encoding a first antigenic polypeptide wherein a dosage of between 10 μg/kg and 400 μg/kg of the nucleic acid immunogenic composition and/or vaccine is administered to the subject. In some aspects the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 ug, 100-400 μg, 200-400 μg, or 300-400 μg per dose. In some aspects, the nucleic acid immunogenic composition and/or vaccine is administered to the subject by intradermal or intramuscular injection. In some aspects, the nucleic acid immunogenic composition and/or vaccine is administered to the subject on day zero. In some aspects, a second dose of the nucleic acid immunogenic composition and/or vaccine is administered to the subject on day twenty one. In some aspects, a dosage of 25 micrograms of the RNA polynucleotide is included in the nucleic acid immunogenic composition and/or vaccine administered to the subject. In some aspects, a dosage of 100 micrograms of the RNA polynucleotide is included in the nucleic acid immunogenic composition and/or vaccine administered to the subject. In some aspects, a dosage of 50 micrograms of the RNA polynucleotide is included in the nucleic acid immunogenic composition and/or vaccine administered to the subject. In some aspects, a dosage of 75 micrograms of the RNA polynucleotide is included in the nucleic acid v immunogenic composition and/or accine administered to the subject. In some aspects, a dosage of 150 micrograms of the RNA polynucleotide is included in the nucleic acid immunogenic composition and/or vaccine administered to the subject. In some aspects, a dosage of 400 micrograms of the RNA polynucleotide is included in the nucleic acid immunogenic composition and/or vaccine administered to the subject. In some aspects, a dosage of 200 micrograms of the RNA polynucleotide is included in the nucleic acid immunogenic composition and/or vaccine administered to the subject. In some aspects, the RNA polynucleotide accumulates at a 100 fold higher level in the local lymph node in comparison with the distal lymph node. In other aspects the nucleic acid immunogenic composition and/or vaccine is chemically modified and in other aspects the nucleic acid immunogenic composition and/or vaccine is not chemically modified.

Aspects of the invention provide a nucleic acid immunogenic composition and/or vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some aspects, the stabilization element is a histone stem-loop. In some aspects, the stabilization element is a nucleic acid sequence having increased GC content relative to wild type sequence.

Aspects of the invention provide nucleic acid immunogenic composition and/or vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some aspects, the antibody titer produced by the mRNA immunogenic compositions and/or vaccines of the invention is a neutralizing antibody titer. In some aspects the neutralizing antibody titer is greater than a protein vaccine. In other aspects the neutralizing antibody titer produced by the mRNA immunogenic compositions and/or vaccines of the invention is greater than an adjuvanted protein vaccine. In yet other aspects the neutralizing antibody titer produced by the mRNA immunogenic compositions and/or vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

Also provided are nucleic acid immunogenic compositions and/or vaccines comprising one or more RNA polynucleotides having an open reading frame encoding a first antigenic polypeptide, wherein the RNA polynucleotide is present in a formulation for in vivo administration to a host for eliciting a longer lasting high antibody titer than an antibody titer elicited by an mRNA immunogenic composition and/or vaccine having a stabilizing element or formulated with an adjuvant and encoding the first antigenic polypeptide. In some aspects, the RNA polynucleotide is formulated to produce neutralizing antibodies within one week of a single administration. In some aspects, the adjuvant is selected from a cationic peptide and an immunostimulatory nucleic acid. In some aspects, the cationic peptide is protamine.

Aspects provide nucleic acid immunogenic compositions and/or vaccines comprising one or more RNA polynucleotides having an open reading frame comprising at least one chemical modification or optionally no modified nucleotides, the open reading frame encoding a first antigenic polypeptide, wherein the RNA polynucleotide is present in the formulation for in vivo administration to a host such that the level of antigen expression in the host significantly exceeds a level of antigen expression produced by an mRNA immunogenic composition and/or vaccine having a stabilizing element or formulated with an adjuvant and encoding the first antigenic polypeptide.

Other aspects provide nucleic acid immunogenic compositions and/or vaccines comprising one or more RNA polynucleotides having an open reading frame comprising at least one chemical modification or optionally no modified nucleotides, the open reading frame encoding a first antigenic polypeptide, wherein the immunogenic composition and/or vaccine has at least 10 fold less RNA polynucleotide than is required for an unmodified mRNA immunogenic composition and/or vaccine to produce an equivalent antibody titer. In some aspects, the RNA polynucleotide is present in a dosage of 25-100 micrograms.

Aspects of the invention also provide a unit of use immunogenic composition and/or vaccine, comprising between 10 μg and 400 μg of one or more RNA polynucleotides having an open reading frame comprising at least one chemical modification or optionally no modified nucleotides, the open reading frame encoding a first antigenic polypeptide, and a pharmaceutically acceptable carrier or excipient, formulated for delivery to a human subject. In some aspects, the immunogenic composition and/or vaccine further comprises a cationic lipid nanoparticle.

Aspects of the invention provide methods of creating, maintaining or restoring antigenic memory to an antigen, such as an antigen of a virus strain in an individual or population of individuals comprising administering to said individual or population an antigenic memory booster nucleic acid immunogenic composition and/or vaccine comprising (a) at least one RNA polynucleotide, said polynucleotide comprising at least one chemical modification or optionally no modified nucleotides and two or more codon-optimized open reading frames, said open reading frames encoding a set of reference antigenic polypeptides, and (b) optionally a pharmaceutically acceptable carrier or excipient. In some aspects, the immunogenic composition and/or vaccine is administered to the individual via a route selected from the group consisting of intramuscular administration, intradermal administration and subcutaneous administration. In some aspects, the administering step comprises contacting a muscle tissue of the subject with a device suitable for injection of the composition. In some aspects, the administering step comprises contacting a muscle tissue of the subject with a device suitable for injection of the composition in combination with electroporation.

Aspects of the invention provide methods of administering an immunogenic composition and/or vaccine to a subject (e.g., vaccinating a subject) comprising administering to the subject, for example, a single dosage of between 25 μg/kg and 400 μg/kg of a nucleic acid immunogenic composition and/or vaccine comprising one or more RNA polynucleotides having an open reading frame encoding a first antigenic polypeptide in an effective amount to administer to or vaccinate the subject.

Other aspects provide nucleic acid immunogenic compositions and/or vaccines comprising one or more RNA polynucleotides having an open reading frame comprising at least one chemical modification, the open reading frame encoding a first antigenic polypeptide, wherein the immunogenic composition and/or vaccine has at least 10 fold less RNA polynucleotide than is required for an unmodified mRNA immunogenic composition and/or vaccine to produce an equivalent antibody titer. In some aspects, the RNA polynucleotide is present in a dosage of 25-100 micrograms.

Other aspects provide nucleic acid immunogenic compositions and/or vaccines comprising an LNP formulated RNA polynucleotide having an open reading frame comprising no nucleotide modifications (unmodified), the open reading frame encoding a first antigenic polypeptide, wherein the immunogenic composition and/or vaccine has at least 10 fold less RNA polynucleotide than is required for an unmodified mRNA immunogenic composition and/or vaccine not formulated in a LNP to produce an equivalent antibody titer. In some aspects, the RNA polynucleotide is present in a dosage of 25-100 micrograms.

The data presented in the Examples demonstrate significant enhanced qualities and attributes that may produce immune responses using the formulations of the invention. Both chemically modified and unmodified RNA immunogenic compositions and/or vaccines are useful according to the invention.

In other aspects the invention encompasses a method of treating an elderly subject age 60 years or older comprising administering to the subject a nucleic acid immunogenic composition and/or vaccine comprising one or more RNA polynucleotides having an open reading frame encoding an virus antigenic polypeptide in an effective amount to vaccinate the subject. In other aspects the invention encompasses a method of treating a young subject age 17 years or younger comprising administering to the subject a nucleic acid immunogenic composition and/or vaccine comprising one or more RNA polynucleotides having an open reading frame encoding an virus antigenic polypeptide in an effective amount to vaccinate the subject. In other aspects the invention encompasses a method of treating an adult subject comprising administering to the subject a nucleic acid immunogenic composition and/or vaccine comprising one or more RNA polynucleotides having an open reading frame encoding a virus antigenic polypeptide in an effective amount to administer to or vaccinate the subject.

In some aspects the invention relates to a method of administering (e.g., vaccinating) a subject with a combination immunogenic composition and/or vaccine including at least two nucleic acid sequences encoding antigens wherein the dosage for the vaccine is a combined therapeutic dosage wherein the dosage of each individual nucleic acid encoding an antigen is a sub therapeutic dosage. In some aspects, the combined dosage is 25 micrograms of the RNA polynucleotide in the nucleic acid immunogenic composition and/or vaccine administered to the subject. In some aspects, the combined dosage is 100 micrograms of the RNA polynucleotide in the nucleic acid immunogenic composition and/or vaccine administered to the subject. In some aspects the combined dosage is 50 micrograms of the RNA polynucleotide in the nucleic acid immunogenic composition and/or vaccine administered to the subject. In some aspects, the combined dosage is 75 micrograms of the RNA polynucleotide in the nucleic acid immunogenic composition and/or vaccine administered to the subject. In some aspects, the combined dosage is 150 micrograms of the RNA polynucleotide in the nucleic acid immunogenic composition and/or vaccine administered to the subject. In some aspects, the combined dosage is 400 micrograms of the RNA polynucleotide in the nucleic acid immunogenic composition and/or vaccine administered to the subject. In some aspects, an immunogenic composition comprising one lipid nanoparticle encapsulated mRNA molecule encoding HA is monovalent and has a dose selected from any one of 1 μg mRNA, 2 μg RNA, 5 μg RNA, and 20 μg RNA.

In some aspects, an immunogenic composition comprises a first lipid nanoparticle encapsulated mRNA molecule encoding HA, a second lipid nanoparticle encapsulated mRNA molecule encoding HA, a third lipid nanoparticle encapsulated mRNA molecule encoding NA, and a fourth lipid nanoparticle encapsulated mRNA molecule encoding NA, wherein the total dose is up to 20 μg RNA

In preferred aspects, immunogenic compositions and/or vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary aspects, antibody titer is expressed as the inverse of the greatest dilution (in a serial dilution) that still gives a positive result. In exemplary aspects, antibody titer is determined or measured by enzyme-linked immunosorbent assay (ELISA). In exemplary aspects, antibody titer is determined or measured by neutralization assay, e.g., by microneutralization assay. In certain aspects, antibody titer measurement is expressed as a ratio, such as 1:40, 1:100, etc.

In exemplary aspects of the invention, an efficacious immunogenic composition and/or vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary aspects, the antibody titer is produced or reached by 10 days following vaccination, by 20 days following vaccination, by 30 days following vaccination, by 40 days following vaccination, or by 50 or more days following vaccination. In exemplary aspects, the titer is produced or reached following a single dose of the immunogenic composition and/or vaccine administered to the subject. In other aspects, the titer is produced or reached following multiple doses, e.g., following a first and a second dose (e.g., a booster dose.) In exemplary aspects of the invention, antigen-specific antibodies are measured in units of ug/ml or are measured in units of IU/L (International Units per liter) or mIU/ml (milli International Units per ml). In exemplary aspects of the invention, an efficacious immunogenic composition and/or vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary aspects of the invention, an efficacious immunogenic composition and/or vaccine produces >10 mIU/ml, >20 mIU/ml, >50 mIU/ml, >100 mIU/ml, >200 mIU/ml, >500 mIU/ml or >1000 mIU/ml. In exemplary aspects, the antibody level or concentration is produced or reached by 10 days following vaccination, by 20 days following vaccination, by 30 days following vaccination, by 40 days following vaccination, or by 50 or more days following vaccination. In exemplary aspects, the level or concentration is produced or reached following a single dose of the immunogenic composition and/or vaccine administered to the subject. In other aspects, the level or concentration is produced or reached following multiple doses, e.g., following a first and a second dose (e.g., a booster dose.) In exemplary aspects, antibody level or concentration is determined or measured by enzyme-linked immunosorbent assay (ELISA). In exemplary aspects, antibody level or concentration is determined or measured by neutralization assay, e.g., by microneutralization assay.

### VI. Freezing or Lyophilization

Lyophilization may be carried out by freezing a sample in a first step and subsequently drying the sample in one or more steps via sublimation, optionally by reducing the surrounding pressure and/or by heating the sample so that the solvent sublimes directly from the solid phase to the gas phase. In some aspects, a method of lyophilization includes providing a composition comprising lipid nanoparticles encapsulating or associated with RNA and at least one cryoprotectant, and blank LNPs. In some aspects, the method further includes freeze-drying the composition in a freeze dryer, which refers to an instrument that allows lyophilization of lipid or semi-liquid formulations. Such instruments are available in the art.

As used herein, the term “cryoprotectant” typically refers to an excipient, which partially or totally replaces the hydration sphere around a molecule and thus prevents catalytic and/or hydrolytic processes. In some aspects, the cryoprotectant is a blank LNP or liposome, wherein the LNP or liposome does not encapsulate RNA. In some aspects, the cryoprotectant is a disaccharide (e.g., sucrose). In a preferred aspect, the composition provided in step a) comprises i) lipid nanoparticles encapsulating or associated with RNA; ii) least one cryoprotectant, wherein the cryoprotectant is a carbohydrate; and iii) lipid nanoparticles or liposomes that do not have RNA encapsulated or associated therein. In a another preferred aspect, the composition provided in step a) comprises i) lipid nanoparticles encapsulating or associated with RNA; and ii) an effective amount of least one cryoprotectant, wherein the cryoprotectant is a disaccharide, and wherein the effective amount of the cryoprotectant is at least about 2% w/v to 30% w/v (e.g., at least, at most, in between any two of, or exactly 2, 3, 4, 5, 6, 7, 8, 9, 10, 11, 12, 13, 14, 15, 16, 17, 18, 19, 20, 21, 22, 23, 24, 25, 26, 27, 28, 29, or 30% w/v/) of the composition. In other embodiments, the cryoprotectant is present at least about 2% to 50% w/v.

Exemplary carbohydrates may comprise, without being limited thereto, any carbohydrate suitable for the preparation of a pharmaceutical composition, preferably, without being limited thereto, monosaccharides, such as e.g., glucose, fructose, galactose, sorbose, mannose preferably means unbound or unconjugated, e.g., the mannose is not covalently bound to the at least one RNA, or in other words, the mannose is unconjugated, preferably with respect to the at least one RNA), etc., and mixtures thereof; disaccharides, such as e.g., lactose, maltose, sucrose, trehalose, cellobiose, etc., and mixtures thereof; polysaccharides, such as raffinose, melezitose, maltodextrins, dextrans, dextrins, cellulose, starches, etc., and mixtures thereof; and alditols, such as glycerol, mannitol, xylitol, maltitol, lactitol, xylitol sorbitol, pyranosyl sorbitol, myoinositol, etc., and mixtures thereof. Examples of sugars that are preferably comprised in the composition include lactose, mannose, mannitol, sucrose or trehalose. Specific examples of cryoprotectants include glyceraldehyde, erythrose, threose, ribose, lyxose, xylose, arabinose, allose, talose, gulose, glucose, altrose, mannose, galactose, idose, erythrulose, ribulose, psicose, fructose, sorbose, tagatose, erythritol, glycerol, isomaltol, lactitol, maltitol, mannitol, sorbitol, xylitol, inositol, sucrose, lactulose, lactose, maltose, trehalose, cellobiose, kojibiose, nigerose, isomaltose, isotrehalose, neotrehalose, sophorose, laminaribiose, gentiobiose, turanose, maltulose, palatinose, gentiobiose, mannobiose, melibiose, melibiulose, neolactose, galactosucrose, scillabiose, neohesperidose, rutinose, rutinulose, vicianose, xylobiose, primeverose, trehalosamine, maltitol, lactosamine, lactitol, sucralose, raffinose, panose, maltotriose, melezitose, gentianose, stachyose, cyclodextrin, hydroxypropyl-β-cyclodextrin, dextran, polyvinylpyrrolidone, polyethylene glycol and the like, preferably disaccharide, further preferably non-reducing disaccharide, most preferably sucrose. Preferably, the sugar is sucrose. In some aspects, the sugar has a high water displacement activity and a high glass transition temperature. In some aspects, the sugar is preferably hydrophilic but not hygroscopic. In some aspects, the sugar has a low tendency to crystallize. In some aspects, the cryoprotectant is selected from any one of mannitol, sucrose, glucose, mannose and trehalose. In some aspects, further components may be used as cryoprotectant. Particularly alcohols such as PEG, mannitol, sorbitol, cyclodextrin, DMSO, amino acids and proteins such as proline, glycine, phenylanaline, arginine, serine, and albumin and gelatine may be used as cryoprotectant. Additionally metal ions, surfactants and salts as defined below may be used as cryoprotectant. Furthermore polymers may be used as cryoprotectant, particularly polyvinylpyrrolidone.

In some aspects, the weight ratio of the RNA in the composition to the cryoprotectant, preferably a carbohydrate, more preferably a sugar, even more preferably sucrose, in said composition is preferably in a range from about 1:2000 to about 1:10, more preferably from about 1:1000 to about 1:100. Most preferably, the weight ratio of the at least one RNA in the composition to the cryoprotectant, preferably a carbohydrate, more preferably a sugar, even more preferably sucrose, in said liquid is in a range from about 1:250 to about 1:10 and more preferably in a range from about 1:100 to about 1:10 and most preferably in a range from about 1:100 to about 1:50. In some aspects, the cryoprotectant comprises 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% w/v, or more, or any range or value derivable therein, of the composition.

In some aspects, the composition may further include any one of the following: cryoprotectants, lyoprotectants, bulking agents, preservatives, antioxidants, metal chelators, antimicrobial agents, colorants, carriers, fillers, film formers, redispersants and disintegrants. Moreover, in some aspects, the composition may further include any one of the following excipients, such as defoamers, surfactants, viscosity enhancing agents, force control agents or the like.

The composition comprising at least one RNA and at least one lyoprotectant is in some aspects characterized by a glass transition temperature (Tg), which is in some aspects equal to or higher than 60° C., in some aspects equal to or higher than 70° C., and in some aspects equal to or higher than 80° C. In some aspects, the glass transition temperature of composition is in a range from 50° C. to 200° C., in some aspects from 60° C. to 120° C., and in some aspects from 70° C. to 100° C. and in some aspects from about 78° C. to about 88° C. In preferred aspects, the composition is characterized by a residual moisture content, which is in some aspects in the range from about 0.1% (w/w) to about 10% (w/w), in some aspects in the range from about 1% (w/w) to about 8% (w/w), and in some aspects in the range from about 2% (w/w) to about 5% (w/w), and in some aspects in the range from about 3% (w/w) to 4%, e.g., 3% (w/w)+2% (w/w), or 3% (w/w)+1% (w/w). In some aspects, the residual water content of the composition is equal to or less than 10% (w/w), in some aspects, equal to or less than 7% (w/w), in some aspects, equal to or less than 5% (w/w), and in some aspects, equal to or less than 4% (w/w). As used herein, the term “residual moisture content” (or “residual moisture”) refers to the total amount of solvent present in the composition. Said total amount of residual solvents in the composition may be determined using any suitable method known in the art. For example, methods for determining the residual moisture content may include the Karl-Fischer-titrimetric technique or the thermal gravimetric analysis (TGA) method. In some aspects, the residual solvent comprised in the composition is water or an essentially aqueous solution and the residual moisture content is determined by the Karl-Fischer-titrimetric technique.

The composition is preferably suitable as storage-stable form of lipid nanoparticles encapsulating RNA. The storage stability of the RNA may be determined through determination of the relative (structural) integrity and the biological activity after a given storage period, e.g., via time-course in vitro expression studies. The relative integrity may be determined as the percentage of full-length RNA (i.e. non-degraded RNA) with respect to the total amount of RNA (i.e. full-length RNA and degraded RNA fragments (which may appear as smears in gel electrophoresis), preferably after deduction of the LOD (3×background noise), for example, by using QuantityOne software from BioRad. In some aspects, the composition allows longer storage at temperatures from −80° C. to 60° C. than the corresponding composition, comprising LNPs encapsulating or associated with RNA in the absence of blank LNPs, in WFI or other injectable solutions. In some aspects, the composition may be stored at room temperature. In some aspects, the composition is stored with or without shielding gas. In some aspects, single doses of the composition are packaged and sealed. Alternatively, multiple doses may be packaged in one packaging unit.

The composition including the lipid nanoparticles encapsulating RNA may be stored for at least about 2 hours to 2 years. In some aspects, the RNA or encapsulated RNA is stored for equal to any one of, at least any one of, at most any one of, or between any two of at least about 2 hours, 4 hours to 8 weeks, 6 hours to seven weeks, 12 hours, 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 1 year, 2 years, or any range or value derivable therein. The composition including the lipid nanoparticles encapsulating RNA may be stored at a temperature of about room temperature to about −90° C. For example, the RNA or encapsulated RNA may be stored at a temperature below room temperature, at or below 4° C., at or below 0° C., at or below −20° C., at or below −60° C., at or below −70° C., at or below −80° C., or at or below −90° C. In some aspects, the composition including the lipid nanoparticles encapsulating RNA is stored at a temperature of equal to any one of, at least any one of, at most any one of, or between any two of about 20° C., 15° C., 10° C., 5° C., 0° C., −10° C., −20° C., −30° C., −40° C., −50° C., −60° C., −70° C., −80° C., or −90° C., or any range or value derivable therein.

In some aspects, the relative integrity of the at least one RNA in the composition is at least 70%, more preferably at least 75%, at least 80%, at least 85%, at least 90% or at least 95% after storage at room temperature for preferably at least one week, more preferably for at least one month, even more preferably for at least 6 months and most preferably for at least one year. In some aspects, the biological activity of the at least one RNA of the composition after storage at room temperature, is at least 70%, at least 75%, at least 80%, at least 85%, at least 90% or at least 95% of the biological activity of the RNA prior to lyophilization. The biological activity may be determined by analysis of the amounts of protein expressed from reconstituted LNPs encapsulating or associated with RNA and amounts of protein expressed from LNPs encapsulating or associated with RNA prior to lyophilization, respectively, e.g., after transfection into a mammalian cell line or into a subject. Alternatively, the biological activity may be determined by measuring the induction of an (adaptive or innate) immune response in a subject.

In some aspects, the RNA encapsulated LNP is lyophilized by: a) providing a composition having RNA encapsulated LNP and at least one cryoprotectant; b) loading the composition into a freeze drying chamber of a freeze dryer; c) cooling the composition to a freezing temperature; d) freezing the composition at the freezing temperature to obtain a frozen composition; e) reducing the pressure in the freeze drying chamber to a pressure below atmospheric pressure; f) drying the frozen composition obtained in step d) in order to obtain a lyophilized composition comprising the RNA encapsulated LNP and at least one cryoprotectant; g) equilibrating the pressure in the freeze drying chamber to atmospheric pressure and removing the lyophilized composition comprising RNA encapsulated LNP and the at least one cryoprotectant from the freeze drying chamber. In some aspects, the drying step f) is performed at a pressure below about 200 mbar. In some aspects, the pressure in the freeze drying chamber is between 3 to 200 mbar. In some aspects, the pressure in the freeze drying chamber is between 30 to 200 mbar. In some aspects, the pressure in the freeze drying chamber is between 3 to 100 mbar. In some aspects, the pressure in the freeze drying chamber is between 45 to 150 mbar.

### EXAMPLES

The following examples are included to demonstrate aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure.

### Example 1: Influenza modRNA

The Examples are based on the influenza modRNA, unless specified otherwise. The specific construct (Washington modRNA) is the only active ingredient in the immunogenic composition. The drug substance is formulated in 10 mM HEPES buffer, 0.1 mM EDTA at pH 7.0 and stored at −20±5° C.

In addition to the codon-optimized sequence encoding the antigen, the RNA contains common structural elements optimized for mediating high RNA stability and translational efficiency (5′-cap, 5′UTR, 3′-UTR, poly(A)-tail; see table and sequences below). Furthermore, an intrinsic signal peptide (sec) is part of the open reading frame and is translated as an N-terminal peptide. The RNA does not contain any uridines; instead of uridine, the modified N1-methylpseudouridine is used in RNA synthesis.

The specific constructs each comprise the elements shown below in Table 1:

TABLE 1
Construct Elements

| Element | Description | Position |
|---|---|---|
| 5′-cap | A modified 5′-cap1 structure (m⁷G⁺m3′-5′-ppp-5′-Am) | 1-2 |
| 5′-UTR | 5′-untranslated region derived from human alpha-globin | 3-54 |
| | RNA with an optimized Kozak sequence | |
| 3′-UTR | The 3′ untranslated region comprises two sequence | |
| | elements derived from the amino-terminal enhancer of | |
| | split (AES) mRNA and the mitochondrial encoded 12S | |
| | ribosomal RNA to confer RNA stability and high total | |
| | protein expression. | |
| poly(A) | A 110-nucleotide poly(A)-tail consisting of a | |
| | stretch of 30 adenosine residues, | |
| | followed by a 10-nucleotide linker | |
| | sequence and another 70 adenosine residues. | |

### Sequences of Elements

Cap and 5′-UTR: GAGAAψAAAC ψGψψ CψGGψCCCCA CAGACψCAGA GAGAACCCGC CACC (SEQ ID NO:1), where the bolded and underlined text corresponds to the cap and the unmodified text corresponds to the 5′-UTR.

| | |
|---|---|
| (SEQ ID NO: 2) | |
| 3′-UTR: CΨCGAGCΨGGΨ ACΨGCAΨGCA CGCAAΨGCΨA GCΨGCCCCΨΨ | |
| ΨCCCGΨCCΨG GGΨACCCCGA GΨCΨCCCCCG ACCΨCGGGΨC CCAGGΨPAΨGC | |
| ΨCCCACCΨCC ACCΨGCCCCA CΨCACCACCΨ CΨGCΨAGΨΨC CAGACACCΨC | |
| CCAAGCACGC AGCAAΨGCAG CΨCAAAACGC ΨΨ | GCCΨAGC CACACCCCCA |
| CGGGAAACAG CAGΨGAΨΨAA CCΨΨΨAGCAA ΨAAACGAAAG ΨΨΨAACΨAAG | |
| CΨAΨACΨAAC CCCAGGGΨΨG GΨCAAΨΨΨCG ΨGCCAGCCAC ACCCΨGGAGC | |
| ΨAGC | |
| Poly(A) tail: | |
| (SEQ ID NO: 3) | |
| AAAAAA AAAAAAAAAA AAAAAAAAAA AAAAGCAΨAΨ GACΨAAAAAA | |
| AAAAAAAAAA AAAAAAAAAA AAAAAAAAAA AAAAAAAAAA AAAAAAAAAA | |
| AAAAAAAAAA AAAA | |

As used herein, Y represents 1-methyl-3′-pseudouridylyl.

The 5′-cap analog (m₂⁷·3′^{-OMe}Gppp(m₁²′^{-O})ApG) for production of RNA containing a cap1 structure is shown below

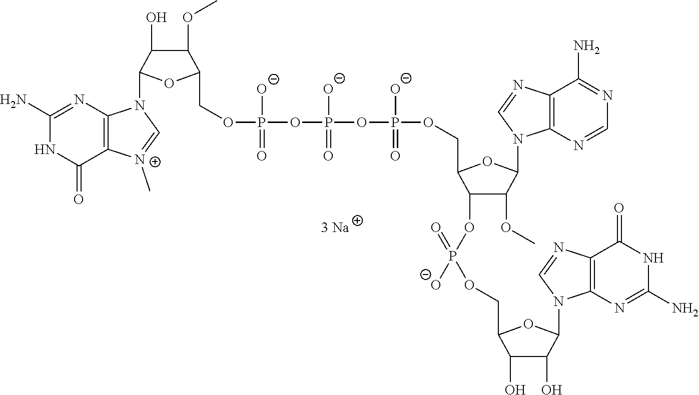 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

The above structure corresponds to Trilink's CleanCap AG (3′OMe)-m27,3′-OGppp (m12′-O) ApG. This molecule is identical to the natural RNA cap structure in that it starts with a guanosine methylated at N7, and is linked by a 5′ to 5′ triphosphate linkage to the first coded nucleotide of the transcribed RNA (in this case, an adenosine). This guanosine is also methylated at the 3′ hydroxyl of the ribose to mitigate possible reverse incorporation of the cap molecule. Finally, the 2′ hydroxyl of the ribose on the adenosine is methylated, conferring a Cap1 structure—by contrast, leaving this as a 2′ hydroxyl would give this a Cap0 structure. Cap1 structures should provide superior transcription to RNA's with a Cap0 structure in eukaryotes.

The influenza modRNA vaccine candidates may encode the HA protein derived from A/Wisconsin/588/2019 (H1N1), A/Cambodia/e0826360/2020 (H3N2), B/Washington/02/2019 (B/Victoria-lineage) and B/Phuket/3073/2013 (B/Yamagata lineage), which are the recommended vaccine strains for the cell culture-based influenza vaccines for the Northern Hemisphere 2021-2022 season. The number of A nucleotides present in the poly(A)-tail in the sequences preferably reflect how it would be in the final RNA after linearization with BspQ1 (or its isoschizomer): 30A-linker-70A. In the transcribed RNA with Cap 1 structure, the first two nucleotides in the mRNA sequence (AG) are actually provided by the CLEANCAP reagent and the 2′ hydroxyl of the ribose on the first adenosine is methylated.

The cap1 structure (i.e., containing a 2′-O-methyl group on the penultimate nucleoside of the 5′-end of the RNA chain) is incorporated into the drug substance by using a respective cap analog during in vitro transcription. For RNAs with modified uridine nucleotides, the cap1 structure is superior to other cap structures, since cap1 is not recognized by cellular factors such as IFIT1 and, thus, cap1-dependent translation is not inhibited by competition with eukaryotic translation initiation factor 4E. In the context of IFIT1 expression, mRNAs with a cap1 structure give higher protein expression levels.

In some preferred embodiments, the Influenza vaccine drug substance is a single-stranded, 5′-capped mRNA that is translated into the respective protein (the encoded antigen) which corresponds to the Hemagglutinin (HA) protein from Influenza strains either A/Wisconsin/588/2019 H1N1, A/Cambodia/e0826360/2020, B/Washington/02/2019 or B/Phuket/3073/2013. the general structure of the antigen-encoding RNA is determined by the respective nucleotide sequence of the DNA used as template for in vitro RNA transcription. In addition to the codon-optimized sequence encoding the antigen, the RNA contains common structural elements optimized for mediating high RNA stability and translational efficiency (5′-cap, 5′UTR, 3′-UTR, poly(A)-tail; see below). The RNA does not contain any uridines; instead of uridine the modified N1-methylpseudouridine is used in RNA synthesis.

The manufacturing process comprises RNA synthesis via an in vitro transcription (IVT) step followed by DNase I and proteinase K digestion steps, purification by ultrafiltration/diafiltration (UFDF), final filtration, dispense into an appropriate container, and storage at −20° C. A platform approach to the IVT, digestion, and purification process steps was used in the production of the four modRNAs. The mRNA clinical batches were prepared at a scale of 37.6 L starting volume for IVT. All the material was purified by a single 2-stage UFDF to produce mRNA drug substance.

The influenza modRNA immunogenic composition is comprised of one or more nucleoside-modified mRNAs that encode the full-length HA glycoprotein derived from seasonal human influenza strains. The modRNA is formulated with 2 functional and 2 structural lipids, which protect the modRNA from degradation and enable transfection of the modRNA into host cells after IM injection. Influenza HA is the most abundant envelope glycoprotein on the surface of influenza A and B virions.

The LNP formulation contains 2 functional lipids, ALC-0315 and ALC-0159, and 2 structural lipids, DSPC (1,2-distearoyl-sn-glycero-3-phosphocholine) and cholesterol. The physicochemical properties and the structures of the 4 lipids are shown in the Table below.

Lipid nanoparticles were prepared and tested according to the general procedures described in U.S. Pat. No. 9,737,619 (PCT Pub. No. WO2015/199952) and U.S. Pat. No. 10,166,298 (WO 2017/075531) and WO2020/146805. Briefly, cationic lipid, DSPC, cholesterol and PEG-lipid were solubilized in ethanol at a molar ratio of about 47.5:10:40.7:1.8. Lipid nanoparticles (LNPs) were prepared at a preferred total lipid to mRNA weight ratio of approximately 10:1 to 30:1. The mRNA was diluted in buffer. Syringe pumps were used to mix the ethanolic lipid solution with the mRNA aqueous solution. The ethanol was then removed and the external buffer replaced with another buffer (e.g., Tris) by dialysis. Finally, the lipid nanoparticles were filtered.

TABLE 2
Lipids in the LNP Formulation

| | | | Physical | |
|---|---|---|---|---|
| Lipid | Molecular | | state and | |
| (CAS | weight | Molecular | storage | |
| number) | [Da] | formula | condition | Chemical name (synonyms) and structure |
| ALC-0315 | 766 | C₄₈H₉₅NO₅ | Liquid (oil) | (4-hydroxybutyl)azanediyl)bis(hexane-6,1- |
| (not | | | −20° C. | diyl)bis(2-hexyldecanoate) |
| applicable) | | | | 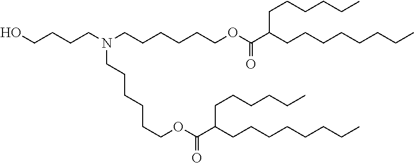 [SMILES: CCCCCCCCC(CCCCCC)C(=O)OCCCCCCN(CCCCO)CCCCCCOC(=O)C(CCCCCC)CCCCCCCC] |
| ALC-0159 | ~2400- | C₃₀H₆₀NO | Solid | 2-[(polyethylene glycol)-2000]-N,N- |
| (1849616- | 2600 | (C₂H₄O)ᵤOCH₃ | −20° C. | ditetradecyclacetamide |
| 42-7) | | n = 45-50 | | 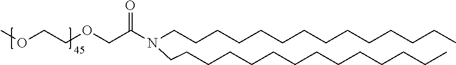 |
| DSPC | 790 | C₄₄H₈₈NO₈P | Solid | 1,2-Distearoyl-sn-glycero-3-phosphocholine |
| (816-94-4) | | | −20° C. | |
| | | | | 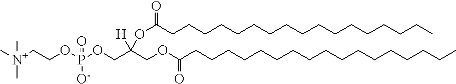 [SMILES: CCCCCCCCCCCCCCCCCC(=O)OCC(COP(=O)([O-])OCC[N+](C)(C)C)OC(=O)CCCCCCCCCCCCCCCCC] |
| Cholesterol (57-88-5) | 387 | C₂₇H₄₆O | Solid −20° C. | 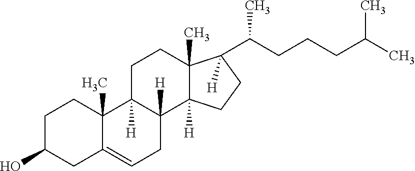 |

CAS = Chemical Abstracts Service; DSPC = 1,2-distearoyl-sn-glycero-3-phosphocholine.

### Example 2: Compositions Subjected to Freeze-Thawing or Freeze-Drying

The example formulation contains a mixture of (a) mRNA drug product (DP) including lipid nanoparticles containing an influenza modRNA (i.e., a RNA polynucleotide comprising a 5′ terminal cap, a 5′ UTR, a 3′UTR, and a 3′ polyadenylation tail, encoding an influenza antigen, wherein the RNA polynucleotide comprises at least one modified nucleoside, such as, for example, N1-methylpseudouridine, and preferably wherein 100% of the uracil in the open reading frame is a N1-methylpseudouridine) coded for the Washington 2019 hemagglutinin (mRNA LNPs) and (b) lipid nanoparticles excluding nucleic acid (blank LNPs), dispersed in a sucrose and Tris solution (pH 7.4). As used in the Examples herein, the term “blank LNPs” refers to a lipid nanoparticle that comprises the lipids listed in Table 2, wherein the lipid nanoparticle does not encapsulate any nucleic acid.

The following exemplary example illustrates studies to conduct multiple freeze-thaw cycling experiments (−80° C.↔+20° C.) or freeze-drying experiments with flu mRNA formulations (0.001 to 0.5 mg/mL mRNA) as shown in Table 3 containing 10.3% vs. 20.5% w/v Sucrose in the absence and presence of blank lipid nanoparticles, as shown below in Table 5.

Freeze-drying was conducted with and without annealing at a temperature above the glass transition temperature of the maximally freeze concentrated solution (Tg′) and below the ice melting temperature.

TABLE 3
Formulations for Freeze-Thawing
or Freeze-Drying using Flu modRNA

| | | | | | Target lipids | |
|---|---|---|---|---|---|---|
| | | | | | equivalent to | |
| | | | | Inclusion | 0.1 or 0.5 | Blank |
| | mRNA, | Tris, | Sucrose, | of Blank | mg/mL | LNP:mRNA |
| F# | mg/mL | mM | % w/v | LNPs | mRNA DP | LNP |
| 1 | 0.5 | 10 | 10.3 | No | NA | NA |
| 2 | 0.1 | 10 | 10.3 | No | NA | NA |
| 3 | 0.01 | 10 | 10.3 | No | NA | NA |
| 4 | 0.001 | 10 | 10.3 | No | NA | NA |
| 5 | 0.1 | 10 | 10.3 | Yes | 0.5 | 1:5 |
| 6 | 0.01 | 10 | 10.3 | Yes | 0.5 | 1:50 |
| 7 | 0.001 | 10 | 10.3 | Yes | 0.5 | 1:500 |
| 8 | 0.01 | 10 | 10.3 | Yes | 0.1 | 1:10 |
| 9 | 0.001 | 10 | 10.3 | Yes | 0.1 | 1:100 |
| 10 | 0.5 | 10 | 20.5 | No | NA | NA |
| 11 | 0.1 | 10 | 20.5 | No | NA | NA |
| 12 | 0.01 | 10 | 20.5 | No | NA | NA |
| 13 | 0.001 | 10 | 20.5 | No | NA | NA |
| 14 | 0.01 | 10 | 20.5 | Yes | 0.1 | 1:10 |
| 15 | 0.001 | 10 | 20.5 | Yes | 0.1 | 1:100 |

### Example 3: Exemplary Lyophilization Cycles

Exemplary methods of lyophilizing LNPs are known in the art. For example, the composition described herein comprising lipid nanoparticles may be lyophilized, wherein aliquots of 75 μl may be dispensed into sterile 2R glass vials (Type 1). The vials are partially stoppered with a freeze drying rubber stopper. The vials are loaded into a freeze dryer and dried under the following conditions. The lyophilization cycle under controlled freezing (with or without annealing) and controlled drying conditions are preferably optimized. Exemplary lyophilization steps are described in Table 4 below.

TABLE 4
Exemplary Lyophilization Steps

| | | Temperature | Pressure | Duration |
|---|---|---|---|---|
| Step | Description | (shelves) | Pirani | (hh:mm) |
| 1 | Loading | <−70° C. | atm | 00:00 |
| 4 | Evacuation | <−50° C. | 63 μbar | ~00:20 |
| | | (shelves cooled | | |
| | | with liquid | | |
| | | nitrogen) | | |
| 5 | Primary drying | <−50° C. → 40° C. | 63 μbar | 18:30 |
| | | heating only | | |
| | | controlled by | | |
| | | final | | |
| | | temperature | | |
| 9 | Secondary | 40° C. shelves | 45 μbar | 06:00 |
| | drying | results in | | |
| | | approximately | | |
| | | 20° C. in the | | |
| | | product | | |
| 10 | Nitrogen back- | 40° C. | n.a. | — |
| | fill | | | |
| 11 | Vial closure | 40° C. | n.a. | — |
| 12 | Aeration | 40° C. | atm | — |

The vials may be sealed by crimping an aluminum cap over the stopper and the neck of the vial. Afterwards, the samples are stored at 5° C., 25° C./60% relative humidity (R.H.), or 40° C./75% R.H. and analyzed for relative integrity after 5, 8, 13, 24, and 40 weeks (3 samples each). The relative integrity of the mRNA comprised in the lyophilized compositions are determined via agarose gel electrophoresis. Specifically, the relative integrity are determined by measuring the signal intensities corresponding to full-length mRNA and all other signals, respectively, in a lane of the agarose gel (i.e. in a given sample) and calculating the ratio of the signal intensity for full-length mRNA related to all other signals in that lane.

### Example 4: Impact of Freeze-Thaw Cycling on Lipid Nanoparticles Containing Flu modRNA

Blank LNPs (comprised of the lipids according to Table 2) were added to a composition comprising the LNPs encapsulating Flu modRNA (0.5, 0.1, 0.01, and 0.001 mg/mL, Washington strain, said LNPs encapsulating Flu modRNA comprising the lipids according to Table 2) such that the lipid content of the resulting formulation was equivalent to that of a 0.5 and 0.1 mg/ml mRNA drug product. A separate batch of blank LNPs (comprised of the lipids according to Table 2) was prepared and mixed with a batch of mRNA-containing LNPs and the formulation buffer to achieve the target lipids and mRNA concentrations in the final formulations. 5× freeze-thaw (FT) cycling was conducted between −80° C. and RT (2.25 mL fill).

As shown in FIGS. 1-12, the inclusion of the blank LNPs mitigated the increase in size and PDI and the decrease in % encapsulation post-freeze-thawing observed in the dispersions that do not contain blank LNPs. The effect of blank LNPs was evident in the more dilute compositions.

TABLE 5
Formulation attributes prior to Freeze-Thawing

| | | | | | Target lipids | | | | |
|---|---|---|---|---|---|---|---|---|---|
| | | | | | equivalent to | | | | |
| | | | | Inclusion | 0.1 or 0.5 | Z- | | | |
| | mRNA, | Tris, | Sucrose, | of Blank | mg/mL | avg, | | % | Concn, |
| F# | mg/mL | mM | % w/v | LNPs | mRNA DP | nm | PDI | Encap | mg/mL |
| 1 | 0.5 | 10 | 10.3 | No | NA | 53 | 0.19 | 96 | 0.505 |
| 2 | 0.1 | 10 | 10.3 | No | NA | 51 | 0.10 | 94 | 0.101 |
| 3 | 0.01 | 10 | 10.3 | No | NA | 52 | 0.14 | 93 | 0.010 |
| 4 | 0.001 | 10 | 10.3 | No | NA | 66 | 0.28 | 87 | 0.001 |
| 5 | 0.1 | 10 | 10.3 | Yes | 0.5 | 45 | 0.12 | 97 | 0.101 |
| 6 | 0.01 | 10 | 10.3 | Yes | 0.5 | 49 | 0.23 | 97 | 0.010 |
| 7 | 0.001 | 10 | 10.3 | Yes | 0.5 | 48 | 0.23 | 97 | 0.001 |
| 8 | 0.01 | 10 | 10.3 | Yes | 0.1 | 53 | 0.19 | 96 | 0.011 |
| 9 | 0.001 | 10 | 10.3 | Yes | 0.1 | 51 | 0.10 | 96 | 0.001 |
| Blank | 0 | 10 | 10.3 | Yes | 0.5 | 55 | 0.12 | NA | NA |
| Blank | 0 | 10 | 10.3 | Yes | 0.1 | 57 | 0.14 | NA | NA |
| 10 | 0.5 | 10 | 20.5 | No | NA | 42 | 0.72 | 96 | 0.494 |
| 11 | 0.1 | 10 | 20.5 | No | NA | 56 | 0.24 | 100 | 0.101 |
| 12 | 0.01 | 10 | 20.5 | No | NA | 59 | 0.26 | 92 | 0.011 |
| 13 | 0.001 | 10 | 20.5 | No | NA | 52 | 0.31 | 89 | 0.001 |
| 14 | 0.01 | 10 | 20.5 | Yes | 0.1 | 54 | 0.31 | 95 | 0.010 |
| 15 | 0.001 | 10 | 20.5 | Yes | 0.1 | 54 | 0.34 | 94 | 0.001 |

The inclusion of blank LNPs mitigated the increase in size on freeze-thaw, especially at the lower mRNA dose compositions (0.01 and 0.001 mg/mL).

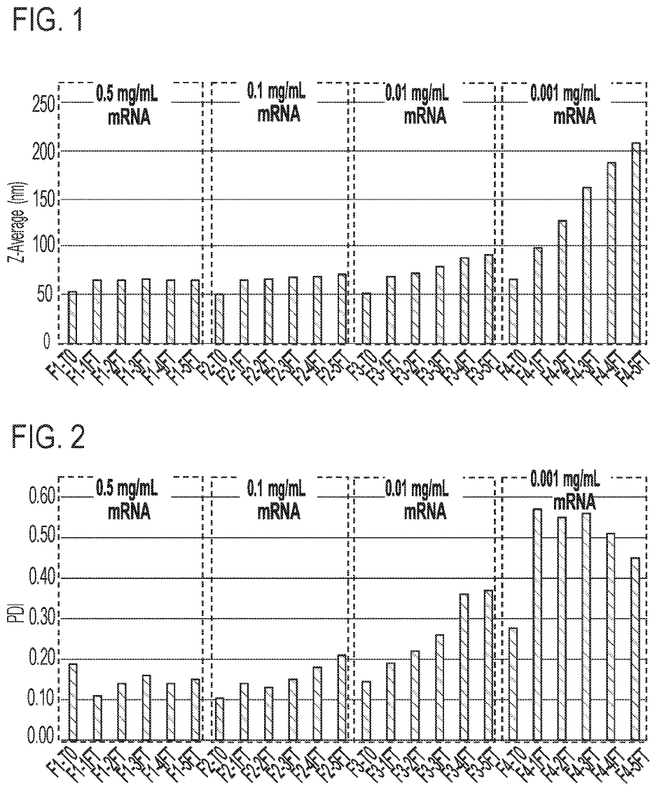

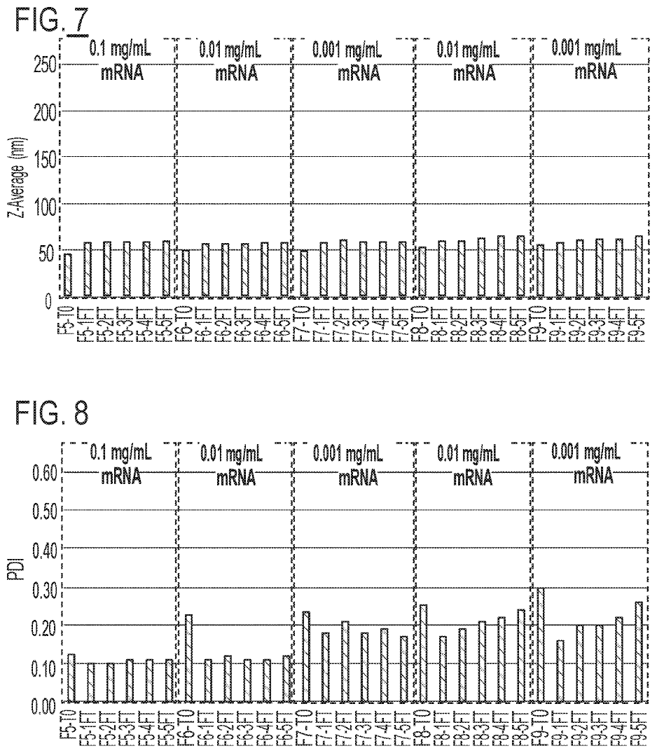

A direct, side-by-side comparison of values obtained for the size (Z-average in nm) (FIG. 1, FIG. 7), PDI (FIG. 2, FIG. 8), and encapsulation efficiency (Encapsulation %) (FIG. 3, FIG. 9) for flu mRNA formulations containing 10.3% w/v Sucrose in the presence (FIGS. 7-9) and absence (FIGS. 1-3) of blank LNPs is shown below in Table 6.

TABLE 6
Summary of the 10.3% w/v Sucrose flu mRNA formulation
attributes with and without blank LNPs

| | | Z-Average, nm | | PDI | | % Encapsulation | |
|---|---|---|---|---|---|---|---|
| mRNA, | Sucrose, | −blank | +blank | −blank | +blank | −blank | +blank |
| mg/mL | % w/v | LNPs | LNPs | LNPs | LNPs | LNPs | LNPs |
| 0.5 | 10.3 | 53-65 | NA | 0.11-0.19 | NA | 96-97 | NA |
| 0.1 | | 51-71 | 45-59 | 0.10-0.21 | 0.10-0.12 | 94-97 | 97-98 |
| 0.01 | | 52-91 | 49-57 | 0.14-0.37 | 0.23-0.11 | 90-95 | 97-98 |
| | | | 52-64* | | 0.17-0.25 | | 96-97 |
| 0.001 | | 66-206 | 48-60 | 0.28-0.45 | 0.17-0.23 | 50-87 | 96-97 |
| | | | 55-64 | | 0.16-0.30 | | 84-96 |

*Bolded values correspond to lower target lipid concentration equivalent to 0.1 mg/mL mRNA drug product

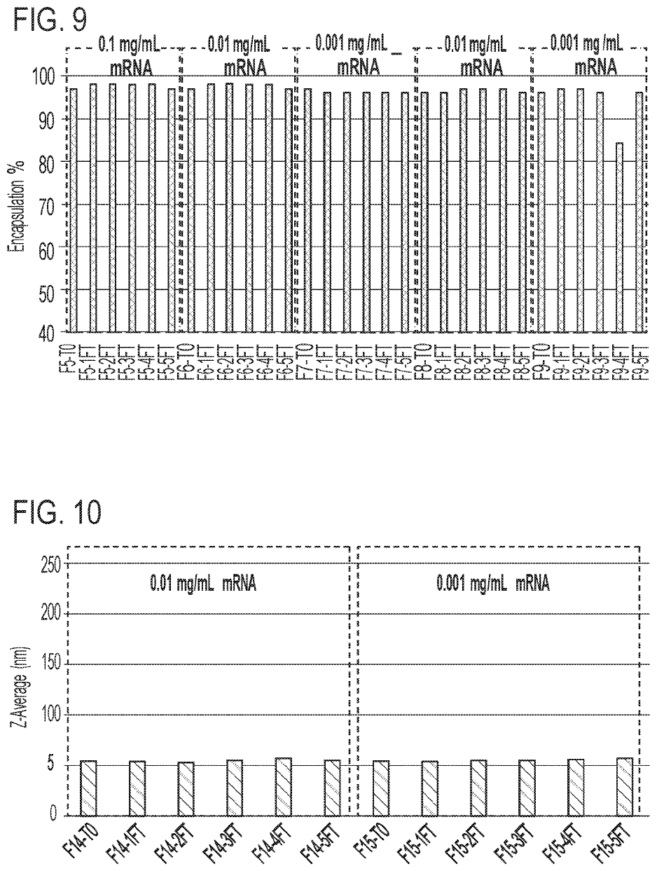

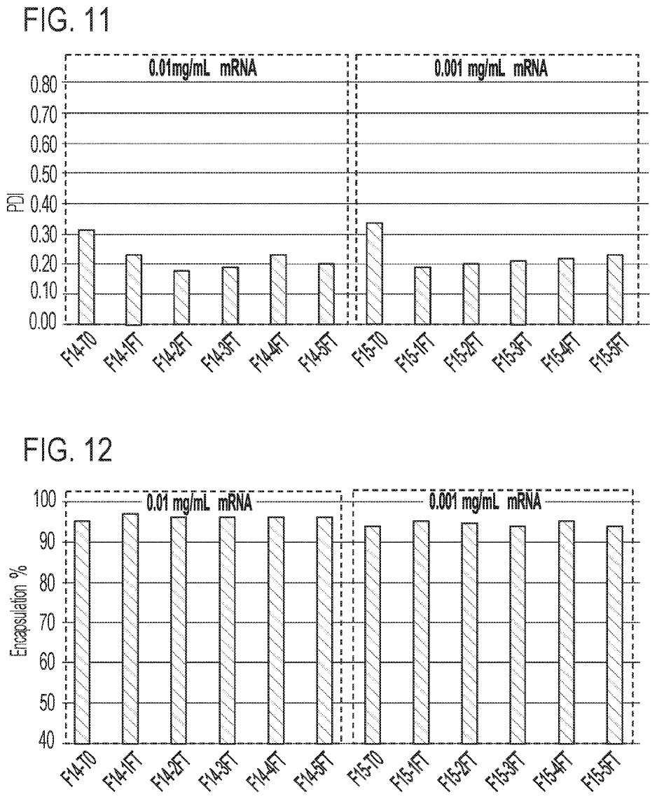

Similarly, as shown in FIGS. 10-12, the inclusion of blank LNPs at a concentration to raise the total lipid concentration equivalent to that of a formulation containing 0.1 mg/ml mRNA drug product without blank LNPs (F #14-15) in flu mRNA formulations containing 20.5% w/v Sucrose mitigated the increase in size (FIG. 10), PDI (FIG. 11), and encapsulation efficiency (FIG. 12) observed over increasing cycles of freeze-thaw, especially in the case of the more dilute formulations comprising 0.01 and 0.001 mg/ml mRNA.

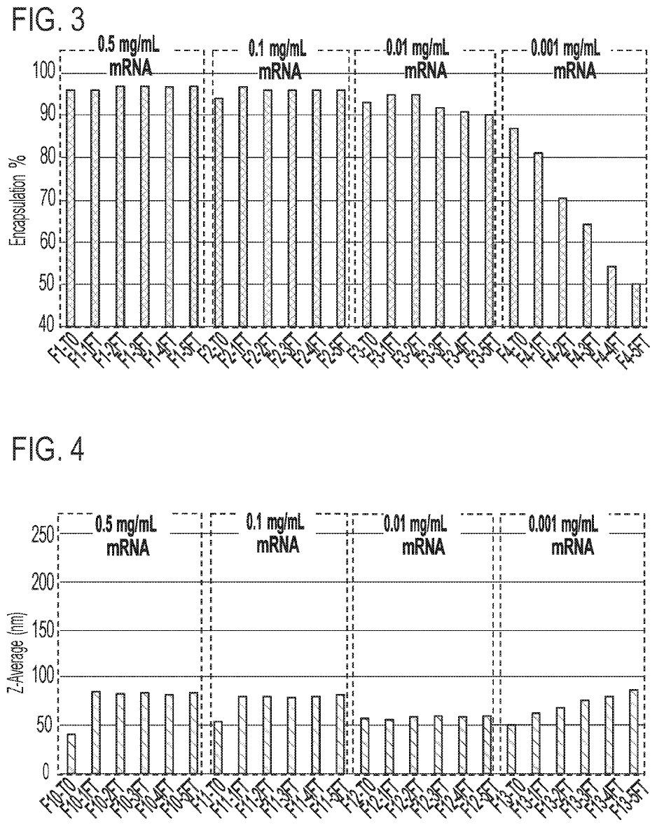

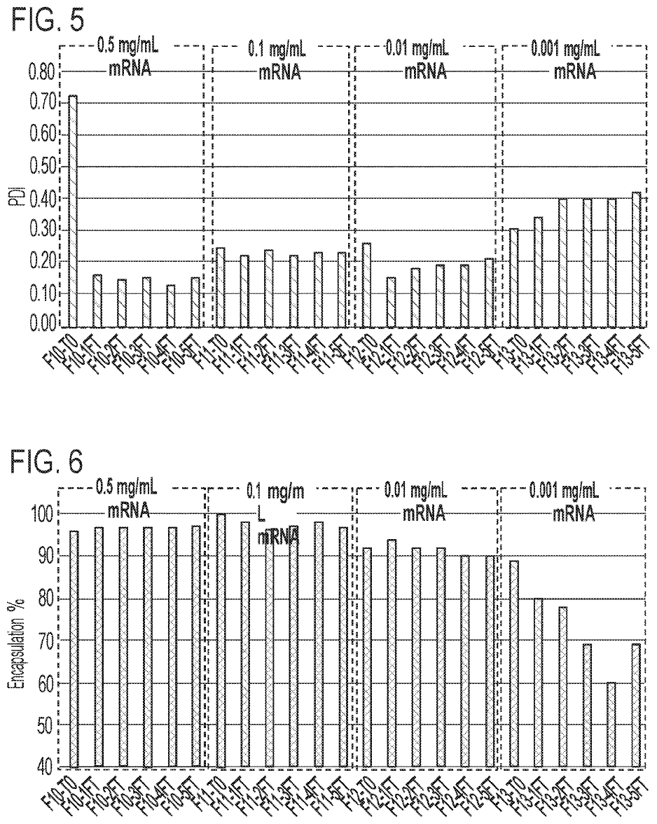

A direct, side-by-side comparison of values obtained for the size (Z-average in nm) (FIG. 4, FIG. 10), PDI (FIG. 5, FIG. 11), and encapsulation efficiency (Encapsulation %) (FIG. 6, FIG. 12) for flu mRNA formulations containing 20.5% w/v Sucrose in the presence (FIGS. 10-12) and absence (FIGS. 4-6) of blank LNPs is shown below in Table 7.

TABLE 7
Summary of the 20.5% w/v Sucrose flu mRNA formulation
attributes with and without blank LNPs

| | | Z-Average, nm | | PDI | | % Encapsulation | |
|---|---|---|---|---|---|---|---|
| mRNA, | Sucrose, | −blank | +blank | −blank | +blank | −blank | +blank |
| mg/mL | % w/v | LNPs | LNPs | LNPs | LNPs | LNPs | LNPs |
| 0.5 | 20.5 | 42-87 | NA | 0.13-0.16-0.72 | NA | 96-97 | NA |
| 0.1 | | 56-85 | NA | 0.22-0.24 | NA | 97-100 | NA |
| 0.01 | | 58-62 | 53-57 | 0.15-0.26 | 0.18-0.31 | 90-94 | 95-97 |
| 0.001 | | 52-90 | 54-57 | 0.31-0.42 | 0.19-0.34 | 60-89 | 94-95 |

The inclusion of 20.5% Sucrose alone (F #10-13, -blank LNPs) (FIGS. 4-6) did not provide any additional advantage for preservation of the chemical and physical characteristics of the flu mRNA formulations over the course of five freeze-thaw cycles compared to using 10.3% w/v Sucrose (F #1-4, -blank LNPs) (FIGS. 1-3). Additionally, the inclusion of blank LNPs in the flu mRNA formulations was more effective in preventing an increase in size and PDI and a decrease in % encapsulation.

### Example 5: Impact of Freeze-Drying on Lipid Nanoparticles Containing Flu modRNA

Blank LNPs (comprised of the lipids according to Table 2) were added to a composition comprising the LNPs encapsulating Flu modRNA (0.5, 0.1, 0.01, and 0.001 mg/mL, Washington strain, said LNPs encapsulating Flu modRNA comprising the lipids according to Table 2) such that the lipid content of the resulting formulation was equivalent to that of a 0.5 and 0.1 mg/mL mRNA drug product. A separate batch of blank LNPs (comprised of the lipids according to Table 2) was prepared and mixed with a batch of mRNA-containing LNPs and the formulation buffer to achieve the target lipids and mRNA concentrations in the final formulations.

Freeze-drying was conducted with and without annealing (0.3 mL fill), and formulation attributes were measured for the cakes reconstituted in saline vs. sterile water for injection (sWFI).

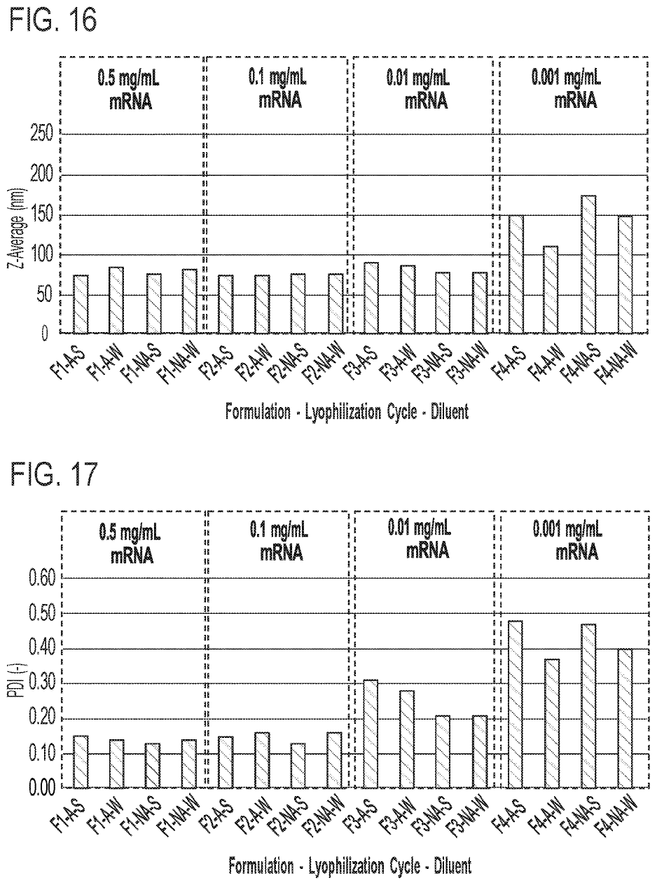

As shown in FIGS. 16-27, the inclusion of the blank LNPs in annealed (A) and non-annealed (NA) freeze-dried mRNA-containing LNP samples reconstituted in saline(S) or sterile water (W) mitigated the increase in size and PDI and the decrease in % encapsulation post-reconstitution observed in the dispersions that do not contain blank LNPs. The effect of blank LNPs was evident in the more dilute composition (0.01 and 0.001 mg/mL).

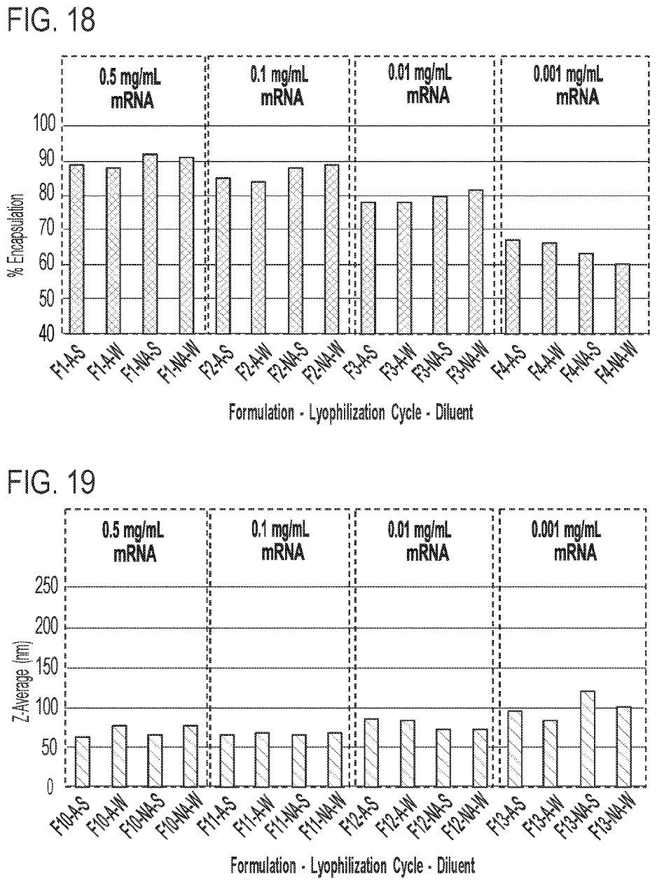

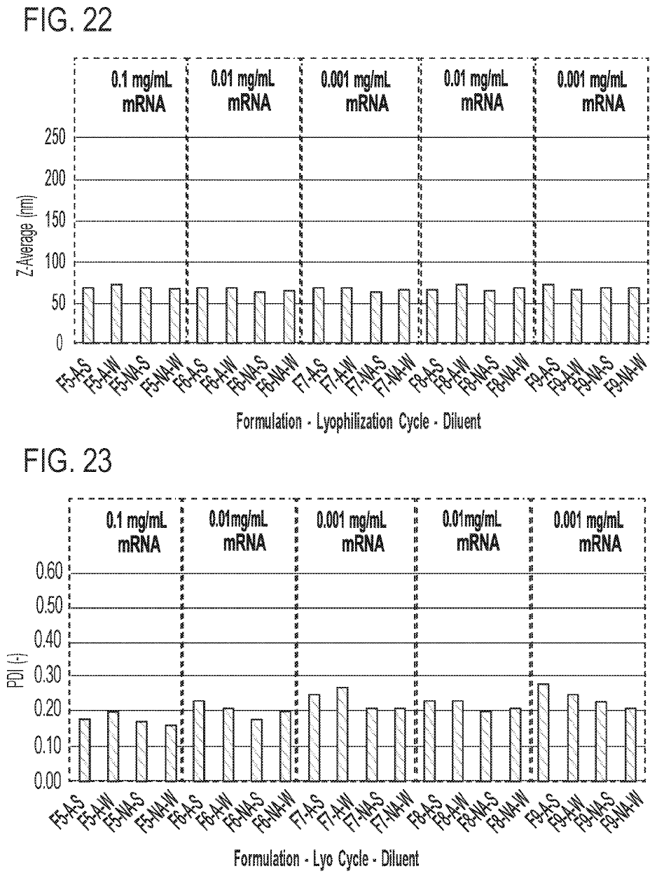

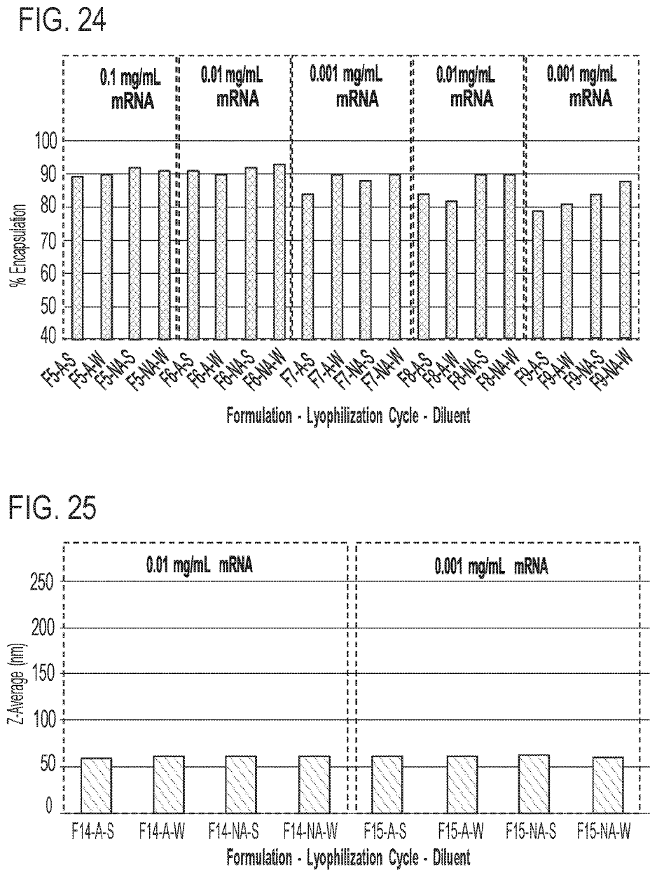

A direct, side-by-side comparison of values obtained for the size (Z-average in nm) (FIG. 16, FIG. 22), PDI (FIG. 17, FIG. 23), and encapsulation efficiency (Encapsulation %) (FIG. 18, FIG. 24) for flu mRNA formulations containing 10.3% w/v Sucrose in the presence (FIGS. 22-24) and absence (FIGS. 16-18) of blank LNPs is shown below in Table 8.

TABLE 8
Summary of 10.3% w/v Sucrose flu mRNA formulation attributes
with and without blank LNPs post freeze-drying

| | | Z-Average, nm | | PDI | | % Encapsulation | |
|---|---|---|---|---|---|---|---|
| mRNA, | Sucrose, | −blank | +blank | −blank | +blank | −blank | +blank |
| mg/mL | % w/v | LNPs | LNPs | LNPs | LNPs | LNPs | LNPs |
| 0.5 | 10.3 | 75-85 | NA | 0.13-0.15 | NA | 88-92 | NA |
| 0.1 | | 74-77 | 68-72 | 0.13-0.16 | 0.16-0.20 | 84-89 | 89-92 |
| 0.01 | | 79-91 | 61-68 | 0.21-0.31 | 0.18-0.23 | 78-82 | 90-93 |
| | | | 65-70* | | 0.20-0.23* | | 82-90* |
| 0.001 | | 111-176 | 64-69 | 0.37-0.48 | 0.21-0.27 | 60-67 | 84-90 |
| | | | 67-70* | | 0.21-0.28* | | 79-88* |

*Bolded values correspond to lower target lipid concentration equivalent to 0.1 mg/mL mRNA drug product

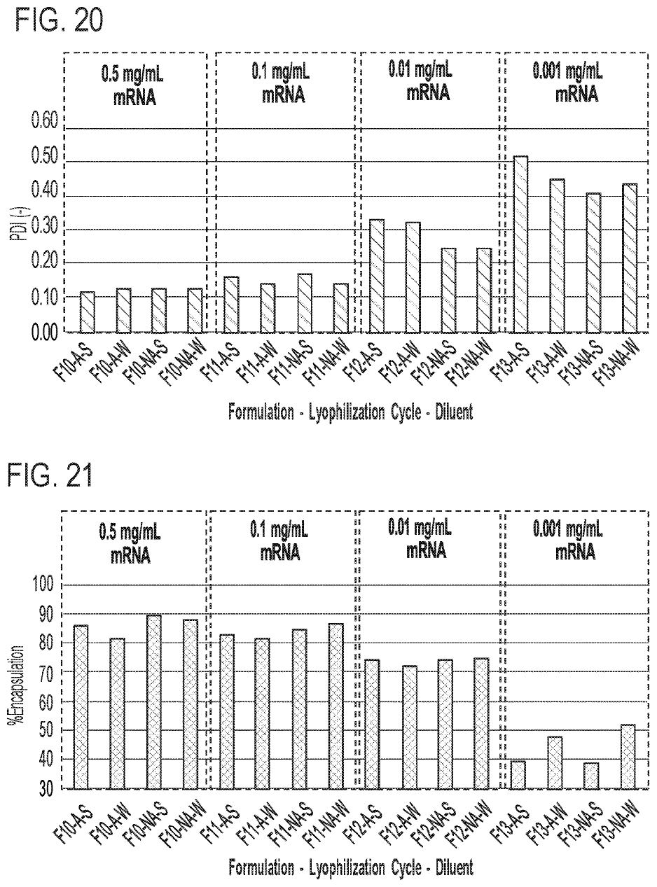

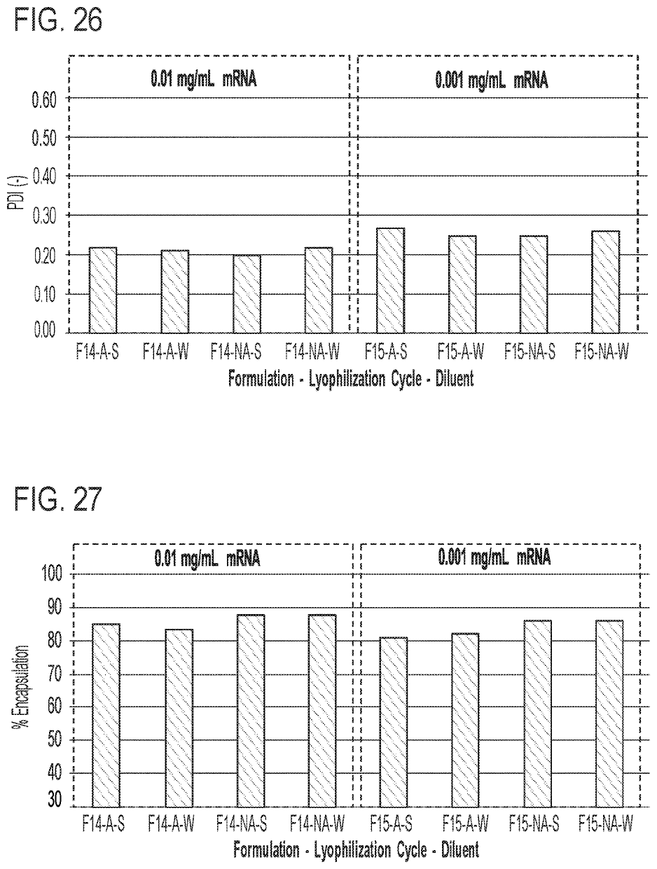

Similarly, as shown in FIGS. 25-27, the inclusion of blank LNPs at a concentration to raise the total lipid concentration equivalent to that of a formulation containing 0.1 mg/ml mRNA drug product without blank LNPs (F #14-15) in flu mRNA formulations containing 20.5% w/v Sucrose mitigated the increase in size (FIG. 25), PDI (FIG. 26), and encapsulation efficiency (FIG. 27) observed after reconstitution, especially in the case of the more dilute formulations comprising 0.01 and 0.001 mg/ml mRNA. A direct, side-by-side comparison of values obtained for the size (Z-average in nm) (FIG. 19, FIG. 25), PDI (FIG. 20, FIG. 26), and encapsulation efficiency (Encapsulation %) (FIG. 21, FIG. 27) for flu mRNA formulations containing 20.5% w/v Sucrose in the presence (FIGS. 25-27) and absence (FIGS. 19-21) of blank LNPs is shown below in Table 9.

TABLE 9
Summary of 20.5% w/v Sucrose flu mRNA formulation attributes
with and without blank LNPs post freeze-drying

| | | Z-Average, nm | | PDI | | % Encapsulation | |
|---|---|---|---|---|---|---|---|
| mRNA, | Sucrose, | −blank | +blank | −blank | +blank | −blank | +blank |
| mg/mL | % w/v | LNPs | LNPs | LNPs | LNPs | LNPs | LNPs |
| 0.5 | 20.5 | 75-85 | NA | 0.13-0.15 | NA | 88-92 | NA |
| 0.1 | | 64-69 | NA | 0.14-0.17 | NA | 82-87 | NA |
| 0.01 | | 72-85 | 59-62 | 0.25-0.33 | 0.20-0.22 | 72-75 | 83-88 |
| 0.001 | | 84-120 | 61-63 | 0.41-0.52 | 0.25-0.27 | 39-52 | 81-86 |

The inclusion of 20.5% Sucrose alone (F #10-13, -blank LNPs) (FIGS. 19-21) did not provide any additional advantage for preservation of the chemical and physical characteristics of the flu mRNA formulations after reconstitution compared to using 10.3% w/v Sucrose (F #1-4, -blank LNPs) (FIGS. 16-18). Additionally, the inclusion of blank LNPs in the flu mRNA formulations was more effective in preventing an increase in size and PDI and a decrease in % encapsulation.

### Example 6: saRNA LNP Formulation with Blank LNPs

Self-amplifying RNA encapsulated LNPs (said LNPs prepared according to Table 2) were diluted to assess impact on particle size and polydispersion index (PDI) pre- and post-freeze thaw conditions. It was observed that as the LNP concentration decreases upon dilution, a greater percentage of the PEG may be dissociating, resulting in larger particle sizes and PDI changes post-freeze thaw. See Table 10, Table 11, and Table 12.

TABLE 10
Formulation attributes pre- and post-freeze-thaw for
various dilutions of saRNA LNPs

| | T=0 (liquid) | 1× F/T −80° C. | 3× F/T −80° C. |
|---|---|---|---|
| Concentration (mg/ml) | Size/PDI | Size/PDI | Size/PDI |
| 0.06 | 63.4/0.15 | 64.9/0.16 | 69.1/0.19 |
| 0.03 | 64.0/0.16 | 65.8/0.16 | 71.4/0.21 |
| 0.01 | 62.9/0.14 | 66.1/0.19 | 77.5/0.26 |
| 0.005 | 62.3/0.17 | 67.3/0.23 | 83.3/0.34 |
| 0.001 | 61.5/0.22 | 68.7/0.31 | 95.2/0.64 |

To address the size and PDI changes post-freeze/thaw, the saRNA-encapsulating LNPs were diluted with “blank” or “empty” LNPs (prepared according to Table 2), which do not encapsulate RNAs, to obtain saRNA concentrations below 10 μg/ml. PS80, 1% PEG or 20% sucrose as an excipient can prevent the particle size and PDI change post freeze thaw (F/T) at lowest concentration.

TABLE 11
Effect of excipients on formulation attributes pre-and post-freeze-thaw

| | Particle size | | PDI | |
|---|---|---|---|---|
| | Liquid | 3x F/T | Liquid | 3x F/T |
| 0.05% PS80 | | | | |
| 0.03 | 82.56 | 85.89 | 0.091 | 0.068 |
| 0.01 | 82.83 | 86.17 | 0.096 | 0.088 |
| 0.001 | 82.68 | 72.88 | 0.2 | 0.255 |
| 0.1% PEG 400 | | | | |
| 0.03 | 82.61 | 86.34 | 0.071 | 0.118 |
| 0.01 | 82.07 | 89.78 | 0.06 | 0.149 |
| 0.001 | 82.43 | 100.3 | 0.145 | 0.237 |
| 1% PEG 400 | | | | |
| 0.03 | 88.75 | 89.38 | 0.098 | 0.088 |
| 0.01 | 90.59 | 93.97 | 0.077 | 0.129 |
| 0.001 | 87.76 | 94.31 | 0.135 | 0.165 |
| 20% Sucrose 20S | | | | |
| 0.03 | 70.99 | 73.82 | 0.08 | 0.071 |
| 0.01 | 71.56 | 73.5 | 0.092 | 0.086 |
| 0.001 | 69.33 | 69.23 | 0.184 | 0.192 |

TABLE 12
Effect of empty LNPs on formulation attributes pre-and post-freeze-thaw

| | Particle size | | PDI | |
|---|---|---|---|---|
| | Liquid | 3x F/T | Liquid | 3x F/T |
| 10% Sucrose Dilution | | | | |
| with empty LNPs | | | | |
| 0.03 | 82.69 | 88.43 | 0.094 | 0.115 |
| 0.01 | 68.79 | 75.71 | 0.101 | 0.141 |
| 0.001 | 58.42 | 65.2 | 0.065 | 0.089 |
| 20% Sucrose Dilution | | | | |
| with empty LNPs | | | | |
| 0.03 | 83.84 | 88.32 | 0.07 | 0.089 |
| 0.01 | 51.53 | 58.25 | 0.079 | 0.133 |
| 0.001 | 55.45 | 58.33 | 0.112 | 0.12 |
| F1: Control 1.8% ALC-0159 | | | | |
| No empty LNPs included | | | | |
| 0.03 | 82.69 | 88.43 | 0.094 | 0.115 |
| 0.01 | 82.72 | 90.69 | 0.085 | 0.123 |
| 0.001 | 81.82 | 94.88 | 0.117 | 0.229 |

Using empty LNPs to dilute below 10 ug/ml reduces changes post freeze-thaw. Using empty LNPs preferably dilutes the RNA and not the remainder of the components in the formulation.

### Example 7: VSV-G saRNA LNP Lyophilization

VSV-g saRNA LNP at 60 μg/ml in 10 mM Tris and 10% Sucrose formulation was lyophilized. The 2 ml vials were filled at 0.5 mL and reconstituted with water at 0.475 ml in this feasibility assessment. The VSVG saRNA used herein does not comprise modified nucleosides other than the 5′ cap.

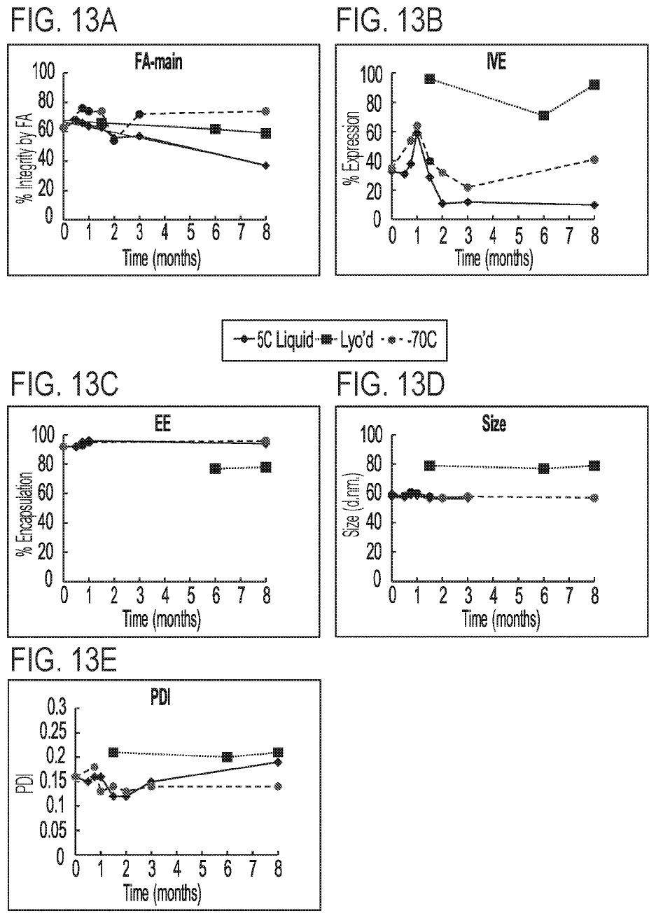

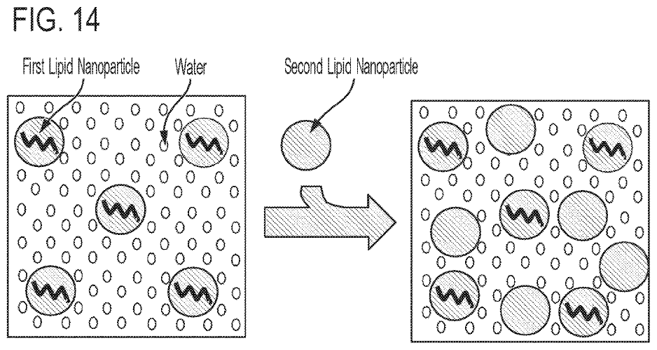

The lyophilized VSVG saRNA LNP DP in Tris/Suc appears to be stable over 8 months at 5° C. See FIG. 13A-13E.

### Example 8: Influenza saRNA LNP Lyophilization

Influenza HA saRNA LNP at 10 μg/ml in the matrices below were lyophilized. The HA saRNA molecules used herein do not comprise modified nucleosides other than the 5′ cap. The 2 mL vials were filled at 0.35 mL and reconstituted with water or saline at 0.33 mL in this feasibility assessment (see Table 13).

TABLE 13
Effect of sucrose on formulation attributes pre-and post-lyophilization

| | | | | Water | Saline |
|---|---|---|---|---|---|
| | | | | Recon | Recon |
| Formu- | | Conc. | Pre-Lyo | Post-Lyo | Post-Lyo |
| lation | LNP Matrix | (ug/ml) | Size/PDI | Size/PDI | Size/PDI |
| A2 | 10 mM Tris in 10% | 10 | 68.1/0.22 | 80.2/0.23 | 84.7/0.26 |
| | Sucrose | | | | |
| B2 | 10 mM Tris in 20% | 10 | 63.0/0.21 | 69.8/0.21 | 72.9/0.25 |
| | Sucrose | | | | |

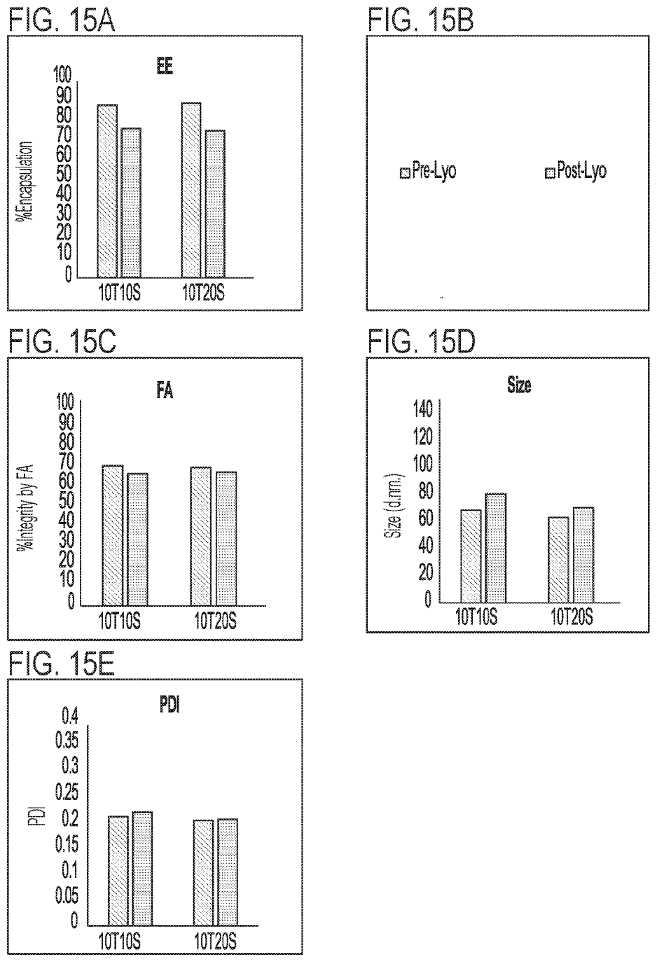

The lyophilized matrices yielded elegant cakes, and the size and PDI post-reconstitution were favorable with water reconstitution. See FIG. 15A-15E.

Post-lyophilization % expression on IVE was assessed in both VSVG and HA saRNA formulations. Post-lyophilization % expression increase on IVE has been observed in both VSVG and HA saRNA formulations.

### Example 9: Stabilization of Low mRNA Concentration Formulations using Various Colloidal Stabilizers

The effect of blank LNPs or liposomes on stabilization of low mRNA concentration formulations was tested.

Blank LNPs (comprised of ALC-0159, DSPC, cholesterol, and a cationic lipid comprising MC3 or A9; see Table 14 below) were added to a composition comprising the LNPs encapsulating Flu modRNA (0.3, 0.1, 0.01, and 0.001 mg/mL, Washington strain, said LNPs encapsulating Flu modRNA comprising ALC-0159, DSPC, cholesterol, and a cationic lipid comprising MC3 or A9) such that the lipid content of the resulting formulation was equivalent to that of a 0.5, 0.3, and 0.1 mg/ml mRNA drug product. A separate batch of the blank LNPs (comprised of ALC-0159, DSPC, cholesterol, and a cationic lipid comprising MC3 or A9) was prepared and mixed with the batch of mRNA-containing LNPs and the formulation buffer to achieve the target lipids and mRNA concentrations in the final formulations.

TABLE 14
Cationic Lipids for Blank LNPs

| | | | Physical | |
|---|---|---|---|---|
| Lipid | Mol. | | State & | |
| (CAS | Wt. | Molecular | Storage | |
| number) | [Da] | Formula | Condition | Chemical name and structure |
| DLin- | 642.1 | C₄₃H₇₉NO₂ | Liquid | (6Z,9Z,28Z,31Z)-Heptatriaconta- |
| MC3- | | | −20° C. | 6,9,28,31-tetraen-19-yl 4- |
| DMA | | | | (dimethylamino)butanoate |
| (MC3) (1224606- 06-7) | | | | 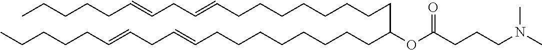 [SMILES: CCCCC/C=C/C/C=C/CCCCCCCCC(CCCCCCCC/C=C/C/C=C/CCCCC)OC(=O)CCCN(C)C] |
| A9 | 905.5 | C₅₇H₁₁₂N₂O₅ | Liquid | 1,19-Bis(2-butyloctyl) 10-[3- |
| (2036272- | | | −20° C. | (dimethylamino) propyl](1- |
| 50-9) | | | | oxononyl)amino]nonadecanedioate |
| | | | | 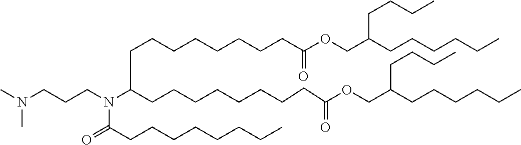 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] |

Liposomes (comprised of ALC-0159, DSPC, and cholesterol) were added to a composition comprising the LNPs encapsulating Flu modRNA (0.1, 0.01, and 0.001 mg/mL, Washington strain, said LNPs encapsulating Flu modRNA comprising ALC-0159, DSPC, cholesterol, and a cationic lipid comprising MC3 or ALC-0315) such that the lipid content of the resulting formulation was equivalent to that of a 0.5 and 0.1 mg/ml mRNA drug product. A separate batch of the liposomes (comprised of ALC-0159, DSPC, and cholesterol) was prepared and mixed with the batch of mRNA-containing LNPs and the formulation buffer to achieve the target lipids and mRNA concentrations in the final formulations.

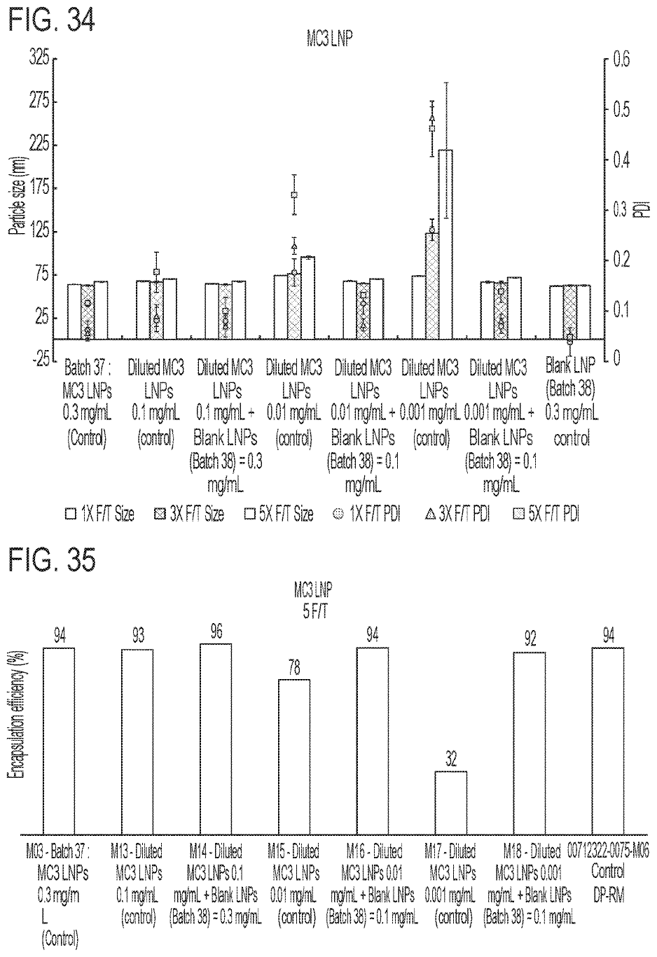

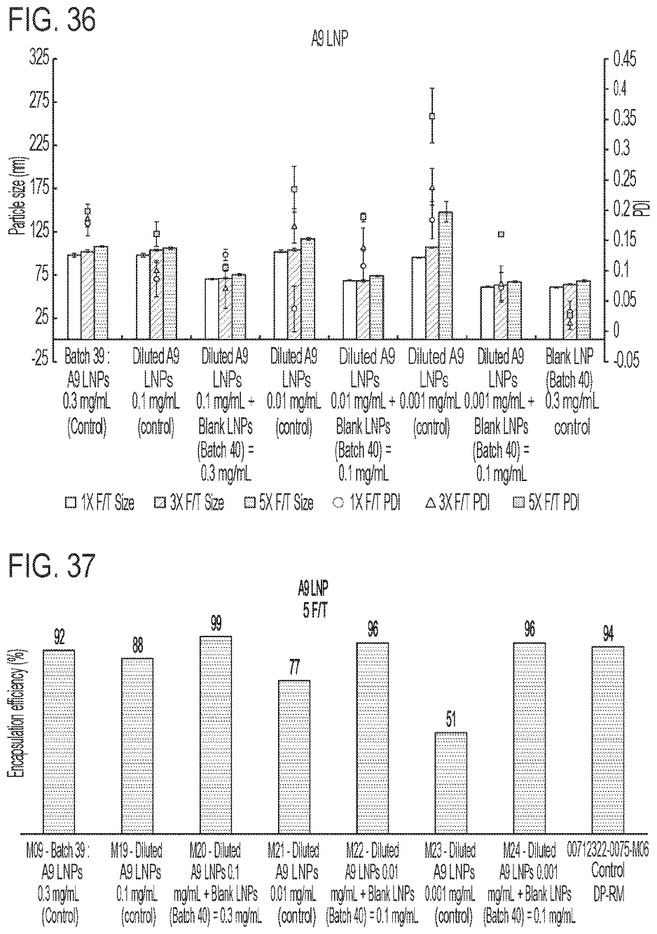

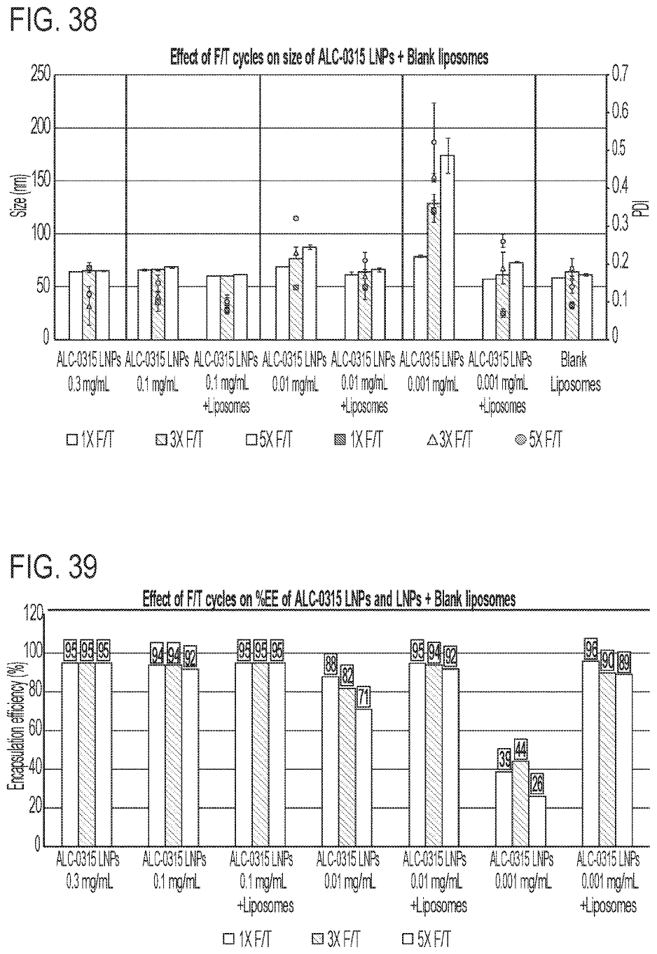

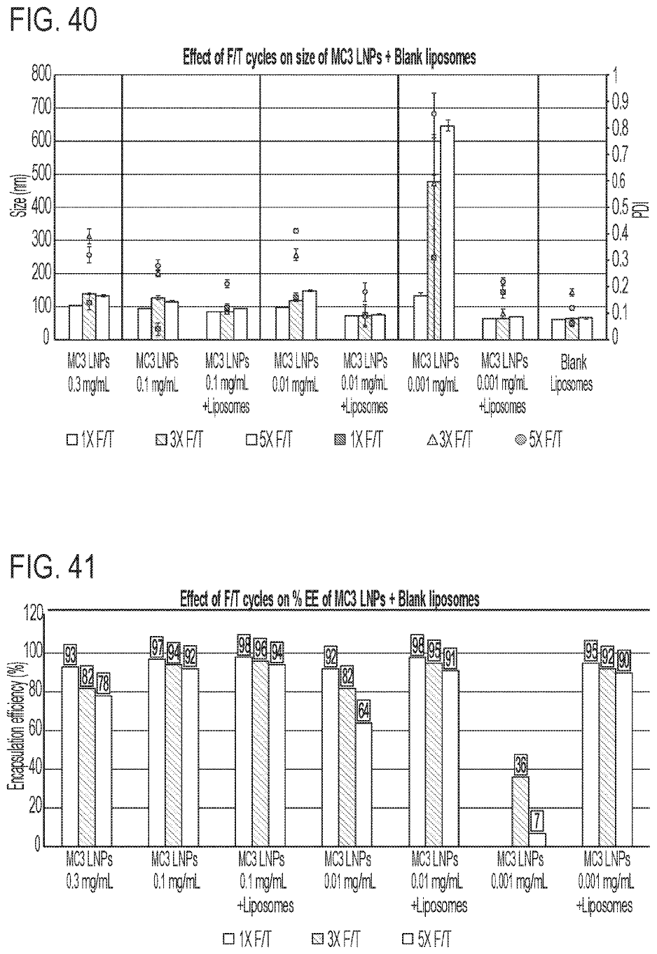

5× freeze-thaw (FT) cycling was conducted between −70° C. and 25° C. (0.3 mL fill). As shown in FIGS. 34-37, the inclusion of blank LNPs (comprised of ALC-0159, DSPC, cholesterol, and a cationic lipid comprising MC3 or A9) mitigated the increase in size and PDI and the decrease in % encapsulation for LNPs encapsulating Flu modRNA comprising ALC-0159, DSPC, cholesterol, and a cationic lipid comprising MC3 or A9 post-freeze-thawing observed in the dispersions that do not contain blank LNPs. As shown in FIGS. 38-41, inclusion of liposomes also mitigated increase in size/PDI and decrease in % encapsulation for LNPs encapsulating Flu modRNA comprising ALC-0159, DSPC, cholesterol, and a cationic lipid comprising MC3 or ALC-0315 during freeze-thawing.

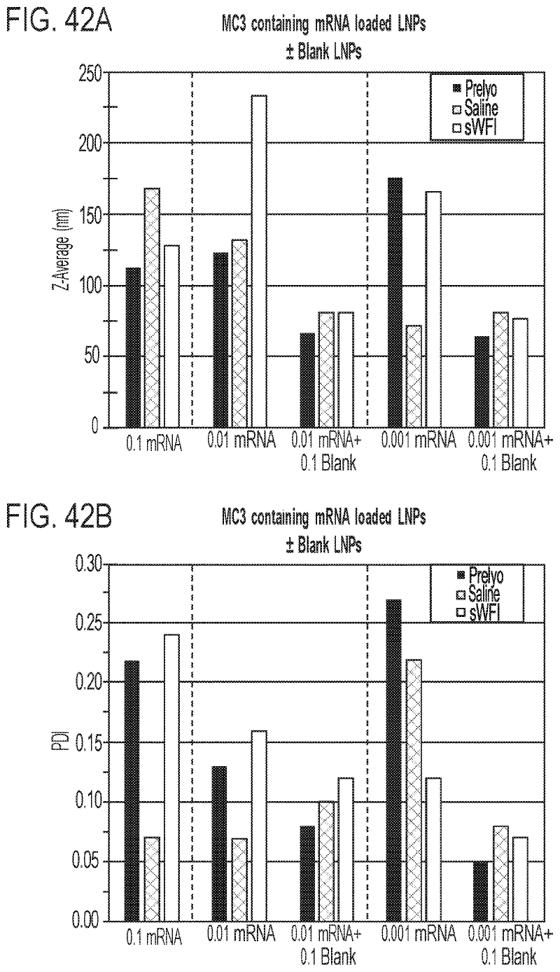

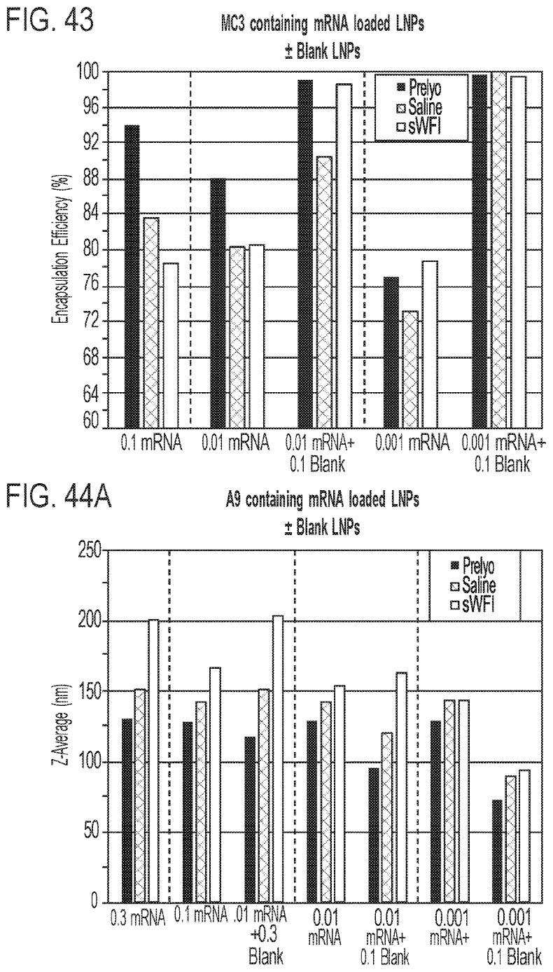

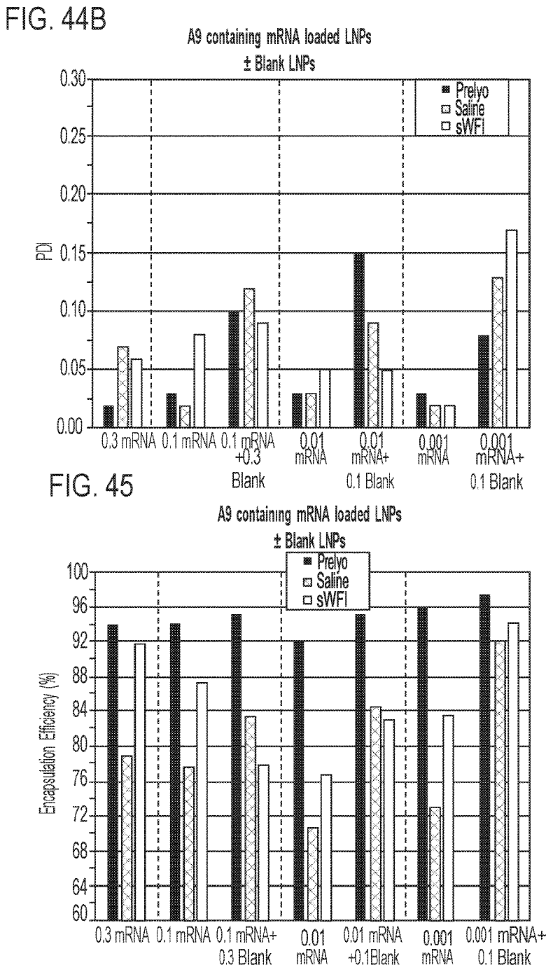

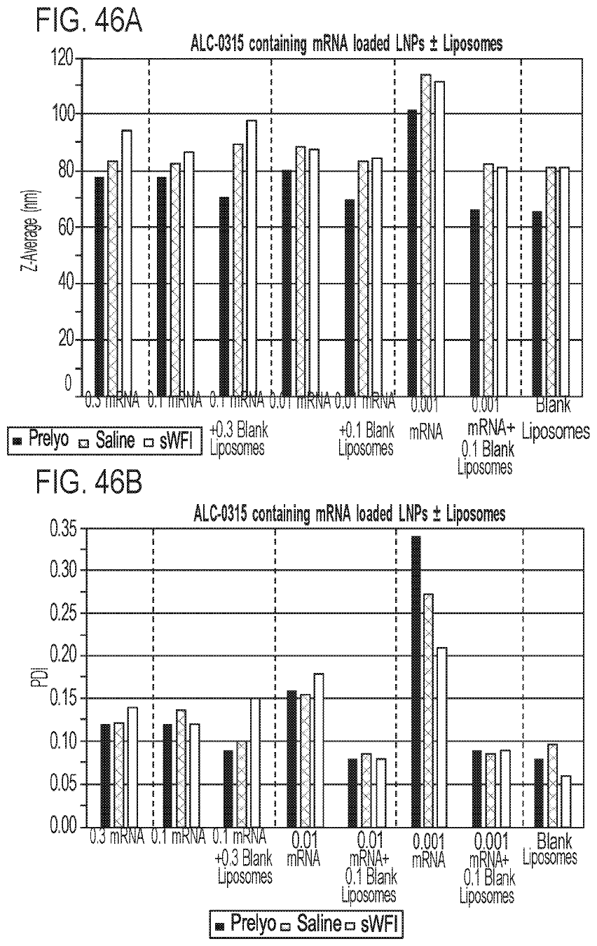

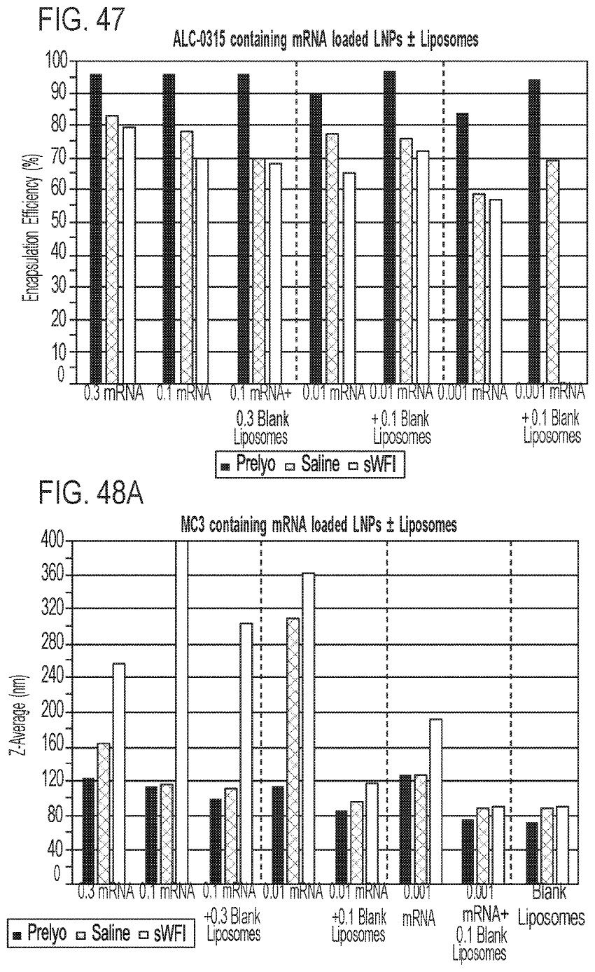

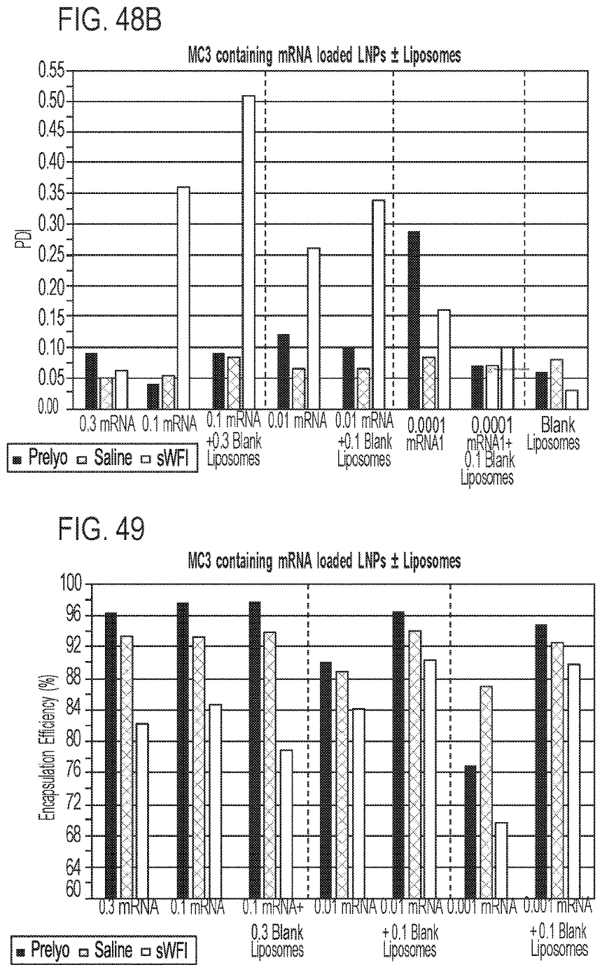

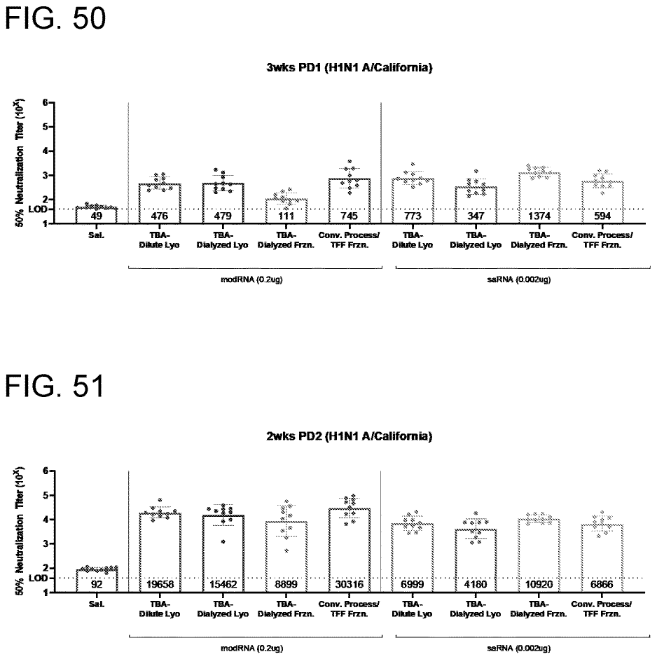

In addition to free-thawing, freeze-drying was conducted with and without annealing (0.3 mL fill), and product attributes were measured for the cakes reconstituted in saline vs. sterile water for injection. As shown in FIGS. 42-45, the inclusion of blank LNPs (comprised of ALC-0159, DSPC, cholesterol, and a cationic lipid comprising MC3 or A9) mitigated the increase in size and PDI and the decrease in % encapsulation for LNPs encapsulating Flu modRNA comprising ALC-0159, DSPC, cholesterol, and a cationic lipid comprising MC3 or A9 during reconstitution after freeze-drying compared to dispersions that do not contain blank LNPs. As shown in FIGS. 46-49, inclusion of liposomes also mitigated increase in size/PDI and decrease in % encapsulation for LNPs encapsulating Flu modRNA comprising ALC-0159, DSPC, cholesterol, and a cationic lipid comprising MC3 or ALC-0315 during reconstitution after freeze-drying.

Results were consistent for all blank LNPs tested having a cationic lipid comprising any one of the lipids selected from, for example, MC3 and A9. In the case of co-formulations of mRNA-containing LNPs and liposomes, a decrease in size/PDI was observed post freeze-drying similar to the observations with the blank LNPs. Liposomes prevented a decrease in % encapsulation of the mRNA-containing LNPs, though to a lesser extent than blank LNPs.

### Example 10: Stabilization of Low mRNA Concentration Formulations using Blank LNPs and/or Increased Sucrose Concentration

The effect of blank LNPs and/or higher sucrose concentrations on stabilization of low mRNA concentration formulations was tested.

Blank LNPs (comprised of the lipids according to Table 2) were added to a composition comprising the LNPs encapsulating Flu modRNA (0.01 and 0.005 mg/mL, Washington strain, said LNPs encapsulating Flu modRNA comprising the lipids according to Table 2) such that the lipid content of the resulting formulation was equivalent to that of a 0.1 and 0.2 mg/ml mRNA drug product. A separate batch of blank LNPs (comprised of the lipids according to Table 2) was prepared and mixed with a batch of mRNA-containing LNPs and the formulation buffer to achieve the target lipids and mRNA concentrations in the final formulations. modRNA-containing formulations tested are summarized in Table 15.

Sucrose at a concentration of 10.3%, 15.4%, or 20.5% w/v was added to a composition comprising the LNPs encapsulating Flu modRNA (0.01 and 0.005 mg/mL, Washington strain, said LNPs encapsulating Flu modRNA comprising the lipids according to Table 2).

4× freeze-thaw (FT) cycling was conducted between −70° C. and 25° C. (2.25 mL fill).

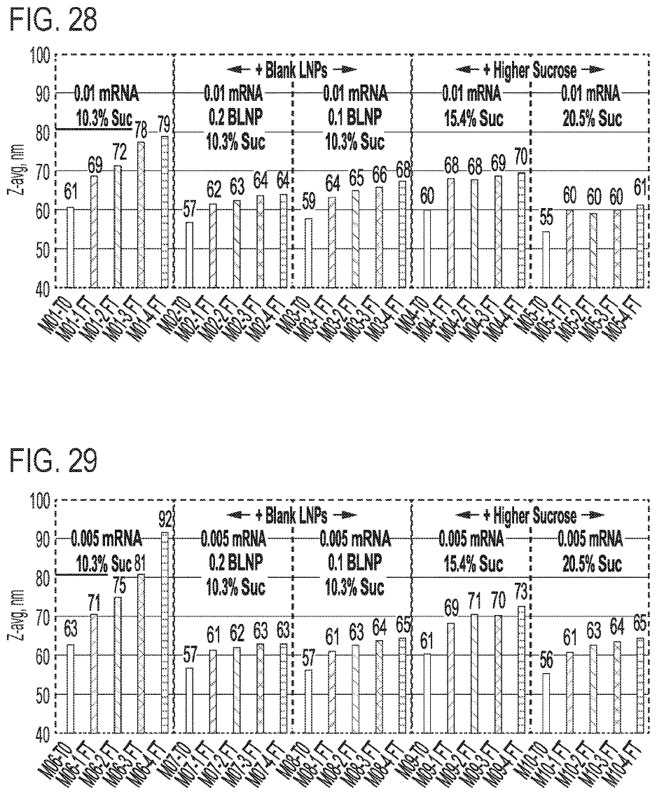

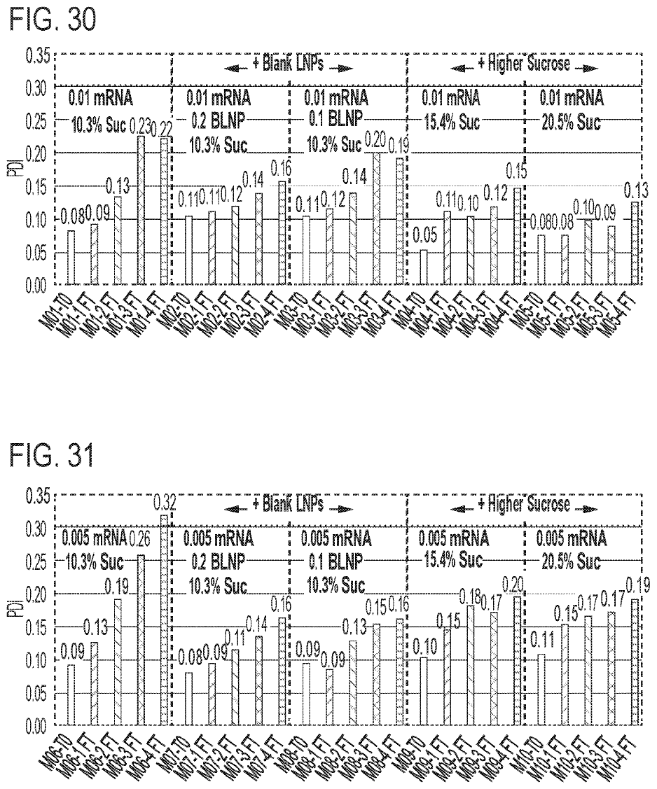

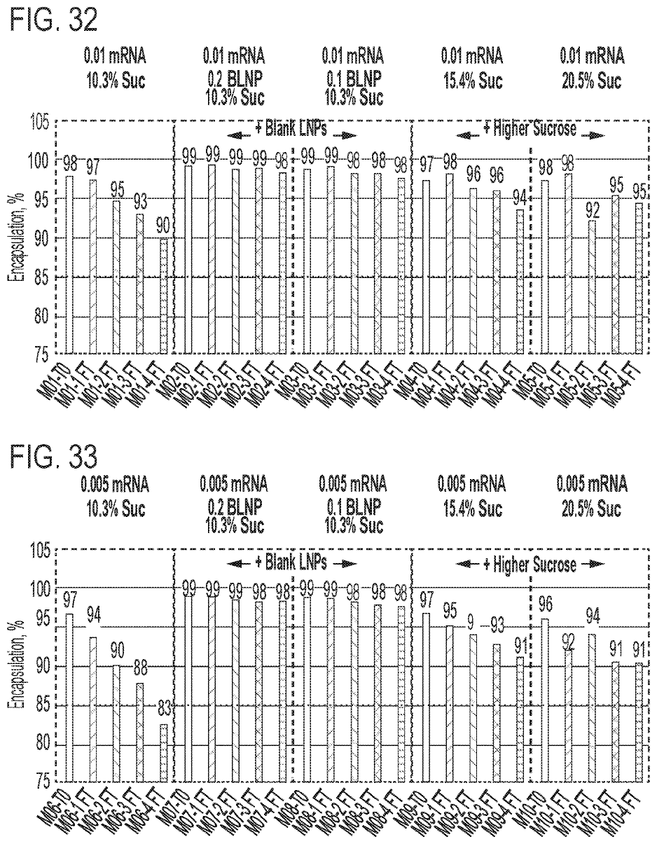

As shown in FIGS. 28-33, the inclusion of the blank LNPs or 20.5% sucrose mitigated the increase in size and PDI and the decrease in % encapsulation post-freeze-thawing observed in the dispersions that do not contain blank LNPs.

TABLE 15
Tested modRNA-containing formulations

| Formulation | Description of | | Formulation |
|---|---|---|---|
| # | LNP-modRNA | Formulation description | Buffer |
| 1 | 0.01 mg/mL Flu modRNA in | 0.01 mg/mL mRNA in | 10 mM Tris/ |
| | Tris-10.3% w/v Sucrose, post | Tris/Sucrose after 4X FT, | 10.3% w/v |
| | 4x FT | 2.25 mL fill volume, | Sucrose |
| | | −80° C. frozen storage | |
| 2 | 0.01 mg/mL Flu modRNA in | 0.01 mg/mL mRNA in | 10 mM Tris/ |
| | Tris-10.3% w/v Sucrose | Tris/Sucrose with target lipids | 10.3% w/v |
| | Target lipids eq. to 0.2 | eq. to 0.2 mg/mL mRNA, after | Sucrose |
| | mg/mL DP, post 4x FT | 4X FT, | |
| | | 2.25 mL fill volume, −80° C. | |
| | | frozen storage | |
| 3 | 0.01 mg/mL Flu modRNA in | 0.01 mg/mL mRNA in | 10 mM Tris/ |
| | Tris-10.3% w/v Sucrose | Tris/Sucrose with target lipids | 10.3% w/v |
| | Target lipids eq. to 0.1 | eq. to 0.1 mg/mL DP, after 4X | Sucrose |
| | mg/mL DP, post 4x FT | FT, 2.25 mL fill volume, | |
| | | −80° C. frozen storage | |
| 4 | 0.01 mg/mL Flu modRNA in | 0.01 mg/mL mRNA in Tris/ | 10 mM Tris/ |
| | Tris-15.4% w/v Sucrose, post | 15.4% w/v Sucrose after 4X | 15.4% w/v |
| | 4x FT | FT, 2.25 mL fill volume, −80° C. | Sucrose |
| | | frozen storage | |
| 5 | 0.01 mg/mL Flu modRNA in | 0.01 mg/mL mRNA in Tris/ | 10 mM Tris/ |
| | Tris-20.5% w/v Sucrose, post | 20.5% w/v Sucrose after 4X | 20.5% w/v |
| | 4x FT | FT, 2.25 mL fill volume, −80° C. | Sucrose |
| | | frozen storage | |
| 6 | 0.005 mg/mL Flu modRNA in | 0.005 mg/mL mRNA in | 10 mM Tris/ |
| | Tris-10.3% w/v Sucrose, post | Tris/Sucrose after 4X FT, | 10.3% w/v |
| | 4x FT | 2.25 mL fill volume, | Sucrose |
| | | −80° C. frozen storage | |
| 7 | 0.005 mg/mL Flu modRNA in | 0.005 mg/mL mRNA in | 10 mM Tris/ |
| | Tris-10.3% w/v Sucrose | Tris/Sucrose with target lipids | 10.3% w/v |
| | Target lipids eq. to 0.2 | eq. to 0.2 mg/mL DP, after 4X | Sucrose |
| | mg/mL DP, post 4x FT | FT, | |
| | | 2.25 mL fill volume, −80° C. | |
| | | frozen storage | |
| 8 | 0.005 mg/mL Flu modRNA in | 0.005 mg/mL mRNA in | 10 mM Tris/ |
| | Tris-10.3% w/v Sucrose | Tris/Sucrose with target lipids | 10.3% w/v |
| | Target lipids eq. to 0.1 | eq. to 0.1 mg/mL DP, after 4X | Sucrose |
| | mg/mL DP, post 4x FT | FT, 2.25 mL fill volume, | |
| | | −80° C. frozen storage | |
| 9 | 0.005 mg/mL Flu modRNA in | 0.005 mg/mL mRNA in Tris/ | 10 mM Tris/ |
| | Tris-15.4% w/v Sucrose, post | 15.4% w/v Sucrose after 4X | 15.4% w/v |
| | 4x FT | FT, 2.25 mL fill volume, −80° C. | Sucrose |
| | | frozen storage | |
| 10 | 0.005 mg/mL Flu modRNA in | 0.005 mg/mL mRNA in Tris/ | 10 mM Tris/ |
| | Tris-20.5% w/v Sucrose, post | 20.5% w/v Sucrose after 4X | 20.5% w/v |
| | 4x FT | FT, 2.25 mL fill volume, −80° C. | Sucrose |
| | | frozen storage | |
| 11 | Blank LNPs (frozen) | Blank LNPs (Control sample) | 10 mM Tris/ |
| | Target lipids eq. to 0.2 mg/mL | Target lipids eq. to 0.2 mg/mL | 10.3% w/v |
| | DP | DP | Sucrose |
| 12 | Blank LNPs (frozen) | Blank LNPs (Control sample) | 10 mM Tris/ |
| | Target lipids eq. to 0.1 mg/mL | Target lipids eq. to 0.1 mg/mL | 10.3% w/v |
| | DP | DP | Sucrose |
| 13 | 0.01 mg/mL Flu modRNA in | 0.01 mg/mL mRNA in | 10 mM Tris/ |
| | Tris-Sucrose, | Tris/Sucrose, 3.5 mL fill | 10.3% w/v |
| | (unfrozen) | volume, | Sucrose |
| | | 2-8° C. storage | |
| 14 | 0.005 mg/mL Flu modRNA in | 0.005 mg/mL mRNA in | 10 mM Tris/ |
| | Tris-Sucrose, | Tris/Sucrose, 3.5 mL fill | 10.3% w/v |
| | (unfrozen) | volume, | Sucrose |
| | | 2-8° C. storage | |
| 15 | 0.1 mg/mL Flu modRNA in | 0.1 mg/mL mRNA in Tris/10% | 10 mM Tris/ |
| | Tris-Sucrose | Sucrose, 0.075 mL fill volume | 10% Sucrose |
| | (frozen control) | −80° C. frozen control | |

In more detail, the drug products (DPs) of Groups 1-10 are supplied frozen and have completed 4× freeze-thaw cycles at the time of dose preparation. Groups 1-5 correspond to the DP formulations containing 0.01 mg/mL Flu modRNA. Groups 6-10 correspond to the DP formulations containing 0.005 mg/mL Flu modRNA. The DPs of Groups 11-12 (blank LNPs in Tris-10.3% w/v Sucrose) are supplied frozen and completed 1× at the time of dose preparation. The dilution plan for groups 11-12 (Blank LNPs) is developed to target the same amount of lipids as provided by Groups 7 and 8 (containing 0.005 mg/ml mRNA and targeting lipids equivalent to a 0.2 or 0.1 mg/mL mRNA containing DP). Groups 13-14 are supplied as a liquid and are held at 2-8° C. Group 15 is used as a frozen study control. A previously frozen, pristine vial is thawed, sub-aliquoted into single use tubes, and refrozen.

HAI and neutralization assays are conducted as readouts. All neutralization samples are heat inactivated and frozen/stored at −80° C.

### Example 11: Study Testing Co-Formulated Flu modRNA and Blank LNPs Versus Flu modRNA LNPs at Higher Sucrose Concentrations (Prime and Boost)

This mouse study will provide in-vivo data to enable the development of formulation development strategies around the use of blank lipid nanoparticles (LNPs) versus a higher sucrose concentration. The objectives of the study include: (1) investigating the effect of co-formulated (mRNA and blank LNPs) on the immunogenicity of low concentration Flu modRNA (Washington strain) containing Tris-10.3% w/v Sucrose formulations when compared to mRNA LNPs only; (2) investigating the effect of blank LNPs on immunogenicity in the absence of mRNA; (3) investigating the effect of higher sucrose concentrations (15.4% w/v vs. 20.5% w/v) on the immunogenicity of low concentration Flu modRNA containing Tris-Sucrose formulations; (4) comparing the effect of unfrozen vs. frozen drug product containing mRNA LNPs (in Tris-10.3% w/v Sucrose) post completion of 4× freeze-thaw cycling on immunogenicity. Mice will be immunized with different modRNA-containing formulations summarized in Table 16, with further details on the test articles and diluent provided in Table 17. Sera collected at 21 days post prime and 14 days post boost (occurring on day 28) will be evaluated by serology testing (HAI, 1-day neutralization, D21, 42). Statistical differences between groups can be determined by using 10 mice per group. Mice are Balb/c female mice aged 11-13 weeks at study start. Tables 16-19 refer to the same 15 formulations and saline control, respectively.

TABLE 16
Formulations containing modRNA

| | | | Dose | Dose Vol / | Vax | Bleed |
|---|---|---|---|---|---|---|
| Gp# | Mice | Description of RNA DP^{†} | (μg) | Route | (Day) | (Day) |
| 1 | 10 | 0.01 mg/mL Flu modRNA in | 0.2 | 50 μl / IM | 0, 28 | 21, 42 |
| | | Tris-10.3% w/v Sucrose, post 4x FT | | | | |
| 2 | 10 | 0.01 mg/mL Flu modRNA in | 0.2 | 50 μl / IM | 0, 28 | 21, 42 |
| | | Tris-10.3% w/v Sucrose | | | | |
| | | Target lipids eq. to 0.2 mg/mL DP, | | | | |
| | | post 4x FT | | | | |
| 3 | 10 | 0.01 mg/mL Flu modRNA in Tris- | 0.2 | 50 μl / IM | 0, 28 | 21, 42 |
| | | 10.3% w/v Sucrose | | | | |
| | | Target lipids eq. to 0.1 mg/mL DP, | | | | |
| | | post 4x FT | | | | |
| 4 | 10 | 0.01 mg/mL Flu modRNA in | 0.2 | 50 μl / IM | 0, 28 | 21, 42 |
| | | Tris-15.4% w/v Sucrose, post 4x FT | | | | |
| 5 | 10 | 0.01 mg/mL Flu modRNA in | 0.2 | 50 μl / IM | 0, 28 | 21, 42 |
| | | Tris-20.5% w/v Sucrose, post 4x FT | | | | |
| 6 | 10 | 0.005 mg/mL Flu modRNA in | 0.2 | 50 μl / IM | 0, 28 | 21, 42 |
| | | Tris-10.3% w/v Sucrose, post 4x FT | | | | |
| 7 | 10 | 0.005 mg/mL Flu modRNA in | 0.2 | 50 μl / IM | 0, 28 | 21, 42 |
| | | Tris-10.3% w/v Sucrose | | | | |
| | | Target lipids eq. to 0.2 mg/mL DP, | | | | |
| | | post 4x FT | | | | |
| 8 | 10 | 0.005 mg/mL Flu modRNA in | 0.2 | 50 μl / IM | 0, 28 | 21, 42 |
| | | Tris-10.3% w/v Sucrose | | | | |
| | | Target lipids eq. to 0.1 mg/mL DP, | | | | |
| | | post 4x FT | | | | |
| 9 | 10 | 0.005 mg/mL Flu modRNA in | 0.2 | 50 μl / IM | 0, 28 | 21, 42 |
| | | Tris-15.4% w/v Sucrose, post 4x FT | | | | |
| 10 | 10 | 0.005 mg/mL Flu modRNA in | 0.2 | 50 μl / IM | 0, 28 | 21, 42 |
| | | Tris-20.5% w/v Sucrose, post 4x FT | | | | |
| 11 | 10 | Blank LNPs in Tris-10.3% w/v | — | 50 μl / IM | 0, 28 | 21, 42 |
| | | Sucrose (frozen) | | | | |
| | | Target lipids eq. to 0.2 mg/mL DP | | | | |
| 12 | 10 | Blank LNPs in Tris-10.3% w/v | — | 50 μl / IM | 0, 28 | 21, 42 |
| | | Sucrose (frozen) | | | | |
| | | Target lipids eq. to 0.1 mg/mL DP | | | | |
| 13 | 10 | 0.01 mg/mL Flu modRNA in | 0.2 | 50 μl / IM | 0, 28 | 21, 42 |
| | | Tris-10.3% w/v Sucrose (liquid) | | | | |
| 14 | 10 | 0.005 mg/mL Flu modRNA in | 0.2 | 50 μl / IM | 0, 28 | 21, 42 |
| | | Tris-10.3% w/v Sucrose (liquid) | | | | |
| 15 | 10 | 0.1 mg/mL Flu modRNA in | 0.2 | 50 μl / IM | 0, 28 | 21, 42 |
| | | Tris-10.3% w/v Sucrose 1x FT | | | | |
| 16 | 5 | Saline | — | 50 μl / IM | 0, 28 | 21, 42 |

^{†}Groups 1-10 are supplied frozen and will complete 4x F/T at the time of dose preparation.
Groups 11-12 (blank LNPs in Tris-10.3% w/v Sucrose) are supplied frozen and will complete 1x F/T at the time of dose preparation.
Groups 13-14 are supplied as a liquid and will be held at 2-8° C.
Group 15 will serve as a study bridging control, and is supplied frozen

TABLE 17
Test articles and diluent

| | | | | Vials#/ |
|---|---|---|---|---|
| | | | | Storage |
| | | | Formulation | (including |
| Item | Test | | Matrix and | contingency |
| # | articles/Diluent | DP Description^{†} | information | vials) |
| 1 | 0.01 mg/mL Flu | 0.01 mg/mL mRNA in | 0.01 mg/mL of | 5 vials/−80° C. |
| | modRNA in Tris- | Tris/Sucrose after 4X FT, | modRNA LNP in | (1 prime, 1 |
| | 10.3% w/v | 2.25 mL fill volume, | 10 mM | boost, and 3 |
| | Sucrose | −80° C. frozen storage (i.e., | Tris/10.3% w/v | contingency) |
| | | blank LNPs not included) | Sucrose, pH 7.4 | |
| 2 | 0.01 mg/mL Flu | 0.01 mg/mL mRNA in | 0.01 mg/mL of | 5 vials/−80° C. |
| | modRNA in Tris- | Tris/Sucrose with target | modRNA LNP in | (1 prime, 1 |
| | 10.3% w/v | lipids eq. to 0.2 mg/mL | 10 mM | boost, and 3 |
| | Sucrose | DP, after 4X FT, 2.25 mL | Tris/10.3% w/v | contingency) |
| | | fill volume, −80° C. frozen | Sucrose, pH 7.4 | |
| | | storage (i.e., blank LNPs | | |
| | | included) | | |
| 3 | 0.01 mg/mL Flu | 0.01 mg/mL mRNA in | 0.01 mg/mL of | 5 vials/−80° C. |
| | modRNA in Tris- | Tris/Sucrose with target | modRNA LNP in | (1 prime, 1 |
| | 10.3% w/v | lipids eq. to 0.1 mg/mL | 10 mM | boost, and 3 |
| | Sucrose | DP, after 4X FT, 2.25 mL | Tris/10.3% w/v | contingency) |
| | | fill volume, | Sucrose, pH 7.4 | |
| | | −80° C. frozen storage (i.e., | | |
| | | blank LNPs included) | | |
| 4 | 0.01 mg/mL Flu | 0.01 mg/mL mRNA in Tris/ | 0.01 mg/mL of | 5 vials/−80° C. |
| | modRNA in Tris- | Intermediate Sucrose after | modRNA LNP in | (1 prime, 1 |
| | 15.4% w/v | 4X FT, 2.25 mL fill volume, | 10 mM | boost, and 3 |
| | Sucrose | −80° C. frozen storage (i.e., | Tris/15.4% w/v | contingency) |
| | | blank LNPs not included) | Sucrose, pH 7.4 | |
| 5 | 0.01 mg/mL Flu | 0.01 mg/mL mRNA in | 0.01 mg/mL of | 5 vials/−80° C. |
| | modRNA in Tris- | Tris/High Sucrose after 4X | modRNA LNP in | (1 prime, 1 |
| | 20.5% w/v | FT, 2.25 mL fill volume, | 10 mM | boost, and 3 |
| | Sucrose | −80° C. frozen storage (i.e., | Tris/20.5% w/v | contingency) |
| | | blank LNPs not included) | Sucrose, pH 7.4 | |
| 6 | 0.005 mg/mL | 0.005 mg/mL mRNA in | 0.005 mg/mL of | 5 vials/−80° C. |
| | Flu modRNA in | Tris/Sucrose after 4X FT, | modRNA LNP in | (1 prime, 1 |
| | Tris- | 2.25 mL fill volume, | 10 mM | boost, and 3 |
| | 10.3% w/v | −80° C. frozen storage (i.e., | Tris/10.3% w/v | contingency) |
| | Sucrose | blank LNPs not included) | Sucrose, pH 7.4 | |
| 7 | 0.005 mg/mL | 0.005 mg/mL mRNA in | 0.005 mg/mL of | 5 vials/−80° C. |
| | Flu modRNA in | Tris/Sucrose with target | modRNA LNP in | (1 prime, 1 |
| | Tris-10.3% w/v | lipids eq. to 0.2 mg/mL | 10 mM | boost, and 3 |
| | Sucrose | DP, after 4X FT, 2.25 mL | Tris/10.3% w/v | contingency) |
| | | fill volume, | Sucrose, pH 7.4 | |
| | | −80° C. frozen storage (i.e., | | |
| | | blank LNPs included) | | |
| 8 | 0.005 mg/mL | 0.005 mg/mL mRNA in | 0.005 mg/mL of | 5 vials/−80° C. |
| | Flu modRNA in | Tris/Sucrose with target | modRNA LNP in | (1 prime, 1 |
| | Tris-10.3% w/v | lipids eq. to 0.1 mg/mL | 10 mM | boost, and 3 |
| | Sucrose | DP, after 4X FT, 2.25 mL | Tris/10.3% w/v | contingency) |
| | | fill volume, −80° C. frozen | Sucrose, pH 7.4 | |
| | | storage (i.e., blank LNPs | | |
| | | included) | | |
| 9 | 0.005 mg/mL | 0.005 mg/mL mRNA in | 0.005 mg/mL of | 4 vials/−80° C. |
| | Flu modRNA in | Tris/Intermediate Sucrose | modRNA LNP in | (1 prime, 1 |
| | Tris-15.4% w/v | after 4X FT, 2.25 mL fill | 10 mM | boost, and 2 |
| | Sucrose | volume, −80° C. frozen | Tris/15.4% w/v | contingency) |
| | | storage (i.e., blank LNPs not | Sucrose, pH 7.4 | |
| | | included) | | |
| 10 | 0.005 mg/mL Flu | 0.005 mg/mL mRNA in | 0.005 mg/mL of | 6 vials/−80° C. |
| | modRNA in | Tris/High Sucrose after | modRNA LNP in | (1 prime, 1 |
| | Tris-20.5% w/v | 4X FT, 2.25 mL fill volume, | 10 mM | boost, and 4 |
| | Sucrose | −80° C. frozen storage (i.e., | Tris/20.5% w/v | contingency) |
| | | blank LNPs not included) | Sucrose, pH 7.4 | |
| 11 | Blank LNPs | LNP with lipids equivalent | LNP in 10 mM | 5 vials/−80° C. |
| | Target lipids eq. | to 0.2 mg/mL DP and no | Tris/10.3% w/v | (1 prime, 1 |
| | to 0.2 mg/mL | mRNA in Tris/Sucrose, | Sucrose, pH 7.4 | boost, and 3 |
| | DP | 2.25 mL fill volume, | | contingency) |
| | | −80° C. frozen storage | | |
| 12 | Blank LNPs | LNP with lipids equivalent | LNP in 10 mM | 5 vials/−80° C. |
| | Target lipids eq. | to 0.1 mg/mL DP and no | Tris/10.3% w/v | (1 prime, 1 |
| | to 0.1 mg/mL | mRNA in Tris/Sucrose, | Sucrose, pH 7.4 | boost, and 3 |
| | DP | 2.25 mL fill volume, | | contingency) |
| | | −80° C. frozen storage | | |
| 13 | 0.01 mg/mL Flu | 0.01 mg/mL mRNA in | 0.01 mg/mL of | 3 vials/2-8° C. |
| | modRNA in | Tris/Sucrose, 3.5 mL fill | modRNA LNP in | (1 prime, 1 |
| | Tris-10.3% w/v | volume, | 10 mM | boost, and 1 |
| | Sucrose (liquid) | 2-8° C. storage (i.e., blank | Tris/10.3% w/v | contingency) |
| | | LNPs not included) | Sucrose, pH 7.4 | |
| 14 | 0.005 mg/mL | 0.005 mg/mL mRNA in | 0.005 mg/mL of | 3 vials/2-8° C. |
| | Flu modRNA in | Tris/Sucrose, 3.5 mL fill | modRNA LNP in | (1 prime, 1 |
| | Tris-10.3% w/v | volume, | 10 mM | boost, and 1 |
| | Sucrose (liquid) | 2-8° C. storage (i.e., blank | Tris/10.3% w/v | contingency) |
| | | LNPs not included) | Sucrose, pH 7.4 | |
| 15 | 0.1 mg/mL Flu | 0.1 mg/mL mRNA in | 0.1 mg/mL of | 2 aliquots/ |
| | modRNA in Tris- | Tris/Sucrose, | modRNA LNP in | −80° C. |
| | 10.3% w/v | −80° C. frozen control, 1X | 10 mM | (1prime/1 |
| | Sucrose | previously thawed and | Tris/10.3% w/v | boost) |
| | | refrozen aliquots | Sucrose, | |
| | | 0.075 mL fill volume | pH 7.4 | |
| | | −80° C. storage (i.e., blank | | |
| | | LNPs not included) | | |
| 16 | Saline | Saline | 0.9% NaCl in | 3 aliquots, RT |
| | | | water | |

^{†}Groups 1-10 are supplied frozen and will complete 4x F/T at the time of dose preparation.
Groups 11-12 (blank LNPs in Tris-10.3% w/v Sucrose) are supplied frozen and will complete 1x F/T at the time of dose preparation.
Groups 13-14 are supplied as a liquid and will be held at 2-8° C.
Group 15 will serve as a study bridging control, and is supplied frozen

TABLE 18
Analytical Test Results of the Test Articles described in Tables 16 and 17

| | | | | mRNA | | Size, | | |
|---|---|---|---|---|---|---|---|---|
| | | | | Integrity | | nm / | % Cells | |
| | | | Measured | by FA, | | PDI | positive | |
| | Group | Description | concentration, | %/ | Encapsulation, | by | by IVE | Endotoxin, |
| | # | of DP | mg/mL | LMS, % | % | DLS | (160 ng) | EU/mL† |
| Drug product | 1 | 0.01 mg/mL | 0.0116 | 83 / | 90 | 79/ | 81 | NMT |
| containing | | Flu modRNA | | NMT | | 0.22 | | 0.1 |
| 0.01 mg/mL | | in Tris-10.3% | | 3 | | | | |
| Flu modRNA | | w/v Sucrose, | | | | | | |
| with or without | | post 4x FT | | | | | | |
| blank LNPs or | | (i.e., blank | | | | | | |
| higher sucrose | | LNPs not | | | | | | |
| concentrations | | included) | | | | | | |
| | 2 | 0.01 mg/mL | 0.0123 | 80 / | 98 | 64/ | 71 | 0.61 |
| | | Flu modRNA | | NMT | | 0.16 | | |
| | | in Tris-10.3% | | 3 | | | | |
| | | w/v Sucrose, | | | | | | |
| | | Target lipids | | | | | | |
| | | eq. to 0.2 | | | | | | |
| | | mg/mL DP, | | | | | | |
| | | post 4x FT | | | | | | |
| | | (i.e., blank | | | | | | |
| | | LNPs | | | | | | |
| | | included) | | | | | | |
| | 3 | 0.01 mg/mL | 0.0118 | 75 / | 98 | 68/ | 81 | 0.12 |
| | | Flu modRNA | | NMT | | 0.19 | | |
| | | in Tris-10.3% | | 3 | | | | |
| | | w/v Sucrose | | | | | | |
| | | Target lipids | | | | | | |
| | | eq. to 0.1 | | | | | | |
| | | mg/mL DP, | | | | | | |
| | | post 4x FT | | | | | | |
| | | (i.e., blank | | | | | | |
| | | LNPs | | | | | | |
| | | included) | | | | | | |
| | 4 | 0.01 mg/mL | 0.0120 | 83 / | 94 | 70/ | 81 | NMT |
| | | Flu modRNA | | NMT | | 0.15 | | 1.00 |
| | | in Tris-15.4% | | 3 | | | | |
| | | w/v Sucrose, | | | | | | |
| | | post 4x FT | | | | | | |
| | 5 | 0.01 mg/mL | 0.0118 | 83 / | 95 | 61/ | 83 | NMT |
| | | Flu modRNA | | NMT | | 0.13 | | 1.00 |
| | | in Tris-20.5% | | 3 | | | | |
| | | w/v Sucrose, | | | | | | |
| | | post 4x FT | | | | | | |
| | | (i.e., blank | | | | | | |
| | | LNPs not | | | | | | |
| | | included) | | | | | | |
| Drug product | 6 | 0.005 mg/mL | 0.0058 | 83 / | 83 | 92/ | 90 | NMT |
| containing | | Flu modRNA | | NMT | | 0.32 | | 1.00 |
| 0.005 mg/mL | | in Tris-10.3% | | 3 | | | | |
| Flu modRNA | | w/v Sucrose, | | | | | | |
| with or without | | post 4x FT | | | | | | |
| blank LNPs or | | (i.e., blank | | | | | | |
| higher sucrose | | LNPs not | | | | | | |
| concentrations | | included) | | | | | | |
| | 7 | 0.005 mg/mL | 0.0061 | 81 / 4 | 98 | 63/ | 41 | 0.34 |
| | | Flu modRNA | | | | 0.16 | | |
| | | in Tris-10.3% | | | | | | |
| | | w/v Sucrose, | | | | | | |
| | | Target lipids | | | | | | |
| | | eq. to 0.2 | | | | | | |
| | | mg/mL DP, | | | | | | |
| | | post 4x FT | | | | | | |
| | | (i.e., blank | | | | | | |
| | | LNPs | | | | | | |
| | | included) | | | | | | |
| | 8 | 0.005 mg/mL | 0.0059 | 82 / | 98 | 65/ | 72 | 0.54 |
| | | Flu modRNA | | NMT | | 0.16 | | |
| | | in Tris-10.3% | | 3 | | | | |
| | | w/v Sucrose, | | | | | | |
| | | Target lipids | | | | | | |
| | | eq. to 0.1 | | | | | | |
| | | mg/mL DP, | | | | | | |
| | | post 4x FT | | | | | | |
| | | (i.e., blank | | | | | | |
| | | LNPs | | | | | | |
| | | included) | | | | | | |
| | 9 | 0.005 mg/mL | 0.0056 | 82 / | 91 | 73/ | 90 | NMT |
| | | Flu modRNA | | NMT | | 0.20 | | 1.00 |
| | | in Tris-15.4% | | 3 | | | | |
| | | w/v Sucrose, | | | | | | |
| | | post 4x FT | | | | | | |
| | | (i.e., blank | | | | | | |
| | | LNPs not | | | | | | |
| | | included) | | | | | | |
| | 10 | 0.005 mg/mL | 0.0050 | 81 / | 91 | 65/ | 90 | NMT |
| | | Flu modRNA | | NMT | | 0.19 | | 1.00 |
| | | in Tris-20.5% | | 3 | | | | |
| | | w/v Sucrose, | | | | | | |
| | | post 4x FT | | | | | | |
| | | (i.e., blank | | | | | | |
| | | LNPs not | | | | | | |
| | | included) | | | | | | |
| | | | | mRNA | | Size, | | |
| | | | | Integrity | | nm / | % Cells | |
| | | | Measured | by FA, | | PDI | positive | |
| | Group | Description | concentration, | %/ | Encapsulation, | by | by IVE | Endotoxin, |
| | # | of DP | mg/mL | LMS, % | % | DLS | (160 ng) | EU/mL^{†} |
| Blank LNP | 11 | Blank LNPs | NA | NA | NA | 56/ | 5 | 0.83 |
| Control | | in Tris-10.3% | | | | 0.10 | | |
| samples | | w/v Sucrose, | | | | | | |
| | | Target lipids | | | | | | |
| | | eq. to 0.2 | | | | | | |
| | | mg/mL DP | | | | | | |
| | 12 | Blank LNPs | NA | NA | NA | 57/ | 6 | 0.31 |
| | | in Tris-10.3% | | | | 0.10 | | |
| | | w/v Sucrose, | | | | | | |
| | | Target lipids | | | | | | |
| | | eq. to 0.1 | | | | | | |
| | | mg/mL DP | | | | | | |
| Liquid drug | 13 | 0.01 mg/mL | 0.013 | 86 / | 98 | 61/ | 83 | NMT |
| product | | Flu modRNA | | NMT | | 0.08 | | 0.1 |
| | | in Tris-10.3% | | 3 | | | | |
| | | w/v Sucrose, | | | | | | |
| | | (liquid) (i.e., | | | | | | |
| | | blank LNPs | | | | | | |
| | | not included) | | | | | | |
| | 14 | 0.005 mg/mL | 0.006 | 87 / | 97 | 63/ | 95 | NMT |
| | | Flu modRNA | | NMT | | 0.09 | | 1.00 |
| | | in Tris-10.3% | | 3 | | | | |
| | | w/v Sucrose, | | | | | | |
| | | (liquid) (i.e., | | | | | | |
| | | blank LNPs | | | | | | |
| | | not included) | | | | | | |
| Study Bridging | 15 | 0.1 mg/mL | 0.109 | 94 | 94 | 70/ | 94 | 0.88 |
| Control | | Flu modRNA | | | | 0.16 | (125 ng) | |
| | | in Tris-10.3% | | | | | 91 | |
| | | w/v Sucrose, | | | | | (63 ng) | |
| | | Frozen control | | | | | | |

Results: Large Boost in Neutralization Titers (geometric mean titers, GMT) were Observed 2 Weeks post dose 2 for modRNA+Blank LNPs (see, e.g., Groups 2, 3, 7, and 8); Unfrozen Material (Groups 13 and 14) Elicits Antibody Titers 5 (0.005 mg) to 20 (0.01 mg)-Fold Lower than 4×F/T Material.

TABLE 19
Observed neutralization titer and IVE two weeks post dose 2

| Gp# | Mice | Description of RNA DP^{†} | GMT | IVE |
|---|---|---|---|---|
| 1 | 10 | 0.01 mg/mL Flu modRNA in | 4726 | 81 |
| | | Tris-10.3% w/v Sucrose, post 4x FT | | |
| 2 | 10 | 0.01 mg/mL Flu modRNA in | 29824 | 71 |
| | | Tris-10.3% w/v Sucrose | | |
| | | Target lipids eq. to 0.2 mg/mL DP, post 4x FT | | |
| 3 | 10 | 0.01 mg/mL Flu modRNA in Tris-10.3% w/v | 22881 | 81 |
| | | Sucrose Target lipids eq. to 0.1 mg/mL DP, | | |
| | | post 4x FT | | |
| 4 | 10 | 0.01 mg/mL Flu modRNA in | 9397 | 81 |
| | | Tris-15.4% w/v Sucrose, post 4x FT | | |
| 5 | 10 | 0.01 mg/mL Flu modRNA in | 3464 | 83 |
| | | Tris-20.5% w/v Sucrose, post 4x FT | | |
| 6 | 10 | 0.005 mg/mL Flu modRNA in | 1637 | 90 |
| | | Tris-10.3% w/v Sucrose, post 4x FT | | |
| 7 | 10 | 0.005 mg/mL Flu modRNA in | 10886 | 41 |
| | | Tris-10.3% w/v Sucrose | | |
| | | Target lipids eq. to 0.2 mg/mL DP, post 4x FT | | |
| 8 | 10 | 0.005 mg/mL Flu modRNA in | 25297 | 72 |
| | | Tris-10.3% w/v Sucrose | | |
| | | Target lipids eq. to 0.1 mg/mL DP, post 4x FT | | |
| 9 | 10 | 0.005 mg/mL Flu modRNA in | 2776 | 90 |
| | | Tris-15.4% w/v Sucrose, post 4x FT | | |
| 10 | 10 | 0.005 mg/mL Flu modRNA in | 1881 | 90 |
| | | Tris-20.5% w/v Sucrose, post 4x FT | | |
| 11 | 10 | Blank LNPs in Tris-10.3% w/v Sucrose (frozen) | 41 | 5 |
| | | Target lipids eq. to 0.2 mg/mL DP | | |
| 12 | 10 | Blank LNPs in Tris-10.3% w/v Sucrose (frozen) | 44 | 6 |
| | | Target lipids eq. to 0.1 mg/mL DP | | |
| 13 | 10 | 0.01 mg/mL Flu modRNA in | 237 | 83 |
| | | Tris-10.3% w/v Sucrose (liquid)(unfrozen) | | |
| 14 | 10 | 0.005 mg/mL Flu modRNA in | 306 | 95 |
| | | Tris-10.3% w/v Sucrose (liquid)(unfrozen) | | |
| 15 | 10 | 0.1 mg/mL Flu modRNA in | 373072 | n/a |
| | | Tris-10.3% w/v Sucrose 1x FT | | |
| 16 | 5 | Saline | 40 | n/a |

### Example 12: Use of an Alternative Organic Solvent (T-Butanol) in Producing LNPs

Studies were conducted to assess whether an alternative organic solvent, tert-butyl alcohol also referred to herein as TBA and/or t-butanol) can be used to produce LNPs. Studies were also conducted to assess use of histidine in high concentrations as an aqueous phase acidifier for saRNA.

It was observed that the use of t-butanol as the organic solvent allows for lyophilization to take place without a buffer exchanging step prior to drug product formulation, as the t-butanol may be condensed during freeze-drying. Histidine (present from acidification) is then diluted along with the saRNA, followed by a cryoprotectant spike to yield an acceptable pre-lyophilized drug product formulation.

As described below, saRNA-encapsulating LNPs were manufactured using t-butanol as the organic solvent. Lyophilization feasibility assessments (2 mL vials filled at 0.5 mL and reconstituted with RNAse free water) were then conducted.

In an exemplary process, the process used a 1:7 flow rate ratio (organic:aqueous) with t-butanol as the organic solvent. This solvent can be condensed during lyophilization but was observed to yield a turbid intermediate when a 1:3 flow rate ratio is used. The aqueous phase remains a part of the final drug product as the intermediate is not buffer exchanged as is typically done to remove the organic solvent. In the present exemplary processes, histidine was used as an exemplary acidification buffer. Succinate, citrate, acetate, and acetic acid may be acceptable substitutes for histidine as these buffers may be condensed during lyophilization, and tris could be added as the pH controlling buffer during the process step that adds cryoprotectant, e.g., sucrose.

### Example 13: Exemplary Process-Formulation F5

For general preparation of an saRNA solution, the saRNA was diluted with water and subsequently buffered in an acidification buffer, preferably histidine, at a pH preferably about 5.5.

In one embodiment, the saRNA was diluted with water and subsequently buffered in 190 mM histidine, pH 5.5. Separately, a lipids solution included an ionizable lipid, a polymer-conjugated lipid, such as PEG-conjugated lipid, a phospholipid, such as DSPC, and a sterol, such as cholesterol. In a clean and sterile container, 50-60 mM lipids were prepared with t-butanol. Accordingly, t-butanol was used as a solvent for the lipid solution and the saRNA preparation was in an acidification buffer of histidine.

The process of preparing lipid nanoparticles (LNPs) having an saRNA payload included a mixing system including a P-727 T-mixer as an exemplary T-mixer. The saRNA solution in the histidine acidification buffer flowed at a rate of 70 ml/min, and the lipids solution in t-butanol flowed at a rate of 10 ml/min into the P727 T-mixer to give a post-mix intermediate of saRNA-encapsulating LNPs in histidine, pH 5.5, 12.5% t-butanol. The intermediate was subsequently diluted with water and then diluted with about 20% sucrose 1:1. The resulting solution of LNPs underwent final filtration by PES filter and prepared for lyophilization. The final pre-lyophilization matrix for formulation F5 included: 10 μg/mL saRNA-encapsulating LNPs in 10 mM Histidine. 10% sucrose and 0.75% t-butanol.

TABLE 20
Formulation attributes for F5

| | | | | | Post- |
|---|---|---|---|---|---|
| | FRR | Post Mix Int | Pre-Lyo | Post-Lyo | Lyo |
| Formulation | (ml/min) | (Size/PDI) | (Size/PDI) | (Size/PDI) | EE % |
| F5 | 1:7 | 85.4/0.19 | 85.8/0.17 | 106.7/0.16 | 97 |
| | (10:70) | | | | |

EE % refers to encapsulation efficiency; FRR refers to flow rate ratio.

### Example 14: Altering saRNA Dilution

Follow-up experiments assessed reduced LNP dilutions resulting in higher pre-lyophilization t-butanol concentrations. Reduced histidine concentrations were also assessed during the acidification.

TABLE 21
Formulation attributes for F6 and F7

| | Flow rate ratio | Post Mix Int | Pre-Lyo | Post-Lyo |
|---|---|---|---|---|
| Formulation | (ml/min) | (Size/PDI) | (Size/PDI) | (Size/PDI) |
| F6 | 1:7 (10:70) | 88.9/0.19 | 89.0/0.18 | 124.7/0.09 |
| F7 | 1:7 (10:70) | 51.4/0.17 | 60.3/0.08 | 93.2/0.12 |

Formulation F6 refers to an LNP formulation having 20 ug/ml saRNA in 20 mM Histidine, 10% Sucrose, and 1.5% t-butanol.

Formulation F7 refers to an LNP formulation having 20 ug/ml saRNA in 10 mM Histidine, 10% Sucrose, and 1.5% t-butanol.

### Example 15: Exemplary Process-Formulation F6

In another embodiment, the saRNA was diluted with water and subsequently buffered in about 230-240 mM histidine, pH 5.5. Separately, a lipids solution included an ionizable lipid, a polymer-conjugated lipid, such as PEG-conjugated lipid, a phospholipid, such as DSPC, and a sterol, such as cholesterol. In a clean and sterile container, 50-60 mM lipids were prepared with t-butanol. Accordingly, t-butanol was used as a solvent for the lipid solution and the saRNA preparation was in an acidification buffer of histidine.

The process of preparing LNPs having an saRNA payload included a mixing system including a P-727 T-mixer as an exemplary T-mixer. The saRNA solution in the histidine acidification buffer flowed at a rate of 70 ml/min, and the lipids solution in t-butanol flowed at a rate of 10 ml/min into the P727 T-mixer to give a post-mix intermediate of saRNA-encapsulating LNPs in histidine, pH 5.5, and t-butanol. The intermediate was subsequently diluted with water and then diluted with about 20% sucrose in water 1:1. The resulting solution of LNPs underwent final filtration by PES filter and prepared for lyophilization. The final pre-lyophilization matrix for formulation F6 included: 20 μg/mL saRNA-encapsulating LNPs in 20 mM Histidine, 10% sucrose and 1.5% t-butanol.

### Example 16: Exemplary Process-Formulation F7

In another embodiment, the saRNA was diluted with water and subsequently buffered in about 120 mM histidine, pH 5.5. Separately, a lipids solution included an ionizable lipid, a polymer-conjugated lipid, such as PEG-conjugated lipid, a phospholipid, such as DSPC, and a sterol, such as cholesterol. In a clean and sterile container, 50-60 mM lipids were prepared with t-butanol. Accordingly, t-butanol was used as a solvent for the lipid solution and the saRNA preparation was in an acidification buffer of histidine.

The process of preparing LNPs having an saRNA payload included a mixing system including a P-727 T-mixer as an exemplary T-mixer. The saRNA solution in the histidine acidification buffer flowed at a rate of 70 ml/min, and the lipids solution in t-butanol flowed at a rate of 10 ml/min into the P727 T-mixer to give a post-mix intermediate of saRNA-encapsulating LNPs in histidine, pH 5.5, and t-butanol. The intermediate was subsequently diluted with water and then diluted with about 20% sucrose in water 1:1. The resulting solution of LNPs underwent final filtration by PES filter and prepared for lyophilization. The final pre-lyophilization matrix for formulation F7 included: 20 μg/mL saRNA-encapsulating LNPs in 10 mM Histidine, 10% sucrose and 1.5% t-butanol.

### Example 17: Altering the saRNA Dilution

Follow up experiments assessed reduced LNP dilution resulting in higher pre-lyo t-butanol concentrations. Reduced histidine concentrations were also assessed during the acidification.

Formulation A refers to a formulation prepared by Process A described below, having an aqueous phase of 0.2 mg/ml saRNA in 100 mM histidine, pH 5.5.

Formulation B refers to a formulation prepared by Process B described below, having an aqueous phase of 0.2 mg/ml saRNA in 150 mM histidine, pH 5.5.

TABLE 22
Formulation attributes for A and B

| | Flow | Post Mix | Pre-Lyo | Post-Lyo | Post-Lyo |
|---|---|---|---|---|---|
| Formu- | rate ratio | Int (Size/ | (Size/ | (Size/ | (Size/ |
| lation | (ml/min) | PDI) | PDI) | PDI) | PDI) |
| A-10% Suc. | 1:7 (10:70) | 58.4/0.09 | 58.0/0.12 | 91.8/0.13 | 96.5/0.09 |
| A-20% Suc. | 1:7 (10:70) | | 58.7/0.12 | 77.1/0.12 | 74.4/0.13 |
| B-10% Suc. | 1:7 (10:70) | 72.5/0.16 | 70.9/0.15 | 107.9/0.11 | 107.4/0.08 |
| B-20% Suc. | 1:7 (10:70) | | 69.6/0.16 | 81.5/0.17 | 82.1/0.13 |

The saRNA lot used for these studies exhibited a fragment analyzer result of 84% integrity. Formulation A-10% suc. refers to 20 ug/ml saRNA in 10 mM Histidine, 10% Sucrose, and 1.43% t-butanol, as prepared by Process A.

Formulation A-20% suc. refers to 20 ug/ml saRNA in 10 mM Histidine, 20% Sucrose, and 1.43% t-butanol, as prepared by Process A.

Formulation B-10% suc. refers to 20 ug/ml saRNA in 15 mM Histidine, 10% Sucrose, and 1.43% t-butanol, as prepared by Process B.

Formulation B-20% suc. refers to 20 ug/ml saRNA in 15 mM Histidine, 20% Sucrose, and 1.43% t-butanol, as prepared by Process B.

In Process A, the saRNA was diluted with water and subsequently buffered in about 100 mM histidine, pH 5.5. Separately, a lipids solution included an ionizable lipid, a polymer-conjugated lipid, such as PEG-conjugated lipid, a phospholipid, such as DSPC, and a sterol, such as cholesterol. In a clean and sterile container, 50-60 mM lipids were prepared with t-butanol. Accordingly, t-butanol was used as a solvent for the lipid solution and the saRNA preparation was in an acidification buffer of histidine. The process of preparing LNPs having an saRNA payload included a mixing system including a P-727 T-mixer as an exemplary T-mixer. The saRNA solution in the histidine acidification buffer flowed at a rate of 70 ml/min, and the lipids solution in t-butanol flowed at a rate of 10 ml/min into the P727 T-mixer to give a post-mix intermediate of saRNA-encapsulating LNPs in 80-90 mM histidine, pH 5.5, and 12.5% t-butanol. The intermediate was subsequently diluted with water and then diluted with about 20% sucrose or 40% sucrose in water 1:1. The resulting solution of LNPs underwent final filtration by PES filter and prepared for lyophilization. The final pre-lyophilization matrix for formulation A included: 20 μg/mL saRNA-encapsulating LNPs in 10 mM Histidine, 10% sucrose/20% sucrose (depending on sucrose dilution) and about 1.4%-1.5% t-butanol.

In Process B, the saRNA was diluted with water and subsequently buffered in about 150 mM histidine, pH 5.5. Separately, a lipids solution included an ionizable lipid, a polymer-conjugated lipid, such as PEG-conjugated lipid, a phospholipid, such as DSPC, and a sterol, such as cholesterol. In a clean and sterile container, 50-60 mM lipids were prepared with t-butanol. Accordingly, t-butanol was used as a solvent for the lipid solution and the saRNA preparation was in an acidification buffer of histidine. The process of preparing LNPs having an saRNA payload included a mixing system including a P-727 T-mixer as an exemplary T-mixer. The saRNA solution in the histidine acidification buffer flowed at a rate of 70 ml/min, and the lipids solution in t-butanol flowed at a rate of 10 ml/min into the P727 T-mixer to give a post-mix intermediate of saRNA-encapsulating LNPs in 130-140 mM histidine, pH 5.5, and 12.5% t-butanol. The intermediate was subsequently diluted with water and then diluted with about 20% sucrose or 40% sucrose in water 1:1. The resulting solution of LNPs underwent final filtration by PES filter and prepared for lyophilization. The final pre-lyophilization matrix for formulation B included: 20 μg/mL saRNA-encapsulating LNPs in 15 mM Histidine, 10% sucrose/20% sucrose (depending on sucrose dilution) and about 1.4%-1.5% t-butanol.

Process A and Process B use a 1:7 flow rate ratio (organic:aqueous phase) with t-butanol as the organic solvent. T-butanol can be condensed during lyophilization but yielded a turbid intermediate when a 1:3 flow rate ratio was used. See Example 20. The aqueous phase remains a part of the final drug product as the intermediate is not buffer exchanged in the Process.

As shown in the tables of data below, these exemplified processes resulted in post-lyophilization LNP size changes being limited to about 20 nm when compared to the LNP size pre-lyophilization. The processes also resulted in low polydispersity indexes (PDIs) less than 0.15. Encapsulation of the RNA payload surprisingly remained at or greater than 95%, when compared to the encapsulation of the RNA payload pre-lyophilization. Moreover, RNA integrity surprisingly remained high as the RNA-encapsulating LNPs underwent limited processing and hold times (Table 23).

TABLE 23
Formulation attributes for process A and process B

| | Size (d · | | % Encap- | | % Integrity | |
|---|---|---|---|---|---|---|
| | nm) / PDI | | sulation | | (Main/LMS) | |
| | Pre- | Post- | Pre- | Post- | Pre- | Post- |
| Formulation | Lyo | Lyo | Lyo | Lyo | Lyo | Lyo |
| A-10% Suc. | 67 / 0.13 | 90 / 0.12 | >98 | 96 | 80 / ≤3 | 78 / ≤3 |
| A-20% Suc. | 70 / 0.12 | 83 / 0.10 | 98 | 95 | 80 / ≤3 | 79 / ≤3 |
| B-10% Suc. | 82 / 0.13 | 103 / 0.10 | >98 | 96 | 79 / ≤3 | 75 / ≤3 |
| B-20% Suc. | 81 / 0.12 | 87 / 0.14 | 97 | 96 | 80 / ≤3 | 79 / ≤3 |

As shown in Tables 24 and 25 below, IVE changes post-lyophilization surprisingly remained minimal as EC50s in the suspension cell method are within the average range typically exhibited for RNA-encapsulating LNPs that undergo tangential flow filtration (TFF)-buffer exchange.

TABLE 24
In vitro Expression (IVE) data

| | IVE (EC50/% Positive) | |
|---|---|---|
| Formulation | Pre-Lyo | Post-Lyo |
| A-10% Suc. | 11/81 | 35/58 |
| A-20% Suc. | 16/84 | 30/62 |
| B-10% Suc. | 9/86 | 31/65 |
| B-20% Suc. | 12/82 | 31/64 |

TABLE 25
In vitro Expression (IVE) data, set B

| | IVE (EC50/% Positive) Set B | |
|---|---|---|
| | Pre-Lyo (Original | |
| Formulation | Process and Form.) | Post-Lyo |
| A-10% Suc. | 33/53 | 78/48 |
| | 23/58 | 74/49 |
| A-20% Suc. | | 53/54 |
| | | 62/51 |
| B-10% Suc. | | 33/54 |
| | | 38/51 |
| B-20% Suc. | | 55/52 |
| | | 108/44 |

### Example 18: Studies on Modified mRNA and on saRNA, 40 μg/Ml Drug Product

Modified mRNA payloads and saRNA encoding GFP were used to generate materials according to the exemplary processes, targeting a 40 ug/ml pre-lyophilization concentration. Limited particle size, encapsulation, and integrity changes were observed for respective LNPs produced by the processes described. The materials resulted in positive GFP expression on IVE.

The pre-lyophilization matrix of modified mRNA payload encoding GFP (modGFP) included 40 ug/ml modGFP in 10 mM Histidine, 20% Sucrose, and 2.86% t-butanol.

The pre-lyophilization matrix of saRNA payload encoding GFP (saRNA GFP) included 40 ug/ml saRNA GFP in 20 mM Histidine, 20% Sucrose, and 2.86% t-butanol.

TABLE 26
Formulation attributes of 40 ug/mL pre-lyophilization products

| | Size (d · | | % Encap- | | % Integrity | | GFP | |
|---|---|---|---|---|---|---|---|---|
| | nm) / PDI | | sulation | | (Main/LMS) | | Expression | |
| | Pre- | Post- | Pre- | Post- | Pre- | Post- | Pre- | Post- |
| Formulation | Lyo | Lyo | Lyo | Lyo | Lyo | Lyo | Lyo | Lyo |
| modGFP | 59 / 0.10 | 87 / 0.13 | >98 | 98 | 90 | 89 | Positive | Positive |
| saRNA GFP | 88 / 0.10 | 95 / 0.11 | >98 | 96 | 58 | 55 | Positive | Positive |

In the process to produce modRNA-encapsulating LNPs for a 40 μg/ml drug product concentration, the modRNA was diluted with water and subsequently buffered in about 50 mM histidine, pH 5.5. Separately, a lipids solution included an ionizable lipid, a polymer-conjugated lipid, such as PEG-conjugated lipid, a phospholipid, such as DSPC, and a sterol, such as cholesterol. In a clean and sterile container, 50-60 mM lipids were prepared with t-butanol. Accordingly, t-butanol was used as a solvent for the lipid solution and the modRNA preparation was in an acidification buffer of histidine. The process of preparing LNPs having an modRNA payload included a mixing system including a P-727 T-mixer as an exemplary T-mixer. The modRNA solution in the histidine acidification buffer flowed at a rate of 70 ml/min, and the lipids solution in t-butanol flowed at a rate of 10 ml/min into the P727 T-mixer to give a post-mix intermediate of modRNA-encapsulating LNPs in 40-50 mM histidine, pH 5.5, and 12.5% t-butanol. The intermediate was subsequently diluted with water and then diluted with about 40% sucrose in water 1:1. The resulting solution of LNPs underwent final filtration by PES filter and prepared for lyophilization. The final pre-lyophilization matrix for the modRNA included: 40 μg/mL modRNA-encapsulating LNPs in 10 mM Histidine, 20% sucrose and about 2-3% t-butanol.

In the process to produce saRNA-encapsulating LNPs for a 40 μg/ml drug product concentration, the saRNA was diluted with water and subsequently buffered in about 100 mM histidine, pH 5.5. Separately, a lipids solution included an ionizable lipid, a polymer-conjugated lipid, such as PEG-conjugated lipid, a phospholipid, such as DSPC, and a sterol, such as cholesterol. In a clean and sterile container, 50-60 mM lipids were prepared with t-butanol. Accordingly, t-butanol was used as a solvent for the lipid solution and the saRNA preparation was in an acidification buffer of histidine. The process of preparing LNPs having an saRNA payload included a mixing system including a P-727 T-mixer as an exemplary T-mixer. The saRNA solution in the histidine acidification buffer flowed at a rate of 70 ml/min, and the lipids solution in t-butanol flowed at a rate of 10 ml/min into the P727 T-mixer to give a post-mix intermediate of saRNA-encapsulating LNPs in 80-90 mM histidine, pH 5.5, and 12.5% t-butanol. The intermediate was subsequently diluted with water and then diluted with about 40% sucrose in water 1:1. The resulting solution of LNPs underwent final filtration by PES filter and prepared for lyophilization. The final pre-lyophilization matrix for saRNA included: 40 μg/mL saRNA-encapsulating LNPs in 10 mM Histidine, 20% sucrose and about 2-3% t-butanol.

### Example 19: LNPs Made Using Alternative Cationic Lipid Compositions

Additional LNP formulations were made using alternative lipid compositions and molar ratios and Process A described above in Example 17. The cationic lipids below were used with the molar ratio 50:38.5:10:1.5 (Cat-Lipid:Chol:DSPC:DMG-PEG).

Drug product formulation Dlin-DMA-MC3 had an N/P ratio of 6 and included 0.2 mg/ml saRNA in 100 mM Histidine, pH 5.5, 52 mM Lipid in t-butanol, 1:7 flow rate ratio

Drug product formulation R-DODMA had an N/P ratio of 12 and included 0.1 mg/ml saRNA in 100 mM Histidine, pH 5.5, 52 mM Lipid in t-butanol, 1:7 flow rate ratio

Drug product formulation DOTAP had an N/P ratio of 12 and included 0.1 mg/ml saRNA in 100 mM Histidine, pH 5.5, 52 mM Lipid in t-butanol, 1:7 flow rate ratio

Pre-lyophilization composition for the three drug products described above included 10-20 ug/ml RNA in 10 mM Histidine, 20% Sucrose, and 1.4-1.5% t-butanol.

Results shown below (Table 27).

TABLE 27
Formulation attributes for alternative cationic lipid compositions

| | Organic | Aqueous | FRR | Pre-Lyo | Post-Lyo |
|---|---|---|---|---|---|
| Formulation | (mM) | (mg/ml) | (ml/min) | (Size/PDI) | (Size/PDI) |
| Dlin-DMA- | 52.42 | 0.2 | 1:7 | 53.8/0.11 | 108.7/0.19 |
| MC3 | | | (10:70) | | |
| R-DODMA | 52.42 | 0.1 | 1:7 | 40.4/0.24 | 92.8/0.26 |
| | | | (10:70) | | |
| DOTAP | 52.42 | 0.1 | 1:7 | 50.4/0.26 | 64.0/0.25 |
| | | | (10:70) | | |

### Example 20: Flow Rate Ratio Assessments

Initial experiments pointed to flow rate ratio assessments to reduce the particle size of the RNA-encapsulating LNPs produced using t-butanol. Size increases of the LNPs post-lyophilization may be due to the presence of 1.5% t-butanol.

Histidine aqueous phase concentration used in preparing the formulations below: 200 mM.

TABLE 28
Formulation attributes with varying flow rate ratio (FRR)

| | Flow rate | Post Mix | | |
|---|---|---|---|---|
| | ratio | Intermediate | Pre-Lyo | Post-Lyo |
| Formulation | (ml/min) | (Size/PDI) | (Size/PDI) | (Size/PDI) |
| F1 | 1:3 (30:90) | N/A (turbid) | N/A | N/A |
| F2 | 1:7 (10:70) | 67.5/0.03 | 67.7/0.08 | 111.1/0.09 |

Resulting Formulation 2 included 10 ug/ml saRNA in 21 mM Histidine, 10% Sucrose, and 1.5% t-butanol.

### Example 21: Flu HA California

RNA lipid nanoparticle (LNP) generation using t-butanol (TBA) as the organic solvent was performed, followed by lyophilization feasibility assessments. The use of TBA as the organic solvent allows for lyophilization to occur without a buffer exchange step prior to drug product formulation as the TBA can be condensed during freeze-drying. Histidine (present from the acidification) can then be diluted along with the RNA followed by a cryoprotectant spike to yield a pre-lyophilized drug product formulation. saRNA was diluted to 1.0 mg/ml with water and then a T mixer was used to combine 0.4 mg/mL RNA in 50 or 100 mM Histidine (pH 5.5) at 300 mL/min with 94.59 mM lipids in t-butanol at 50 ml/min. The intermediate was then diluted from ~0.343 mg/mL to 0.08 mg/mL with water, followed by another dilution of the 0.08 mg/mL intermediate with 10 or 20 mM histidine in 60% sucrose at a 1:3 ratio. The final filtration then occurred with a PES filter (followed by fill and lyophilize) resulting in a final pre-lyo matrix of 60 ugl/mL RNA in 10 or 20 mM histidine, with 15% sucrose and 2.5% TBA.

Experiments were performed to assess the mixer with a range of flow rate ratios as shown in Table 29 (with the aqueous phase containing 0.2 mg/mL influenza California saRNA in 100 mM histidine at pH 5.5). Also assessed here were higher sucrose concentrations (20%) and saline as a reconstitution diluent. 0.2 mg/mL saRNA was targeted vs. the 0.1905 mg/mL done previously. The matching lipid was used (55.17 mM for N/P 6) with the flow rate ratio (and rates) unchanged. Both 100 mM and 150 mM Histidine were investigated here which provides a range of buffer concentration for the DP (10-15 mM) and a range for the buffer concentration in the acidifier (100-150 mM). 20% Sucrose appears to reduce the particle size change post-lyophilization compared to 10% Sucrose. Saline can be used as a reconstitution diluent as it does not significantly change the drug product size.

TABLE 29
Formulation attributes for California saRNA
with varying flow rate ratios - lyophilized

| | Flow | | | Post-Lyo | FA | IVE |
|---|---|---|---|---|---|---|
| | Rate | TBA | Encapsu- | (Size/ | (Main/ | (EC50/ |
| Formulation | Ratio | % | lation | PDI) | LMS) | % Posi) |
| F3 | 1:5 | 2.00 | 91 | 129 / 0.11 | 76 / 4 | 21 / 86 |
| F4 | 1:6 | 1.67 | 95 | 105 / 0.14 | 77 / 5 | 20 / 89 |
| F5 | 1:7 | 1.43 | 97 | 110 / 0.16 | 77 / 6 | 30 / 74 |
| F6 | 1:8 | 1.25 | 97 | 117 / 0.18 | 74 / 6 | 29 / 78 |
| F7 | 1:9 | 1.11 | 96 | 126 / 0.20 | 74 / 6 | 29 / 75 |
| F3 - | 1:5 | N/A | 94 | 121 / 0.11 | 74 / 6 | 23 / 83 |
| Dialyzed | | | | | | |
| F4 - | 1:6 | | 93 | 102 / 0.15 | 74 / 7 | 32 / 74 |
| Dialyzed | | | | | | |
| F5 - | 1:7 | | 89 | 105 / 0.18 | 73 / 7 | 45 / 69 |
| Dialyzed | | | | | | |
| F6 - | 1:8 | | 94 | 115 / 0.19 | 73 / 7 | 37 / 74 |
| Dialyzed | | | | | | |
| F7 - | 1:9 | | 94 | 122 / 0.18 | 72 / 7 | 56 / 61 |
| Dialyzed | | | | | | |

* Aqueous phase: 0.2 mg/mL saRNA in 100 mM histidine (pH 5.5); F3-F7 were lyophilized with TBA present

Cali modRNA was then used in a mixer with a 350 mL/min total flow rate and an aqueous phase of 0.4 mg/mL modRNA in 50 mM histidine (pH 5.5). Results are shown in Table 30.

TABLE 30
Formulation attributes for California modRNA - post-lyophilization

| Formulation (lyophilized | Flow Rate | | Pre-Lyo | Post-Lyo |
|---|---|---|---|---|
| with TBA present) | Ratio | TBA % | (Size/PDI) | (Size/PDI) |
| 80 ug/ml in 10 mM His | 1:6 | 3.333 | 52/0.06 | 137/0.10 |
| and 15% Sucrose | | | | |
| 60 ug/ml in 10 mM His | 1:6 | 2.5 | 56/0.05 | 101/0.09 |
| and 15% Sucrose | | | | |
| 40 ug/ml in 10 mM His | 1:6 | 1.667 | 57/0.05 | 84/0.10 |
| and 15% Sucrose | | | | |
| 20 ug/ml in 10 mM His | 1:6 | 0.833 | 58/0.04 | 92/0.11 |
| and 15% Sucrose | | | | |

Cali saRNA was then used in a mixer with a 50:300 mL/min flow rate ratio and an aqueous phase of 0.4 mg/mL saRNA in 100 mM histidine (pH 5.5). Results are shown in Table 31.

TABLE 31
Formulation attributes for California saRNA - post-lyophilization

| Formulation (lyophilized | Flow Rate | | Pre-Lyo | Post-Lyo |
|---|---|---|---|---|
| with TBA present) | Ratio | TBA % | (Size/PDI) | (Size/PDI) |
| 80 ug/ml in 10 mM His | 1:6 | 3.333 | 109/0.17 | 181/0.14 |
| and 15% Sucrose | | | | |
| 60 ug/ml in 10 mM His | 1:6 | 2.5 | 109/0.15 | 146/0.12 |
| and 15% Sucrose | | | | |
| 40 ug/ml in 10 mM His | 1:6 | 1.667 | 108/0.16 | 130/0.11 |
| and 15% Sucrose | | | | |
| 20 ug/ml in 10 mM His | 1:6 | 0.833 | 107/0.16 | 116/0.16 |
| and 15% Sucrose | | | | |

The immunogenicity of lyophilized and frozen modRNA and saRNA LNPs encapsulating Flu HA (California) formulated using the alternative organic solvent process was tested (as compared to the immunogenicity of modRNA and saRNA LNP drug product generated using the conventional process). The effect of lyophilization on immunogenicity of buffer exchanged material from the alternative organic solvent process compared to frozen buffer exchanged controls was also evaluated. Mice were immunized with different LNP Flu HA modRNA and saRNA materials. Sera was collected at day 20 post prime and day 42 (14 days post boost) and evaluated by serology testing (haemagglutination inhibition [HAI] and 1D neutralization). Statistical differences between groups were determined using 10 mice per group. ModRNA and saRNA were formulated using both standard and alternative processes to yield desired modRNA and saRNA LNPs at target conditions (−80C or lyophilized −5C). These LNPs were dosed as shown in Table 32 to assess process and formulation impact on immunogenicity. 90 BALB/c female mice were used at approximately 11-13 weeks of age with in vivo delivery of prime in the left leg and boost in the right leg. The study schedule included Vaccination #1 at week 0, day 0; a submandibular bleed at week 3, day 20; Vaccination #2 at week 4, day 28; and a terminal bleed (cardiac puncture at week 6, day 42.

TABLE 32
Lyophilized modRNA and saRNA flu lipid nanoparticles

| | | | | | | Vax | Bleed | |
|---|---|---|---|---|---|---|---|---|
| Grp | N | Description | Final DP | Dose | Volume | (Day) | (Day) | Condition |
| 1 | 10 | Saline | — | N/A | 0.05 mL | 0, 28 | 20, 42 | N/A |
| 2 | 10 | modRNA | 0.06 | 0.2 ug | 0.05 mL | 0, 28 | 20, 42 | 5° C. |
| | | TBA-Dilute | mg/mL | | | | | (Lyophilized) |
| | | Lyophilized | modCali | | | | | |
| | | (5° C. - | HA in | | | | | |
| | | Lyophilized) | 10 mM | | | | | |
| | | | Histidine | | | | | |
| | | | and 15% | | | | | |
| | | | Sucrose, | | | | | |
| | | | pH 5.5 | | | | | |
| 3 | 10 | modRNA | 0.06 | 0.2 ug | 0.05 mL | 0, 28 | 20, 42 | 5° C. |
| | | TBA- | mg/mL | | | | | (Lyophilized) |
| | | Dialyzed | modCali | | | | | |
| | | Lyophilized | HA in | | | | | |
| | | (5° C. - | 10 mM | | | | | |
| | | Lyophilized) | Histidine | | | | | |
| | | | and 15% | | | | | |
| | | | Sucrose, | | | | | |
| | | | pH 5.5 | | | | | |
| 4 | 10 | modRNA | 0.06 | 0.2 ug | 0.05 mL | 0, 28 | 20, 42 | −80° C. |
| | | TBA- | mg/mL | | | | | (Frozen) |
| | | Dialyzed | modCali | | | | | |
| | | Frozen | HA in | | | | | |
| | | (−80° C. - | 10 mM | | | | | |
| | | Frozen) | Histidine | | | | | |
| | | | and 15% | | | | | |
| | | | Sucrose, | | | | | |
| | | | pH 5.5 | | | | | |
| 5 | 10 | modRNA | 0.1 | 0.2 ug | 0.05 mL | 0, 28 | 20, 42 | −80° C. |
| | | Conventional | mg/mL | | | | | (Frozen) |
| | | Process/TFF | modCali | | | | | |
| | | Frozen | HA in | | | | | |
| | | (−80° C. - | 10 mM | | | | | |
| | | Frozen) | Tris and | | | | | |
| | | | 300mM | | | | | |
| | | | Sucrose, | | | | | |
| | | | pH 7.4 | | | | | |
| 6 | 10 | saRNA TBA- | 0.02 | 0.002 ug | 0.05 mL | 0, 28 | 20, 42 | 5° C. |
| | | Dilute | mg/mL | | | | | (Lyophilized) |
| | | Lyophilized | saCali | | | | | |
| | | (5° C. - | HA in | | | | | |
| | | Lyophilized) | 20 mM | | | | | |
| | | | Histidine | | | | | |
| | | | and 15% | | | | | |
| | | | Sucrose, | | | | | |
| | | | pH 5.5 | | | | | |
| 7 | 10 | saRNA TBA- | 0.06 | 0.002 ug | 0.05 mL | 0, 28 | 20, 42 | 5° C. |
| | | Dialyzed | mg/mL | | | | | (Lyophilized) |
| | | Lyophilized | saCali | | | | | |
| | | (5° C. - | HA in | | | | | |
| | | Lyophilized) | 20 mM | | | | | |
| | | | Histidine | | | | | |
| | | | and 15% | | | | | |
| | | | Sucrose, | | | | | |
| | | | pH 5.5 | | | | | |
| 8 | 10 | saRNA TBA- | 0.06 | 0.002 ug | 0.05 mL | 0, 28 | 20, 42 | −80° C. |
| | | Dialyzed | mg/mL | | | | | (Frozen) |
| | | Frozen | saCali | | | | | |
| | | (−80° C. - | HA in | | | | | |
| | | Frozen) | 20 mM | | | | | |
| | | | Histidine | | | | | |
| | | | and 15% | | | | | |
| | | | Sucrose, | | | | | |
| | | | pH 5.5 | | | | | |
| 9 | 10 | saRNA | 0.06 | 0.002 ug | 0.05 mL | 0, 28 | 20, 42 | −80° C. |
| | | Conventional | mg/mL | | | | | (Frozen) |
| | | Process/TFF | saCali | | | | | |
| | | Frozen | HA in | | | | | |
| | | (−80° C. - | 10 mM | | | | | |
| | | Frozen) | Tris and | | | | | |
| | | | 10% | | | | | |
| | | | Sucrose, | | | | | |
| | | | pH 7.4 | | | | | |

At three weeks post-dose 1, lyophilized modRNA using TBA as the organic solvent induced titers that trend lower than the conventional frozen control (FIG. 50). Titers from diluted or dialyzed lyophilized modRNA were comparable and frozen TBA-dialyzed modRNA induced titers approximately 7-fold lower than the control. Lyophilized saRNA using TBA and diluted induced titers slightly higher than conventional frozen control and TBA-dialyzed/lyophilized. Frozen TBA-dialyzed saRNA induced titers approximately 2-fold higher than the control. At two weeks post-dose 2, lyophilized modRNA using TBA as the organic solvent induced titers that trend lower than the conventional frozen control (FIG. 51). Titers from diluted or dialyzed lyophilized modRNA were comparable and frozen TBA-dialyzed modRNA induced titers approximately 3-fold lower than the control. Lyophilized saRNA using TBA and diluted induced comparable titers to conventional frozen control, and frozen TBA-dialyzed saRNA induced titers 1.5-fold higher than control.

All the methods disclosed and claimed herein can be made and executed without undue experimentation in light of the present disclosure. While the compositions and methods of this disclosure have been described in terms of preferred aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

Embodiments of the invention are further described in the following numbered embodiments:
E1) An immunogenic composition comprising (a) a first lipid nanoparticle; (b) a second lipid nanoparticle; (c) a cryoprotectant; and (d) tert-butanol; wherein the first lipid nanoparticle comprises i) a cationic lipid, ii) a neutral lipid and/or a phospholipid, iii) a steroid, and iv) a polymer conjugated lipid; wherein the first lipid nanoparticle encapsulates a ribonucleic acid (RNA) polynucleotide having an open reading frame encoding at least one polypeptide of interest, wherein the at least one polypeptide of interest comprises an antigen, preferably wherein the antigen is an influenza antigen; and wherein the second lipid nanoparticle does not encapsulate a nucleic acid.
E2) The composition according to embodiment 1, wherein the cationic lipid comprises ALC-0315, SM102, DLinDMA, DLin-MC3-DMA, DLin-KC2-DMA, A9, C12-200, L5, or derivatives and/or combinations thereof.
E3) The composition according to embodiment 1 or 2, wherein the neutral lipid and/or phospholipid comprises DSPC, DPPC, DMPC, DOPC, POPC, DOPE, SM, or derivatives and/or combinations thereof.
E4) The composition according to any one of embodiments 1 to 3, wherein the steroid comprises cholesterol, alpha-tocopherol, or derivatives and/or combinations thereof.
E5) The immunogenic composition according to any one of embodiments 1 to 4, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E6) The composition according to embodiment 1, wherein the total ratio of the first lipid nanoparticle and the second lipid nanoparticle is in the range of 1:1 to 1:4999.
E7) The composition according to any one embodiments 1 to 6, wherein at least 60% of the RNA in the composition is fully encapsulated in or associated with the first lipid nanoparticle.
E8) The composition according to any one of embodiments 1 to 7, wherein the second lipid nanoparticle comprises i) a cationic lipid, ii) a neutral lipid and/or a phospholipid, iii) a steroid, and iv) a polymer conjugated lipid.
E9) The composition according to embodiment 8, wherein the cationic lipid comprises ALC-0315, SM102, DLinDMA, DLin-MC3-DMA, DLin-KC2-DMA, A9, C12-200, L5, or derivatives and/or combinations thereof.
E10) The composition according to embodiment 8 or 9, wherein the neutral lipid and/or phospholipid comprises DSPC, DPPC, DMPC, DOPC, POPC, DOPE, SM, or derivatives and/or combinations thereof.
E11) The composition according to any one of embodiments 8 to 10, wherein the steroid comprises cholesterol, alpha-tocopherol, or derivatives and/or combinations thereof.
E12) The composition according to any one of embodiments 8 to 11, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E13) The composition according to any one of embodiments 8 to 12, wherein the i) cationic lipid, ii) neutral lipid and/or phospholipid, iii) steroid, and iv) polymer conjugated lipid of the second lipid nanoparticle are the same as the i) cationic lipid, ii) neutral lipid and/or phospholipid, iii) steroid, and iv) polymer conjugated lipid of the first lipid nanoparticle.
E14) The composition according to any one of embodiments 8 to 12, wherein one or more of the i) cationic lipid, ii) neutral lipid and/or phospholipid, iii) steroid, and/or iv) polymer conjugated lipid of the second lipid nanoparticle are different from the i) cationic lipid, ii) neutral lipid and/or phospholipid, iii) steroid, and/or iv) polymer conjugated lipid of the first lipid nanoparticle.
E15) The composition according to any one of embodiments 1 to 7, wherein the second lipid nanoparticle is a liposome.
E16) The composition according to embodiment 15, wherein the liposome comprises i) a phospholipid and/or a neutral lipid, ii) a steroid, and iii) a polymer conjugated lipid.
E17) The composition according to embodiment 15 or 16, wherein the liposome further comprises a cationic lipid.
E18) The composition according to embodiment 17, wherein the cationic lipid comprises ALC-0315, SM102, DLinDMA, DLin-MC3-DMA, DLin-KC2-DMA, A9, C12-200, L5, or derivatives and/or combinations thereof.
E19) The composition according to any one of embodiments 16 to 18, wherein the neutral lipid and/or phospholipid comprises DSPC, DPPC, DMPC, DOPC, POPC, DOPE, SM, or derivatives and/or combinations thereof.
E20) The composition according to any one of embodiments 16 to 19, wherein the steroid comprises cholesterol, alpha-tocopherol, or derivatives and/or combinations thereof.
E21) The composition according to any one of embodiments 16 to 20, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E22) The composition according to any one of embodiments 1 to 21, wherein the composition is liquid.
E23) The composition according to any one embodiments 1 to 22, wherein the composition is frozen.
E24) The composition according to any one of embodiments 1 to 23, wherein the cryoprotectant comprises a disaccharide.
E25) The composition according to any one of embodiments 1 to 24, wherein the cryoprotectant comprises sucrose.
E26) The composition according to any one of embodiments 1 to 25, wherein the cryoprotectant comprises sucrose, and wherein the composition comprises at least about 2% w/v to 30% w/v sucrose.
E27) The composition according to any one of embodiments 1 to 26, wherein the cryoprotectant comprises sucrose, and wherein the composition comprises at least about 10% w/v to 25% w/v sucrose.
E28) The composition according to any one of embodiments 1 to 27, wherein the cryoprotectant comprises sucrose, and wherein the composition comprises at least about 10.3% w/v to 20.5% w/v sucrose.
E29) The composition according to any one of embodiments 1 to 28, wherein the concentration of the cryoprotectant is 5 to 600 mg/mL in the composition before freezing.
E30) The composition according to any one embodiments 1 to 29, wherein the first and second lipid nanoparticles comprise between 40 and 50 molar percentage of the cationic lipid relative to total moles of all lipid components in the first and second lipid nanoparticles.
E31) The composition according to any one embodiments 1 to 30, wherein the composition further comprises a pharmaceutically acceptable buffer.
E32) The composition according to any one embodiments 1 to 31, wherein the second lipid nanoparticle has a size that is 50% less than the first lipid nanoparticle.
E33) The composition according to any one embodiments 1 to 31, wherein the second lipid nanoparticle has a size that is 50% greater than the first lipid nanoparticle.
E34) The composition according to any one of embodiments 1 to 33, wherein the mixture of the first lipid nanoparticle and the second lipid nanoparticle after freeze-thaw cycling has an average diameter size preferably in the range of 20 to 180 nm, more preferably in the range of 30 to 150 nm, and most preferably in the range of 40 to 120 nm.
E35) The composition according to any one of embodiments 1 to 34, wherein the composition has a water content of less than about 10% of the total composition.
E36) The composition according to any one of embodiments 1 to 35, wherein the composition has a water content between about 0.1% and 10% of the total composition.
E37) The composition according to any one of embodiments 1 to 36, wherein the composition is configured to be stable for at least about two weeks after storage as a frozen liquid composition or as a lyophilized composition at temperatures less than or equal to refrigerated storage.
E38) The composition according to any one of embodiments 1 to 37, wherein the composition is configured to be stable for at least 1 month after storage as a frozen liquid composition or as a lyophilized composition at temperatures less than or equal to refrigerated storage.
E39) The composition according to any one of embodiments 1 to 38, wherein the composition is configured to be stable for at least about two weeks after storage as a frozen liquid composition or as a lyophilized composition at temperatures less than or equal to refrigerated storage.
E40) The composition according to any one of embodiments 1 to 39, wherein the composition is configured to be stable for at least about four weeks after storage as a frozen liquid composition or as a lyophilized composition at temperatures less than or equal to refrigerated storage.
E41) The composition according to any one of embodiments 1 to 40, wherein the composition is configured to be stable for about 2 weeks to about 1 month, 2 months, 3 months, 4 months, 5 months, 6 months, 1 year, or 2 years after storage as a frozen liquid composition or as a lyophilized composition at temperatures less than or equal to refrigerated storage.
E42) The composition according to any one of embodiments 1 to 41, wherein the composition is configured to have at least 50%, 60%, 65%, 70%, 75%, 80%, 85%, 90% or 95% of the RNA intact at least about two weeks after storage as a frozen liquid composition or as a lyophilized composition at temperatures less than or equal to refrigerated storage.
E43) The composition according to any one of embodiments 1 to 42, wherein the composition is configured to have at least 60%, 65%, 70%, 75%, 80%, 85%, 90% or 95% of the RNA intact at least 1 month after storage as a frozen liquid composition or as a lyophilized composition at temperatures less than or equal to refrigerated storage.
E44) The composition according to any one of embodiments 1 to 43, wherein the composition is configured to have at least 60%, 65%, 70%, 75%, 80%, 85%, 90% or 95% of the RNA intact about 2 weeks to about 1 month, 2 months, 3 months, 4 months, 5 months, 6 months, 1 year, or 2 years after storage as a frozen liquid composition or as a lyophilized composition at temperatures less than or equal to refrigerated storage.
E45) The composition according to any one of embodiments 1 to 44, wherein the composition is configured to have at least 80% of the RNA intact after about two weeks of storage as a frozen liquid composition or as a lyophilized composition at temperatures less than or equal to refrigerated storage.
E46) The composition according to any one of embodiments 1 to 45, wherein the concentration of the RNA is in a range from about 10 μg/ml to about 10 mg/ml, preferably in a range from about 0.1 μg/mL to 0.5 mg/mL.
E47) The composition according to any one of embodiments 1 to 46, wherein the RNA has an RNA integrity of at least about 50% or greater, preferably of at least about 60% or greater, more preferably of at least about 70% or greater, most preferably of at least about 80% or greater, as compared to a composition comprising the first lipid nanoparticle and not comprising the second lipid nanoparticle, when measured under identical conditions.
E48) The composition according to any one of embodiments 1 to 47, wherein the RNA has an RNA integrity of at least about 50% greater, preferably of at least about 60% greater, more preferably of at least about 70% greater, most preferably of at least about 80% greater, than a composition comprising the first lipid nanoparticle and not comprising the second lipid nanoparticle, when measured under identical conditions.
E49) The composition according to any one of embodiments 1 to 48, wherein the composition comprises greater than 60% more encapsulated RNA, preferably greater than 70% more encapsulated RNA, more preferably greater than 80% more encapsulated RNA, and most preferably greater than 90% more encapsulated RNA, as compared to a composition comprising the first lipid nanoparticle and not comprising the second lipid nanoparticle, when measured under identical conditions.
E50) The composition according to any one of embodiments 1 to 49, wherein the composition comprises greater than 60% encapsulated RNA, preferably greater than 70% encapsulated RNA, more preferably greater than 80% encapsulated RNA, and most preferably greater than 90% encapsulated RNA, as compared to a composition comprising the first lipid nanoparticle and not comprising the second lipid nanoparticle, when measured under identical conditions.
E51) The composition according to any one of embodiments 1 to 50, wherein the RNA has been purified by at least one purification step, and wherein the first lipid nanoparticle has been purified by at least one purification step, preferably by at least one step of tangential flow filtration (TFF) and/or at least one step of clarification and/or at least one step of filtration.
E52) The composition according to any one of embodiments 1 to 51, wherein the RNA is a purified RNA, preferably an RP-HPLC purified RNA and/or a tangential flow filtration (TFF) purified RNA.
E53) The composition according to any one of embodiments 1 to 52, wherein the potency of the composition decreases less than about 30%, preferably less than about 20%, more preferably less than about 10%, as compared to a composition comprising the first lipid nanoparticle and not comprising the second lipid nanoparticle, when measured under identical conditions.
E54) The composition according to any one of embodiments 1 to 53, wherein the composition comprises an effective amount of RNA to produce the polypeptide of interest in a cell.
E55) The composition according to any one of embodiments 1 to 54, wherein the RNA further comprises a modified nucleotide.
E56) The composition according to any one of embodiments 1 to 55, wherein the RNA comprises a modified nucleotide comprising N1-Methylpseudourodine-5′-triphosphate (m1ψTP).
E57) The composition according to any one of embodiments 1 to 56, wherein the RNA comprises a translatable region encoding the antigen and comprises a modified nucleoside comprising 1-methyl-pseudouridine.
E58) The composition according to any one of embodiments 1 to 57, wherein the RNA comprises an open reading frame encoding at least one influenza virus antigenic polypeptide or an immunogenic fragment thereof.
E59) The composition according to any one of embodiments 1 to 58, wherein the RNA further comprises a 5′ cap analog.
E60) The composition according to any one of embodiments 1 to 59, wherein the RNA further comprises a 5′ cap analog and wherein the 5′ cap analog comprises m27,3″ ^{OMe}Gppp (m12′-0) ApG.
E61) The composition according to any one of embodiments 1 to 60, wherein the antigen is influenza hemagglutinin 1 (HA1), hemagglutinin 2 (HA2), an immunogenic fragment of HA1 or HA2, or a combination of any two or more of the foregoing.
E62) The composition according to any one of embodiments 1 to 61, wherein the RNA encodes at least two antigenic polypeptides or immunogenic fragments thereof, wherein a first antigen is HA1, HA2, or a combination of HA1 and HA2, and wherein a second antigen is selected from the group consisting of neuraminidase (NA), nucleoprotein (NP), matrix protein 1 (M1), matrix protein 2 (M2), non-structural protein 1 (NS1), and non-structural protein 2 (NS2).
E63) The composition according to any one of embodiments 1 to 62, wherein the RNA encodes at least two antigenic polypeptides or immunogenic fragments thereof, wherein a first antigen is HA1, HA2, or a combination of HA1 and HA2, and wherein a second antigen is neuraminidase (NA).
E64) The composition according to any one of embodiments 1 to 60, wherein the antigen is a polypeptide or an immunogenic fragment thereof from an arenavirus; an astrovirus; a bunyavirus; a calicivirus; a coronavirus; a filovirus; a flavivirus; a hepadnavirus; a hepevirus; an orthomyxovirus; a paramyxovirus; a picornavirus; a reovirus; a retrovirus; a rhabdovirus; a togavirus; or a combination of any two or more of the foregoing.
E65) The composition according to any one of embodiments 1 to 60, wherein the antigen is a polypeptide or an immunogenic fragment thereof from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 01 57:H7, 01 1 1 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBOV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowled, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B1 9, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, Yersinia pseudotuberculosis, or a combination of any two or more of the foregoing.
E66) The composition according to any one of embodiments 1 to 65, wherein the open reading frame is codon-optimized.
E67) The composition according to any one of embodiments 1 to 66, wherein the composition comprises ALC-0315 (4-hydroxybutyl) azanediyl)bis(hexane-6,1-diyl)bis(2-hexyldecanoate).
E68) The composition according to any one of embodiments 1 to 67, wherein the composition comprises ALC-0159 (2-[(polyethylene glycol)-2000]-N,N-ditetradecylacetamide).
E69) The composition according to any one of embodiments 1 to 68, wherein the composition comprises 1,2-Distearoyl-sn-glycero-3-phosphocholine (DSPC).
E70) The composition according to any one of embodiments 1 to 69, wherein the composition comprises cholesterol.
E71) The composition according to any of embodiments 1 to 70, wherein the composition has been freeze-thawed at least 2 times.
E72) The composition according to any of embodiments 1 to 71, wherein the composition has been freeze-thawed at least 3 times.
E73) The composition according to any of embodiments 1 to 72, wherein the composition has been freeze-thawed at least 4 times.
E74) The composition according to any of embodiments 1 to 73, wherein the composition has been freeze-thawed at least 5 times.
E75) A method of producing a polypeptide of interest in a cell, comprising administering a composition according to any one of embodiments 1-74, wherein the composition produces an increased amount of the polypeptide, as compared to a composition comprising the first lipid nanoparticle and not comprising the second lipid nanoparticle, when measured under identical conditions.
E76) The method according to embodiment 75, wherein the composition is administered to a mammal.
E77) The method according to any one of embodiments 75 to 76, wherein the composition is administered to a human.
E78) The method according to any one of embodiments 75 to 77, wherein the composition is administered to a mammal at risk of having influenza.
E79) A method of increasing the stability of a composition comprising a first lipid nanoparticle, the first lipid nanoparticle comprising i) a cationic lipid, ii) a neutral lipid and/or phospholipid, iii) a steroid, iv) a polymer conjugated lipid, and v) a ribonucleic acid (RNA) polynucleotide encapsulated in the first lipid nanoparticle, the method comprising contacting the composition with a second lipid nanoparticle, wherein the second lipid nanoparticle does not encapsulate a ribonucleic acid (RNA) polynucleotide.
E80) The method according to embodiment 79, wherein the cationic lipid comprises ALC-0315, SM102, DLinDMA, DLin-MC3-DMA, DLin-KC2-DMA, A9, C12-200, L5, or derivatives and/or combinations thereof.
E81) The method according to embodiment 79 or 80, wherein the neutral lipid and/or phospholipid comprises DSPC, DPPC, DMPC, DOPC, POPC, DOPE, SM, or derivatives and/or combinations thereof.
E82) The method according to any one of embodiments 79 to 81, wherein the steroid comprises cholesterol, alpha-tocopherol, or derivatives and/or combinations thereof.
E83) The method according to any one of embodiments 79 to 82, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E84) The method of any one of embodiments 79 to 83, wherein the second lipid nanoparticle comprises i) a cationic lipid, ii) a neutral lipid and/or a phospholipid, iii) a steroid, and iv) a polymer conjugated lipid.
E85) The method according to embodiment 84, wherein the cationic lipid comprises ALC-0315, SM102, DLinDMA, DLin-MC3-DMA, DLin-KC2-DMA, A9, C12-200, L5, or derivatives and/or combinations thereof.
E86) The method according to embodiment 84 or 85, wherein the neutral lipid and/or phospholipid comprises DSPC, DPPC, DMPC, DOPC, POPC, DOPE, SM, or derivatives and/or combinations thereof.
E87) The method according to any one of embodiments 84 to 86, wherein the steroid comprises cholesterol, alpha-tocopherol, or derivatives and/or combinations thereof.
E88) The method according to any one of embodiments 84 to 87, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E89) The method according to any one of embodiments 84 to 88, wherein the i) cationic lipid, ii) neutral lipid and/or phospholipid, iii) steroid, and iv) polymer conjugated lipid of the second lipid nanoparticle are the same as the i) cationic lipid, ii) neutral lipid and/or phospholipid, iii) steroid, and iv) polymer conjugated lipid of the first lipid nanoparticle.
E90) The method according to any one of embodiments 84 to 88, wherein one or more of the i) cationic lipid, ii) neutral lipid and/or phospholipid, iii) steroid, and/or iv) polymer conjugated lipid of the second lipid nanoparticle are different from the i) cationic lipid, ii) neutral lipid and/or phospholipid, iii) steroid, and/or iv) polymer conjugated lipid of the first lipid nanoparticle.
E91) The method according to embodiment 79, wherein the second lipid nanoparticle comprises a liposome.
E92) The method according to embodiment 91, wherein the liposome comprises i) a phospholipid and/or a neutral lipid, ii) a steroid, and/or iii) a polymer conjugated lipid.
E93) The method according to embodiment 92, wherein the liposome further comprises a cationic lipid.
E94) The method according to embodiment 93, wherein the cationic lipid comprises ALC-0315, SM102, DLinDMA, DLin-MC3-DMA, DLin-KC2-DMA, A9, C12-200, L5, or derivatives and/or combinations thereof.
E95) The method according to any one of embodiments 92 to 94, wherein the neutral lipid and/or phospholipid comprises DSPC, DPPC, DMPC, DOPC, POPC, DOPE, SM, or derivatives and/or combinations thereof.
E96) The method according to any one of embodiments 92 to 95, wherein the steroid comprises cholesterol, alpha-tocopherol, or derivatives and/or combinations thereof.
E97) The method according to any one of embodiments 92 to 96, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E98) The method of any one of embodiments 79 to 97, wherein the stability increase comprises the storage stability of the composition when frozen.
E99) The method of any one of embodiments 79 to 98, wherein the stability increase comprises the stability of the composition when thawed after being frozen.
E100) The method of any one of embodiments 79 to 99, wherein the stability increase comprises the stability of the composition when the composition has been freeze-thawed at least 2 times.
E101) The method of any one of embodiments 79 to 100, wherein the stability increase comprises the stability of the composition when the composition has been freeze-thawed at least 3 times.
E102) The method of any one of embodiments 79 to 101, wherein the stability increase comprises the stability of the composition when the composition has been freeze-thawed at least 4 times.
E103) The method of any one of embodiments 79 to 102, wherein the stability increase comprises the stability of the composition when the composition has been freeze-thawed at least 5 times.
E104) The method of any one of embodiments 79 to 103, wherein contacting the composition with a second lipid nanoparticle forms the immunogenic composition of any one of embodiments 1 to 74.
E105) A method of increasing the stability of a composition comprising a first lipid nanoparticle and a second lipid nanoparticle, the first lipid nanoparticle comprising i) a cationic lipid, ii) a neutral lipid and/or a phospholipid, iii) a steroid, iv) a polymer conjugated lipid, and v) a ribonucleic acid (RNA) polynucleotide encapsulated in the first lipid nanoparticle, the second lipid nanoparticle lacking a ribonucleic acid (RNA) polynucleotide encapsulated in the second lipid nanoparticle, and the method comprising purifying the composition to remove a first portion of a plurality of the second lipid nanoparticle from the composition before freezing, wherein a second portion of the plurality of the second lipid nanoparticle remains in the composition.
E106) The method according to embodiment 105, wherein the cationic lipid comprises ALC-0315, SM102, DLinDMA, DLin-MC3-DMA, DLin-KC2-DMA, A9, C12-200, L5, or derivatives and/or combinations thereof.
E107) The method according to embodiment 105 or 106, wherein the neutral lipid and/or phospholipid comprises DSPC, DPPC, DMPC, DOPC, POPC, DOPE, SM, or derivatives and/or combinations thereof.
E108) The method according to any one of embodiments 105 to 107, wherein the steroid comprises cholesterol, alpha-tocopherol, or derivatives and/or combinations thereof.
E109) The method according to any one of embodiments 105 to 108, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E110) The method of any one of embodiments 105 to 109, wherein the second lipid nanoparticle comprises i) a cationic lipid, ii) a neutral lipid and/or a phospholipid, iii) a steroid, and iv) a polymer conjugated lipid.
E111) The method according to embodiment 110, wherein the cationic lipid comprises ALC-0315, SM102, DLinDMA, DLin-MC3-DMA, DLin-KC2-DMA, A9, C12-200, L5, or derivatives and/or combinations thereof.
E112) The method according to embodiment 110 or 111, wherein the neutral lipid and/or phospholipid comprises DSPC, DPPC, DMPC, DOPC, POPC, DOPE, SM, or derivatives and/or combinations thereof.
E113) The method according to any one of embodiments 110 to 112, wherein the steroid comprises cholesterol, alpha-tocopherol, or derivatives and/or combinations thereof.
E114) The method according to any one of embodiments 110 to 113, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E115) The method according to any one of embodiments 110 to 114, wherein the i) cationic lipid, ii) neutral lipid and/or phospholipid, iii) steroid, and iv) polymer conjugated lipid of the second lipid nanoparticle are the same as the i) cationic lipid, ii) neutral lipid and/or phospholipid, iii) steroid, and iv) polymer conjugated lipid of the first lipid nanoparticle.
E116) The method according to any one of embodiments 110 to 114, wherein one or more of the i) cationic lipid, ii) neutral lipid and/or phospholipid, iii) steroid, and/or iv) polymer conjugated lipid of the second lipid nanoparticle are different from the i) cationic lipid, ii) neutral lipid and/or phospholipid, iii) steroid, and/or iv) polymer conjugated lipid of the first lipid nanoparticle.
E117) The method according to embodiment 105, wherein the second lipid nanoparticle comprises a liposome.
E118) The method according to embodiment 117, wherein the liposome comprises i) a phospholipid and/or a neutral lipid, ii) a steroid, and/or iii) a polymer conjugated lipid.
E119) The method according to embodiment 118, wherein the liposome further comprises a cationic lipid.
E120) The method according to embodiment 119, wherein the cationic lipid comprises ALC-0315, SM102, DLinDMA, DLin-MC3-DMA, DLin-KC2-DMA, A9, C12-200, L5, or derivatives and/or combinations thereof.
E121) The method according to any one of embodiments 118 to 120, wherein the neutral lipid and/or phospholipid comprises DSPC, DPPC, DMPC, DOPC, POPC, DOPE, SM, or derivatives and/or combinations thereof.
E122) The method according to any one of embodiments 118 to 121, wherein the steroid comprises cholesterol, alpha-tocopherol, or derivatives and/or combinations thereof.
E123) The method according to any one of embodiments 118 to 122, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E124) The method of any one of embodiments 105 to 123, wherein the stability increase comprises the storage stability of the composition when frozen.
E125) The method of and one of embodiments 105 to 124, wherein the stability increase comprises the stability of the composition when thawed after being frozen.
E126) The method of any one of embodiments 105 to 125, wherein the stability increase comprises the stability of the composition when the composition has been freeze-thawed at least 2 times.
E127) The method of any one of embodiments 105 to 126, wherein the stability increase comprises the stability of the composition when the composition has been freeze-thawed at least 3 times.
E128) The method of any one of embodiments 105 to 127, wherein the stability increase comprises the stability of the composition when the composition has been freeze-thawed at least 4 times.
E129) The method of any one of embodiments 105 to 128, wherein the stability increase comprises the stability of the composition when the composition has been freeze-thawed at least 5 times.
E130) The method of any one of embodiments 105 to 129, wherein purifying the composition to remove a first portion of a plurality of the second lipid nanoparticle from the composition before freezing forms the immunogenic composition of any one of embodiments 1 to 74.
E131) An immunogenic composition comprising (a) a first lipid nanoparticle; and an effective amount of a cryoprotectant; wherein the first lipid nanoparticle comprises i) a cationic lipid, ii) a neutral lipid and/or a phospholipid, iii) a steroid, and iv) a polymer conjugated lipid; wherein the first lipid nanoparticle encapsulates a ribonucleic acid (RNA) polynucleotide having an open reading frame encoding at least one polypeptide of interest, wherein the at least one polypeptide of interest comprises an antigen, preferably wherein the antigen is an influenza antigen; wherein the cryoprotectant comprises a saccharide; and wherein the effective amount of the cryoprotectant is at least about 2% w/v to 30% w/v of the composition.
E132) The composition according to embodiment 131, wherein the cationic lipid comprises ALC-0315, SM102, DLinDMA, DLin-MC3-DMA, DLin-KC2-DMA, A9, C12-200, L5, or derivatives and/or combinations thereof.
E133) The composition according to embodiment 131 or 132, wherein the neutral lipid and/or phospholipid comprises DSPC, DPPC, DMPC, DOPC, POPC, DOPE, SM, or derivatives and/or combinations thereof.
E134) The composition according to any one of embodiments 131 to 133, wherein the steroid comprises cholesterol, alpha-tocopherol, or derivatives and/or combinations thereof.
E135) The composition according to any one of embodiments 131 to 134, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E136) The composition according to any one of embodiments 131 to 135, wherein the cryoprotectant comprises a disaccharide.
E137) The composition according to any one of embodiments 131 to 136, wherein the cryoprotectant comprises sucrose.
E138) The composition according to any one of embodiments 131 to 137, wherein the composition comprises at least about 10% w/v to 25% w/v of the cryoprotectant.
E139) The composition according to any one of embodiments 131 to 138, wherein the composition comprises at least about 10.3% w/v to 20.5% w/v of the cryoprotectant.
E140) The composition according to any one of embodiments 131 to 139, wherein the concentration of the cryoprotectant is 5 to 600 mg/mL in the composition before freezing.
E141) The composition according to any one of embodiments 131 to 140, further comprising a second lipid nanoparticle, wherein the second lipid nanoparticle does not encapsulate an RNA polynucleotide.
E142) The composition according to embodiment 141, wherein the second lipid nanoparticle comprises i) a cationic lipid, ii) a neutral lipid and/or a phospholipid, iii) a steroid, and iv) a polymer conjugated lipid.
E143) The composition according to embodiment 142, wherein the cationic lipid comprises ALC-0315, SM102, DLinDMA, DLin-MC3-DMA, DLin-KC2-DMA, A9, C12-200, L5, or derivatives and/or combinations thereof.
E144) The composition according to embodiment 142 or 143, wherein the neutral lipid and/or phospholipid comprises DSPC, DPPC, DMPC, DOPC, POPC, DOPE, SM, or derivatives and/or combinations thereof.
E145) The composition according to any one of embodiments 142 to 144, wherein the steroid comprises cholesterol, alpha-tocopherol, or derivatives and/or combinations thereof.
E146) The composition according to any one of embodiments 142 to 145, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E147) The composition according to any one of embodiments 142 to 146, wherein the i) cationic lipid, ii) neutral lipid and/or phospholipid, iii) steroid, and iv) polymer conjugated lipid of the second lipid nanoparticle are the same as the i) cationic lipid, ii) neutral lipid and/or phospholipid, iii) steroid, and iv) polymer conjugated lipid of the first lipid nanoparticle.
E148) The composition according to any one of embodiments 142 to 146, wherein one or more of the i) cationic lipid, ii) neutral lipid and/or phospholipid, iii) steroid, and/or iv) polymer conjugated lipid of the second lipid nanoparticle are different from the i) cationic lipid, ii) neutral lipid and/or phospholipid, iii) steroid, and/or iv) polymer conjugated lipid of the first lipid nanoparticle.
E149) The composition according to embodiment 141, wherein the second lipid nanoparticle is a liposome.
E150) The composition according to embodiment 149, wherein the liposome comprises i) a phospholipid and/or a neutral lipid, ii) a steroid, and iii) a polymer conjugated lipid.
E151) The composition according to embodiment 150, wherein the liposome further comprises a cationic lipid.
E152) The composition according to embodiment 151, wherein the cationic lipid comprises ALC-0315, SM102, DLinDMA, DLin-MC3-DMA, DLin-KC2-DMA, A9, C12-200, L5, or derivatives and/or combinations thereof.
E153) The composition according to any one of embodiments 150 or 152, wherein the neutral lipid and/or phospholipid comprises DSPC, DPPC, DMPC, DOPC, POPC, DOPE, SM, or derivatives and/or combinations thereof.
E154) The composition according to any one of embodiments 150 to 153, wherein the steroid comprises cholesterol, alpha-tocopherol, or derivatives and/or combinations thereof.
E155) The composition according to any one of embodiments 150 to 154, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E156) The composition according to any one of embodiments 141 to 155, wherein the total ratio of the first lipid nanoparticle and the second lipid nanoparticle is in the range of 1:1 to 1:4999.
E157) The composition according to any one embodiments 141 to 156, wherein at least 60% of the RNA in the composition is fully encapsulated in or associated with the first lipid nanoparticle.
E158) The composition according to any one embodiments 141 to 157, wherein the first and second lipid nanoparticles comprise between 40 and 50 molar percentage of the cationic lipid relative to total moles of all lipid components in the first and second lipid nanoparticles.
E159) The composition according to any one embodiments 141 to 158, wherein the second lipid nanoparticle has a size that is 50% less than the first lipid nanoparticle.
E160) The composition according to any one embodiments 141 to 158, wherein the second lipid nanoparticle has a size that is 50% greater than the first lipid nanoparticle.
E161) The composition according to any one of embodiments 141 to 160, wherein the mixture of the first lipid nanoparticle and the second lipid nanoparticle after freeze-thaw cycling has an average diameter size preferably in the range of 20 to 180 nm, more preferably in the range of 30 to 150 nm, and most preferably in the range of 40 to 120 nm.
E162) The composition according to any one of embodiments 141 to 161, wherein inclusion of the second lipid nanoparticle further increases the stability of the composition compared to a composition not comprising the second lipid nanoparticle, when measured under identical conditions.
E163) The composition according to any one embodiments 131 to 162, wherein the composition is liquid.
E164) The composition according to any one of embodiments 131 to 163, wherein the composition is frozen or as a lyophilized composition at temperatures less than or equal to refrigerated storage.
E165) The composition according to any one embodiments 131 to 164, 235, or 254, wherein the composition further comprises a pharmaceutically acceptable buffer.
E166) The composition according to any one of embodiments 131 to 165, wherein the composition has a water content of less than about 10% of the total composition.
E167) The composition according to any one of embodiments 131 to 166, wherein the composition has a water content between about 0.1% and 10% of the total composition.
E168) The composition according to any one of embodiments 131 to 167, wherein the composition is configured to be stable for at least about two weeks after storage as a frozen liquid composition or as a lyophilized composition at temperatures less than or equal to refrigerated storage.
E169) The composition according to any one of embodiments 131 to 168, wherein the composition is configured to be stable for at least 1 month after storage as a frozen liquid composition or as a lyophilized composition at temperatures less than or equal to refrigerated storage.
E170) The composition according to any one of embodiments 131 to 169, wherein the composition is configured to be stable for at least about two weeks after storage as a frozen liquid composition or as a lyophilized composition at temperatures less than or equal to refrigerated storage.
E171) The composition according to any one of embodiments 131 to 170, wherein the composition is configured to be stable for at least about four weeks after storage as a frozen liquid composition or as a lyophilized composition at temperatures less than or equal to refrigerated storage.
E172) The composition according to any one of embodiments 131 to 171, wherein the composition is configured to be stable for about 2 weeks to about 1 month, 2 months, 3 months, 4 months, 5 months, 6 months, 1 year, or 2 years after storage as a frozen liquid composition or as a lyophilized composition at temperatures less than or equal to refrigerated storage.
E173) The composition according to any one of embodiments 131 to 172, wherein the composition is configured to have at least 50%, 60%, 65%, 70%, 75%, 80%, 85%, 90% or 95% of the RNA intact at least about two weeks after storage as a frozen liquid composition or as a lyophilized composition at temperatures less than or equal to refrigerated storage.
E174) The composition according to any one of embodiments 131 to 173, wherein the composition is configured to have at least 60%, 65%, 70%, 75%, 80%, 85%, 90% or 95% of the encapsulated RNA intact at least 1 month after storage as a frozen liquid composition or as a lyophilized composition at temperatures less than or equal to refrigerated storage.
E175) The composition according to any one of embodiments 131 to 174, wherein the composition is configured to have at least 60%, 65%, 70%, 75%, 80%, 85%, 90% or 95% of the RNA intact about 2 weeks to about 1 month, 2 months, 3 months, 4 months, 5 months, 6 months, 1 year, or 2 years after storage as a frozen liquid composition or as a lyophilized composition at temperatures less than or equal to refrigerated storage.
E176) The composition according to any one of embodiments 131 to 175, wherein the composition is configured to have at least 80% of the RNA intact after about two weeks of storage as a frozen liquid composition or as a lyophilized composition at temperatures less than or equal to refrigerated storage.
E177) The composition according to any one of embodiments 131 to 176, wherein the concentration of the RNA is in a range from about 10 μg/ml to about 10 mg/ml, preferably in a range from about 0.1 μg/mL to 0.5 mg/mL.
E178) The composition according to any one of embodiments 131 to 177, wherein the RNA has an RNA integrity of at least about 50% or greater, preferably of at least about 60% or greater, more preferably of at least about 70% or greater, most preferably of at least about 80% or greater, as compared to a composition comprising the first lipid nanoparticle and not comprising the effective amount of the cryoprotectant, when measured under identical conditions.
E179) The composition according to any one of embodiments 131 to 178, wherein the RNA has an RNA integrity of at least about 50% greater, preferably of at least about 60% greater, more preferably of at least about 70% greater, most preferably of at least about 80% greater, than a composition comprising the first lipid nanoparticle and not comprising the effective amount of the cryoprotectant, when measured under identical conditions.
E180) The composition according to any one of embodiments 131 to 179, wherein the composition comprises greater than 60% more encapsulated RNA, preferably greater than 70% more encapsulated RNA, more preferably greater than 80% more encapsulated RNA, and most preferably greater than 90% more encapsulated RNA, as compared to a composition comprising the first lipid nanoparticle and not comprising the effective amount of the cryoprotectant, when measured under identical conditions.
E181) The composition according to any one of embodiments 131 to 180, wherein the composition comprises greater than 60% encapsulated RNA, preferably greater than 70% encapsulated RNA, more preferably greater than 80% encapsulated RNA, and most preferably greater than 90% encapsulated RNA, as compared to a composition comprising the first lipid nanoparticle and not comprising the effective amount of the cryoprotectant, when measured under identical conditions.
E182) The composition according to any one of embodiments 131 to 181, wherein the RNA has been purified by at least one purification step, and wherein the first lipid nanoparticle has been purified by at least one purification step, preferably by at least one step of tangential flow filtration (TFF) and/or at least one step of clarification and/or at least one step of filtration.
E183) The composition according to any one of embodiments 131 to 182, wherein the RNA is a purified RNA, preferably an RP-HPLC purified RNA and/or a tangential flow filtration (TFF) purified RNA.
E184) The composition according to any one of embodiments 131 to 183, wherein the potency of the composition decreases less than about 30%, preferably less than about 20%, more preferably less than about 10%, as compared to a composition comprising the first lipid nanoparticle and not comprising the effective amount of the cryoprotectant, when measured under identical conditions.
E185) The composition according to any one of embodiments 131 to 184, wherein the composition comprises an effective amount of RNA to produce the polypeptide of interest in a cell.
E186) The composition according to any one of embodiments 131 to 185, wherein the RNA further comprises a modified nucleotide.
E187) The composition according to any one of embodiments 131 to 186, wherein the RNA comprises a modified nucleotide comprising N1-Methylpseudourodine-5′-triphosphate (m1ψTP).
E188) The composition according to any one of embodiments 131 to 187, wherein the RNA comprises a translatable region encoding the antigen and comprises a modified nucleoside comprising 1-methyl-pseudouridine.
E189) The composition according to any one of embodiments 131 to 188, wherein the RNA comprises an open reading frame encoding at least one influenza virus antigenic polypeptide or an immunogenic fragment thereof.
E190) The composition according to any one of embodiments 131 to 189, wherein the RNA further comprises a 5′ cap analog.
E191) The composition according to any one of embodiments 131 to 190, wherein the RNA further comprises a 5′ cap analog and wherein the 5′ cap analog comprises m₂⁷·3′^{-OMe}Gppp(m₁2′^{-O}) ApG.

E192) The composition according to any one of embodiments 131 to 191, wherein the antigen is influenza hemagglutinin 1 (HA1), hemagglutinin 2 (HA2), an immunogenic fragment of HA1 or HA2, or a combination of any two or more of the foregoing.

E193) The composition according to any one of embodiments 131 to 192, wherein the RNA encodes at least two antigenic polypeptides or immunogenic fragments thereof, wherein a first antigen is HA1, HA2, or a combination of HA1 and HA2, and wherein a second antigen is selected from the group consisting of neuraminidase (NA), nucleoprotein (NP), matrix protein 1 (M1), matrix protein 2 (M2), non-structural protein 1 (NS1), and non-structural protein 2 (NS2).

E194) The composition according to any one of embodiments 131 to 193, wherein the RNA encodes at least two antigenic polypeptides or immunogenic fragments thereof, wherein a first antigen is HA1, HA2, or a combination of HA1 and HA2, and wherein a second antigen is neuraminidase (NA).

E195) The composition according to any one of embodiments 131 to 191, wherein the antigen is a polypeptide or an immunogenic fragment thereof from an arenavirus; an astrovirus; a bunyavirus; a calicivirus; a coronavirus; a filovirus; a flavivirus; a hepadnavirus; a hepevirus; an orthomyxovirus; a paramyxovirus; a picornavirus; a reovirus; a retrovirus; a rhabdovirus; a togavirus; or a combination of any two or more of the foregoing.

E196) The composition according to any one of embodiments 131 to 191, wherein the antigen is a polypeptide or an immunogenic fragment thereof from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 01 57:H7, 01 1 1 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBOV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowled, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B1 9, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, Yersinia pseudotuberculosis, or a combination of any two or more of the foregoing.
E197) The composition according to any one of embodiments 131 to 194, wherein the open reading frame is codon-optimized.
E198) The composition according to any one of embodiments 131 to 197, wherein the composition comprises ALC-0315 (4-hydroxybutyl) azanediyl)bis(hexane-6,1-diyl)bis(2-hexyldecanoate).
E199) The composition according to any one of embodiments 131 to 198, wherein the composition comprises ALC-0159 (2-[(polyethylene glycol)-2000]-N,N-ditetradecylacetamide).
E200) The composition according to any one of embodiments 131 to 199, wherein the composition comprises 1,2-Distearoyl-sn-glycero-3-phosphocholine (DSPC).
E201) The composition according to any one of embodiments 131 to 200, wherein the composition comprises cholesterol.
E202) The composition according to any of embodiments 131 to 201, wherein the composition has been freeze-thawed at least 2 times.
E203) The composition according to any of embodiments 131 to 202, wherein the composition has been freeze-thawed at least 3 times.
E204) The composition according to any of embodiments 131 to 203, wherein the composition has been freeze-thawed at least 4 times.
E205) The composition according to any of embodiments 131 to 204, wherein the composition has been freeze-thawed at least 5 times.
E206) A method of producing a polypeptide of interest in a cell, comprising administering a composition according to any one of embodiments 131-205, wherein the composition produces an increased amount of the polypeptide, as compared to a composition comprising the first lipid nanoparticle and not comprising the effective amount of the cryoprotectant, when measured under identical conditions.
E207) The method according to embodiment 206, wherein the composition is administered to a mammal.
E208) The method according to any one of embodiments 206 to 207, wherein the composition is administered to a human.
E209) The method according to any one of embodiments 206 to 208, wherein the composition is administered to a mammal at risk of having influenza.
E210) A method of increasing the stability of a composition comprising a first lipid nanoparticle, the first lipid nanoparticle comprising i) a cationic lipid, ii) a neutral lipid and/or a phospholipid, iii) a steroid, iv) a polymer conjugated lipid, and v) a ribonucleic acid (RNA) polynucleotide encapsulated in the first lipid nanoparticle, the method comprising contacting the composition with an effective amount of a cryoprotectant, wherein the cryoprotectant comprises a saccharide, and wherein the effective amount of the cryoprotectant is at least about 2% w/v to 30% w/v of the composition.
E211) The method according to embodiment 210, wherein the cationic lipid comprises ALC-0315, SM102, DLinDMA, DLin-MC3-DMA, DLin-KC2-DMA, A9, C12-200, L5, or derivatives and/or combinations thereof.
E212) The method according to embodiment 210 or 211, wherein the neutral lipid and/or phospholipid comprises DSPC, DPPC, DMPC, DOPC, POPC, DOPE, SM, or derivatives and/or combinations thereof.
E213) The method according to any one of embodiments 210 to 212, wherein the steroid comprises cholesterol, alpha-tocopherol, or derivatives and/or combinations thereof.
E214) The method according to any one of embodiments 210 to 213, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E215) The method according to any one of embodiments 210-214, wherein the cryoprotectant comprises a disaccharide.
E216) The method according to any one of embodiments 210 to 215, wherein the cryoprotectant comprises sucrose.
E217) The method according to any one of embodiments 210 to 216, wherein the effective amount of the cryoprotectant is at least about 10% w/v to 25% of the composition.
E218) The method according to any one of embodiments 210 to 217, wherein the effective amount of the cryoprotectant is at least about 10.3% w/v to 20.5% of the composition.
E219) The method according to any one of embodiments 210 to 218, wherein the concentration of the cryoprotectant is 5 to 600 mg/mL in the composition before freezing.
E220) The method of any one of embodiments 210 to 219, wherein the composition further comprises a second lipid nanoparticle, and wherein the second lipid nanoparticle does not encapsulate an RNA polynucleotide.
E221) The method of embodiment 220, wherein the second lipid nanoparticle comprises i) a cationic lipid, ii) a neutral lipid and/or a phospholipid, iii) a steroid, and iv) a polymer conjugated lipid.
E222) The method according to embodiment 221, wherein the cationic lipid comprises ALC-0315, SM102, DLinDMA, DLin-MC3-DMA, DLin-KC2-DMA, A9, C12-200, L5, or derivatives and/or combinations thereof.
E223) The method according to embodiment 221 or 222, wherein the neutral lipid and/or phospholipid comprises DSPC, DPPC, DMPC, DOPC, POPC, DOPE, SM, or derivatives and/or combinations thereof.
E224) The method according to any one of embodiments 221 to 223, wherein the steroid comprises cholesterol, alpha-tocopherol, or derivatives and/or combinations thereof.
E225) The method according to any one of embodiments 221 to 224, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E226) The method according to any one of embodiments 221 to 225, wherein the i) cationic lipid, ii) neutral lipid and/or phospholipid, iii) steroid, and iv) polymer conjugated lipid of the second lipid nanoparticle are the same as the i) cationic lipid, ii) neutral lipid and/or phospholipid, iii) steroid, and iv) polymer conjugated lipid of the first lipid nanoparticle.
E227) The method according to any one of embodiments 221 to 225, wherein one or more of the i) cationic lipid, ii) neutral lipid and/or phospholipid, iii) steroid, and/or iv) polymer conjugated lipid of the second lipid nanoparticle are different from the i) cationic lipid, ii) neutral lipid and/or phospholipid, iii) steroid, and/or iv) polymer conjugated lipid of the first lipid nanoparticle.
E228) The method according to embodiment 220, wherein the second lipid nanoparticle comprises a liposome.
E229) The method according to embodiment 228, wherein the liposome comprises i) a phospholipid and/or a neutral lipid, ii) a steroid, and/or iii) a polymer conjugated lipid.
E230) The method according to embodiment 229, wherein the liposome further comprises a cationic lipid.
E231) The method according to embodiment 230, wherein the cationic lipid comprises ALC-0315, SM102, DLinDMA, DLin-MC3-DMA, DLin-KC2-DMA, A9, C12-200, L5, or derivatives and/or combinations thereof.
E232) The method according to any one of embodiments 229 to 223, wherein the neutral lipid and/or phospholipid comprises DSPC, DPPC, DMPC, DOPC, POPC, DOPE, SM, or derivatives and/or combinations thereof.
E233) The method according to any one of embodiments 229 to 232, wherein the steroid comprises cholesterol, alpha-tocopherol, or derivatives and/or combinations thereof.
E234) The method according to any one of embodiments 229 to 233, wherein the polymer conjugated lipid comprises PEG-c-DOMG, PEG-DMG, PEG-DLPE, PEG-DMPE, PEG-DPPC, PEG-DSPE, or derivatives and/or combinations thereof.
E235) The method of any one of embodiments 210 to 234, wherein the stability increase comprises the storage stability of the composition when frozen.
E236) The method of any one of embodiments 210 to 235, wherein the stability increase comprises the stability of the composition when thawed after being frozen.
E237) The method of any one of embodiments 210 to 236, wherein the stability increase comprises the stability of the composition when the composition has been freeze-thawed at least 2 times.
E238) The method of any one of embodiments 210 to 237, wherein the stability increase comprises the stability of the composition when the composition has been freeze-thawed at least 3 times.
E239) The method of any one of embodiments 210 to 238, wherein the stability increase comprises the stability of the composition when the composition has been freeze-thawed at least 4 times.
E240) The method of any one of embodiments 210 to 239, wherein the stability increase comprises the stability of the composition when the composition has been freeze-thawed at least 5 times.
E241) The method of any one of embodiments 210 to 240, wherein contacting the composition with an effective amount of the cryoprotectant forms the immunogenic composition of any one of embodiments 131 to 205.
E242) The composition according to any one embodiments 1 to 22, wherein the composition is lyophilized.
E243) The composition according to any one of embodiments 1 to 22, wherein the composition is lyophilized and reconstituted.
E244) The composition according to any one of embodiments 1 to 36, wherein the composition is configured to be stable for at least about two weeks after storage as a lyophilized composition at temperatures less than or equal to refrigerated storage.
E245) The composition according to any one of embodiments 1 to 37, wherein the composition is configured to be stable for at least 1 month after storage as a frozen liquid composition.
E246) The composition according to any one of embodiments 1 to 38, wherein the composition is configured to be stable for at least about two weeks after storage as a frozen liquid composition.
E247) The composition according to any one of embodiments 1 to 39, wherein the composition is configured to be stable for at least about four weeks after storage as a lyophilized composition at temperatures less than or equal to refrigerated storage.
E248) The composition according to any one of embodiments 1 to 40, wherein the composition is configured to be stable for about 2 weeks to about 1 month, 2 months, 3 months, 4 months, 5 months, 6 months, 1 year, or 2 years after storage as a lyophilized composition at temperatures less than or equal to refrigerated storage.
E249) The composition according to any one of embodiments 1 to 41, wherein the composition is configured to have at least 50%, 60%, 65%, 70%, 75%, 80%, 85%, 90% or 95% of the RNA intact at least about two weeks after storage as a lyophilized composition at temperatures less than or equal to refrigerated storage.
E250) The composition according to any one of embodiments 1 to 42, wherein the composition is configured to have at least 60%, 65%, 70%, 75%, 80%, 85%, 90% or 95% of the RNA intact at least 1 month after storage as a lyophilized composition at temperatures less than or equal to refrigerated storage.
E251) The composition according to any one of embodiments 1 to 43, wherein the composition is configured to have at least 60%, 65%, 70%, 75%, 80%, 85%, 90% or 95% of the RNA intact about 2 weeks to about 1 month, 2 months, 3 months, 4 months, 5 months, 6 months, 1 year, or 2 years after storage as a lyophilized composition at temperatures less than or equal to refrigerated storage.
E252) The composition according to any one of embodiments 1 to 44, wherein the composition is configured to have at least 80% of the RNA intact after about two weeks of storage as a lyophilized composition at temperatures less than or equal to refrigerated storage.
E253) The composition according to any one embodiments 131 to 162, wherein the composition is lyophilized.
E254) The composition according to any one of embodiments 131 to 163, wherein the composition is lyophilized and reconstituted.

1. A composition comprising tert-butanol and a lipid nanoparticle (LNP) comprising i) a cationic lipid, ii) a neutral lipid and/or a phospholipid, iii) a steroid, and iv) a polymer conjugated lipid.

2. The composition according to claim 1, wherein the lipid nanoparticle encapsulates a ribonucleic acid (RNA) polynucleotide having an open reading frame encoding at least one polypeptide of interest.

3. The composition according to claim 2, wherein the at least one polypeptide of interest comprises an antigen.

4. The composition according to any one of claims 1-3, wherein the cationic lipid comprises ALC-0315, SM-102, DLinDMA, DLin-MC3-DMA, DLin-KC2-DMA, A9, C12-200, L5, or derivatives and/or combinations thereof.

5. The composition according to any one of claims 1-4, wherein the neutral lipid and/or phospholipid comprises DSPC, DPPC, DMPC, DOPC, POPC, DOPE, SM, or derivatives and/or combinations thereof.

6. The composition according to any one of claims 1 to 5, wherein the steroid comprises cholesterol, alpha-tocopherol, or derivatives and/or combinations thereof.

7. The composition according to any one of claims 1 to 6, wherein the polymer conjugated lipid comprises PEG-c-DOMG, PEG-DMG, PEG-DLPE, PEG-DMPE, PEG-DPPC, PEG-DSPE, or derivatives and/or combinations thereof.

8. The composition according to any one of claims 1 to 7, further comprising a cryoprotectant.

9. The composition according to claim 8, wherein the cryoprotectant comprises sucrose, and wherein the composition comprises at least about 2% w/v to 50% w/v sucrose.

10. The composition according to any one of claims 1 to 8, further comprising any one buffer selected from histidine, succinate, citrate, acetate, and succinate.

11. The composition according to any one of claims 1 to 10, further comprising histidine.

12. The composition according to any one of claims 1 to 11, wherein the composition is liquid.

13. The composition according to any one of claims 1 to 12, wherein the composition is frozen.

14. The composition according to any one of claims 1 to 13, wherein the composition is lyophilized.

15. The composition according to any one of claims 1 to 14, wherein the composition is lyophilized and reconstituted.

16. The composition according to any one of claims 3 to 15, wherein the RNA polynucleotide comprises a translatable region encoding the antigen and comprises a modified nucleoside comprising 1-methyl-pseudouridine.

17. The composition according to any one of claims 3 to 16, wherein the RNA polynucleotide is a self-amplifying RNA comprising a translatable region encoding the antigen.

18. The composition according to any one of claims 2 to 17, wherein the RNA polynucleotide comprises an open reading frame encoding at least one influenza virus antigenic polypeptide or an immunogenic fragment thereof.

19. The composition according to any one of claims 2 to 18, wherein the composition comprises greater than 70% more encapsulated RNA.

20. The composition according to any of claims 1 to 19, wherein the composition is immunogenic.

21. The composition according to any one of claims 2-20, wherein the RNA polynucleotide has an integrity of at least 80% prior to lyophilization, as measured by a fragment analyzer.

22. The composition according to any one of claims 2-21, wherein the RNA polynucleotide has an integrity of at least 80% after lyophilization, as measured by a fragment analyzer.

23. The composition according to any one of claims 1-22, wherein the LNP has a polydispersity index of 0.05-0.15 after lyophilization.

24. The composition according to any one of claims 1-23, wherein the tert-butanol is at most 5% w/v of the composition.

25. The composition according to any one of claims 1-23, comprising about 10 ug/ml saRNA in 15-25 mM histidine, about 10% sucrose, and about 1.5% tert-butanol.

26. The composition according to any one of claims 1-23, comprising about 10 ug/ml saRNA in 10-20 mM histidine, about 10% sucrose, and about 0.75% tert-butanol.

27. The composition according to any one of claims 1-23, comprising about 10 ug/ml saRNA in about 10 mM histidine, about 10% sucrose, and about 0.75% tert-butanol.

28. The composition according to any one of claims 1-23, comprising about 20 ug/ml saRNA in about 20 mM histidine, about 10% sucrose, and about 1.5% tert-butanol.

29. The composition according to any one of claims 1-23, comprising about 20 ug/ml saRNA in about 10 mM histidine, about 10% sucrose, and about 1.5% tert-butanol.

30. The composition according to any one of claims 1-23, comprising about 20 ug/ml saRNA in about 10 mM histidine, about 10% sucrose, and about 1.4% tert-butanol.

31. The composition according to any one of claims 1-23, comprising about 20 ug/ml saRNA in about 10 mM histidine, about 20% sucrose, and about 1.4% tert-butanol.

32. The composition according to any one of claims 1-23, comprising about 20 ug/ml saRNA in about 15 mM histidine, about 10% sucrose, and about 1.4% tert-butanol.

33. The composition according to any one of claims 1-23, comprising about 20 ug/ml saRNA in about 15 mM histidine, about 20% sucrose, and about 1.4% tert-butanol.

34. The composition according to any of claims 2 to 33, wherein the RNA-encapsulating LNP is 50-80 nm in size prior to lyophilization.

35. The composition according to any of claims 2 to 34, wherein the RNA-encapsulating LNP is 80-120 nm in size after lyophilization.

36. A method for producing a composition comprising an RNA-encapsulating lipid nanoparticle (LNP) comprising the step of mixing a first composition comprising t-butanol, an ionizable lipid, a polymer-conjugated lipid, phospholipid, and a sterol with a second composition having a pH 4-6 comprising an RNA polynucleotide, aqueous solvent, and buffer, thereby producing a composition comprising the RNA-encapsulating lipid nanoparticle and t-butanol.

37. The method according to claim 36, wherein the method does not comprise a step of exchanging an aqueous phase of the RNA-encapsulating LNPs with a buffer by any one of dialysis, ultrafiltration, and dilution.

38. The method according to any one of claims 36-37, wherein the second composition comprises 100-250 mM histidine.

39. The method according to any one of claims 36-38, wherein the first and second compositions are not mixed using a flowrate ratio of 1:1-1:3.

40. The method according to any one of claims 36-39, wherein the first and second compositions are mixed using a flowrate of 1:7.

41. The method according to any one of claims 36-40, further comprising a step of mixing the RNA-encapsulating LNP with a cryoprotectant.

42. The method according to claim 41, wherein the cryoprotectant is sucrose.

43. The method according to any one of claims 36-41, wherein the method yields a mixture of RNA-encapsulating LNP having an encapsulation efficiency of at least 70%.

44. The method according to any one of claims 36-41, wherein the RNA in the second composition has an integrity of at least 80%, as measured by a fragment analyzer.

45. The method according to any one of claims 36-41, wherein the RNA polynucleotide in the RNA-encapsulating LNP has an integrity of at least 80%, as measured by a fragment analyzer.

46. The method according to any one of claims 36-41, wherein the RNA-encapsulating LNP has a polydispersity index of 0.05-0.15 after lyophilization.

47. The method according to any one of claims 36-46, wherein the t-butanol is at most 5% w/v of the composition.

48. A method of producing a polypeptide of interest in a cell, comprising administering a composition according to any one of claims 1-35.

49. The method according to claim 36, wherein the composition is administered to a mammal.
